(19) United States
(12) Patent Application Publication
(10) Pub. No.: US 2026/0258362 A1
(43) Pub. Date: Sep. 3, 2026

(54) CELLS AND METHODS OF PREPARATION

(71) Applicant: LIFT BioSciences Ltd, Brighton (GB)

(72) Inventors: Alex Blyth, Brighton (GB); Oxana POLYAKOVA, Brighton (GB); Aoife MCGINLEY, Brighton (GB); Samuel FLORENCE, Brighton (GB); Mihil PATEL, Brighton (GB); Durva PATEL, Brighton (GB); Andrew WILLIS, Brighton (GB)

(73) Assignee: LIFT BioSciences Ltd, Brighton (GB)

(21) Appl. No.: 19/163,658

(22) PCT Filed: Mar. 8, 2024
(86) PCT No.: PCT/GB2024/050639

§ 371 Date: Sep. 9, 2025

(30) Foreign Application Priority Data
Mar. 10, 2023 (GB) 2303583.5
Jul. 21, 2023 (GB) 2311283.2
Jul. 21, 2023 (GB) 2311284.0
Jul. 22, 2023 (GB) 2311285.7
Jul. 24, 2023 (GB) 2311352.5

(51) Int. Cl. C12N 5/0789; A61K 35/28; A61P 35/00; C12M 1/00; C12N 5/0786

(52) U.S. Cl. CPC C12N 5/0647; A61K 35/28; A61P 35/00; C12M 47/04; C12N 5/0645; C12N 2500/25; C12N 2501/145; C12N 2501/22; C12N 2501/2303; C12N 2501/25; C12N 2501/26; C12N 2501/998

(57) ABSTRACT

A method of preparing cells for therapeutic use is provided, in which a population of progenitor cells is cultured in cell culture conditions that promote differentiation of the progenitor cells comprising the presence of: G-CSF, GM-CSF, IL-3 and TNF; to produce a population of granulopoietic cells. The granulopoietic cells produced may be useful in methods of treatment, including the treatment of cancer and infections, and to modulate inflammatory cell responses.

### CROSS-REFERENCE TO RELATED APPLICATIONS

This is a U.S. National Stage application which claims priority to and the benefit of the earlier filing of International Application No. PCT/GB2024/050639, filed on Mar. 8, 2024, which claims priority to GB Application No. 2303583.5, filed on Mar. 10, 2023; GB Application No. 2311284.0, filed on Jul. 21, 2023; GB Application No. 2311283.2, filed on Jul. 21, 2023; GB Application No. 2311285.7, filed on Jul. 22, 2023; and GB Application No. 2311352.5, filed on Jul. 24, 2023, each of which are incorporated by reference herein in their entirety.

The present invention relates to methods of preparing cells for therapeutic use, and to populations of cells prepared by such methods. The invention also relates to populations of granulopoietic cells, and to pharmaceutical compositions comprising such populations of cells. The populations of granulopoietic cells and pharmaceutical compositions may be suitable for use in modulating (e.g. amplifying) a therapeutic immune response, particularly in modulating non-granulocytic immune responses, and/or in giving rise to cells with cytocidal activity. The populations of granulopoietic cells and pharmaceutical compositions may be of use in contexts such as the treatment of cancer or of infection. The invention further relates to cell culture media.

Immunotherapies can act to amplify the native immune response of a cell or host to achieve a therapeutic aim. They are becoming increasingly important for use in a broad range of therapeutic contexts.

Host therapeutic immune responses often involve several types of immune cell and play a vital role in the body's fight against cancer, infections and virtually all other diseases. However, a subject's native therapeutic immune response is not always enough to eradicate disease. For example, tumours may be adapted to be immunologically “cold”, and may create an immunosuppressive tumour microenvironment (TME) that can render native anti-tumour therapeutic immune responses ineffective.

To successfully eradicate a tumour (e.g. cancer), a variety of different types of immune cells typically need to work together. However, in some cases, a subject's own immune cells may be defective meaning there is a need for a variety of different types of immune cells from an alternative source. There are currently difficulties in manufacturing such cell combinations. Additionally or alternatively, such conventional cell combinations may have adverse immunogenic effects.

Cell based therapies may also make use of the cytocidal capacity of cells, and their ability to kill cells, such as cancer cells, infected cells, or cellular infectious agents, that play important roles in disease. Cells with cytocidal activity, or with the capacity to generate such cytocidal cells, thus represent therapeutically important targets.

Accordingly, there is a need for improved immunotherapies, particularly cell therapies, as well as methods of producing cells that may be used in said immunotherapies.

The present invention addresses one or more of the above-mentioned problems.

The inventors have identified conditions that can be used to generate large populations of granulopoietic cells that have highly desirable therapeutic capabilities. These cells are capable of modulating (such as amplifying) and, in suitable embodiments, serve to modulate or amplify, the therapeutic immune response of non-granulocytic immune cells, and also of giving rise to cells with useful cytocidal activities. Such compositions and populations of cells are thus able to modulate therapeutic immune responses (and can thus modulate, for example amplify, a host therapeutic immune response) and/or generate cells with direct cytocidal activity after administration to a subject.

In a first aspect, the invention provides a method of preparing cells for therapeutic use, the method comprising:
culturing a population of progenitor cells in cell culture conditions that promote differentiation of the progenitor cells comprising the presence of:
   G-CSF,
   GM-CSF,
   IL-3 and
   TNF;
to produce a population of granulopoietic cells.

A method of the first aspect of the invention may optionally comprise a further step of purifying the population of granulopoietic cells produced, and/or formulating this population of cells for medical use.

In a second aspect, the invention provides a population of granulopoietic cells prepared for therapeutic use by a method of the first aspect of the invention.

In a further aspect, the invention provides a cell culture medium for use in a method in accordance with the first aspect of the invention, the medium comprising: G-CSF; GM-CSF; IL-3; and TNF. A cell culture medium in accordance with this aspect of the invention may be referred to as a “differentiation medium”.

The populations of granulopoietic cells produced by the methods of the invention may optionally be harvested once produced. For the purposes of the present disclosure, “harvesting” of cells may be taken to encompass suspension of the cells, isolation of the cells, or separation of the cells.

The populations of granulopoietic cells produced by the methods of the invention may optionally be cryopreserved once produced. It is known that granulocytes, such as neutrophils, do not respond well to cryopreservation, with low levels of viable cells remaining after a frozen population of cells has been thawed. In contrast, the granulopoietic cells of the present invention are well adapted to cryopreservation, with high levels of viable cells being obtained after the freezing and thawing process. Accordingly, the granulopoietic cell populations of the invention offer significant advantages, as compared to mature granulocytic cells, in applications in which it is desired to cryopreserve cells before their use for therapy.

The populations of granulopoietic cells produced by the methods of the invention may optionally be formulated for medical use once produced. Methods suitable for formulation of cell populations that are to be used therapeutically will be well known to those skilled in the art, and may be used in the formulation of the granulopoietic cell populations of the invention, optionally to give rise to pharmaceutical compositions of the invention.

Characteristics of the populations of granulopoietic cells produced, and also of the populations of progenitor cells that may be used in such methods, are considered in more detail elsewhere in the specification.

Optionally, the cell culture conditions that promote differentiation of the progenitor cells may further comprise the presence of at least one cytokine selected from the group consisting of: SCF, and TPO. Suitably, the cell culture conditions comprise the presence of both SCF and TPO.

Similarly, a differentiation medium of the invention may further comprise at least one cytokine selected from the group consisting of: SCF, and TPO. Suitably, such a cell culture medium comprises both SCF and TPO.

The following paragraphs set out details of useful embodiments of the methods of the first aspect of the invention. These include useful embodiments of the progenitor cells that may be used as starting material, the granulopoietic cells that may be produced by the methods, and the cell culture conditions that may be employed. Except for any examples where it is noted otherwise, considerations set out in respect of cell culture conditions that promote differentiation which may be used in the methods of the first aspect of the invention are also applicable as suitable embodiments of a differentiation cell culture medium of the invention.

Cell culture conditions that promote differentiation used in the methods of the first aspect of the invention may comprise Iscove's modified Dulbecco's medium (IMDM) as a cell culture medium. Similarly, a cell culture medium of the invention may also comprise IMDM. In either case, in a suitable embodiment, the IMDM is a form of the medium that comprises high glucose, glutamine, HEPES, sodium pyruvate, and may optionally contain phenol red.

The methods of the first aspect of the invention, or a differentiation medium of the invention, make use of the cytokine granulocyte colony stimulating factor (G-CSF) as a supplement.

Suitably, the G-CSF is provided at a concentration of 0.013 μg/mL, or more. For example, the G-CSF may be provided at a concentration of 0.016 μg/mL, or more, 0.02 μg/mL, or more, 0.03 μg/mL, or more, or 0.065 μg/mL, or more.

Suitably, the G-CSF is provided at a concentration of 0.65 μg/mL, or less. For example, the G-CSF may be provided at a concentration of 0.52 μg/mL, or less, 0.39 μg/mL, or less, or 0.26 μg/mL, or less.

Suitably, the G-CSF is provided at a concentration of approximately 0.013 μg/mL to 0.65 μg/mL, 0.016 μg/mL to 0.52 μg/mL, 0.02 μg/mL to 0.39 μg/mL, 0.03 μg/mL to 0.26 μg/mL, or 0.065 μg/mL to 0.195 μg/mL. In a suitable embodiment, the G-CSF is provided at a concentration of approximately 0.13 μg/mL. Indeed, in a suitable embodiment, the G-CSF is provided at a concentration of 0.13 μg/mL.

Examples of suitable forms of G-CSF that may be used in this manner include the product 5 produced by Peprotech, and the GMP product produced by BioLegend, details of which are set out in Table 2.

The methods of the first aspect of the invention, or a differentiation medium of the invention, make use of the cytokine granulocyte-macrophage colony stimulating factor (GM-CSF) as a supplement.

Suitably, the GM-CSF is provided at a concentration of 0.001 μg/mL, or more. For example, the GM-CSF may be provided at a concentration of 0.00125 μg/mL, or more, 0.00167 μg/mL, or more, 0.0025 μg/mL, or more, or 0.005 μg/mL, or more.

Suitably, the GM-CSF is provided at a concentration of 0.05 μg/mL, or less. For example, the GM-CSF may be provided at a concentration of 0.04 μg/mL, or less, 0.03 μg/mL, or less, or less, or 0.02 μg/mL, or less.

20 Suitably, the GM-CSF is provided at a concentration of approximately 0.001 μg/mL to 0.05 μg/mL, 00.125 μg/mL to 0.04 μg/mL, 0.00167 μg/mL to 0.03 μg/mL, 0.0025 μg/mL to 0.02 μg/mL, or 0.005 μg/mL to 0.015 μg/mL. In a suitable embodiment, the GM-CSF is provided at a concentration of approximately 0.01 μg/mL. Indeed, in a suitable embodiment, the GM-CSF is provided at a concentration of 0.01 μg/mL.

Examples of suitable forms of GM-CSF that may be used in this manner include the products produced by Peprotech and BioTechne, and the GMP product produced by BioTechne, details of which are set out in Table 2.

The methods of the first aspect of the invention, or a differentiation medium of the invention, make use of the cytokine interleukin-3 (IL-3) as a supplement.

Suitably, the IL-3 is provided at a concentration of 0.013 μg/mL, or more. For example, the IL-3 may be provided at a concentration of 0.016 μg/mL, or more, 0.02 μg/mL, or more, 0.03 μg/mL, or more, or 0.065 μg/mL, or more.

Suitably, the IL-3 is provided at a concentration of 0.65 μg/mL, or less. For example, the IL-3 may be provided at a concentration of 0.52 μg/mL, or less, 0.39 μg/mL, or less, or 0.26 μg/mL, or less.

Suitably, the IL-3 is provided at a concentration of approximately 0.013 μg/mL to 0.65 μg/mL, 0.016 μg/mL to 0.52 μg/mL, 0.02 μg/mL to 0.39 μg/mL, 0.03 μg/mL to 0.26 μg/mL, or 0.065 μg/mL to 0.195 μg/mL. In a suitable embodiment, the IL-3 is provided at a concentration of approximately 0.13 μg/mL. Indeed, in a suitable embodiment, the IL-3 is provided at a concentration of 0.13 μg/mL.

Examples of suitable forms of IL-3 that may be used in this manner include the product produced by PeproTech, and the GMP product produced by PeproTech or BioTechne, details of which are set out in Table 2.

In a suitable embodiment, GM-CSF and IL-3 are provided to the cells for a period of between 12 and 72 hours, suitably a period of 48 hours during the cell culture conditions. For example, GM-CSF and IL-3 may be provided to the cells for the final 48 hours of the period for which they are in culture. GM-CSF and IL-3 may be provided to the cells on the fourth and fifth days of cell culture conditions that promote differentiation of the progenitor cells. GM-CSF and IL-3 may be provided to the cells on the third and fourth days of cell culture conditions that promote differentiation of the progenitor cells.

The methods of obtaining a granulopoietic cell make use of the cytokine tumour necrosis factor (TNF) as a supplement. The terms TNF and TNF-alpha are used interchangeably herein.

Suitably, the TNF is provided at a concentration of 0.0001 μg/mL, or more. For example, the TNF may be provided at a concentration of 0.000125 μg/mL, or more, 0.000167 μg/mL, or more, 0.00025 μg/mL, or more, or 0.0005 μg/mL, or more.

Suitably, the TNF is provided at a concentration of 0.005 μg/mL, or less. For example, the TNF may be provided at a concentration of 0.004 μg/mL, or less, 0.003 μg/mL, or less, or 0.002 μg/mL, or less.

Suitably, the TNF is provided at a concentration of approximately 0.0001 μg/mL to 0.005 μg/mL, 0.000125 μg/mL to 0.004 μg/mL, 0.000167 μg/mL to 0.003 μg/mL, 0.00025 μg/mL to 0.002 μg/mL, or 0.0005 μg/mL to 0.0015 μg/mL. In a suitable embodiment, the TNF is provided at a concentration of approximately 0.001 μg/mL. Indeed, in a suitable embodiment, the TNF is provided at a concentration of 0.001 μg/mL.

Examples of suitable forms of TNF that may be used in this manner include the product produced by PeproTech, and the GMP product produced by BioTechne, details of which are set out in Table 2.

In a suitable embodiment, the TNF is provided to the cells for a period of between 12 and 36 hours, suitably a period of 24 hours during the cell culture conditions. For example, the TNF may be provided to the cells for the final 24 hours of the period for which they are in culture. The TNF may be provided to the cells on the fourth to fifth days of cell culture conditions that promote differentiation of the progenitor cells. The TNF may be provided to the cells on the fifth day of cell culture conditions that promote differentiation of the progenitor cells. The TNF may be provided to the cells on the fourth day of cell culture conditions that promote differentiation of the progenitor cells.

The methods of the first aspect of the invention, or a differentiation medium of the invention, may optionally make use of the cytokine stem cell factor (SCF) as a supplement.

Suitably, the SCF is provided at a concentration of 0.013 μg/mL, or more. For example, the SCF may be provided at a concentration of 0.016 μg/mL, or more, 0.02 μg/mL, or more, 0.03 μg/mL, or more, or 0.065 μg/mL, or more.

Suitably, the SCF is provided at a concentration of 0.65 μg/mL, or less. For example, the SCF may be provided at a concentration of 0.52 μg/mL, or less, 0.39 μg/mL, or less, or 0.26 μg/mL, or less.

Suitably, the SCF is provided at a concentration of approximately 0.013 μg/mL to 0.65 μg/mL, 0.016 μg/mL to 0.52 μg/mL, 0.02 μg/mL to 0.39 μg/mL, 0.03 μg/mL to 0.26 μg/mL, or 0.065 μg/mL to 0.195 μg/mL. In a suitable embodiment, the SCF is provided at a concentration of approximately 0.13 μg/mL. Indeed, in a suitable embodiment, the SCF is provided at a concentration of 0.13 μg/mL.

Examples of suitable forms of SCF that may be used in this manner include the product produced by Peprotech, and the GMP product produced by PeproTech or BioTechne, details of which are set out in Table 2.

The methods of the first aspect of the invention, or a differentiation medium of the invention, may optionally make use of the cytokine thrombopoietin (TPO) as a supplement.

Suitably, the TPO is provided at a concentration of 0.013 μg/mL, or more. For example, the TPO may be provided at a concentration of 0.016 μg/mL, or more, 0.02 μg/mL, or more, 0.03 μg/mL, or more, or 0.065 μg/mL, or more.

Suitably, the TPO is provided at a concentration of 0.65 μg/mL, or less. For example, the TPO may be provided at a concentration of 0.52 μg/mL, or less, 0.39 μg/mL, or less, or 0.26 μg/mL, or less.

Suitably, the TPO is provided at a concentration of approximately 0.013 μg/mL to 0.65 μg/mL, 0.016 μg/mL to 0.52 μg/mL, 0.02 μg/mL to 0.39 μg/mL, 0.03 μg/mL to 0.26 μg/mL, or 0.065 μg/mL to 0.195 μg/mL. In a suitable embodiment, the TPO is provided at a concentration of approximately 0.13 μg/mL. Indeed, in a suitable embodiment, the TPO is provided at a concentration of 0.13 μg/mL.

Examples of suitable forms of TPO that may be used in this manner include the product produced by Peprotech, and the GMP products produced by BioTechne or Peprotech, details of which are set out in Table 2.

In a suitable embodiment, the cell culture conditions used in culturing the population of progenitor cells to produce granulopoietic cells further comprise the presence of at least one supplement selected from the group consisting of: insulin transferrin selenium (ITS), and human serum albumin (HSA). In a suitable embodiment, such cell culture condition comprise the presence of both ITS and HSA. Suitably, both ITS and HSA are present in a differentiation medium of the invention.

The methods of the first aspect of the invention, or a differentiation medium of the invention, may suitably make use of insulin at a concentration of between about 0.1 g/L and about 5 g/L, for example at a concentration of approximately 1.0 g/L, as a supplement. These methods and cell culture media may suitably make use of transferrin at a concentration of between about 0.01 g/L and about 2.5 g/L, for example at a concentration of approximately 0.55 g/L as a supplement. Suitably such methods and cell culture media may make use of selenium at a concentration of between about 0.0001 g/L and about 0.003 g/L, for example at a concentration of approximately 0.00067 g/L, as a supplement.

The methods of the first aspect of the invention, or a differentiation medium of the invention, may optionally make use of HSA as a supplement.

Suitably, the HSA may be provided at a concentration of between 0.1% and 5%. For example, HSA provided as a supplement may be provided at a concentration of approximately 1%.

Suitably, the cell culture conditions that promote differentiation of the progenitor cells used in a method of the invention, or a differentiation medium of the invention, may comprise: GM-CSF; and G-CSF; and SCF; and TPO; and IL-3; and TNF; and ITS; and HSA. The cell culture medium may comprise IMDM, optionally with Glutamax supplementation.

Thus, in a suitable embodiment, the cell culture conditions that promote differentiation of the progenitor cells used in a method of the invention, or a differentiation medium of the invention, may comprise: GM-CSF at a concentration of approximately 0.01 μg/mL; and G-CSF at a concentration of approximately 0.13 μg/mL; and SCF at a concentration of approximately 0.13 μg/mL; and TPO at a concentration of approximately 0.13 μg/mL; and IL-3 at a concentration of approximately 0.13 μg/mL; and TNF at a concentration of approximately 0.001 μg/mL; and 1× ITS; and HSA at approximately 1%. The cell culture medium may comprise IMDM, optionally with Glutamax supplementation.

A method of the invention may comprise culturing a population of progenitor cells in cell culture conditions that promote differentiation of the progenitor cells for any suitable period of time. For example, the progenitor cells may be cultured for 1 day, 2 days, 3 days, 4 days, 5 days, 6 days, 7 days, 8 days, 9 days, 10 days, 11 days, 12 days, 13 days, 14 days or 15 days in conditions to produce a population of granulopoietic cells. Methods in accordance with the first aspect of the invention may comprise culturing the population of progenitor cells in cell culture conditions that promote differentiation of the progenitor cells for a period of 1 to 7 days. For example, such methods may comprise culturing the cells in the relevant conditions for a period of 4 to 7 days. In a suitable embodiment, such methods may comprise culturing the cells for approximately 1 day, or for approximately 2 days, or for approximately 3 days, or for approximately 4 days, or for approximately 5 days, or for approximately 6 days, or for approximately 7 days. The progenitor cells may be cultured for 1-10 days, 2-9 days, 3-8 days, 4-7 days, or 5-6 days in conditions to produce a population of granulopoietic cells. Suitably the progenitor cells are cultured for 4, 5 or 6 days in conditions to produce a population of granulopoietic cells. In a suitable embodiment, the progenitor cells are cultured for 4 days in conditions to produce a population of granulopoietic cells. In a suitable embodiment, the progenitor cells are cultured for 5 days in conditions to produce a population of granulopoietic cells. In a suitable embodiment, the progenitor cells are cultured for 6 days in conditions to produce a population of granulopoietic cells.

In a suitable embodiment of a method of the invention, progenitor cells may be cultured at an initial seeding density of between approximately 1×10⁵ and 10×10⁶ cells per cm².

Methods of the invention may involve expansion of the number of cells present in the culture, such that the number of granulopoietic cells yielded by the method is larger than the number of progenitor cells present at the beginning of the method. In a suitable embodiment, the number of granulopoietic cells in the population produced may be increased, as compared to the number of progenitor cells present at the beginning of the method, by at least 1-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, or at least 15-fold, The methods set out in the Examples achieve a population of granulopoietic cells that is approximately 3.5-fold larger than the initial population of progenitor cells.

In a suitable embodiment, a method of the first aspect of the invention is practiced in respect of a population of progenitor cells that has been produced by in vitro expansion of a population of stem cells. Accordingly, such a method of the invention may further comprise a step of culturing a population of stem cells in cell culture conditions to produce the population of progenitor cells.

In a suitable embodiment, a method of the first aspect of the invention further comprises a step of culturing a population of stem cells in cell culture conditions to produce the population of progenitor cells:
wherein the cell culture conditions for producing the progenitor cells comprise the presence of
   SCF,
   Flt-3 Ligand,
   IL-3,
   IL-6, and
   TPO.

The number of progenitor cells produced in such a method may be markedly expanded as compared to the number of stem cells present at the start of the cell culture conditions. Merely by way of example, such an embodiment of a method of the invention may achieve an expansion of progenitor cell numbers that is at least 50-fold, at least 75-fold, at least 100-fold, at least 150-fold, at least 200-fold, at least 250-fold, at least 300-fold, or at least 350-fold, or more, as compared to the number of stem cells at the start of the cell culture conditions. The Examples set out details of a protocol that the inventors have used to achieve an approximately 75-fold increase in progenitor cell numbers, as compared to the starting stem cell population.

The invention also provides a cell culture medium, for use in a method of the invention, comprising SCF; Flt-3 Ligand; IL-3; IL-6; and TPO. A cell culture medium in accordance with this aspect of the invention may be referred to as an “expansion medium”.

Accordingly, a method of preparing cells for therapeutic use in accordance with such embodiments of the invention may comprise:
a) culturing a population of stem cells in cell culture conditions for producing progenitor cells comprising the presence of:
   SCF,
   Flt-3 Ligand,
   IL-3,
   IL-6, and
   TPO;
to produce a population of progenitor cells; and
b) culturing the population of progenitor cells in cell culture conditions that promote differentiation of the progenitor cells comprising the presence of:
   G-CSF,
   GM-CSF,
   IL-3 and
   TNF;
to produce a population of granulopoietic cells; and optionally
c) harvesting the granulopoietic cells.

The total increase in number of cells achieved by such a method of the invention, representing the change in cell numbers from the initial population of stem cells to the population of granulopoietic cells produced, may be at least 50-fold, at least 100-fold, at least 150-fold, at least 200-fold, at least 250-fold, at least 300-fold, at least 350-fold, at least 400-fold, at least 450-fold, at least 500-fold, at least 550-fold, at least 600-fold, at least 650-fold, at least 700-fold, at least 750-fold, at least 800-fold, at least 850-fold, at least 900-fold, at least 950-fold, at least 1000-fold, at least 1050-fold, at least 1100-fold, at least 1150-fold, at least 1200-fold, at least 1250-fold, or at least 1300-fold. The Examples set out details of a protocol that the inventors have used to achieve greater than 250-fold increase in granulopoietic cell numbers, as compared to the starting stem cell population.

A method in accordance with such embodiments of the invention may involve a total period of time in culture of between 10 and 25 days, for example of between 11 and 20 days, such as 12 days, 13 days, 14 days, 15 days, 06 days, 17 days, 18 days, or 19 days.

SCF may optionally be provided as a supplement in embodiments of the methods of the invention comprising a step of producing a population of progenitor cells, as well as in an expansion medium of the invention.

Suitably, the SCF is provided at a concentration of 0.02 μg/mL, or more. For example, the SCF may be provided at a concentration of 0.025 μg/mL, or more, 0.03 μg/mL, or more, 0.05 μg/mL, or more, or 0.1 μg/mL, or more.

Suitably, the SCF is provided at a concentration of 1 μg/mL, or less. For example, the SCF may be provided at a concentration of 0.8 μg/mL, or less, 0.6 μg/mL, or less, or 0.4 μg/mL, or less.

Suitably, the SCF is provided at a concentration of approximately 0.02 μg/mL to 1 μg/mL, 0.025 μg/mL to 0.8 μg/mL, 0.03 μg/mL to 0.6 μg/mL, 0.05 μg/mL to 0.4 μg/mL, or 0.1 μg/mL to 0.3 μg/mL. In a suitable embodiment, the SCF is provided at a concentration of approximately 0.2 μg/mL. Indeed, in a suitable embodiment, the SCF is provided at a concentration of 0.2 μg/mL.

The forms of SCF discussed above are also suitable for use in such embodiments.

Flt-3 ligand (F3L) may optionally be provided as a supplement in embodiments of the methods of the invention comprising a step of producing a population of progenitor cells, as well as in an expansion medium of the invention.

Suitably, the F3L is provided at a concentration of 0.02 μg/mL, or more. For example, the F3L may be provided at a concentration of 0.025 μg/mL, or more, 0.03 μg/mL, or more, 0.05 μg/mL, or more, or 0.1 μg/mL, or more.

Suitably, the F3L is provided at a concentration of 1 μg/mL, or less. For example, the F3L may be provided at a concentration of 0.8 μg/mL, or less, 0.6 μg/mL, or less, or 0.4 μg/mL, or less.

Suitably, the F3L is provided at a concentration of approximately 0.02 μg/mL to 1 μg/mL, 0.025 μg/mL to 0.8 μg/mL, 0.03 μg/mL to 0.6 μg/mL, 0.05 μg/mL to 0.4 μg/mL, or 0.1 μg/mL to 0.3 μg/mL. In a suitable embodiment, the F3L is provided at a concentration of approximately 0.2 μg/mL. Indeed, in a suitable embodiment, the F3L is provided at a concentration of 0.2 μg/mL.

Examples of suitable forms of F3L that may be used in this manner include the product produced by Peprotech, and the GMP product produced by PeproTech or BioTechne, details of which are set out in Table 2.

IL-3 may optionally be provided as a supplement in embodiments of the methods of the invention comprising a step of producing a population of progenitor cells, as well as in an expansion medium of the invention.

Suitably, the IL-3 is provided at a concentration of 0.0015 μg/mL, or more. For example, the IL-3 may be provided at a concentration of 0.0019 μg/mL, or more, 0.0025 μg/mL, or more, 0.00375 μg/mL, or more, or 0.0075 μg/mL, or more.

Suitably, the IL-3 is provided at a concentration of 0.075 μg/mL, or less. For example, the IL-3 may be provided at a concentration of 0.06 μg/mL, or less, 0.045 μg/mL, or less, or 0.03 μg/mL, or less.

Suitably, the IL-3 is provided at a concentration of approximately 0.0015 μg/mL to 0.075 μg/mL, 0.0019 μg/mL to 0.06 μg/mL, 0.0025 μg/mL to 0.045 μg/mL, 0.00375 μg/mL to 0.03 μg/mL, or 0.0075 μg/mL to 0.0225 μg/mL. In a suitable embodiment, the IL-3 is provided at a concentration of approximately 0.015 μg/mL. Indeed, in a suitable embodiment, the IL-3 is provided at a concentration of 0.015 μg/mL.

The forms of IL-3 discussed above are suitable for use in such embodiments.

Interleukin 6 (IL-6) may optionally be provided as a supplement in embodiments of the methods of the invention comprising a step of producing a population of progenitor cells, as well as in an expansion medium of the invention.

Suitably, the IL-6 is provided at a concentration of 0.0015 μg/mL, or more. For example, the IL-6 may be provided at a concentration of 0.0019 μg/mL, or more, 0.0025 μg/mL, or more, 0.00375 μg/mL, or more, or 0.0075 μg/mL, or more.

Suitably, the IL-6 is provided at a concentration of 0.075 μg/mL, or less. For example, the IL-6 may be provided at a concentration of 0.06 μg/mL, or less, 0.045 μg/mL, or less, or 0.03 μg/mL, or less.

Suitably, the IL-6 is provided at a concentration of approximately 0.0015 μg/mL to 0.075 μg/mL, 0.0019 μg/mL to 0.06 μg/mL, 0.0025 μg/mL to 0.045 μg/mL, 0.00375 μg/mL to 0.03 μg/mL, or 0.0075 μg/mL to 0.0225 μg/mL. In a suitable embodiment, the IL-6 is provided at a concentration of approximately 0.015 μg/mL. Indeed, in a suitable embodiment, the IL-6 is provided at a concentration of 0.015 μg/mL.

Examples of suitable forms of IL-6 that may be used in this manner include the product produced by PeproTech, and the GMP product produced by PeproTech or BioTechne, details of which are set out in Table 2.

TPO may optionally be provided as a supplement in embodiments of the methods of the invention comprising a step of producing a population of progenitor cells, as well as in an expansion medium of the invention.

Suitably, the TPO is provided at a concentration of 0.002 μg/mL, or more. For example, the TPO may be provided at a concentration of 0.0025 μg/mL, or more, 0.003 μg/mL, or more, 0.005 μg/mL, or more, or 0.01 μg/mL, or more.

Suitably, the TPO is provided at a concentration of 0.1 μg/mL, or less. For example, the TPO may be provided at a concentration of 0.08 μg/mL, or less, 0.06 μg/mL, or less, or 0.04 μg/mL, or less.

Suitably, the TPO is provided at a concentration of approximately 0.002 μg/mL to 0.1 μg/mL, 0.0025 μg/mL to 0.08 μg/mL, 0.003 μg/mL to 0.06 μg/mL, 0.005 μg/mL to 0.04 μg/mL, or 0.01 μg/mL to 0.03 μg/mL. In a suitable embodiment, the TPO is provided at a concentration of approximately 0.02 μg/mL. Indeed, in a suitable embodiment, the TPO is provided at a concentration of 0.02 μg/mL.

The forms of TPO discussed above are also suitable for use in these embodiments.

Suitably, the cell culture conditions that promote production of progenitor cells used in a method of the invention, or an expansion medium of the invention, may comprise: SCF; and Flt-3 Ligand; and IL-3; and IL-6; and TPO; and ITS; and HSA. The cell culture medium may comprise IMDM, optionally with Glutamax supplementation.

Thus, in a suitable embodiment, the cell culture conditions that promote production of progenitor cells used in a method of the invention, or an expansion medium of the invention, may comprise: SCF at a concentration of approximately 0.2 μg/mL; and Flt-3 Ligand at a concentration of approximately 0.2 μg/mL; and IL-3 at a concentration of approximately 0.015 μg/mL; and IL-6 at a concentration of approximately 0.015 μg/mL; and TPO at a concentration of approximately 0.02 μg/mL; and 1× ITS; and HSA at approximately 1%. The cell culture medium may comprise IMDM, optionally with Glutamax supplementation.

Stem cells that may be employed in such methods of the invention, as a starting material for the production of progenitor cells (and ultimately granulopoietic cells) include, but are not limited to, haematopoietic stem cells (HSCs). Further details of suitable stem cells, and sources of stem cells, are provided elsewhere in this specification, and (without limitation) include cord blood and mobilized blood.

In a suitable embodiment, the cell culture conditions used in culturing the stem cells to produce progenitor cells further comprise the presence of at least one supplement selected from the group consisting of: ITS, and HSA. In a suitable embodiment, such cell culture conditions comprise the presence of both ITS and HSA. Suitably, both ITS and HSA are present in an expansion medium of the invention.

ITS may be provided as a supplement in embodiments of the methods of the invention comprising a step of producing a population of progenitor cells, as well as in an expansion medium of the invention.

Such embodiments of the methods of the first aspect of the invention, or an expansion medium of the invention, may suitably make use of insulin at a concentration of between about 0.1 g/L and about 5 g/L, for example at a concentration of approximately 1.0 g/L, as a supplement. These methods and cell culture media may suitably make use of transferrin at a concentration of between about 0.01 g/L and about 2.5 g/L, for example at a concentration of approximately 0.55 g/L as a supplement. Suitably such methods and cell culture media may make use of selenium at a concentration of between about 0.0001 g/L and about 0.003 g/L, for example at a concentration of approximately 0.00067 g/L, as a supplement.

HSA may be provided as a supplement in embodiments of the methods of the invention comprising a step of producing a population of progenitor cells, as well as in an expansion medium of the invention.

Suitably, the HSA may be provided at a concentration of between 0.1% and 5%. For example, HSA provided as a supplement may be provided at a concentration of approximately 1%.

In embodiments of the methods of the invention in which stem cells are cultured to yield progenitor cells, this may involve expansion of the number of cells present in the culture.

A method of the invention may comprise culturing a population of stem cells in cell culture conditions to produce a population of progenitor cells for any suitable period of time. For example, the cells may be cultured for 1 day, 2 days, 3 days, 4 days, 5 days, 6 days, 7 days, 8 days, 9 days, 10 days, 11 days, 12 days, 13 days, 14 days or 15 days in conditions to produce a population of progenitor cells. Preferably the cells are cultured for 8 or 9 days in conditions to produce a population of progenitor cells. The stem cells may be cultured for 1-15 days, 1-10 days, 2-14 days, 3-13 days, 4-12 days, 5-11 days, 6-10 days, 7-9 days or 8-9 days in conditions to produce a population of progenitor cells. Preferably, the stem cells, such as HSCs, are cultured for 8-9 days in conditions to produce a population of progenitor cells.

In suitable embodiments of such methods of the invention, stem cells are cultured in conditions to produce the population of progenitor cells for a period of 6 to 10 days. For example, such methods may comprise culturing the cells for a period of 7 to 8 days. In a suitable embodiment, such methods may comprise culturing the cells in cell culture conditions to produce a population of progenitor cells for approximately 6 days, or for approximately 7 days, or for approximately 8 days, or for approximately 9 days, or for approximately 10 days.

Accordingly, a method of the invention for preparing cells for therapeutic use may comprise:
(a) culturing a population of stem cells in cell culture conditions for producing progenitor cells comprising the presence of SCF, FLT-3, TPO, IL-3, IL-6, ITS and HSA for 6-10 days, or preferably 8 days, to produce a population of progenitor cells; and
(b) culturing the population of progenitor cells in cell culture conditions that promote differentiation of the progenitor cells to obtain a population of granulopoietic cells.

A suitable method of the invention for preparing cells for therapeutic use may comprise:
(a) culturing a population of stem cells in cell culture conditions for producing progenitor cells comprising the presence of IMDM, SCF, FLT-3, TPO, IL-3, IL-6, ITS and HSA for 6-10 days, or preferably 8 days, to produce a population of progenitor cells; and
(b) culturing the population of progenitor cells in cell culture conditions that promote differentiation of the progenitor cells comprising IMDM, G-CSF, GM-CSF, IL-3, and TNF for 1-6 days, or preferably 5 days, to obtain a population of granulopoietic cells.

Such a method of the invention for preparing cells for therapeutic use may comprise:
(a) culturing a population of stem cells in cell culture conditions for producing progenitor cells comprising the presence of IMDM, SCF, FLT-3, TPO, IL-3, IL-6, ITS and HSA for 6-10 days, or preferably 8 days, to produce a population of progenitor cells; and
(b) culturing the population of progenitor cells in cell culture conditions that promote differentiation of the progenitor cells to obtain a population of granulopoietic cells.

For example, a method of the invention for preparing cells for therapeutic use may comprise:
(a) culturing a population of stem cells in cell culture conditions for producing progenitor cells comprising the presence of IMDM, SCF, FLT-3, TPO, IL-3, IL-6, ITS and HSA for 6-10 days, or preferably 8 days, to produce a population of progenitor cells; and
(b) culturing the population of progenitor cells in cell culture conditions that promote differentiation of the progenitor cells comprising IMDM, SCF, TPO, GCSF, ITS and HSA for 1-6 days, or preferably 5 days, to obtain a population of granulopoietic cells.

Appropriately supplemented cell culture medium may be replaced or replenished at any suitable time during the culture of the stem cells in conditions for producing progenitor cells. For example, the cell culture medium may be replenished on day 1, day 2, day 3, day 4, day 5, day 6, day 7, day 8, day 9, day 10, day 11, day 12, day 13, day 14, or day 15 of culture of the stem cells. Suitably, the cell culture medium is replenished on day 1 and day 6 of culture of the stem cells. The cell culture medium may be replaced on day 1, day 2, day 3, day 4, day 5, day 6, day 7, day 8, day 9, day 10, day 11, day 12, day 13, day 14, or day 15 of culture of the stem cells. Preferably, the cell culture medium is replaced on day 4 of culture of the stem cells.

Stem cells, such as HSCs, from which progenitor cells are to be produced may be seeded at any suitable cell density. For example, the stem cells may be seeded at a density of 1×10⁵ cells/mL-1×10⁶ cells/mL, 2.5×10⁵ cells/mL-1×10⁶ cells/mL, 3×10⁵ cells/mL-8×10⁵ cells/mL or 4×10⁵ cells/mL-6×10⁵ cells/mL, preferably 5×10⁵ cells/mL. The stem cells may be seeded at a density of 1×10⁵ cells/cm²-1×10⁶ cells/cm², 2.5×10⁵ cells/cm²-1×10⁶ cells/cm², 3×10⁵ cells/cm²-8×10⁵ cells/cm² or 4×10⁵ cells/cm²-6×10⁵ cells/cm², preferably 5×10⁵ cells/cm². In a suitable embodiment, the stem cells (such as HSCs) are seeded at a density of 5×10⁵ cells/mL and 5×10⁵ cells/cm².

The cells may be seeded in any suitable culture vessel. For example, the cells may be seeded in a G-Rex 6M or G-Rex 10M culture vessel. The cells may be transferred to a new culture vessel at any suitable time. The cells may be sequentially transferred into cell culture vessels of increasing surface area. Such transfers may take place on day 1, day 2, day 3, day 4, day 5, day 6, day 7, day 8, day 9, day 10, day 11, day 12, day 13, day 14 or day 15 of the culture to produce progenitor cells. For example, the stem cells (such as HSCs) may be transferred from a smaller G-Rex to a G-Rex 100M on day 1, day 2, day 3, day 4, day 5, day 6, day 7, day 8, day 9, day 10, day 11, day 12, day 13, day 14 or day 15 of the culture to produce progenitor cells. For example, the stem cells (such as HSCs) may be transferred to a G-Rex 100M, or a larger cell culture vessel such as a G-Rex 500M, on day 4 of expansion. In a suitable embodiment, progenitor cells may be transferred to a new culture vessel on day 1, day 2, day 3, day 4, day 5, day 6, day 7, day 8, day 9 or day 10 of the culture conditions that promote differentiation of progenitor cells to granulopoietic cells.

In accordance with such embodiments, a suitable method of preparing cells for therapeutic use may comprise:
(a) seeding stem cells (such as HSCs) at 5×10⁵ cells/mL and 5×10⁵ cells/cm²;
(b) culturing the cells in cell culture medium comprising IMDM, SCF, FLT-3, TPO, IL-3, IL-6, ITS and HSA for 8 days to obtain a population of progenitor cells, wherein the cell culture medium comprising IMDM, SCF, FLT-3, TPO, IL-3, IL-6, ITS and HSA is replenished on day 1 and day 6 of such culture, and wherein the cell culture medium comprising IMDM, SCF, FLT-3, TPO, IL-3, IL-6, ITS and HSA is replaced on day 4 of such culture;
(c) culturing the population of progenitor cells in a cell culture medium comprising IMDM, SCF, TPO, GCSF, ITS and HSA for 5-6 days to obtain a population of granulopoietic cells, wherein the cell culture medium comprising IMDM, SCF, TPO, GCSF, ITS and HSA is replenished on day 3 of differentiation.

A suitable method of preparing cells for therapeutic use may comprise:
(a) seeding stem cells (such as HSCs) at 5×10⁵ cells/mL and 5×10⁵ cells/cm²;
(b) culturing the cells in cell culture medium comprising IMDM, SCF, FLT-3, TPO, IL-3, IL-6, ITS and HSA for 8 days to obtain a population of progenitor cells, wherein the cell culture medium comprising IMDM, SCF, FLT-3, TPO, IL-3, IL-6, ITS and HSA is replenished on day 1 and day 6 of such culture, and wherein the cell culture medium comprising IMDM, SCF, FLT-3, TPO, IL-3, IL-6, ITS and HSA is replaced on day 4 of such culture;
(c) culturing the population of progenitor cells in a cell culture medium comprising IMDM, SCF, TPO, G-CSF, ITS and HSA for 5-6 days to obtain a population of granulopoietic cells, wherein the cell culture medium comprising IMDM, SCF, TPO, GCSF, ITS, HAS, GM-CSF, IL-3 and TNF is replenished on day 3 of differentiation.

The inventors have also identified methods by which granulopoietic cells may be primed, in order to amplify properties of the cells that increase their therapeutic utility. In particular, priming of the granulopoietic cells by such methods may amplify their cytocidal activity in a manner that may increase their therapeutic utility.

Accordingly, the third aspect of the invention provides a method of priming granulopoietic cells for therapeutic use, the method comprising culturing a population of granulopoietic cells in the presence of GM-CSF, and optionally one or more cytokines selected from the group consisting of: TNF, IFN-α, IFN-β, IL-15, and IL-18.

A method in accordance with the first aspect of the invention may also comprise a step of priming the granulopoietic cells for therapeutic use, by a method comprising culturing the population of granulopoietic cells in the presence of GM-CSF, and optionally one or more cytokines selected from the group consisting of: TNF, IFN-α, IFN-β, IL-15, and IL-18.

A method of the third or first aspect of the invention comprising a step of priming granulopoietic cells may optionally comprise a further step of purifying the population of primed granulopoietic cells produced, and/or formulating this population of primed cells for medical use.

The fourth aspect of the invention provides a population of primed granulopoietic cells obtainable by a method in accordance with the third aspect of the invention. The population of primed granulopoietic cells may be obtained by a method of the third aspect of the invention. The population of primed granulopoietic cells may be as defined elsewhere in the present disclosure (for example with reference to biological activity of the primed cells, or their expression of particular markers).

GM-CSF may be used in cell culture conditions for a priming step at a concentration of 1-1000 ng/ml, 2-500 ng/ml, 3-250 ng/ml, 4-200 ng/ml. GM-CSF may be used at a concentration of 5-150 ng/mL, for example at a concentration of 10-130 ng/ml.

TNF may be used in cell culture conditions for a priming step at a concentration of 0.001-10 ng/ml, 0.002-5 ng/mL, 0.003-2.5 ng/ml, 0.004-2 ng/mL. TNF may be used at a concentration of 0.005-1.5 ng/ml, for example at a concentration of 0.01-1 ng/mL.

IFN-α may be used in cell culture conditions for a priming step at a concentration of 1-100 ng/ml, 2-50 ng/ml, 3-25 ng/ml, 4-20 ng/ml. IFN-α may be used at a concentration of 5-15 ng/ml, for example at a concentration of 10 ng/ml.

IFN-β may be used in cell culture conditions for a priming step at a concentration of 1-100 ng/ml, 2-50 ng/ml, 3-25 ng/mL, 4-20 ng/ml. IFN-β may be used at a concentration of 5-15 ng/ml, for example at a concentration of 10 ng/ml.

IL-15 may be used in cell culture conditions for a priming step at a concentration of 1-100 ng/ml, 2-50 ng/ml, 3-25 ng/mL, 4-20 ng/mL. IL-15 may be used at a concentration of 5-15 ng/ml, for example at a concentration of 10 ng/ml.

IL-18 may be used in cell culture conditions for a priming step at a concentration of 1-100 ng/mL, 2-50 ng/ml, 3-25 ng/mL, 4-20 ng/ml. IL-18 may be used at a concentration of 5-15 ng/ml, for example at a concentration of 10 ng/ml.

IL-3 may be used in cell culture conditions for a priming step at a concentration of 1-1000 ng/mL, 2-500 ng/mL, 3-250 ng/ml, 4-200 ng/ml. IL-3 may be used at a concentration of 5-150 ng/ml, for example at a concentration of 10-130 ng/ml.

In suitable embodiments, priming involves culturing a population of granulopoietic cells in the presence of GM-CSF at a concentration of approximately 130 ng/ml, and optionally one or more cytokines selected from the group consisting of: TNF at a concentration of approximately 0.01-1.0 ng/mL, IFN-α at a concentration of approximately 10 ng/ml, IFN-β at a concentration of approximately 10 ng/ml, IL-15 at a concentration of approximately 10 ng/ml, IL-18 at a concentration of approximately 10 ng/ml, and IL-3 at a concentration of approximately 130 ng/ml.

In a suitable embodiment, cells undergoing priming may be cultured in the presence of GM-CSF, G-CSF, SCF, TPO, and IL-15. Merely by way of example, cells may be cultured in the presence of GM-CSF at a concentration of approximately 10 ng/mL, G-CSF at a concentration of approximately 130 ng/ml, SCF at a concentration of approximately 130 ng/ml, TPO at a concentration of approximately 130 ng/mL, and IL-15 at a concentration of approximately 10 ng/mL.

In a suitable embodiment, cells undergoing priming may be cultured in the presence of GM-CSF, G-CSF, SCF, TPO, and TNF. Merely by way of example, cells may be cultured in the presence of GM-CSF at a concentration of approximately 100 ng/mL, G-CSF at a concentration of approximately 130 ng/mL, SCF at a concentration of approximately 130 ng/ml, TPO at a concentration of approximately 130 ng/ml, and TNF at a concentration of approximately 10 ng/ml.

In a suitable embodiment, cells undergoing priming may be cultured in the presence of GM-CSF and IL-3. Merely by way of example, cells may be cultured in the presence of GM-CSF at a concentration of approximately 130 ng/ml and IL-3 at a concentration of approximately 130 ng/mL.

In a suitable embodiment, cells undergoing priming may be cultured in the presence of GM-CSF and IL-15. Merely by way of example, cells may be cultured in the presence of GM-CSF at a concentration of approximately 130 ng/mL and IL-15 at a concentration of approximately 10 ng/mL.

In a suitable embodiment, cells undergoing priming may be cultured in the presence of GM-CSF and IL-18. Merely by way of example, cells may be cultured in the presence of GM-CSF at a concentration of approximately 130 ng/ml and IL-18 at a concentration of approximately 10 ng/mL.

In a suitable embodiment, cells undergoing priming may be cultured in the presence of GM-CSF and IL-16. Merely by way of example, cells may be cultured in the presence of GM-CSF at a concentration of approximately 130 ng/ml and IL-16 at a concentration of approximately 10 ng/mL.

In a suitable embodiment, cells undergoing priming may be cultured in the presence of GM-CSF and TNF. Merely by way of example, cells may be cultured in the presence of GM-CSF at a concentration of approximately 130 ng/ml and TNF at a concentration of approximately 1 ng/mL.

In a suitable embodiment, cells undergoing priming may be cultured in the presence of GM-CSF, G-CSF, SCF, TPO, and IFN-α. Merely by way of example, cells may be cultured in the presence of GM-CSF at a concentration of approximately 130 ng/mL, G-CSF at a concentration of approximately 130 ng/ml, SCF at a concentration of approximately 130 ng/mL, TPO at a concentration of approximately 130 ng/ml, and IFN-α at a concentration of approximately 10 ng/mL.

The priming step may last any suitable period of time. For example, the priming step may be last for 1 hour, 2 hours, 3 hours, 6 hours, 12 hours, 18 hours, 24 hours, 30 hours, 36 hours, 42 hours, 48 hours, 54 hours, 60 hours, 72 hours, 78 hours, 84 hours, 90 hours, or 96 hours. The priming step may last for 1-96 hours, 2-90 hours, 3-84 hours, 6-78 hours, 12-72 hours, 18-54 hours, or 24-48 hours. Suitably the priming may comprise culture incorporating the cytokines discussed above, for example at the concentrations set out above, for a period of one, two or three days. In particular, the priming may comprise culture incorporating the priming cytokine combinations referred to for two days.

A priming step may suitably be incorporated at any appropriate stage of a method of the invention. That said, priming will typically occur during the period in which the progenitor cells are cultured in conditions that promote differentiation of the progenitor cells into granulopoietic cells. For example, priming may begin on the first day of culture of the progenitor cells, the second day of culture of the progenitor cells, the third day of culture of the progenitor cells, the fourth day of culture of the progenitor cells, or on the fifth day of culture of the progenitor cells in conditions that promote their differentiation into granulopoietic cells.

Alternatively, in a suitable embodiment, a priming step may occur after the granulopoietic cells have been produced, and optionally after the granulopoietic cells have been harvested. For example, priming may occur before or after cryopreservation of a population of granulopoietic cells in accordance with the invention.

Merely by way of example, in the case of priming steps practiced for two days, the priming may take place on days 3 and 4 of the culture conditions that promote differentiation of the progenitor cells into granulopoietic cells, on days 4 and 5 of such culture, or on days 5 and 6 of such culture. For the avoidance of doubt, any of the priming protocols described above may suitably be practiced on days 3 and 4, days 4 and 5, or days 5 and 6 of the culture conditions that promote differentiation of progenitor cells into granulopoietic cells.

The priming steps developed by the inventors do not appear to significantly influence the immunomodulatory ability of the populations of granulopoietic cells. Accordingly, in embodiments where it is exclusively desired to make use of the granulopoietic cells' immunomodulatory activities, it may preferred to exclude priming steps from the methods by which the granulopoietic cell populations are produced.

The methods of the first aspect of the invention result in the production of populations of granulopoietic cells, which are the subject of the second aspect of the invention. Similarly, the methods of the third aspect of the invention relate to the priming of granulopoietic cells, and result in populations of cells in accordance with the fourth aspect of the invention. Granulopoietic cells are also used in the pharmaceutical compositions, medical uses, and methods of treatment of the invention. Except for where the context may require otherwise, the following definitions are applicable to granulopoietic cells in each of these aspects of the invention, or in any other situation in which granulopoietic cells, or populations of such cells, are referred to.

Any of the cells or populations of cells disclosed herein may be derived from a mammal, such as a human, non-human primate, mouse, rat, dog, cat, horse, or cow. Suitably, the cell or population of cells is derived from a human. Thus, the cell may be a human cell, or the population of cells may be a population of human cells. In particular, a granulopoietic cell, or population of granulopoietic cells, disclosed herein may be derived from a mammal, such as a human, non-human primate, mouse, rat, dog, cat, horse, or cow. Suitably, the granulopoietic cell or population of granulopoietic cells is derived from a human. Thus, the granulopoietic cell may be a human granulopoietic cell. The population of granulopoietic cells may be a population of human granulopoietic cells.

In order to be considered “granulopoietic” in the terms of the present invention, a cell must be capable of giving rise to granulocytes (for example, neutrophils), or to granulocyte precursor cells of the granulocytic lineage. Indeed, a suitable granulopoietic cell may give rise to such cells. For the avoidance of doubt, granulocytes themselves are to be considered “granulopoietic” for the purposes of the present invention, though in many embodiments the granulopoietic cells will not be granulocytes, but rather cells capable of giving rise to granulocytes. Suitably, granulopoietic cells in the context of the present invention may be taken as excluding other cell lineages, for example excluding monocyte lineages and/or lymphocyte lineages.

Populations of suitable granulopoietic cells in the context of the present invention may be defined with reference to their expression of different markers. The skilled person will be well-aware of suitable methods by which cells may be characterised and/or isolated, and if desired enriched, on the basis of their expression of specific profiles of cell surface markers.

The following definitions, based upon suitable markers expression profiles, may be used singly or in combination to identify suitable populations of granulopoietic cells.

Unless specified otherwise (for example, in lists reciting “or” or “and/or”), references in the present disclosure to cells being positive or negative for expression of a number of specified markers should be taken as requiring the cells in question to have the recited expression (either positive or negative) of each of the markers referred to. Thus, by way of example, reference to a cell, or population of cells, as “CD15+ CD66b+” should be taken as meaning that the cell is positive for the expression of both CD15 and CD66b, and that the population of cells comprises cells that are CD15+ as well as cells that are CD66b+.

The present disclosure includes definitions of populations, or subpopulations, of cells with reference to a recited expression (either positive or negative) of a number of specified markers.

In a suitable embodiment, such definitions may be taken as requiring that the population, or subpopulation, in question comprises cells that are positive or negative (as required by the definition) for the recited markers. For example, in the case of a population defined as positive for expression of first marker, negative for expression of a second marker, and positive for expression of a third marker, this requirement may be met by a cell population that comprises cells positive for the first marker, while also comprising cells negative for the second marker, and further comprising cells positive for the third marker. In such an embodiment, the population, or subpopulation, of cells may be heterogeneous in respect of cells that have the recited expression (whether positive or negative). Suitably, cells that each exhibit the required expression in respect of each of the recited markers may make up the largest group of cells within such a population, or subpopulation. Suitably, cells that each exhibit the required expression in respect of each of the recited markers may make up the majority of cells within such a population, or subpopulation. Suitably, cells that each exhibit the required expression in respect of each of the recited markers may provide at least 55%, at least 60%, at least 65%, at least 70%, at least 75%, at least 80%, at least 85%, at least 90%, at least 95%, at least 96%, at least 97%, at least 98%, or at least 99% of cells within such a population, or subpopulation.

In one embodiment, in a given population or subpopulation, a cell in that population or subpopulation may express at least 2, 3, 4, or 5 of the recited markers. In one embodiment, in a given population or subpopulation, each of the cells in the population or subpopulation may express at least 2, 3, 4, or 5 of the recited markers.

In a suitable embodiment, such definitions may be taken as requiring that the population, or subpopulation, in question consists of cells that are positive or negative (as required by the definition) for the recited markers. In such an embodiment, the population, or subpopulation, of cells is homogeneous in respect of cells that have the recited expression (whether positive or negative).

In a suitable embodiment, a population of granulopoietic cells comprises cells that are “Lin-” (which is to say negative for a cocktail of common leukocyte lineage markers, defined for the present purposes as negative for expression of each of CD3, CD16, CD19, CD20, CD14 and CD56). For example, a suitable population of granulopoietic cells may comprise at least 45%, at least 50%, at least 55%, at least 60%, at least 65%, at least 70%, at least 75%, at least 80%, or at least 85% Lin-cells. By way of example, a suitable population of granulopoietic cells may comprise at least 90% Lin-cells. A suitable population of granulopoietic cells may comprise approximately 95-99% Lin-cells. Suitably, a population of granulopoietic cells comprises approximately 97% Lin-cells.

Alternatively, or additionally, a suitable population of granulopoietic cells comprises CD34+ cells. For example, such a population of granulopoietic cells may comprise less than 50%, less than 45%, less than 40%, or less than 35% CD34+ cells. By way of example, such a population of granulopoietic cells may comprise less than 30% CD34+ cells. In such an embodiment, the proportion of CD34+ cells may be between approximately 5-25%. Suitably, a population of granulopoietic cells comprises approximately 14% CD34+ cells.

Alternatively, or additionally, a suitable population of granulopoietic cells comprises CD38+ cells. For example, such a population of granulopoietic cells may comprise at least 10%, at least 15%, or at least 20%, CD38+ cells. In such an embodiment, the proportion of CD38+ cells may be between approximately 10% and 80%, such as between approximately 10% and 30%. Suitably, a population of granulopoietic cells comprises approximately 12% CD38+ cells.

Alternatively, or additionally, a suitable population of granulopoietic cells comprises cells with a haematopoietic stem cell (HSC) phenotype (defined for the present purposes as Lin-CD34+CD38−CD45RA−CD90+). For example, such a population of granulopoietic cells may comprise less than 5%, less than 4%, less than 3%, or less than 2% cells with an HSC phenotype. By way of example, such a population of granulopoietic cells may comprise less than 1% cells with an HSC phenotype. A suitable population of granulopoietic cells may comprise approximately 0.01-0.15% cells with an HSC phenotype. Suitably, a population of granulopoietic cells comprises approximately 0.04% cells with an HSC phenotype.

Alternatively, or additionally, a suitable population of granulopoietic cells comprises less than 1% cells with a long-term repopulating haematopoietic stem cell (LT-HSC) phenotype (defined for the present purposes as Lin−CD34+CD38−CD45RA−CD90+CD49f+). For example, such a population of granulopoietic cells may comprise less than 5%, less than 4%, less than 3%, or less than 2% cells with an LT-HSC phenotype. By way of example, such a population of granulopoietic cells may comprise less than 1% cells with an LT-HSC phenotype. A suitable population of granulopoietic cells may comprise approximately 0.01-0.05% cells with an LT-HSC phenotype. Suitably, a population of granulopoietic cells comprises approximately 0.02% cells with an LT-HSC phenotype.

Alternatively, or additionally, a suitable population of granulopoietic cells comprises cells with a lymphoid primed multi potent progenitor (LMPP) phenotype (defined for the present purposes as Lin−CD34+CD38−CD45RA+). For example, such a population of granulopoietic cells may comprise less than 50%, less than 45%, less than 40%, less than 35%, less than 30%, or less than 25% cells with an LMPP phenotype. By way of example, such a population of granulopoietic cells may comprise less than 20% cells with an LMPP phenotype. A suitable population of granulopoietic cells may comprise approximately 2-15% cells with an LMPP phenotype. Suitably, a population of granulopoietic cells comprises approximately 5% cells with an LMPP phenotype.

Alternatively, or additionally, a suitable population of granulopoietic cells comprises cells with a multipotent progenitor (MPP) phenotype (defined for the present purposes as Lin-CD34+CD38−CD45RA−). For example, such a population of granulopoietic cells may comprise less than 30%, less than 25%, less than 20%, or less than 15% cells with an MPP phenotype. By way of example, such a population of granulopoietic cells may comprise less than 10% cells with an MPP phenotype. A suitable population of granulopoietic cells may comprise approximately 1-6% cells with an MPP phenotype. Suitably, a population of granulopoietic cells comprises approximately 2% cells with an MPP phenotype.

In a suitable embodiment, a population of granulopoietic cells may comprise more than 90% Lin-cells (for example, approximately 97% Lin-cells), and/or less than 30% CD34+ cells (for example, approximately 14% CD34+ cells), and/or more than 10% CD38+ cells (for example, approximately 12% CD38+ cells), and/or less than 1% cells with an HSC phenotype as defined above (for example approximately 0.04% cells with an HSC phenotype), and/or less than 1% cells with an LT-HSC phenotype as defined above (for example approximately 0.02% cells with an LT-HSC phenotype), and/or less than 20% cells with an LMPP phenotype as defined above (for example approximately 5% cells with an LMPP phenotype), and/or less than 10% cells with an MPP phenotype as defined above (for example approximately 2.5% cells with an MPP phenotype).

In a suitable embodiment, a population of granulopoietic cells may comprise more than 90% Lin-cells (for example, approximately 97% Lin-cells), and less than 30% CD34+ cells (for example, approximately 14% CD34+ cells), and more than 10% CD38+ cells (for example, approximately 12% CD38+ cells), and less than 1% cells with an HSC phenotype as defined above (for example approximately 0.04% cells with an HSC phenotype), and less than 1% cells with an LT-HSC phenotype as defined above (for example approximately 0.02% cells with an LT-HSC phenotype), and less than 20% cells with an LMPP phenotype as defined above (for example approximately 5% cells with an LMPP phenotype), and less than 10% cells with an MPP phenotype as defined above (for example approximately 2.5% cells with an MPP phenotype).

Alternatively, or additionally, a suitable population of granulopoietic cells may comprise a ratio of CD15− to CD15+ cells that is approximately 1:1.

A suitable population of granulopoietic cells may comprise around 25-75%, or 35-60 CD15− cells. For example, a suitable population of granulopoietic cells may comprise approximately 50% CD15-cells.

A suitable population of granulopoietic cells may comprise around 30-70%, or 40-65%, CD15+ cells. For example, a suitable population of granulopoietic cells may comprise approximately 50% CD15+ cells.

A suitable population of granulopoietic cells may comprise around 5-25%, 5-20%, 7-18%, or 10-15% CD15+CD66b+ cells. For example, a suitable population of granulopoietic cells may comprise approximately 12% CD15+CD66b+ cells.

A suitable population of granulopoietic cells may comprise around less than 30% or less than 25% CD11b+ cells. For example, a suitable population of granulopoietic cells may comprise approximately 10-25% or 15-25% CD11b+ cells, for example approximately 19% CD11b+ cells.

A suitable population of granulopoietic cells may comprise at least 30%, at least 35%, at least 40%, or at least 45% CD71+ cells. For example, a suitable population of granulopoietic cells may comprise approximately 60% CD71+ cells.

A suitable population of granulopoietic cells may comprise around 60-95%, or 65-90% CD49d+ cells. For example, a suitable population of granulopoietic cells may comprise approximately 75% CD49d+ cells.

A suitable population of granulopoietic cells may comprise less than 5%, less than 4%, less than 3%, or less than 2% CD10+ cells A suitable population of granulopoietic cells may comprise around 0.03-2% CD10+ cells. For example, a suitable population of granulopoietic cells may comprise approximately 0.5% CD10+ cells.

A suitable population of granulopoietic cells may comprise around 1-120%, or 2-15% CD177+ cells. A suitable population of granulopoietic cells may comprise approximately 6% CD177+ cells.

A suitable population of granulopoietic cells may comprise less than 20% or less than 15% CD62L+ cells. For example, a suitable population of granulopoietic cells may comprise between approximately 2-15%, for example approximately 8% CD62L+ cells.

A suitable population of granulopoietic cells may comprise around 40-85%, or 50-75%, CD54+ cells. For example, a suitable population of granulopoietic cells may comprise approximately 63% CD54+ cells.

A suitable population of granulopoietic cells may comprise around 2-15%, or around 5-10% CD63+ cells. For example, a suitable population of granulopoietic cells may comprise approximately 7% CD63+ cells.

A suitable population of granulopoietic cells may comprise around 70-90%, or 75-85% CD18+ cells. For example, a suitable population of granulopoietic cells may comprise approximately 80% CD18+ cells.

A suitable population of granulopoietic cells may comprise around 35-55% HLA-DR+ cells. For example, a suitable population of granulopoietic cells may comprise approximately 47% HLA-DR+ cells.

A suitable population of granulopoietic cells may comprise around 6-8% CD115+ cells. For example, a suitable population of granulopoietic cells may comprise approximately 5% CD115+ cells.

A suitable population of granulopoietic cells may comprise around 5-30% CD40+ cells. For example, a suitable population of granulopoietic cells may comprise approximately 15% CD40+ cells.

A suitable population of granulopoietic cells may comprise around 5-30% CD64+ cells. For example, a suitable population of granulopoietic cells may comprise approximately 15% CD64+ cells.

A suitable population of granulopoietic cells may comprise around 20-55% CD32+ cells. For example, a suitable population of granulopoietic cells may comprise approximately 40% CD32+ cells.

A suitable population of granulopoietic cells may comprise around 4-9% CXCR2+ cells. For example, a suitable population of granulopoietic cells may comprise approximately 6% CXCR2+ cells.

A suitable population of granulopoietic cells may comprise around 0.04-1% CD16+ cells. For example, a suitable population of granulopoietic cells may comprise approximately 0.25% CD16+ cells.

A suitable population of granulopoietic cells may comprise around 2-15% CD14+ cells. For example, a suitable population of granulopoietic cells may comprise approximately 8% CD14+ cells.

A suitable population of granulopoietic cells may comprise around 0.5-4% CD68+ cells. For example, a suitable population of granulopoietic cells may comprise approximately 1.5% CD68+ cells.

A suitable population of granulopoietic cells may comprise around 2-18% CD206+ cells. For example, a suitable population of granulopoietic cells may comprise approximately 10% CD206+ cells.

The fifth aspect of the invention provides an isolated population of granulopoietic cells comprising:
more than 90% Lin-cells (for example, approximately 97% Lin-cells);
less than 30% CD34+ cells (for example, approximately 14% CD34+ cells);
more than 30% CD38+ cells (for example, approximately 65% CD38+ cells);
less than 1% cells with an HSC phenotype (for example approximately 0.04% cells with an HSC phenotype);
less than 1% cells with an LT-HSC phenotype (for example approximately 0.02% cells with an LT-HSC phenotype;
less than 20% cells with an LMPP phenotype (for example approximately 5% cells with an LMPP phenotype); and;
less than 10% cells with an MPP phenotype (for example approximately 2.5% cells with an MPP phenotype).

The sixth aspect of the invention provides an isolated population of granulopoietic cells comprising:
a first subpopulation of cells that are CD15+ CD64+ CD18+ CD49d+ CD71+
a second subpopulation of cells that are CD15− CD11b+/− CD18+ CD49d+ CD32+ HLA−DR−.
a third subpopulation of cells that are CD15− CD11b− HLA−DR+ CD18+ CD49d+ and CD71+.

A population of granulopoietic cells in accordance with the sixth aspect of the invention may further comprise a fourth subpopulation of cells that are CD15− CD11b+ HLA−DR+.

It will be appreciated that, having been informed of the markers expressed by these subpopulations of cells, one or more of these subpopulations may readily be isolated from within the populations of cells of the sixth aspect of the invention. This gives rise to further aspects of the invention.

In a seventh aspect, the invention provides a population of granulopoietic cells that are CD15+ CD64+ CD18+ CD49d+ CD71+. A suitable population of such cells (which may also constitute a first subpopulation of cells in the context of the sixth aspect of the invention), may also be positive for one, more than one, or all of the markers selected from the group consisting of: CD177, CD11b, CD71, CD66b, HLA-DR, CD115, CD49d, CD40, CD62L, CD54, CD18, CD34, CXCR4, CD64, CD32, CXCR2, CD38, Mac1, 4-1BBL, OX40L, PD-L1, and CD14. A population of cells of the seventh aspect of the invention may be negative for the markers CD16 and/or CD62L (in addition to the required or optional expression or lack of expression of the other markers discussed above). Suitably the population, or subpopulation, of cells is heterogeneous for the recited marker profile (which may suitably include the optional constituents referred to herein). Suitably, a population, or subpopulation, of cells in accordance with this aspect of the invention is homogeneously positive for CD15, and heterogeneous in respect of the other markers of the recited marker profile (which may suitably include the optional constituents referred to herein). Suitably the population, or subpopulation, of cells is homogeneous for the recited marker profile (which may suitably include the optional constituents referred to herein).

The first subpopulation of cells present in a population of granulopoietic cells in accordance with the sixth aspect of the invention, or cells in accordance with the seventh aspect of the invention, express markers that closely resemble those expressed by committed neutrophil precursors. However, the cells disclosed in accordance with this aspect of the invention are CD64+, and may be CD16− and/or CD62L−. This is in contrast to neutrophil precursors found in the circulation and at times of homeostasis, which are CD64− CD16+ and CD62L+. Expression of CD64 by CD15+ CD64+ CD18+ CD49d+ CD71+ cells thus provides a useful means by which the cells disclosed herein may be distinguished from those that occur naturally, as does a lack of expression of CD16 and/or CD62L. A cell, or a population of cells, that are CD15+ CD64+ CD18+ CD49d+ CD71+ and also CD16− and/or CD62L− can be distinguished as one that has been produced by method in accordance with the invention, rather than a naturally occurring granulopoietic cell, or population of such cells.

The inventors have identified that cells of a first subpopulation of cells present in a population of granulopoietic cells in accordance with the sixth aspect of the invention, or cells of the seventh aspect of the invention, demonstrate cytocidal activity that makes them particularly effective in terms of their medical uses. Indeed, such cells appear to constitute the major source of cytocidal activity in populations of cells in accordance with the sixth aspect of the invention. Thus, such cells may be particularly useful in clinical contexts in which it is required to kill cells (such as cancer cells, infected cells, or cellular infectious agents) in order to achieve a therapeutic effect.

The first subpopulation of cells present in a population of granulopoietic cells in accordance with the sixth aspect of the invention, or cells of the seventh aspect of the invention, may express 4-1BBL and/or OX40L. These markers are ligands for T cells and NK cells, and their expression by these cells may indicate that the cells will have immunomodulatory activities. Similarly, the first subpopulation of cells present in a population of granulopoietic cells in accordance with the sixth aspect of the invention, or cells of the seventh aspect of the invention, may express CD38 and/or CD40 and/or CD54, further co-stimulatory molecules associated with functional interactions with immune cells such as T cells. Accordingly, such cells, or pharmaceutical compositions comprising such cells, may be effective in biological or therapeutic applications utilising the modulation of activity of such non-granulocytic inflammatory cell types.

In addition to expressing markers indicative of immunomodulatory ability, this population of cells also expresses molecules (in particular CD11b, CD18, Mac1 and CD32) that suggest they possess direct cytocidal activity. This may make the suitable for uses in which it is desired to therapeutically kill cells, such as cancerous or infected cells.

In an eighth aspect, the invention provides a population of cells that are CD15− CD11b+/− CD18+ CD49d+ CD32+ HLA−DR−. A suitable population of such cells (which may also constitute a second subpopulation of cells in the context of the sixth aspect of the invention), may also be positive for one, more than one, or all of the markers selected from the group consisting of: CD177, CD11b, CD71, CD66b, CD115, CD49d, CD40, CD62L, CD54, CD18, CD34, CXCR4, CD64, CD32, CXCR2, CD38, Mac1, 4-1BBL, OX40L, PD-L1, and CD14.

Suitably the population, or subpopulation, of cells is heterogeneous for the recited marker profile (which may suitably include the optional constituents referred to herein). Suitably, a population, or subpopulation, of cells in accordance with this aspect of the invention is homogeneously negative for CD15 and HLA-DR, and heterogeneous in respect of the other markers of the recited marker profile (which may suitably include the optional constituents referred to herein). Suitably, a population, or subpopulation, of cells in accordance with this embodiment is homogeneously negative for CD15, HLA-DR and CD11b, and heterogeneous in respect of the other markers of the recited marker profile (which may suitably include the optional constituents referred to herein). Suitably, a population, or subpopulation, of cells in accordance with this embodiment is homogenously positive for CD11b and homogeneously negative for CD15 and HLA-DR, and heterogeneous in respect of the other markers of the recited marker profile (which may suitably include the optional constituents referred to herein). Suitably the population, or subpopulation, of cells is homogeneous for the recited marker profile (which may suitably include the optional constituents referred to herein).

The second subpopulation of cells present in a population of granulopoietic cells in accordance with the sixth aspect of the invention, or cells in accordance with the eighth aspect of the invention, express markers, such as Mac-1 (comprising CD11b and CD18) and CD32, that are consistent with a high capacity for cytotoxic activity. Accordingly, these cells may also be of benefit in medical uses or methods of treatment where direct cytocidal activity is required, such as the killing of cancerous or infected cells. These cells may also express molecules such as 4-1BBL and/or OX40L indicating their potential for immunomodulation, and suitability for use in biological or therapeutic applications requiring such activity. Cells of this group may also express CXCR2, which may be elevated by their exposure to IL-3 during methods in accordance with the invention, a marker that may contribute to heightened chemotaxis (in response to agents such as IL-8) and targeting of these cells into the TME.

In a ninth aspect, the invention provides a population of cells that are CD15− CD11b− HLA−DR+ CD18+ CD49d+ and CD71+. A suitable population of such cells (which may also constitute a third subpopulation of cells in the context of the sixth aspect of the invention), may also be positive for one, more than one, or all of the markers selected from the group consisting of: CD177, CD71, CD66b, CD115, CD49d, CD40, CD62L, CD54, CD18, CD34, CXCR4, CD64, CD32, CXCR2, CD38, Mac1, 4-1BBL, OX40L, PD-L1, and CD14. Suitably the population, or subpopulation, of cells is heterogeneous for the recited marker profile (which may suitably include the optional constituents referred to herein). Suitably, a population, or subpopulation, of cells in accordance with this aspect of the invention is homogeneously negative for CD15 and CD11b and homogenously positive for HLA-DR, and heterogeneous in respect of the other markers of the recited marker profile (which may suitably include the optional constituents referred to herein). Suitably the population, or subpopulation, of cells is homogeneous for the recited marker profile (which may suitably include the optional constituents referred to herein).

The third subpopulation of cells present in a population of granulopoietic cells in accordance with the sixth aspect of the invention, or cells in accordance with the ninth aspect of the invention, express markers indicative of a relatively low level of differentiation. In keeping with this, these cells may also be CD34+. The cells of this group may also express markers, such as 4-1BBL and/or OX40L and/or CD40 and/or CD54 that indicate their suitability for use in applications requiring immunomodulation of non-granulocytic immune cells. While the cells of this group do not express markers indicative of direct cytocidal activity, they may have the capacity to differentiate further, and to express markers such as CD11b and CD15 that would confer such activity. Accordingly, these cells may be employed in medical uses or methods of treatment where in vivo signals would induce such differentiation, leading to the ability to kill deleterious cell types.

In a tenth aspect, the invention provides a population of granulopoietic cells that are CD15− CD11b+ HLA−DR+. A suitable population of such cells (which may also constitute an optional fourth subpopulation of cells in the context of the sixth aspect of the invention), may also be positive for one, more than one, or all of the markers selected from the group consisting of: CD177, CD71, CD66b, CD115, CD49d, CD40, CD62L, CD54, CD18, CD34, CXCR4, CD64, CD32, CXCR2, CD38, Mac1, 4-1BBL, OX40L, PD-L1, and CD14. Suitably the population, or subpopulation, of cells is heterogeneous for the recited marker profile (which may suitably include the optional constituents referred to herein). Suitably, a population, or subpopulation, of cells in accordance with this aspect of the invention is homogeneously negative for CD15 and homogenously positive for HLA-DR and CD11b, and heterogeneous in respect of the other markers of the recited marker profile (which may suitably include the optional constituents referred to herein). Suitably the population, or subpopulation, of cells is homogeneous for the recited marker profile (which may suitably include the optional constituents referred to herein).

These cells in accordance with the tenth aspect of the invention, which may optionally also be present in populations of granulopoietic cells in accordance with the sixth aspect of the invention, express markers that are similar to those that would be expected of activate myeloid cells. The cells may further express markers such as CD14 and/or CD11b and/or CD206. They may be suitable for use in applications in which it is desired to make use of either direct cytocidal or immunomodulatory activity.

A granulopoietic cell of a population produced by a method of the invention, or present in a composition of the invention, may be CD64+, CD16− and/or CD62L−. For example, the granulopoietic cell may be CD64+. The granulopoietic cell may be CD64+ and CD16−. The granulopoietic cell may be CD64+ and CD62L−. The granulopoietic cell may be CD16− and CD62L−. Suitably, the granulopoietic cell is CD64+, CD16− and CD62L−. Expression of CD64, and the lack of expression of CD16 and CD62L by granulopoietic cells of the invention contrasts to neutrophils found in the circulation and at times of homeostasis, which are CD64− CD16+ and CD62L+. Expression of CD64 thus provides a useful means by which the granulopoietic cell disclosed herein may be distinguished from those that occur naturally, as does a lack of expression of CD16 and/or CD62L. A granulopoietic cell that is CD64+, CD16− and/or CD62L− may be distinguished as one that has been produced by a method in accordance with the invention, rather than a naturally occurring granulopoietic cell, or population of such cells.

Thus, in one aspect, the invention provides a granulopoietic cell that is a CD64+ granulopoietic cell, or a population of such cells. Suitably, the CD64+ granulopoietic cell is a CD64+ and CD16− granulopoietic cell. The CD64+ granulopoietic cell may be a CD64+ and CD62L− granulopoietic cell. The CD64+ granulopoietic cell may be a CD64+, CD16− and CD62L− granulopoietic cell. Populations of cells with each of these expression profiles are provided in accordance with this aspect of the invention.

In a related aspect, the invention provides a granulopoietic cell that is a CD16− granulopoietic cell. The CD16− granulopoietic cell may be a CD16− and CD62L− granulopoietic cell.

In another related aspect, the invention provides a granulopoietic cell that is a CD62L− granulopoietic cell.

In an eleventh aspect, the invention provides a pharmaceutical composition comprising a population of granulopoietic cells. Suitably, the population of granulopoietic cells may be a population in accordance with the second, or fourth to tenth, aspects of the invention.

In a twelfth aspect, the invention provides the use of a population of granulopoietic cells in the manufacture of a medicament. Suitably, the population of granulopoietic cells may be a population in accordance with the second, or fourth to tenth, aspects of the invention.

In a thirteenth aspect, the invention provides a pharmaceutical composition of the sixth aspect of the invention, or a population of granulopoietic cells in accordance with the second, or fourth to tenth, aspects of the invention, for use as a medicament.

In a fourteenth aspect, the invention provides a method of treating a disease or disorder in a subject, the method comprising administering a pharmaceutical composition in accordance with the sixth aspect of the invention, or a population of granulopoietic cells in accordance with the second, or fourth to tenth, aspects of the invention, to the subject.

In an aspect, the invention provides a pharmaceutical composition of the invention for use in a method of treating a disease or disorder in a subject.

In an aspect, the invention provides a pharmaceutical composition of the invention for use in medicine.

In an aspect, the invention provides a method of treating a disease or disorder in a subject comprising administering a pharmaceutical composition of the invention to the subject.

In an aspect, the invention provides a pharmaceutical composition of the invention for use in a method of treating cancer in a subject.

In an aspect, the invention provides a method of treating cancer in a subject comprising administering a pharmaceutical composition of the invention to the subject.

In an aspect, the invention provides use of a pharmaceutical composition of the invention in the manufacture of a medicament for treating cancer in a subject.

In an aspect, the invention provides a pharmaceutical composition of the invention for use in a method of treating an infection in a subject.

In an aspect, the invention provides a method of treating an infection in a subject comprising administering a pharmaceutical composition of the invention to the subject.

In an aspect, the invention provides use of a pharmaceutical composition of the invention in the manufacture of a medicament for treating an infection in a subject.

In an aspect, the invention provides a pharmaceutical composition of the invention, for use to amplify a non-granulocytic therapeutic immune response.

In an aspect, the invention provides a method of treatment comprising amplifying a non-granulocytic therapeutic immune response, the method comprising providing a pharmaceutical composition of the invention to a subject in need of such treatment.

In an aspect, the invention provides a pharmaceutical composition of the invention for use in the manufacture of a medicament for use in amplifying a non-granulocytic therapeutic immune response.

The present invention is based, to at least some extent, upon the inventors' development of method capable of generating populations of granulopoietic cells with properties that are highly beneficial in a broad range of therapeutic contexts. The methods of the first aspect of the invention result in the production of populations of granulopoietic cells, which are the subject of the second aspect of the invention. Similarly, the methods of the third aspect of the invention relate to the priming of granulopoietic cells, and result in populations of cells in accordance with the fourth aspect of the invention. Granulopoietic cells are also used in the pharmaceutical compositions, medical uses, and methods of treatment of the invention.

The populations of granulopoietic cells described herein, such as those that may be produced using the methods of the invention, are capable of amplifying the therapeutic immune response of non-granulocytic immune cells. In suitable embodiments, the populations of granulopoietic cells bring about the amplification of an immune response of non-granulocytic immune cells. Such populations of granulopoietic cells, or pharmaceutical compositions comprising such populations, can be used to treat a number of conditions, including (but not limited to) cancer and the treatment of infections. Said populations and compositions can also be used to augment immunotherapeutic treatments in a number of conditions, including (but not limited to) cancer therapies and treatment of infections.

As set out above, the populations of granulopoietic cells may comprise cells that express markers, such as 4-1BBL and/or OX40L and/or CD40 and/or CD54, associated with interaction with non-granulocytic immune cells. Such cells, or pharmaceutical compositions comprising such cells, may be employed in medical uses or methods of treatment requiring beneficial immunomodulatory activity.

Alternatively, or additionally, suitable populations of granulopoietic cells may comprise cells that express markers, such as Mac-1 (or its constituents CD11b and CD18) or CD32, that are indicative of a capacity for direct cytocidal activity. Such cells, or pharmaceutical compositions comprising such cells, may be employed in medical uses or methods of treatment that require killing of cells such as cancerous or infected cells.

The methods of the invention make use of populations of progenitor cells as the “starting material” from which the granulopoietic cells are produced. As noted above, some embodiments of the methods of the invention may also incorporate an optional step of culturing a population of stem cells to produce a population of progenitor cells.

In a similar manner to the populations of granulopoietic cells discussed above, progenitor cells, and populations of progenitor cells, in the context of the present disclosure may usefully be defined by means of their expression of marker profiles and phenotypes. The following definitions, based upon suitable markers expression profiles, may be used singly or in combination to identify suitable populations of progenitor cells. Except for where the context requires otherwise, they should be considered appliable to progenitor cells as referred to in any embodiment of the invention.

In a suitable embodiment, a population of progenitor cells comprises cells that are Lin-(as defined above). For example, a suitable population of progenitor cells may comprise at least 55%, at least 60%, at least 65%, at least 70%, at least 75%, at least 80%, at least 85%, at least 90%, or at least 95% Lin-cells. By way of example, a suitable population of progenitor cells may comprise at least 98% Lin-cells. A suitable population of progenitor cells may comprise approximately 98-99% Lin-cells. Suitably, a population of progenitor cells comprises approximately 99% Lin-cells.

Alternatively, or additionally, a suitable population of progenitor cells comprises CD34+ cells. For example, such a population of progenitor cells may comprise between approximately 5-90%, or approximately 10-85% CD34+ cells. By way of example, such a population of progenitor cells may comprise between approximately 15-80% CD34+ cells. In such an embodiment, the proportion of CD34+ cells may be between approximately 20-70%. Suitably, a population of progenitor cells comprises approximately 43% CD34+ cells.

Alternatively, or additionally, a suitable population of progenitor cells comprises CD38+ cells. For example, such a population of progenitor cells may between approximately 10-65%, approximately 15-60%, or approximately 20-55% CD38+ cells. By way of example, such a population of progenitor cells may comprise between approximately 25-50% CD38+ cells. In such an embodiment, the proportion of CD38+ cells may be between approximately 30% and 41%. Suitably, a population of progenitor cells comprises approximately 35% CD38+ cells.

Alternatively, or additionally, a suitable population of progenitor cells comprises cells with an HSC phenotype. For example, such a population of progenitor cells may comprise less than 5%, less than 4%, less than 3%, or less than 2% cells with an HSC phenotype. By way of example, such a population of progenitor cells may comprise less than 1% cells with an HSC phenotype. A suitable population of progenitor cells may comprise approximately 0.01-0.7% cells with an HSC phenotype. Suitably, a population of progenitor cells comprises approximately 0.3% cells with an HSC phenotype.

Alternatively, or additionally, a suitable population of progenitor cells comprises cells with an LT-HSC phenotype. For example, such a population of progenitor cells may comprise less than 5%, less than 4%, less than 3%, or less than 2% cells with an LT-HSC phenotype. By way of example, such a population of progenitor cells may comprise less than 1% cells with an LT-HSC phenotype. A suitable population of progenitor cells may comprise approximately 0.01-0.03% cells with an LT-HSC phenotype. Suitably, a population of progenitor cells comprises approximately 0.02% cells with an LT-HSC phenotype.

Alternatively, or additionally, a suitable population of progenitor cells comprises cells with an LMPP phenotype. For example, such a population of progenitor cells may comprise less than 70%, less than 65%, less than 60%, less than 55%, less than 50%, or less than 45% cells with an LMPP phenotype. By way of example, such a population of progenitor cells may comprise less than 40% cells with an LMPP phenotype. A suitable population of progenitor cells may comprise approximately 5-30% cells with an LMPP phenotype. Suitably, a population of progenitor cells comprises approximately 13% cells with an LMPP phenotype.

Alternatively, or additionally, a suitable population of progenitor cells comprises cells with an MPP phenotype. For example, such a population of progenitor cells may comprise less than 70%, less than 65%, less than 60%, less than 55%, less than 50%, or less than 45% cells with an MPP phenotype. By way of example, such a population of progenitor cells may comprise less than 40% cells with an MPP phenotype. A suitable population of progenitor cells may comprise approximately 1-35% cells with an MPP phenotype. Suitably, a population of progenitor cells comprises approximately 13% cells with an MPP phenotype.

In a suitable embodiment, a population of progenitor cells may comprise more than 98% Lin-cells (for example, approximately 99% Lin-cells), and/or 15-18% CD34+ cells (for example, approximately 43% CD34+ cells), and/or 25-50% CD38+ cells (for example, approximately 35% CD38+ cells), and/or less than 1% cells with an HSC phenotype as defined above (for example approximately 0.3% cells with an HSC phenotype), and/or less than 1% cells with an LT-HSC phenotype as defined above (for example approximately 0.02% cells with an LT-HSC phenotype), and/or less than 40% cells with an LMPP phenotype as defined above (for example approximately 13% cells with an LMPP phenotype), and/or less than 40% cells with an MPP phenotype as defined above (for example approximately 13% cells with an MPP phenotype).

In a suitable embodiment, a population of progenitor cells may comprise more than 98% Lin-cells (for example, approximately 99% Lin-cells), 15-18% CD34+ cells (for example, approximately 43% CD34+ cells), 25-50% CD38+ cells (for example, approximately 35% CD38+ cells), less than 1% cells with an HSC phenotype as defined above (for example approximately 0.3% cells with an HSC phenotype), less than 1% cells with an LT-HSC phenotype as defined above (for example approximately 0.02% cells with an LT-HSC phenotype), less than 40% cells with an LMPP phenotype as defined above (for example approximately 13% cells with an LMPP phenotype), and less than 40% cells with an MPP phenotype as defined above (for example approximately 13% cells with an MPP phenotype).

Alternatively, or additionally, a suitable population of progenitor cells may comprise a ratio of CD15− to CD15+ cells that is approximately 2:1.

A suitable population of progenitor cells may comprise around 60-95% CD15− cells. For example, a suitable population of progenitor cells may comprise approximately 71% CD15− cells.

A suitable population of progenitor cells may comprise around 10-50% CD15+ cells. For example, a suitable population of progenitor cells may comprise approximately 35% CD15+ cells.

A suitable population of progenitor cells may comprise around 0.02-1% CD15+CD66b+ cells. For example, a suitable population of progenitor cells may comprise approximately 0.04-0.47% or 0.24% CD15+CD66b+ cells.

A suitable population of progenitor cells may comprise less than 20% CD11b+ cells. For example, a suitable population of progenitor cells may comprise approximately 2-6%, or approximately 3% CD11b+ cells.

A suitable population of progenitor cells may comprise around 25-60% CD71+ cells. For example, a suitable population of progenitor cells may comprise approximately 33% CD71+ cells.

A suitable population of progenitor cells may comprise around 90-100% CD49d+ cells. For example, a suitable population of progenitor cells may comprise approximately 95% CD49d+ cells.

A suitable population of progenitor cells may comprise around 0.01-1.5% CD10+ cells. For example, a suitable population of progenitor cells may comprise approximately 0.5% CD10+ cells.

A suitable population of progenitor cells may comprise around 0.25-3% CD177+ cells. For example, a suitable population of progenitor cells may comprise approximately 1% CD177+ cells.

A suitable population of progenitor cells may comprise around 20-60%, or 40-60% CD62L+ cells. For example, a suitable population of progenitor cells may comprise approximately 46% CD62L+ cells.

A suitable population of progenitor cells may comprise around 1-17% CD54+ cells. For example, a suitable population of progenitor cells may comprise approximately 6% CD54+ cells.

A suitable population of progenitor cells may comprise around 2-20% CD63+ cells. For example, a suitable population of progenitor cells may comprise approximately 5% CD63+ cells.

A suitable population of progenitor cells may comprise around 70-90% CD18+ cells. For example, a suitable population of progenitor cells may comprise approximately 87% CD18+ cells.

The populations of granulopoietic cells or pharmaceutical compositions of the invention may be used in a wide range of therapeutic applications, as disclosed herein. In particular, the populations of cells or pharmaceutical compositions may be used to increase activation or recruitment of host immune cells, and particularly of non-granulocytic immune cells, in a manner that enables amplification of a host therapeutic immune response. This realisation allows such compositions to be used to augment immunotherapeutic treatments in a number of conditions, including (but not limited to) cancer therapies. By amplifying the host immune response, the compositions, medical uses and methods of treatment of the invention are able to render otherwise immunologically “cold” tumours “hot”, and so responsive to treatment.

The amplification that occurs in respect of a host therapeutic immune response is not simply due to the generation of elevated numbers of granulocytes and non-granulocytic immune cells, for example as a result of administration of the compositions of the invention. Instead, the granulopoietic cells and compositions comprising said granulopoietic cells are able to markedly increase activation of non-granulocytic immune cells, and particularly T cells, such as CD8, CD4 and γδ T cells; monocytes; macrophages; dendritic cells (DCs) and NK cells. Meanwhile, the non-granulocytic immune cells are able to markedly increase activation of granulopoietic cells. As discussed in further detail below, and as demonstrated in the Examples, this is able to bring about increased expression of degranulation markers, costimulatory molecules, and cytokines by the activated

The inventors' surprising finding that populations of granulopoietic cells in accordance with the present invention are capable of amplifying (and preferably serve to amplify) the therapeutic immune response of non-granulocytic immune cells also gives rise to further aspects and embodiments of the invention. As used herein, an “immune response” encompasses any response of an immune cell to its environment. Immune cells are constantly responding to their environment, including in vitro, and are therefore constantly generating immune responses even during homeostasis. A “therapeutic immune response” may be an immune response which can contribute to eradication of disease. A therapeutic immune response may include increased activation of an immune cell, increased expression of a cell degranulation marker by an immune cell, increased expression of a costimulatory molecule by an immune cell, or increased expression of a cytokine by an immune cell. Such therapeutic immune responses may occur in vitro or in vivo.

Thus, the inventors have surprisingly shown that granulopoietic cells may be capable of promoting (preferably promote) proliferation and/or survival of non-granulocytic immune cells including NK cells and T cells, thereby allowing increased ex vivo expansion of these cell types and improving their in vivo persistence. The inventors have also shown that granulopoietic cells may be capable of increasing (preferably increase) expression of co-stimulatory molecules including 4-1BB and OX40 on non-granulocytic immune cells such as NK cells and T cells including γδ T cells, thereby improving their therapeutic efficacy. The inventors have also surprisingly found that non-granulocytic immune cells may be capable of increasing (preferably increase) expression of co-stimulatory molecules including CD54 on granulopoietic cells, thereby improving the therapeutic efficacy of the granulopoietic cells.

Compositions comprising granulopoietic cells and non-granulocytic immune cells may therefore be useful for therapy. Such compositions comprise cells with amplified therapeutic immune responses, which in turn, may amplify a host therapeutic immune response e.g. after administration to a subject.

Advantageously, such compositions may assist in successfully eradicating a tumour (e.g. cancer) by providing a combination of immune cells suitable for this purpose. This may be particularly advantageous in cases where a subject's own immune cells may be defective. Furthermore, the present invention may allow for the production of such a composition without conventional manufacturing difficulties and/or without adverse immunogenic effects.

Accordingly, in one aspect, the invention provides a composition comprising a population of granulopoietic cells and a non-granulocytic immune cell. Advantageously, such compositions may assist in successfully eradicating a tumour (e.g. cancer) by providing a combination of immune cells suitable for this purpose. This may be particularly advantageous in cases where a subject's own immune cells may be defective. Furthermore, the present invention may allow for the production of such a composition without conventional manufacturing difficulties and/or without adverse immunogenic effects.

T cells comprising an αβ T cell receptor (also referred to as “αβ T cells”) are generally considered the central cell type involved in coordinating immune responses. However, the inventors have surprisingly shown that granulopoietic cells may amplify therapeutic immune responses of non-granulocytic immune cells in the absence of αβ T cells.

Accordingly, in one aspect, the invention provides a composition comprising a population of granulopoietic cells and a non-granulocytic immune cell, wherein the composition does not comprise an αβ T cell. For example, the composition may comprise a population of granulopoietic cells and a terminally differentiated non-granulocytic immune cell, wherein the composition does not comprise an αβ T cell.

In one aspect, the invention provides a composition comprising a granulopoietic cell and a non-granulocytic immune cell, wherein the granulopoietic cell is capable of modulating (preferably modulates) the therapeutic immune response of the non-granulocytic immune cell.

In one aspect, the invention provides a composition comprising a population of granulopoietic cells and a non-granulocytic immune cell, wherein the population of granulopoietic cells is capable of amplifying (preferably amplifies) the therapeutic immune response of the non-granulocytic immune cell.

In one aspect, the invention provides a kit comprising:
(a) the composition according to the invention; or
(b) a population of granulopoietic cells and a non-granulocytic immune cell (e.g. a terminally differentiated non-granulocytic immune cell); and
(c) optionally instructions for the use of the same (e.g. in treating cancer).

In one aspect, the invention provides a method for manufacturing a composition (e.g. a composition of the invention), the method comprising: culturing PBMCs in the presence of granulopoietic cells, thereby forming the composition; and optionally depleting αβ T cells before, during, or after the culturing.

In one aspect, the invention provides a method for manufacturing a composition (e.g. a composition of the invention), the method comprising: culturing αβ T cell-depleted PBMCs under conditions that promote differentiation of progenitor cells present in the αβ T cell-depleted PBMCs into granulopoietic cells, thereby forming the composition.

In one aspect, the invention provides a composition obtainable by a method of the invention.

In one aspect, the invention provides a composition of the invention for use in a method of treating a disease or disorder in a subject.

In one aspect, the invention provides a composition of the invention for use in medicine.

In one aspect, the invention provides a method of treating a disease or disorder in a subject comprising administering a composition of the invention to the subject.

In one aspect, the invention provides use of a composition of the invention in the manufacture of a medicament.

In one aspect, the invention provides a composition of the invention for use in a method of treating cancer in a subject.

In one aspect, the invention provides a method of treating cancer in a subject comprising administering a composition of the invention to the subject.

In one aspect, the invention provides use of a composition of the invention in the manufacture of a medicament for treating cancer in a subject.

In one aspect, the invention provides a composition of the invention for use in a method of treating an infection in a subject.

In one aspect, the invention provides a method of treating an infection in a subject comprising administering a composition of the invention to the subject.

In one aspect, the invention provides use of a composition of the invention in the manufacture of a medicament for treating an infection in a subject.

In a therapeutic application, the composition may modulate (preferably amplifies) a therapeutic immune response of the subject, such as a non-granulocytic therapeutic immune response of the subject.

In one aspect, the invention provides a composition of the invention, for use to modulate a non-granulocytic therapeutic immune response.

In one aspect, the invention provides a method of treatment comprising modulating a non-granulocytic therapeutic immune response, the method comprising providing a composition of the invention to a subject in need of such treatment.

In one aspect, the invention provides a composition of the invention, for use to amplify a non-granulocytic therapeutic immune response.

In one aspect, the invention provides a method of treatment comprising amplifying a non-granulocytic therapeutic immune response, the method comprising providing a composition of the invention to a subject in need of such treatment.

In one aspect, the invention provides a composition of the invention for use in the manufacture of a medicament for use in modulating a non-granulocytic therapeutic immune response.

In one aspect, the invention provides a composition of the invention for use in the manufacture of a medicament for use in amplifying a non-granulocytic therapeutic immune response.

The present invention is based, to at least some extent, upon the inventors' finding that granulopoietic cells described herein may be capable of amplifying (preferably amplify) the therapeutic immune response of non-granulocytic immune cells. Advantageously, this may allow such granulopoietic cells to be combined with non-granulocytic immune cells to provide a composition which can be used to treat a number of conditions, including (but not limited to) cancer. Said compositions may also be used to augment immunotherapeutic treatments in a number of conditions, including (but not limited to) cancer therapies.

Amplification of an immune response (e.g. a therapeutic immune response) may be demonstrated in vitro by one or more of the following: increased activation of immune cells; increased expression of degranulation markers by immune cells; increased expression of costimulatory molecules by immune cells; increased proliferation by immune cells; increased survival by immune cells; increased abundance of immune cells; increased expression of cytokines by immune cells; increased trafficking by immune cells; increased cytocidal activity by immune cells; and/or increased tumour cell killing activity by immune cells.

Said compositions may also be used to increase activation or recruitment of host immune cells, and particularly of non-granulocytic immune cells, in a manner that enables amplification of a host therapeutic immune response. This realisation may allow such compositions to be used to augment immunotherapeutic treatments in a number of conditions, including (but not limited to) cancer. By amplifying the host immune response, the compositions, medical uses and methods of treatment of the invention may be able to render otherwise immunologically “cold” tumours “hot”, and so responsive to treatment.

In one embodiment, the amplification that occurs in respect of a host therapeutic immune response is not simply due to the generation of elevated numbers of granulocytes and non-granulocytic immune cells e.g. as a result of administration of the compositions of the invention. Instead, the granulopoietic cells and compositions comprising said granulopoietic cells may be able to markedly increase activation of non-granulocytic immune cells, and particularly T cells, such as γδ T cells; monocytes; macrophages; and NK cells. Meanwhile, the non-granulocytic immune cells may be able to markedly increase activation of granulopoietic cells. As discussed in further detail below, and as demonstrated in the Examples, this may be able to bring about increased expression of degranulation markers, costimulatory molecules, and cytokines by the activated granulopoietic and non-granulocytic cells. It may also increase proliferation and survival of activated non-granulocytic cells, leading to increased accumulation of such cells. The inventors have also demonstrated that the activated non-granulocytic immune cells may show an increased degree of recruitment into the TME, as well as increased cytocidal activity (particularly increased tumour cell killing activity).

Surprisingly, the inventors have found that these effects may be achieved using granulopoietic cells and/or non-granulocytic immune cells and compositions comprising said cells that are allogeneic with reference to the subject who will receive the population of granulopoietic cells or composition therapeutically.

These properties suggest that granulopoietic cells, including compositions comprising granulopoietic cells and non-granulocytic immune cells, may be used therapeutically in the treatment of cancer, and that such treatment may also be used to augment other cell-based immunotherapies.

Furthermore, the granulopoietic cells of, or to be used in accordance with, the invention may be capable of differentiating (preferably differentiate) into granulocytes with the ability to kill cancer cells. In this way, compositions and treatments in accordance with the invention may be able to achieve a dual mode of action, both amplifying a non-granulocytic immune response, and giving rise to granulocytes that are able to directly kill cancer cells.

The inventors have demonstrated that granulopoietic cells suitable for use in the compositions or medical uses of the invention, or in the methods of the invention may be capable of amplifying (preferably amplify) immune responses through a number of different mechanisms. In particular, the granulopoietic cells may increase activation of immune cells, and increase activities (such as cell trafficking and cytocidal activity) required to achieve a successful therapeutic immune response.

Populations of granulopoietic cells suitable for use in these aspects of the invention may be characterised in accordance with the definitions offered elsewhere in this specification. Such populations of granulopoietic cells may be produced by the methods of preparing cells for therapeutic use set out earlier in this specification.

Preferably, the composition comprises a population of granulopoietic cells that is capable of amplifying (preferably that amplifies) the therapeutic immune response of the non-granulocytic immune cell. Thus, in one aspect, there is provided a composition comprising a population of granulopoietic cells and a non-granulocytic immune cell (e.g. a terminally differentiated non-granulocytic immune cell), wherein the population of granulopoietic cells is capable of amplifying (preferably amplifies) the therapeutic immune response of the non-granulocytic immune cell.

The ability of a population of granulopoietic cells to amplify a therapeutic immune response of a non-granulocytic immune cell may be determined by any suitable means.

For example, the ability of a population of granulopoietic cells to amplify a therapeutic immune response of a non-granulocytic immune cell may be determined by an in vitro assay. For example, the ability of a population of granulopoietic cells to amplify a therapeutic immune response of a non-granulocytic immune cell may be determined by a method comprising:
(a) admixing PBMCs or PBMCs depleted of αβ T cells in the presence of granulopoietic cells to form an admixture;
(b) incubating the admixture;
(c) determining a therapeutic immune response of a non-granulocytic immune cell present in the admixture after the incubation; and
(d) comparing the therapeutic immune response of the non-granulocytic immune cell present in the admixture after the incubation with a reference standard.

A reference standard may be any suitable control. For example, the reference standard may be the corresponding therapeutic immune response of the non-granulocytic immune cell present in the PBMCs or PBMCs depleted of αβ T cells before the admixing. The reference standard may be the corresponding therapeutic immune response of the non-granulocytic immune cell present in the admixture before the incubation. The reference standard may be the corresponding therapeutic immune response of the non-granulocytic immune cell cultured in the absence of granulopoietic cells but otherwise subjected to identical conditions. The reference standard may be the corresponding therapeutic immune response of the non-granulocytic immune cell cultured in the presence of fewer granulopoietic cells but otherwise subjected to identical conditions.

Such a reference standard may be obtainable using cells from the same donor or a different donor to those used in steps (a)-(c). Preferably, the reference standard is obtainable using cells from the same donor as those used in steps (a)-(c).

The population of granulopoietic cells may be considered capable of amplifying a therapeutic immune response of a non-granulocytic immune cell when a therapeutic immune response of the non-granulocytic immune cell present in the admixture after the incubation is increased compared to the reference standard. Thus, the population of granulopoietic cells may be considered capable of amplifying a therapeutic immune response of a non-granulocytic immune cell when a therapeutic immune response of the non-granulocytic immune cell present in the admixture after the incubation is increased compared to the corresponding therapeutic immune response of the non-granulocytic immune cell present in the PBMCs or PBMCs depleted of αβ T cells before the admixing. Preferably, the population of granulopoietic cells is considered capable of amplifying a therapeutic immune response of a non-granulocytic immune cell when a therapeutic immune response of the non-granulocytic immune cell present in the admixture after the incubation is increased compared to the corresponding therapeutic immune response of the non-granulocytic immune cell cultured in the absence of the granulopoietic cells but otherwise subjected to identical conditions. The population of granulopoietic cells may be considered capable of amplifying a therapeutic immune response of a non-granulocytic immune cell when a therapeutic immune response of the non-granulocytic immune cell present in the admixture after the incubation is increased compared to the corresponding therapeutic immune response of the non-granulocytic immune cell cultured in the presence of fewer granulopoietic cells but otherwise subjected to identical conditions.

Preferably, the ability of a population of granulopoietic cells to amplify a therapeutic immune response of a non-granulocytic immune cell is determined by a method comprising:
(a) admixing PBMCs or PBMCs depleted of αβ T cells in the presence of granulopoietic cells to form an admixture;
(b) incubating the admixture;
(c) determining a therapeutic immune response of a non-granulocytic immune cell present in the admixture after the incubation; and
(d) comparing the therapeutic immune response of the non-granulocytic immune cell present in the admixture after the incubation with the corresponding therapeutic immune response of the non-granulocytic immune cell cultured in the absence of the granulopoietic cells but otherwise subjected to identical conditions.

The granulopoietic cells and PBMCs or αβ T cell-depleted PBMCs may be admixed together at any suitable ratio. The granulopoietic cells and PBMCs or αβ T cell-depleted PBMCs may be admixed together at a ratio of 100:1 to 0.01:1 granulopoietic cells to PBMCs or αβ T cell-depleted PBMCs. The population of granulopoietic cells and PBMCs or αβ T cell-depleted

PBMCs may be admixed together at a ratio of 100:1 to 0.01:1; 75:1 to 0.05:1; 50:1 to 0.1:1; 25:1 to 0.2:1; 10:1 to 0.25:1; 5:1 to 0.25:1; 3:1 to 0.25:1; or 2:1 to 0.5:1 granulopoietic cells to PBMCs or αβ T cell-depleted PBMCs. Preferably, the granulopoietic cells and PBMCs are admixed together at a ratio of 3:1 to 0.25:1 granulopoietic cells to PBMCs or αβ T cell-depleted PBMCs.

The granulopoietic cells and PBMCs or αβ T cell-depleted PBMCs may be admixed together at a ratio of less than or equal to 100:1, 75:1, 50:1, 25:1, 10:1, 5:1, 3:1, 2:1, 1:1, 0.5:1, 0.25:1, 0.1:1, 0.05:1 or 0.01:1 granulopoietic cells to PBMCs or αβ T cell-depleted PBMCs. The granulopoietic cells and PBMCs or αβ T cell-depleted PBMCs may be admixed together at a ratio of at least 0.01:1, 0.05:1 0.1:1, 0.25:1, 0.5:1, 1:1, 2:1, 3:1, 5:1, 10:1, 25:1, 50:1, 75:1, or 100:1 granulopoietic cells to PBMCs or αβ T cell-depleted PBMCs. The granulopoietic cells and PBMCs or αβ T cell-depleted PBMCs may be admixed together at a ratio of 100:1, 75:1, 50:1, 25:1, 10:1, 5:1, 3:1, 2:1, 1:1, 0.5:1, 0.25:1, 0.1:1, 0.05:1 or 0.01:1 granulopoietic cells to PBMCs or αβ T cell-depleted PBMCs. Preferably, the granulopoietic cells and PBMCs or αβ T cell-depleted PBMCs are admixed together at a ratio of 2:1, 1:1, or 0.5:1 granulopoietic cells to PBMCs or αβ T cell-depleted PBMCs. For example, the granulopoietic cells and PBMCs or αβ T cell-depleted PBMCs may be admixed together at a ratio of 2:1 granulopoietic cells to PBMCs or αβ T cell-depleted PBMCs. The granulopoietic cells and PBMCs or αβ T cell-depleted PBMCs may be admixed together at a ratio of 1:1 granulopoietic cells to PBMCs or αβ T cell-depleted PBMCs. The granulopoietic cells and PBMCs or αβ T cell-depleted PBMCs may be admixed together at a ratio of 0.5:1 granulopoietic cells to PBMCs or αβ T cell-depleted PBMCs.

The admixture may be incubated for any suitable time. For example, the admixture may be incubated for 1-240 hours. The admixture may be incubated for 1-240 hours; 2-220 hours; 4-200 hours; 8-180 hours; 12-160 hours; 16-140 hours; 20-120 hours; 24-100 hours; 24-96 hours; 48-96 hours; or 48-72 hours. Preferably, the admixture is incubated for 48-96 hours.

The admixture may be incubated for 1, 2, 4, 8, 12, 16, 20, 24, 30, 36, 42, 48, 54, 60, 66, 72, 78, 84, 90, 96, 102, 108, 114, 120, 144, 168, 192, 216, or 240 hours. Preferably, the admixture is incubated for 72 hours.

Accordingly, the ability of a population of granulopoietic cells to amplify a therapeutic immune response of a non-granulocytic immune cell may be determined by a method comprising:
(a) admixing PBMCs or PBMCs depleted of αβ T cells in the presence of granulopoietic cells to form an admixture, wherein the admixture comprises 2:1, 1:1, or 0.5:1 granulopoietic cells to PBMCs or PBMCs depleted of αβ T cells;
(b) incubating the admixture for 72 hours;
(c) determining a therapeutic immune response of a non-granulocytic immune cell present in the admixture after the incubation; and
(d) comparing the therapeutic immune response of the non-granulocytic immune cell present in the admixture after the incubation with a reference standard.

Preferably, the ability of a population of granulopoietic cells to amplify a therapeutic immune response of a non-granulocytic immune cell may be determined by a method comprising:
(a) admixing PBMCs or PBMCs depleted of αβ T cells in the presence of granulopoietic cells to form an admixture, wherein the admixture comprises 2:1, 1:1, or 0.5:1 granulopoietic cells to PBMCs or PBMCs depleted of αβ T cells;
(b) incubating the admixture for 72 hours;
(c) determining a therapeutic immune response of a non-granulocytic immune cell present in the admixture after the incubation; and
(d) comparing the therapeutic immune response of the non-granulocytic immune cell present in the admixture after the incubation with the corresponding therapeutic immune response of the non-granulocytic immune cell cultured in the absence of the granulopoietic cells but otherwise subjected to identical conditions.

The admixture may additionally comprise a CD3 activating agent, such as OKT3.

A therapeutic immune response of a non-granulocytic immune cell may be determined by any suitable means. For example, a therapeutic immune response of a non-granulocytic immune cell may be determined by measuring cell surface markers present on the non-granulocytic immune cell, e.g. using flow cytometry. A therapeutic immune response of a non-granulocytic immune cell may be determined by measuring the level of an activation marker; the level of a degranulation marker; and/or the level of a co-stimulatory marker present on the non-granulocytic immune cell using flow cytometry. A therapeutic immune response of a non-granulocytic immune cell may be determined by measuring proliferation and/or survival of the non-granulocytic immune cell e.g. using flow cytometry.

Accordingly, a therapeutic immune response of a non-granulocytic immune cell may be determined by a method comprising:
(a) washing the non-granulocytic immune cells;
(b) incubating the cells with live/dead stain;
(c) washing the cells in flow cytometry buffer and surface staining the cells with antibodies for measuring the number of non-granulocytic immune cells present; the level of an activation marker; the level of a degranulation marker; and/or the level of a co-stimulatory marker present on the non-granulocytic immune cells;
(d) fixing the cells; and
(e) analysing the cells using a flow cytometer.

The population of granulopoietic cells may be considered capable of amplifying a therapeutic immune response of a non-granulocytic immune cell when the number of non-granulocytic immune cells present; the level of an activation marker; the level of a degranulation marker; and/or the level of a co-stimulatory marker present on the non-granulocytic immune cells is increased compared to a reference standard.

Preferably, a therapeutic immune response of a non-granulocytic immune cell may be determined by a method comprising:
(a) washing the non-granulocytic immune cells (e.g. present in the admixture after the incubation, or present in PBMCs or PBMCs depleted of αβ T cells) in PBS;
(b) incubating the cells with live/dead stain (Fixable Viability Dye eFluor 780; 1:500 dilution) and FcγR block (Human TruStain FcX; 1:50 dilution) for 20 minutes;
(c) washing the cells in flow cytometry buffer and surface staining the cells with antibodies specific for CD3 (OKT3), CD4 (RPA-T4), CD8 (RPA-T8), CD56 (HCD56), CD107a (H4A3), 4-1BB (4B4-1) and/or (preferably and) OX40 (Ber-ACT35), wherein the antibodies are used at 1:50 dilution, with staining performed in 50 μl/sample;
(d) fixing the cells using 100 μl 1× BD CellFix; and
(e) analysing the cells using a flow cytometer (e.g. a MACSQuant 16 (Miltenyi)).

The population of granulopoietic cells may be considered capable of amplifying a therapeutic immune response of a non-granulocytic immune cell when the expression level of CD3 (OKT3), CD4 (RPA-T4), CD8 (RPA-T8), CD56 (HCD56), CD107a (H4A3), 4-1BB (4B4-1) and/or (preferably and) OX40 present on the non-granulocytic immune cells is increased compared to a reference standard.

The data may be analysed using any suitable software, preferably FlowLogic software. The stained cell populations are preferably analysed by gating on single, live cells.

A therapeutic immune response of a non-granulocytic immune cell may be determined by measuring cytokine production by the non-granulocytic immune cell. For example, a therapeutic immune response of a non-granulocytic immune cell may be determined by measuring cytokine production by the non-granulocytic immune cell using ELISA.

Accordingly, a therapeutic immune response of a non-granulocytic immune cell may be determined by a method comprising:
(a) measuring the concentration of a cytokine present in the cell culture supernatant of the non-granulocytic immune cells using an ELISA; and/or
(b) measuring the concentration of a cytokine present in the cell culture supernatant of the non-granulocytic immune cells using LEGENDplex.

The population of granulopoietic cells may be considered capable of amplifying a therapeutic immune response of a non-granulocytic immune cell when the concentration of a cytokine present in the cell culture supernatant of the non-granulocytic immune cells is increased compared to a reference standard.

Preferably, a therapeutic immune response of a non-granulocytic immune cell may be determined by a method comprising:
(a) measuring the concentration of secreted IFN-γ present in the cell culture supernatant of the non-granulocytic immune cells (e.g. present in the admixture after the incubation, or present in PBMCs or PBMCs depleted of αβ T cells) using a quantitative sandwich ELISA (e.g. Abcam; ab174443) according to the manufacturer's instructions; and/or
(b) measuring the concentration of CXCL10 present in the cell culture supernatant of the non-granulocytic immune cells (e.g. present in the admixture after the incubation, or present in PBMCs or PBMCs depleted of αβ T cells) using LEGENDplex (e.g. BioLegend; 740985) according to the manufacturer's instructions.

The population of granulopoietic cells may be considered capable of amplifying a therapeutic immune response of a non-granulocytic immune cell when the concentration of a IFN-γ and/or CXCL10 (preferably and) present in the cell culture supernatant of the non-granulocytic immune cells is increased compared to a reference standard.

A therapeutic immune response of a non-granulocytic immune cell may be determined by measuring cell surface markers present on the non-granulocytic immune cell and/or by measuring cytokine production by the non-granulocytic immune cell. Preferably, a therapeutic immune response of non-granulocytic immune cells is determined by a method comprising:
(a) (i) washing the non-granulocytic immune cells (e.g. present in the admixture after the incubation, or present in PBMCs or PBMCs depleted of αβ T cells) in PBS;
   (ii) incubating the cells with live/dead stain (Fixable Viability Dye eFluor 780; 1:500 dilution) and FcγR block (Human TruStain FcX; 1:50 dilution) for 20 minutes;
   (iii) washing the cells in flow cytometry buffer and surface staining the cells with antibodies specific for CD3 (OKT3), CD4 (RPA-T4), CD8 (RPA-T8), CD56 (HCD56), CD107a (H4A3), 4-1BB (4B4-1) and/or (preferably and) OX40 (Ber-ACT35), wherein the antibodies are used at 1:50 dilution, with staining performed in 50 μl/sample;
   (iv) fixing the cells using 100 μl 1× BD CellFix; and
   (v) analysing the cells using a flow cytometer (e.g. a MACSQuant 16 (Miltenyi)); and/or
(b) (i) measuring the concentration of secreted IFN-γ present in the cell culture supernatant of the non-granulocytic immune cells (e.g. present in the admixture after the incubation, or present in PBMCs or PBMCs depleted of αβ T cells) using a quantitative sandwich ELISA (e.g. Abcam; ab174443) according to the manufacturer's instructions; and/or
   (ii) measuring the concentration of CXCL10 present in the cell culture supernatant of the non-granulocytic immune cells (e.g. present in the admixture after the incubation, or present in PBMCs or PBMCs depleted of αβ T cells) using LEGENDplex (e.g. BioLegend; 740985) according to the manufacturer's instructions.

A therapeutic immune response of a non-granulocytic immune cell may be determined by measuring tumour killing of the non-granulocytic immune cell, e.g. as determined by a method described herein. The population of granulopoietic cells may be considered capable of amplifying a therapeutic immune response of a non-granulocytic immune cell when the level of tumour killing of the non-granulocytic immune cell is increased compared to the reference standard.

The ability of a population of granulopoietic cells to amplify a therapeutic immune response of a non-granulocytic immune cell may be determined by a method comprising:
(a) admixing PBMCs or PBMCs depleted of αβ T cells in the presence of granulopoietic cells to form an admixture, wherein the admixture comprises 2:1, 1:1, or 0.5:1 granulopoietic cells to PBMCs or PBMCs depleted of αβ T cells;
(b) incubating the admixture for 72 hours;
(c) determining a therapeutic immune response of a non-granulocytic immune cell present in the admixture after the incubation by a method comprising:
   (i) washing the non-granulocytic immune cells (e.g. present in the admixture after the incubation, or present in PBMCs or PBMCs depleted of αβ T cells) in PBS;
   (ii) incubating the cells with live/dead stain (Fixable Viability Dye eFluor 780; 1:500 dilution) and FcγR block (Human TruStain FcX; 1:50 dilution) for 20 minutes;
   (iii) washing the cells in flow cytometry buffer and surface staining the cells with antibodies specific for CD3 (OKT3), CD4 (RPA-T4), CD8 (RPA-T8), CD56 (HCD56), CD107a (H4A3), 4-1BB (4B4-1) and/or (preferably and) OX40 (Ber-ACT35), wherein the antibodies are used at 1:50 dilution, with staining performed in 50 μl/sample;
   (iv) fixing the cells using 100 μl 1× BD CellFix; and
   (v) analysing the cells using a flow cytometer (e.g. a MACSQuant 16 (Miltenyi)); and/or determining a therapeutic immune response of a non-granulocytic immune cell present in the admixture after the incubation by a method comprising:
   (i) measuring the concentration of secreted IFN-γ present in the cell culture supernatant of the non-granulocytic immune cells (e.g. present in the admixture after the incubation, or present in PBMCs or PBMCs depleted of αβ T cells) using a quantitative sandwich ELISA (e.g. Abcam; ab174443) according to the manufacturer's instructions; and/or
   (ii) measuring the concentration of CXCL10 present in the cell culture supernatant of the non-granulocytic immune cells (e.g. present in the admixture after the incubation, or present in PBMCs or PBMCs depleted of αβ T cells) using LEGENDplex (e.g. BioLegend; 740985) according to the manufacturer's instructions; and
(d) comparing the therapeutic immune response of the non-granulocytic immune cell present in the admixture after the incubation with a reference standard.

Preferably, the ability of a population of granulopoietic cells to amplify a therapeutic immune response of a non-granulocytic immune cell may be determined by a method comprising:
(a) admixing PBMCs or PBMCs depleted of αβ T cells in the presence of granulopoietic cells to form an admixture, wherein the admixture comprises 2:1, 1:1, or 0.5:1 granulopoietic cells to PBMCs or PBMCs depleted of αβ T cells;
(b) incubating the admixture for 72 hours;
(c) determining a therapeutic immune response of a non-granulocytic immune cell present in the admixture after the incubation by a method comprising:
   (i) washing the non-granulocytic immune cells (e.g. present in the admixture after the incubation, or present in PBMCs or PBMCs depleted of αβ T cells) in PBS;
   (ii) incubating the cells with live/dead stain (Fixable Viability Dye eFluor 780; 1:500 dilution) and FcγR block (Human TruStain FcX; 1:50 dilution) for 20 minutes;
   (iii) washing the cells in flow cytometry buffer and surface staining the cells with antibodies specific for CD3 (OKT3), CD4 (RPA-T4), CD8 (RPA-T8), CD56 (HCD56), CD107a (H4A3), 4-1BB (4B4-1) and/or (preferably and) OX40 (Ber-ACT35), wherein the antibodies are used at 1:50 dilution, with staining performed in 50 μl/sample;
   (iv) fixing the cells using 100 μl 1× BD CellFix; and
   (v) analysing the cells using a flow cytometer (e.g. a MACSQuant 16 (Miltenyi)); and/or determining a therapeutic immune response of a non-granulocytic immune cell present in the admixture after the incubation by a method comprising:
   (i) measuring the concentration of secreted IFN-γ present in the cell culture supernatant of the non-granulocytic immune cells (e.g. present in the admixture after the incubation, or present in PBMCs or PBMCs depleted of αβ T cells) using a quantitative sandwich ELISA (e.g. Abcam; ab174443) according to the manufacturer's instructions; and/or
   (ii) measuring the concentration of CXCL10 present in the cell culture supernatant of the non-granulocytic immune cells (e.g. present in the admixture after the incubation, or present in PBMCs or PBMCs depleted of αβ T cells) using LEGENDplex (e.g. BioLegend; 740985) according to the manufacturer's instructions; and
(d) comparing the therapeutic immune response of the non-granulocytic immune cell present in the admixture after the incubation with the corresponding therapeutic immune response of the non-granulocytic immune cell cultured in the absence of the granulopoietic cells but otherwise subjected to identical conditions.

The ability of a population of granulopoietic cells to increase a plurality of therapeutic immune responses in a non-granulocytic immune cell may indicate that the population of granulopoietic cells is particularly suitable for inclusion in a composition of the invention. Accordingly, the composition may comprise a population of granulopoietic cells which is capable of amplifying (preferably amplifies) the level of CD3, CD4, CD8, CD56, CD107a, 4-1BB and OX40 in the non-granulocytic immune cell compared to the reference standard. The composition may comprise a population of granulopoietic cells which is capable of amplifying (preferably amplifies) the level of CD107a, 4-1BB and OX40 in the non-granulocytic immune cell compared to the reference standard. The composition may comprise a population of granulopoietic cells which is capable of amplifying (preferably amplifies) the level of IFN-γ and CXCL10 in the non-granulocytic immune cell e.g. compared to the reference standard.

The inventors have surprisingly found that a population of granulopoietic cells which may be capable of amplifying (preferably amplifies) a therapeutic immune response of one type of non-granulocytic immune cell may also be capable of amplifying (preferably amplifies) a therapeutic immune response of a different type of non-granulocytic immune cell. Accordingly, a population of granulopoietic cells may be considered capable of amplifying the therapeutic immune response of a non-granulocytic immune cell if the population of granulopoietic cells is capable of amplifying the therapeutic immune response of an NK cell and/or a T cell e.g. as determined using a method described herein. Preferably, a population of granulopoietic cells is considered capable of amplifying the therapeutic immune response of a non-granulocytic immune cell if the population of granulopoietic cells is capable of amplifying the therapeutic immune response of an NK cell e.g. as determined using a method described herein.

The inventors have also shown that granulopoietic cells which are capable of amplifying (preferably amplifies) a particular therapeutic immune response may also be capable of amplifying (preferably amplifies) a different type of therapeutic immune response. For example, a population of granulopoietic cells which is capable of increasing (preferably increases) cell activation may also be capable of increasing (preferably increases) expression of degranulation markers. Thus, a population of granulopoietic cells may be considered capable of amplifying the therapeutic immune response of a non-granulocytic immune cell if the population of granulopoietic cells is capable of increasing (preferably increases) NK cell activation; increasing expression of NK cell degranulation markers; increasing expression of NK cell costimulatory molecules; increasing NK cell proliferation; increasing NK cell survival; increasing expression of cytokines by NK cells; increasing NK cell cytocidal activity; and/or increasing tumour cell killing activity of NK cells. Preferably, a population of granulopoietic cells is considered capable of amplifying the therapeutic immune response of a non-granulocytic immune cell if the population of granulopoietic cells is capable of increasing (preferably increases) the level of CD107a, 4-1BB and/or (preferably and) OX40 in an NK cell e.g. as determined using a method described herein.

A population of granulopoietic cells suitable for use in accordance with the various aspects of the present invention may be able to increase activation of immune cells e.g. non-granulocytic immune cells. In particular, a population of granulopoietic cells may be capable of increasing (preferably increases) activation of the non-granulocytic immune cell present in a composition of the invention. Accordingly, a population of granulopoietic cells may be capable of amplifying a therapeutic immune response of a non-granulocytic immune cell by increasing activation of the non-granulocytic immune cell.

Suitably a population of granulopoietic cells suitable for use in the present invention may increase activation of immune cells (e.g. non-granulocytic immune cells) such that expression by the immune cells of one or more markers of degranulation is increased. Suitably a population of granulopoietic cells suitable for use in the present invention may increase activation of immune cells (e.g. non-granulocytic immune cells) such that expression by the immune cells of one or more costimulatory molecules is increased. Suitably a population of granulopoietic cells suitable for use in the present invention may increase activation of immune cells (e.g. non-granulocytic immune cells) such that proliferation of the immune cells is increased. Suitably a population of granulopoietic cells suitable for use in the present invention may increase activation of immune cells (e.g. non-granulocytic immune cells) such that abundance of the immune cells is increased. Suitably a population of granulopoietic cells suitable for use in the present invention may increase activation of immune cells (e.g. non-granulocytic immune cells) such that survival of the immune cells is increased. Suitably a population of granulopoietic cells suitable for use in the present invention may increase activation of immune cells (e.g. non-granulocytic immune cells) such that expression by the immune cells of one or more cytokines is increased. Suitably a population of granulopoietic cells suitable for use in the present invention may increase activation of immune cells (e.g. non-granulocytic immune cells) such that trafficking of the immune cells is increased. Suitably a population of granulopoietic cells suitable for use in the present invention may increase activation of immune cells (e.g. non-granulocytic immune cells) such that cytocidal activity of the immune cells is increased.

The term “one or more” as used herein may mean at least 2, 3, 4, 5, 6, 7, 8, 9, 10, 15, or 20. In one embodiment, wherein “one or more” precedes a list, “one or more” may mean all of the members of the list. Similarly, the term “at least one” as used herein may mean at least 2, 3, 4, 5, 6, 7, 8, 9, 10, 15, or 20. In one embodiment, wherein “at least one” precedes a list, “at least one” may mean all of the members of the list.

Suitably a population of granulopoietic cells suitable for use in accordance with the present invention may increase activation of immune cells (e.g. non-granulocytic immune cells) by “signal 2” (co-stimulation). Alternatively, or additionally, a population of granulopoietic cells suitable for use in accordance with the present invention may increase activation of immune cells (e.g. non-granulocytic immune cells) by “signal 3” (cytokine stimulation). A population of granulopoietic cells suitable for use in accordance with the present invention may have the capacity to increase activation of immune cells (e.g. non-granulocytic immune cells) by both signal 2 and signal 3.

It is known that signal 2 and signal 3 are both important in generating effective immune responses to tumours, and in overcoming the immunosuppressive effects of the TME. Accordingly, the inventors' data (set out in the Examples) illustrating that granulopoietic cells suitable for use in accordance with the invention may be able to provide these signals may provide a clear indication of their suitability for use in amplifying therapeutic immune responses that may be relevant in the treatment of cancer.

Other useful ways in which relevant populations of granulopoietic cells may be defined are set out below.

Granulopoietic cells suitable for use in the compositions, medical uses and methods of the invention may be defined with reference to their potency. The population of granulopoietic cells may comprise multipotent cells. In a suitable embodiment, the population of granulopoietic cells may comprise unipotent cells.

Suitable populations of granulopoietic cells for use in the various aspects of the invention may be defined with reference to their differentiation state within the granulopoiesis pathway. In a suitable embodiment, the population of granulopoietic cells has a differentiation stage corresponding to that between a myeloblast and a granulocyte. Suitably the population of granulopoietic cells has a differentiation stage corresponding to that between a myeloblast and a band cell. For example, the population of granulopoietic cells may have a differentiation stage corresponding to that between a myeloblast and a metamyelocyte. Suitably the population of granulopoietic cells has a differentiation stage corresponding to that between a myeloblast and a myelocyte. Suitably the population of granulopoietic cells has a differentiation stage corresponding to that between a myeloblast and a promyelocyte.

In a suitable embodiment, the population of granulopoietic cells has a differentiation stage corresponding to a myeloblast. In a suitable embodiment, the population of granulopoietic cells has a differentiation stage corresponding to a promyelocyte. In a suitable embodiment, the population of granulopoietic cells has a differentiation stage corresponding to a myelocyte. In a suitable embodiment, the population of granulopoietic cells has a differentiation stage corresponding to a metamyelocyte. In a suitable embodiment, the population of granulopoietic cells has a differentiation stage corresponding to a band cell.

In a suitable embodiment, the population of granulopoietic cells has a differentiation stage corresponding to a granulocyte.

As set out elsewhere in the specification, populations of granulopoietic cells suitable for use in the various aspects of the invention may be derived from artificial stem cells, such as iPSCs. It will be appreciated that such populations of granulopoietic cells may not be identical with naturally occurring cells of the granulopoietic pathway, but may share structural (e.g. marker expression) or functional (e.g. potency) characteristics with such naturally occurring cells. The reference to cells having differentiation stages “corresponding to” named cell types in this disclosure should be interpreted accordingly.

Suitably the population of granulopoietic cells is selected from the group comprising (or consisting of): a myeloblast; a promyelocyte; a myelocyte; a metamyelocyte; a band cell; and a granulocyte. Suitably the population of granulopoietic cells is selected from the group comprising (or consisting of): a myeloblast; a promyelocyte; a myelocyte; a metamyelocyte; and a band cell. Suitably the population of granulopoietic cells is selected from the group comprising (or consisting of): a myeloblast; a promyelocyte; a myelocyte; and a metamyelocyte. Suitably the population of granulopoietic cells is selected from the group comprising (or consisting of): a myeloblast; a promyelocyte; and a myelocyte. Suitably the population of granulopoietic cells is selected from the group comprising (or consisting of): a myeloblast; and a promyelocyte.

In a suitable embodiment, the population of granulopoietic cells is a myeloblast. In a suitable embodiment, the population of granulopoietic cells is a promyelocyte. In a suitable embodiment, the population of granulopoietic cells is a myelocyte. In a suitable embodiment, the population of granulopoietic cells is a metamyelocyte. In a suitable embodiment, the population of granulopoietic cells is a band cell. In a suitable embodiment, the population of granulopoietic cells is a granulocyte.

Suitably the population of granulopoietic cells may be committed to the neutrophil lineage. In such an embodiment a suitable population of granulopoietic cells may comprise or consist of cells selected from the group comprising (or consisting) of: a neutrophilic promyelocyte; a neutrophilic myelocyte; a neutrophilic metamyelocyte; a neutrophilic band cell; and a neutrophil.

As set out further elsewhere in this specification, populations of granulopoietic cells that may be employed in the various aspects of the invention may also be defined with reference to the granulocytes that they are able to give rise to on differentiation. Suitable examples of populations of granulopoietic cells may be able to give rise to granulocytes that have the ability to kill cancer cells and/or the ability to kill infective agents or cells infected by infective agents. Alternatively, or additionally, suitable populations of granulopoietic cells may be able to give rise to granulocytes that have desirable expression profiles of molecules such as chemokines or costimulatory receptor ligands.

The inventors have surprisingly shown that populations of granulopoietic cells cultured in the presence of non-granulocytic immune cells may have an amplified therapeutic immune response. Accordingly, populations of granulopoietic cells suitable for use in the compositions, medical uses and methods of the invention may be characterised by having an amplified therapeutic immune response. For example, populations of granulopoietic cells suitable for use in the compositions, medical uses and methods of the invention may be characterised by one or more of the following: increased activation; increased expression of degranulation markers; increased expression of costimulatory molecules; increased proliferation; increased survival; increased expression of cytokines; increased cytocidal activity; or increased tumour cell killing activity e.g. compared to the corresponding therapeutic immune response of the non-granulocytic immune cell cultured in the absence of granulopoietic cells as described herein; or compared to a reference standard, as determined by a method described herein.

Preferably, the composition comprises a population of granulopoietic cells characterised by one or more of the following: increased activation; increased expression of degranulation markers; increased expression of costimulatory molecules; increased proliferation; increased survival; increased expression of cytokines; increased cytocidal activity; or increased tumour cell killing activity e.g. compared to the corresponding therapeutic immune response of the population of granulopoietic cells cultured in the absence of a non-granulocytic immune cell as described herein; or compared to a reference standard, as determined by a method described herein.

A population of granulopoietic cells having an amplified therapeutic immune response may be a population of granulopoietic cells having increased activation. Accordingly, the composition may comprise a population of granulopoietic cells having increased activation. Increased activation of populations of granulopoietic cells may be associated with increased expression of one or more markers selected from the group comprising (or consisting) of: CD54, CD40, CD11b, and Mac1. The compositions of the invention may therefore comprise a population of granulopoietic cells having increased expression of CD54, CD40, CD11b, and/or Mac1, e.g. compared to a population of granulopoietic cells not cultured in the presence of a non-granulocytic immune cell but otherwise subjected to identical conditions. The compositions of the invention may comprise a population of granulopoietic cells having increased expression of CD40, e.g. compared to a population of granulopoietic cells not cultured in the presence of a non-granulocytic immune cell but otherwise subjected to identical conditions. The compositions of the invention may comprise a population of granulopoietic cells having increased expression of CD11b, e.g. compared to a population of granulopoietic cells not cultured in the presence of a non-granulocytic immune cell but otherwise subjected to identical conditions. The compositions of the invention may comprise a population of granulopoietic cells having increased expression of Mac1, e.g. compared to a population of granulopoietic cells not cultured in the presence of a non-granulocytic immune cell but otherwise subjected to identical conditions. Preferably, the compositions of the invention comprise a population of granulopoietic cells having increased expression of CD54, e.g. compared to a population of granulopoietic cells not cultured in the presence of a non-granulocytic immune cell but otherwise subjected to identical conditions.

Activation (e.g. as determined by CD54 expression) of such populations of granulopoietic cells may be increased by at least 5%. For example, activation of populations of granulopoietic cells may be increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, or more. Quantification of the increase in activation of populations of granulopoietic cells in accordance with such an embodiment may make use of comparison to an appropriate control, e.g. a population of granulopoietic cells not cultured in the presence of a non-granulocytic immune cell but otherwise subjected to identical conditions.

The compositions of the invention may comprise a population of granulopoietic cells having increased expression of CD54; a population of granulopoietic cells having increased expression of CD40; a population of granulopoietic cells having increased expression of CD11b; and/or a population of granulopoietic cells having increased expression of Mac1, e.g. compared to a population of granulopoietic cells not cultured in the presence of a non-granulocytic immune cell but otherwise subjected to identical conditions. In some embodiments, at least about 20%, 25%, 30%, 35%, 40%, 45%, 50%, or 55% of the populations of granulopoietic cells in the composition express CD54, e.g. as determined by flow cytometry. Preferably at least about 45% of the populations of granulopoietic cells in the composition express CD54, e.g. as determined by flow cytometry.

The population of granulopoietic cells may be obtainable from any suitable source. For example, the population of granulopoietic cells may be obtainable from a sample of PBMCs or a sample of umbilical cord blood. The sample of PBMCs or sample of umbilical cord blood may be obtainable (e.g. obtained) from a donor. Preferably, the population of granulopoietic cells is obtainable (e.g. obtained) from a sample of αβ T cell-depleted PBMCs. The population of granulopoietic cells may be obtainable from (e.g. differentiated in vitro from) a stem cell, such as a haematopoietic stem cell or iPSC.

The term “obtainable” as used herein encompasses the term “obtained”. In one embodiment, “obtainable” means obtained.

The term “donor” as used herein refers to a subject (suitably a human subject) from whom a sample is obtainable (e.g. obtained). Any suitable sample from which a population of granulopoietic cells and/or non-granulocytic immune cell is obtainable may be obtainable from the donor. The donor may be selected based on one or more of the following characteristics: sex, age, medical history, and/or blood group type. A donor may be selected if said donor is a healthy donor. A donor may be selected if said donor does not have cancer and does not have an infection. For example, a donor may be selected if said donor does not have cancer. A donor may be selected if said donor does not have an infection. A donor may be selected if said donor is a male. A donor may be selected if said donor is aged 18-55 and preferably 18-35 (more preferably 18-24). Suitably, a donor may be selected if said donor is a male aged between 18-55 and preferably 18-35 (more preferably 18-24). In another embodiment a donor may be selected if said donor is a female. A donor may be selected if said donor is above the age of 40. Suitably, a donor may be selected if said donor is a female above the age of 40.

The population of granulopoietic cells may be obtainable from a haematopoietic cell. The term “haematopoietic cell” as used herein refers to a cell that is capable of differentiating (preferably differentiates) into a granulopoietic cell or a population of granulopoietic cells. The term “haematopoietic cell” thus encompasses a haematopoietic stem cell, as well as a precursor cell (e.g. differentiated from a haematopoietic stem cell), wherein said precursor cell is capable of differentiating (preferably differentiates) into a granulopoietic cell or a population of granulopoietic cells. The precursor cell may be referred to herein as a “granulopoietic precursor cell”. Suitable examples of such precursor cells have been defined elsewhere in the specification in the context of the production of populations of granulopoietic cells. A haematopoietic cell in accordance with the present invention may relate to a haematopoietic stem cell, a granulopoietic precursor cell or combinations thereof. In one embodiment, a haematopoietic cell is a cell of the haematopoiesis pathway or a cell equivalent thereto. In one embodiment, the haematopoietic cell is an induced pluripotent stem cell (iPSC) or a cell equivalent thereto. A stem cell may be obtainable from umbilical cord blood.

In one embodiment, an iPSC is obtainable from a somatic cell of a donor. Generation of iPSCs is a well-known technique in the art, see Yu et al (2007), Science, 318:1917-1920 the teaching of which is incorporated herein by reference. Accordingly, the population of granulopoietic cells may be obtainable from an induced pluripotent stem cell (iPSC) or haematopoietic stem cell (HSC). Preferably, the population of granulopoietic cells is obtainable from an HSC. The population of granulopoietic cells may be obtainable (e.g. obtained) by a method of obtaining a population of granulopoietic cells described herein.

The granulopoietic cells obtainable (e.g. obtained) by the above methods are capable of amplifying (preferably amplify) the therapeutic immune response of non-granulocytic immune cells. The granulopoietic cells obtainable (e.g. obtained) by the above methods amplify the therapeutic immune response of non-granulocytic immune cells.

The population of granulopoietic cells present in a composition of the invention may be a heterogeneous population of granulopoietic cells, i.e. comprising a plurality of different types or subtypes of granulopoietic cells, or it may be a homogeneous population of granulopoietic cells, i.e. comprising a single type of granulopoietic cell. Preferably, the population of granulopoietic cells is a heterogeneous population of granulopoietic cells.

As used herein, the term “non-granulocytic immune cell” refers to any cell of the immune system, other than a granulocytic cell (e.g. other than a granulocyte). Accordingly, the non-granulocytic immune cell may be any immune cell other than a neutrophil, an eosinophil, or a basophil.

The non-granulocytic immune cell may be a dendritic cell, a blood-derived myeloid cell, a monocyte, a macrophage, a natural killer (NK) cell, a B cell, or a T cell e.g. a γδ T cell. Preferably, the non-granulocytic immune cell is a dendritic cell, a blood-derived myeloid cell, a monocyte, a macrophage, an NK cell, a B cell or a γδ T cell. Particularly preferably, the non-granulocytic immune cell is a γδ T cell (e.g. a Vδ1⁺ or Vδ2⁺ γδ T cell) or an NK cell.

Non-granulocytic immune cells suitable for use in the compositions, medical uses and methods of the invention may be characterised by having an amplified therapeutic immune response. For example, non-granulocytic immune cells suitable for use in the compositions, medical uses and methods of the invention may be characterised by one or more of the following: increased activation; increased expression of degranulation markers; increased expression of costimulatory molecules; increased proliferation; increased survival; increased expression of cytokines; increased cytocidal activity; or increased tumour cell killing activity e.g. compared to the corresponding therapeutic immune response of the non-granulocytic immune cell cultured in the absence of granulopoietic cells; or compared to a reference standard. Preferably, the composition comprises a non-granulocytic immune cell characterised by one or more of the following: increased activation; increased expression of degranulation markers; increased expression of costimulatory molecules; increased proliferation; increased survival; increased expression of cytokines; increased cytocidal activity; or increased tumour cell killing activity e.g. compared to the corresponding therapeutic immune response of the non-granulocytic immune cell cultured in the absence of a population of granulopoietic cells; or compared to a reference standard.

For example, the non-granulocytic immune cell may have increased expression of one or more markers selected from: CD3, CD4, CD8, CD56, CD107a, 4-1BB, and OX40. Preferably, the non-granulocytic immune cell has increased expression of one or more markers selected from: CD107a, 4-1BB, and OX40. The non-granulocytic immune cell may have increased expression of CD107a. The non-granulocytic immune cell may have increased expression of 4-1BB. The non-granulocytic immune cell may have increased expression of OX40. The non-granulocytic immune cell may have increased expression of CXCL10. The non-granulocytic immune cell may secrete increased concentrations of CXLC10. The non-granulocytic immune cell may have increased expression of IFN-γ. The non-granulocytic immune cell may secrete increased concentrations of IFN-γ. The non-granulocytic immune cell may have increased proliferation. The non-granulocytic immune cell may have increased tumour killing ability. The increase may be an increase compared to a non-granulocytic immune cell cultured in the absence of a population of granulopoietic cells but otherwise subjected to identical conditions.

The inventors have surprisingly found that populations of granulopoietic cells as described herein are capable of amplifying (preferably amplify) the therapeutic immune response of NK cells. For example, the inventors have shown that populations of granulopoietic cells as described herein increase NK cell proliferation, thereby overcoming the problem of limited ex vivo expansion of NK cells. The inventors have shown that granulopoietic cells as described herein increase NK cell survival, thereby overcoming the problem of limited in vivo survival of NK cells. In addition, the inventors have shown that populations of granulopoietic cells as described herein potently increase the expression of 4-1BB and OX40 on NK cells, thereby enhancing the cytotoxicity of the NK cells.

Accordingly, the composition may comprise a population of granulopoietic cells and an NK cell. An NK cell may be any suitable NK cell. The NK cell may be an NK cell that is CD3⁻, and CD56⁺. For example, the NK cell may be an NK cell that is CD3⁻, CD56^{dim}, and/or CD16⁺, e.g. CD3⁻, CD56^{dim}, and CD16⁺. The NK cell may be an NK cell that is CD3⁻, CD56^{bright}, and/or CD16⁻, e.g. CD3⁻, CD56^{bright}, and CD16⁻. The NK cell may be an NK cell that is CD3⁻, CD56⁺, CD7⁺, CD127⁻, NKp46⁺, T-bet⁺, and/or Eomes⁺, e.g. CD3⁻, CD56⁺, CD7⁺, CD127⁻, NKp46⁺, T-bet ⁺, and Eomes⁺. Without being bound by theory, it is believed that CD56^{dim}, and CD16⁺ NK cells are predominantly found in the blood, whereas CD56^{bright}, and CD16⁻ NK cells are predominantly found in the lymph. The NK cell may be an NK cell obtainable by the method described in Oyer et al. Biol Blood Marrow Transplant 21 (2015) 632-639, which is herein incorporated by reference in its entirety.

A population of granulopoietic cells suitable for use in accordance with the present invention may increase activation of NK cells. Accordingly, the composition may comprise an NK cell having increased activation. Without limitation, increased activation of NK cells may be associated with one or more of the following: an increase in expression by NK cells of a degranulation marker (including, but not limited to, CD107a); an increase in expression by NK cells of a costimulatory molecule (including, but not limited to, 4-1BB and/or OX40); an increase in expression by NK cells of a cytokine (including, but not limited to IFN-γ and/or TNF); an increase in trafficking of NK cells; an increase in recruitment of NK cells into the TME; an increase in cytocidal activity (including, but not limited to tumour cell killing) by NK cells; an increase in proliferation of NK cells; an increase in survival of NK cells; and an increase in abundance of NK cells. Changes in these properties associated with increased activation of NK cells exposed to populations of granulopoietic cells suitable for use in accordance with the present invention are demonstrated in the Examples. Further relevant considerations in respect of these various properties are set out elsewhere in the present specification.

Activation of NK cells may be increased by at least 5%. For example, activation of NK cells may be increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, or more. Quantification of the increase in activation of NK cells in accordance with such an embodiment may make use of comparison to an appropriate control.

As used herein, the term “an appropriate control” may refer to a non-granulocytic immune cell which has not been cultured in the presence of a population of granulopoietic cells, but has otherwise been subjected to identical conditions.

In some embodiments, at least about 5%, 10%, 15%, 20%, 25%, 30%, 35% or 40% of the NK cells in the composition express 4-1BB, e.g. as determined by flow cytometry. Preferably, at least about 10% of the NK cells in the composition express 4-1BB, e.g. as determined by flow cytometry. In some embodiments, at least about 1%, 2%, 5%, 10%, 15%, 20%, 25%, 30%, or 35% of the NK cells in the composition express OX40, e.g. as determined by flow cytometry. Preferably, at least about 5% of the NK cells in the composition express OX40, e.g. as determined by flow cytometry. Particularly preferably, at least about 10% of the NK cells in the composition express 4-1BB, and at least about 5% of the NK cells in the composition express OX40.

The inventors have surprisingly found that populations of granulopoietic cells as described herein are capable of amplifying (preferably amplify) the therapeutic immune response of T cells. For example, the inventors have shown that populations of granulopoietic cells as described herein increase expression of 4-1BB and OX40 on CD4⁺ and CD8⁺ T cells and increasing expression of 4-1BB and CD25 on γδ T cells, thereby improving the effector function of these cells. Accordingly, the composition may comprise a population of granulopoietic cells and a T cell. A T cell may be any suitable T cell. The T cell may be an αβ T cell or a γδ T cell. An αβ T cell is a T cell which comprises an αβ T cell receptor (TCR) on its cell surface. Meanwhile, a γδ T cell is a T cell which comprises a γδ TCR on its cell surface. Preferably, the T cell is a γδ T cell. Particularly preferably, the γδ T cell is a Vδ1 or Vδ2 γδ T cell. Preferably, the T cell is not an αβ T cell.

A population of granulopoietic cells suitable for use in accordance with the present invention may increase activation of T cells. Accordingly, the composition may comprise a T cell having increased activation. Without limitation, increased activation of T cells may be associated with one or more of the following: an increase in expression by T cells of a degranulation marker (including, but not limited to, CD107a); an increase in expression by T cells of a costimulatory molecule (including, but not limited to, 4-1BB and/or OX40); an increase in expression by T cells of a cytokine; an increase in trafficking of T cells; an increase in recruitment of T cells into the TME; an increase in cytocidal activity (including, but not limited to tumour cell killing) by T cells; an increase in proliferation of T cells; an increase in survival of T cells; and an increase in abundance of T cells. Changes in these properties associated with increased activation of T cells exposed to populations of granulopoietic cells suitable for use in accordance with the present invention are demonstrated in the Examples. Further relevant considerations in respect of these various properties are set out elsewhere in the present specification.

Activation of T cells may be increased by at least 5%. For example, activation of T cells may be increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, or more. Quantification of the increase in activation of T cells in accordance with such an embodiment may make use of comparison to an appropriate control.

A population of granulopoietic cells suitable for use in accordance with the present invention may increase activation of CD8⁺ T cells. Accordingly, the composition may comprise a CD8⁺ T cell having increased activation. Increased activation of CD8⁺ T cells may be associated with one or more of the following: an increase in expression by CD8⁺ T cells of a degranulation marker (including, but not limited to, CD107a); an increase in expression by CD8⁺ T cells of a costimulatory molecule (including, but not limited to, 4-1BB and/or OX40); and an increase in proliferation of CD8⁺ T cells. Further relevant considerations in respect of these various properties are set out elsewhere in the present specification.

Activation of such CD8⁺ T cells may be increased by at least 5%. For example, activation of CD8+ T cells may be increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, or more. Quantification of the increase in activation of CD8+ T cells in accordance with such an embodiment may make use of comparison to an appropriate control.

A population of granulopoietic cells suitable for use in accordance with the present invention may increase activation of CD4⁺ T cells. Accordingly, the composition may comprise a CD4⁺ T cell having increased activation. Increased activation of CD4⁺ T cells may be associated with one or more of the following: an increase in expression by CD4⁺ T cells of a costimulatory molecule (including, but not limited to, 4-1BB and/or OX40); and an increase in proliferation of CD4⁺ T cells. Further relevant considerations in respect of these various properties are set out elsewhere in the present specification.

Activation of such CD4⁺ T cells may be increased by at least 5%. For example, activation of CD4⁺ T cells may be increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, or more. Quantification of the increase in activation of CD4⁺ T cells in accordance with such an embodiment may make use of comparison to an appropriate control.

A population of granulopoietic cells suitable for use in accordance with the present invention may increase activation of γδ T cells (e.g. Vδ1⁺ γδ T cells or Vδ2⁺ γδ T cells). Accordingly, the composition may comprise a γδ T cell (e.g. a Vδ1⁺ γδ T cell or a Vδ2⁺ γδ T cell) having increased activation. Increased activation of Vδ1⁺ γδ T cells may be associated with increased expression of 4-1BB and/or increased expression of CD25 on the cell surface. Increased activation of Vδ2⁺ γδ T cells may be associated with increased expression of 4-1BB on the cell surface. Increased activation of Vδ1⁺ and Vδ2⁺ γδ T cells may be associated with increased proliferation and/or survival of Vδ1 and Vδ2⁺ γδ T cells respectively.

Activation of such γδ T cells may be increased by at least 5%. For example, activation of γδ T cells may be increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, or more. Quantification of the increase in activation of γδ T cells in accordance with such an embodiment may make use of comparison to an appropriate control.

In some embodiments, at least about 0.1%, 0.2%, 0.3%, 0.4%, 0.5%, 1%, 2%, 3%, 4% or 5%, of the Vδ1⁺ γδ T cells in the composition express 4-1BB, e.g. as determined by flow cytometry. Preferably at least about 0.5% of the Vδ1⁺ γδ T cells in the composition express 4-1BB, e.g. as determined by flow cytometry. In some embodiments, at least about 0.1%, 0.2%, 0.3%, 0.4%, 0.5%, 1%, 2%, 3%, 4%, 5%, 10%, 15%, 20%, 25% or 30% of the Vδ1⁺ γδ T cells in the composition express CD25, e.g. as determined by flow cytometry. Preferably at least about 1% of the Vδ1⁺ γδ T cells in the composition express CD25, e.g. as determined by flow cytometry. Preferably, at least about 0.5% of the Vδ1⁺ γδ T cells in the composition express 4-1BB, e.g. as determined by flow cytometry and at least about 1% of the Vδ1⁺ γδ T cells in the composition express CD25, e.g. as determined by flow cytometry. Particularly preferably, at least about 5% of the Vδ1⁺ T cells in the composition express 4-1BB, e.g. as determined by flow cytometry, and at least about 30% of the Vδ1⁺ γδ T cells in the composition express CD25, e.g. as determined by flow cytometry.

In some embodiments, at least about 0.1%, 0.2%, 0.3%, 0.4%, 0.5%, 1%, 2%, 3%, 4%, 5%, or 10% of the Vδ2⁺ γδ T cells in the composition express 4-1BB, e.g. as determined by flow cytometry. Preferably at least about 0.5% of the Vδ2⁺ γδ T cells in the composition express 4- 1BB, e.g. as determined by flow cytometry. Particularly preferably, at least about 10% of the Vδ2⁺ γδ T cells in the composition express 4-1BB, e.g. as determined by flow cytometry.

A dendritic cell may be any suitable dendritic cell. For example, the dendritic cell may be a classical or conventional dendritic cell (CDC), a plasmacytoid dendritic cell (pDC), or a monocyte-derived cell with dendritic cell-like properties (moDC). A cDC may be a type 1 cDC (cDC1) or a type 2 cDC (cDC2). Without being bound by theory, it is believed that cDC1 cells present exogenous antigens on MHC class I to induce naïve CD8⁺ T cells to acquire cytotoxic T cell (CTL) effector function, whereas cDC2 cells prime naïve CD4⁺ T cells through antigen presentation on MHC class II. Meanwhile, pDCs are believed to have a dedicated function of secreting type I interferon (IFN).

Accordingly, a dendritic cell may be a dendritic cell that is CD11c⁺, HLA-DR⁺, and/or CD141⁺, e.g. CD11c⁺, HLA-DR⁺, and CD141+. This expression profile may be characteristic of a cDC1 cell. A dendritic cell may be a dendritic cell (e.g. a cDC1 cell) that is CD11c⁺, HLA-DR⁺, CD141⁺, CLEC9A⁺, and/or CADM1⁺, e.g. CD11c⁺, HLA-DR⁺, CD141⁺, CLEC9A⁺, and CADM1⁺. A dendritic cell may be a dendritic cell that is CD11c⁺, HLA-DR⁺ CD1c⁺, and/or CD11b⁺, e.g. CD11c⁺, HLA-DR⁺, CD1c⁺, and CD11b⁺. This expression profile may be characteristic of a cDC2 cell. A dendritic cell may be a dendritic cell (e.g. a cDC2 cell) that is CD11c⁺, HLA-DR⁺, CD1c+, CD11b⁺, FCER1A⁺, CLEC10A⁺, CD2⁺, CD172A⁺, and/or ILT1⁺, e.g. CD11c⁺, HLA-DR⁺, CD1c⁺, CD11b+, FCER1A⁺, CLEC10A⁺, CD2⁺, CD172A⁺, and ILT1⁺. A dendritic cell may be a dendritic cell that is HLA-DR⁺, CD303⁺, and/or CD123⁺, e.g. HLA-DR⁺, CD303⁺, and CD123⁺. This expression profile may be characteristic of a pDC. A dendritic cell may be a dendritic cell (e.g. a pDC) that is HLA-DR⁺, CD303⁺, CD123⁺, CD11c⁺ (e.g. CD11c^{int}), MHCII⁺ (e.g. MHC1^{lo}), Bst2⁺, and/or B220⁺, e.g. HLA-DR⁺, CD303⁺, CD123⁺, CD11c⁺ (e.g. CD11c^{int}), MHCII⁺ (e.g. MHC^{lo}), Bst2⁺, and B220⁺, such as HLA-DR⁺, CD303⁺, CD123⁺, CD11c^{int}, MHC^{lo}, Bst2⁺, and B220⁺. A dendritic cell may be a dendritic cell that is CD11c⁺, CD11b+, CD1a⁺, and/or CD1c⁺, e.g. CD11c⁺, CD11b⁺, CD1a⁺, and CD1c⁺. This expression profile may be characteristic of an moDC. A dendritic cell may be a dendritic cell (e.g. an moDC) that is CD11c⁺, CD11b⁺, CD1a⁺, CD1c⁺, CD206⁺, CD209⁺, and/or CD172A⁺, CD11c⁺, CD11b⁺, CD1a⁺, CD1c⁺, CD206⁺, CD209⁺, and CD172A⁺.

A population of granulopoietic cells suitable for use in accordance with the present invention may increase activation of dendritic cells. Accordingly, the composition may comprise a dendritic cell having increased activation. Increased activation of dendritic cells may be associated with increased expression of CD83, CD86, and/or CD80.

Activation of such dendritic cells may be increased by at least 5%. For example, activation of dendritic cells may be increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, or more. Quantification of the increase in activation of dendritic cells in accordance with such an embodiment may make use of comparison to an appropriate control.

A monocyte may be any suitable monocyte. For example, the monocyte may be a classical monocyte, an intermediate monocyte, or a nonclassical monocyte. Without being bound by theory, it is believed that classical monocytes are the primary monocyte population responsible for phagocytic activity and have low pro-inflammatory cytokine production; that intermediate monocytes produce pro-inflammatory cytokines such as TNFα, IL-1β and/or IL-6; and that nonclassical monocytes produce anti-inflammatory cytokines and constitutively produce IL-1RA.

Accordingly, a monocyte may be a monocyte that is CD14⁺, CD16⁺ or CD64⁺. For example, a monocyte may be a monocyte that is CD14⁺ (e.g. CD14^{Hi}), CD64⁺, CD62L⁺, TNFR1⁺, TNFR2⁺ (e.g.) TNFR2^{Lo}, CD192⁺ (e.g. CD192^{Hi}), and/or CXCR1⁺ (e.g. CXCR1^{Lo}); such as CD14⁺ (e.g. CD14^{Hi}), CD64⁺, CD62L⁺, TNFR1⁺, TNFR2⁺ (e.g. TNFR2^{Lo}), CD192⁺ (e.g. CD192^{Hi}), and CXCR1⁺ (e.g. CXCR1^{Lo}), e.g. CD14^{Hi}, CD64⁺, CD62L⁺, TNFR1⁺, TNFR2^{Lo}, CD192^{Hi}, and CXCR1^{Lo}. This expression profile may be characteristic of a classical monocyte. A monocyte may be a monocyte that is CD16⁺, CD14⁺ (e.g. CD14^{Hi}), CD64⁺, HLA-DR⁺ (e.g. HLA-DR^{Hi}), TNFR1⁺ (e.g. TNFR1^{Hi}), TNFR2⁺, CD192⁺ (e.g. CD192^{Lo}), CX3CR1⁺ (e.g. CX3CR1^{Hi}), and/or CD195⁺, such as CD16⁺, CD14⁺ (e.g. CD14^{Hi}), CD64⁺, HLA-DR⁺ (e.g. HLA-DR^{Hi}), TNFR1⁺ (e.g. TNFR1^{Hi}), TNFR2⁺, CD192⁺ (e.g. CD192^{Lo}), CX3CR1⁺ (e.g. CX3CR1^{Hi}), and CD195⁺, e.g. CD16⁺, CD14^{Hi}, CD64⁺, HLA-DR^{Hi}, TNFR1^{Hi}, TNFR2⁺, CD192^{Lo}, CX3CR1^{Hi}, and CD195⁺. This expression profile may be characteristic of an intermediate monocyte. A monocyte may be a monocyte that is CD14⁺ (e.g. CD14^{Lo}), CD16⁺ (e.g. CD16^{Hi}), TNFR1⁺ (e.g. TNFR1^{Lo}), and/or TNFR2⁺ (e.g. TNFR2^{Hi}), such as CD14⁺ (e.g. CD14^{Lo}), CD16⁺ (e.g. CD16^{Hi}), TNFR1⁺ (e.g. TNFR1^{Lo}), and TNFR2⁺ (e.g. TNFR2^{Hi}), e.g. CD14^{Lo}, CD16^{Hi}, TNFR1^{Lo}, and TNFR2^{Hi}. This expression profile may be characteristic of a nonclassical monocyte.

A macrophage may be any suitable macrophage. For example, the macrophage may be a classically activated M1 macrophage or an alternatively activated M2 macrophage. Without being bound by theory, it is believed that M1 macrophages show high antigen presentation activity and high production of pro-inflammatory cytokines such as IL-1, IL-6, TNFα, nitric oxide, and reactive oxygen species (ROS). Meanwhile, it is believed that M2 macrophages show low production of inflammatory cytokines such as IL-1, IL-6 and TNFα. It is understood that M1 and M2 macrophages may be further divided into additional subclassifications.

Accordingly, a macrophage may be a macrophage that is CD11b⁺, CD14⁺, CD15⁺, CD16⁺, and/or CD68⁺, CD11b⁺, CD14⁺, CD15⁺, CD16⁺, and CD68⁺. A macrophage may be a macrophage that is CD16⁺, CD32⁺, CD16/CD32⁺, CD64⁺, CD68⁺, CD80⁺, CD86⁺, CD369⁺, Mer⁺ and/or MHC II⁺, e.g. CD16⁺, CD32⁺, CD16/CD32⁺, CD64⁺, CD68⁺, CD80⁺, CD86⁺, CD369⁺, Mert, and MHC II⁺. This expression profile may be characteristic of an M1 macrophage. An M1 macrophage may be characterised by secretion of IFNγ, IL-1α, IL-1β, IL-6, IL-12, IL-23 and/or TNFα, e.g. IFNγ, IL-1a, IL-1B, IL-6, IL-12, IL-23 and TNFα. A macrophage may be a macrophage that is CD115⁺, CD163⁺, CD204⁺, CD206⁺, CD209⁺, FceR1⁺, and/or VSIG4⁺ e.g. CD115⁺, CD163⁺, CD204⁺, CD206⁺, CD209⁺, FceR1⁺, and VSIG4⁺. This expression profile may be characteristic of an M2 macrophage. An M2 macrophage may be characterised by secretion of IDO, IL-10, and/or TGFB, e.g. IDO, IL-10, and TGFB.

A population of granulopoietic cells suitable for use in accordance with the present invention may increase activation of macrophages. Accordingly, the composition may comprise a macrophage having increased activation. Increased activation of macrophages may be associated with increased expression of CD86, CD40 and/or enhanced secretion of TNFα.

Activation of such macrophages may be increased by at least 5%. For example, activation of macrophages may be increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, or more. Quantification of the increase in activation of macrophages in accordance with such an embodiment may make use of comparison to an appropriate control.

A B cell may be any suitable B cell. For example, the B cell may be any B cell that comprises a B cell receptor (BCR). The B cell may be a pro-B cell, a pre-B cell, an immature B cell, a transitional B cell, a naïve B cell, a B1 cell, a memory B cell, or a plasma cell. Preferably, the B cell is a transitional B cell, a naïve B cell, a memory B cell, or a plasma cell.

Accordingly, the B cell may be a B cell that is CD19⁺, CD20⁺, CD34⁺, CD38⁺, and/or CD45R⁺, e.g. CD19⁺, CD20⁺, CD34⁺, CD38⁺, and CD45R⁺. This expression profile may be characteristic of a pro-B cell. The B cell may be a B cell that is CD19⁺, CD20⁺, CD38⁺, CD40⁺, and/or CD45R⁺, e.g. CD19⁺, CD20⁺, CD38⁺, CD40⁺, and CD45R⁺. This expression profile may be characteristic of a pre-B cell. The B cell may be a B cell that is CD19⁺, CD20⁺, CD40⁺, CD45R⁺, and/or IgM⁺, CD19⁺, CD20⁺, CD40⁺, CD45R⁺, and IgM⁺. This expression profile may be characteristic of an immature B cell. The B cell may be a B cell that is CD10⁺, CD19⁺, CD20⁺, CD24^{hi}, and/or CD28^{hi}, e.g. CD10⁺, CD19⁺, CD20⁺, CD24^{hi}, and CD28^{hi}. The B cell may be a B cell that is CD10⁺, CD19⁺, CD20⁺, CD24^{hi}, CD28^{hi}, BCL-2^{lo}, and/or CD27⁻, e.g. CD10⁺, CD19⁺, CD20⁺, CD24^{hi}, CD28^{hi}, BCL-21^{lo}, and CD27⁻. This expression profile may be characteristic of a transitional B cell. The B cell may be a B cell that is CD19⁺, CD20⁺, CD23⁺, CD40⁺, and/or CD150⁺, e.g. CD19⁺, CD20⁺, CD23⁺, CD40⁺, and CD150⁺. The B cell may be a B cell that is CD19⁺, CD20⁺, CD23⁺, CD40⁺, CD150⁺, IgM⁺, and/or IgD⁺, e.g. CD19⁺, CD20⁺, CD23⁺, CD40⁺, CD150⁺, IgM⁺, and IgD⁺. The B cell may be a B cell that is CD19⁺, CD20⁺, CD23⁺, CD40⁺, CD150⁺, IgM⁺, IgD⁺, and/or CD38^{lo}, e.g. CD19⁺, CD20⁺, CD23⁺, CD40⁺, CD150⁺, IgM⁺, IgD⁺, and CD38^{lo}. This expression profile may be characteristic of a naïve B cell. The B cell may be a B cell that is CD19⁺, CD20⁺, CD27⁺, and/or IgM⁺, e.g. CD19⁺, CD20⁺, CD27⁺, and IgM⁺. The B cell may be a B cell that is CD19⁺, CD20⁺, CD27⁺, IgM⁺, and/or IgD^{lo}, e.g. CD19⁺, CD20⁺, CD27⁺, IgM⁺, and IgD^{lo}. This expression profile may be characteristic of a B1 cell. The B cell may be a B cell that is CD19⁺, CD20⁺, CD27⁺, CD40⁺, and/or CD150⁻, e.g. CD19⁺, CD20⁺, CD27⁺, CD40⁺, and CD150⁻. The B cell may be a B cell that is CD19⁺, CD20⁺, CD27⁺, CD40⁺, CD150⁻, IgA⁺, and/or IgG⁺, e.g. CD19⁺, CD20⁺, CD27⁺, CD40⁺, CD150⁻, IgA⁺, and IgG⁺. The B cell may be a B cell that is CD19⁺, CD20⁺, CD27⁺, CD40⁺, CD150⁻, IgA⁺, IgG⁺, CD23^{lo}, and/or CD38⁻, e.g. CD19⁺, CD20⁺, CD27⁺, CD40⁺, CD150⁻, IgA⁺, IgG⁺, CD23^{lo}, and CD38⁻. This expression profile may be characteristic of a memory B cell. The B cell may be a B cell that is CD9^{hi}, CD27^{hi}, CD38^{hi}, CD40⁺, and/or CD95⁺, e.g. CD9^{hi}, CD27^{hi}, CD38^{hi}, CD40⁺, and CD95⁺. The B cell may be a B cell that is CD9^{hi}, CD27^{hi}, CD38^{hi}, CD40⁺, CD95⁺, CXCR4⁺, and/or CD138⁺, e.g. CD9^{hi}, CD27^{hi}, CD38^{hi}, CD40⁺, CD95⁺, CXCR4⁺, and CD138⁺. The B cell may be a B cell that is CD9^{hi}, CD27^{hi}, CD38^{hi}, CD40⁺, CD95⁺, CXCR4⁺, CD138⁺, CD191°, and/or CD20 e.g. CD9^{hi}, CD27^{hi}, CD38^{hi}, CD40⁺, CD95⁺, CXCR4⁺, CD138⁺, CD19^{lo}, and CD20⁻. This expression profile may be characteristic of a plasma cell.

The inventors have surprisingly found that populations of granulopoietic cells as described herein are capable of amplifying (preferably amplify) the therapeutic immune response of blood derived myeloid cells. For example, the inventors have shown that granulopoietic cells as described herein increase survival and/or proliferation of blood derived myeloid cells, thereby overcoming the problem of expanding blood derived myeloid cells ex vivo. Accordingly, the composition may comprise a population of granulopoietic cells and a blood derived myeloid cell. A blood derived myeloid cell may be any suitable blood derived myeloid cell. A blood derived myeloid cell may be a blood derived myeloid cell that is CD11b⁺, CD15⁺, and/or CD14⁺, e.g. CD11b⁺, CD15⁺, and CD14⁺. Preferably, a blood derived myeloid cell is a CD11⁺ blood derived myeloid cell. A population of granulopoietic cells suitable for use in accordance with the present invention may increase activation of blood derived myeloid cells. Accordingly, the composition may comprise a blood derived myeloid cell having increased activation. Increased activation of blood derived myeloid cells may be associated with increased expression of CD11b.

Activation of such blood derived myeloid cells may be increased by at least 5%. For example, activation of blood derived myeloid cells may be increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, or more. Quantification of the increase in activation of macrophages in accordance with such an embodiment may make use of comparison to an appropriate control.

The non-granulocytic immune cell may be a stem, precursor or progenitor cell, for example a stem, precursor or progenitor cell of a dendritic cell, a monocyte, a macrophage, a natural killer (NK) cell, a B cell, or a T cell e.g. a γδ T cell. The non-granulocytic immune cell may be a stem, precursor or progenitor cell of any non-granulocytic immune cell described herein. Preferably, the non-granulocytic immune cell is a terminally differentiated immune cell.

Non-granulocytic immune cells may be obtainable from any suitable source. For example, a non-granulocytic immune cell may be obtainable from a sample of umbilical cord blood, which may be obtainable (e.g. obtained) from a donor. For example, the non-granulocytic immune cell may be obtainable from a sample of PBMCs, which may be obtainable (e.g. obtained) from a donor. Preferably, the non-granulocytic immune cell is obtainable from a sample of αβ T cell-depleted PBMCs. The non-granulocytic immune cell may be obtainable from a sample of αβ T cell-depleted PBMCs obtainable from G-CSF mobilized blood. The non-granulocytic immune cell may be obtainable from (e.g. differentiated in vitro from) a stem cell, such as a haematopoietic stem cell or iPSC.

Without being bound by theory, it is believed that compositions comprising a plurality of different types of non-granulocytic immune cell may have a synergistically amplified therapeutic immune response when combined with a population of granulopoietic cells of the invention. Accordingly, the composition may comprise a population of granulopoietic cells and a plurality of different types of non-granulocytic immune cell. For example, the composition may comprise at least 2, at least 3, at least 4, at least 5, or at least 6, different types of non-granulocytic immune cell. The composition may comprise 2, 3, 4, 5, or 6 different types of non-granulocytic immune cell.

The composition may comprise a plurality of different types of non-granulocytic immune cell selected from: a dendritic cell, a monocyte, a macrophage, a natural killer (NK) cell, a B cell, and a T cell (e.g. a γδ T cell). The composition may comprise a plurality of different types of non-granulocytic immune cell selected from: a dendritic cell, a monocyte, a macrophage, a a natural killer (NK) cell, a B cell, and a γδ T cell. The composition may comprise a plurality of different types of non-granulocytic immune cell selected from: a monocyte, a macrophage, an NK cell, and a γδ T cell. Thus, the composition may comprise a dendritic cell, a monocyte, a macrophage, an NK cell, a B cell and a T cell (e.g. a γδ T cell). The composition may comprise a dendritic cell, a monocyte, a macrophage, an NK cell, a B cell and a γδ T cell. Preferably, the composition comprises a monocyte, a macrophage, a NK cell, and a γδ T cell. Particularly preferably, the composition comprises an NK cell and a γδ T cell. Particularly preferably, the composition comprises an NK cell, and a Vδ1⁺ γδ T cell and/or a Vδ2⁺ γδ T cell, e.g. an NK cell, a Vδ1⁺ γδ T cell and a Vδ2⁺ γδ T cell.

A plurality of different types of non-granulocytic immune cell may be obtainable from any suitable source. For example, a plurality of different types of non-granulocytic immune cell may be obtainable from the same donor or different donors. Preferably, the plurality of different types of non-granulocytic immune cell are obtainable from the same donor. A plurality of different types of non-granulocytic immune cell may be obtainable from a single source or from different sources. Preferably, the plurality of different types of non-granulocytic immune cell are obtainable from a single source. For example, a plurality of different types of non-granulocytic immune cell may be obtainable from a sample of PBMCs. Preferably, the plurality of different types of non-granulocytic immune cells are obtainable from a sample of αβ T cell-depleted PBMCs. The plurality of different types of non-granulocytic immune cell may be obtainable from an iPSC or a population of iPSCs.

The population of granulopoietic cells and non-granulocytic immune cell may be obtainable from the same donor or from different donors. Preferably, the population of granulopoietic cells and non-granulocytic immune cell are obtainable from the same donor. For example, the population of granulopoietic cells and non-granulocytic immune cell may be obtainable from a healthy donor. Preferably, the population of granulopoietic cells and non-granulocytic immune cell are obtainable from a donor who does not have cancer. Obtaining cells from a single donor may be particularly advantageous because it allows for the extraction of the entire innate immune component of that donor. Donors with particularly beneficial innate immune cells (e.g. innate immune cells which are highly cytotoxic to disease stimuli such as cancer cells, or innate immune cells which are particularly good at recruiting other immune cells to diseased tissue) may therefore be selected and their innate immune cells included in the compositions of the invention. The cells in these compositions are expected to have synergistically improved properties (e.g. synergistically improved cytotoxicity and/or synergistically improved recruitment) compared to compositions comprising only a single cell type, which flows at least in part from the synergism between the different cell types present in the composition. In particular, as shown herein, the granulopoietic cells of the invention have shown a particularly surprising propensity to synergistically improve activation, cytotoxicity and/or recruitment of non-granulocytic immune cells.

The population of granulopoietic cells and non-granulocytic immune cell may be obtainable from the same or different sources. For example, the population of granulopoietic cells may be obtainable from a sample of isolated haematopoietic stem cells, and the non-granulocytic immune cell may be obtainable from a sample of isolated PBMCs. Preferably, the population of granulopoietic cells and non-granulocytic immune cell are obtainable from the same source. For example, the population of granulopoietic cells and non-granulocytic immune cell may be obtainable from an iPSC or a population of iPSCs. Preferably, the population of granulopoietic cells and non-granulocytic immune cell are obtainable from a sample of isolated PBMCs, e.g. PBMCs from mobilized blood. Particularly preferably, the population of granulopoietic cells and non-granulocytic immune cell are obtainable from a sample of αβ T cell-depleted PBMCs, e.g. αβ T cell-depleted PBMCs from mobilized blood. Accordingly, in preferred embodiments the population of granulopoietic cells and non-granulocytic immune cell are obtainable from a sample of αβ T cell-depleted PBMCs from mobilized blood obtainable from a single donor. Advantageously, this allows for the composition to be prepared using cells from a single source, thereby providing a significantly streamlined and efficient method of preparing a composition of the invention.

As used herein, the term “mobilized blood” refers to blood circulating through the body that has been treated with mobilizing agent(s) such as Plerixafor and/or G-CSF. The term “mobilizing agent” refers to an agent which aids in the recruitment of CD34⁺ hematopoietic stem and/or progenitor cells from the bone marrow into the blood stream. Accordingly, mobilized blood has a higher concentration of CD34⁺ hematopoietic stem and/or precursor cells compared to non-mobilized blood. Mobilized blood can be collected via leukapheresis and allows for the collection of PBMCs comprising non-granulocytic immune cells and hematopoietic stem and/or precursor cells.

The inventors have found that granulopoietic cells are capable of amplifying (preferably amplify) the therapeutic immune response of non-granulocytic immune cells when present at different ratios. Accordingly, the population of granulopoietic cells and non-granulocytic immune cell may be present in the composition at any suitable ratio. The population of granulopoietic cells and non-granulocytic immune cell may be present at a ratio of 100:1 to 0.01:1 granulopoietic cells to non-granulocytic immune cells. The population of granulopoietic cells and non-granulocytic immune cell may be present at a ratio of 100:1 to 0.01:1; 75:1 to 0.05:1; 50:1 to 0.1:1; 25:1 to 0.2:1; 10:1 to 0.25:1; 5:1 to 0.25:1; 3:1 to 0.25:1; or 2:1 to 0.5:1 granulopoietic cells to non-granulocytic immune cells. Preferably, the population of granulopoietic cells and non-granulocytic cell are present at a ratio of 3:1 to 0.25:1 granulopoietic cells to non-granulocytic immune cells.

The population of granulopoietic cells and non-granulocytic immune cell may be present at a ratio of less than or equal to 100:1, 75:1, 50:1, 25:1, 10:1, 5:1, 3:1, 2:1, 1:1, 0.5:1, 0.25:1, 0.1:1, 0.05:1 or 0.01:1 granulopoietic cells to non-granulocytic immune cells. The population of granulopoietic cells and non-granulocytic immune cell may be present at a ratio of at least 0.01:1, 0.05:1 0.1:1, 0.25:1, 0.5:1, 1:1, 2:1, 3:1, 5:1, 10:1, 25:1, 50:1, 75:1, or 100:1 granulopoietic cells to non-granulocytic immune cells. The population of granulopoietic cells and non-granulocytic immune cell may be present at a ratio of 100:1, 75:1, 50:1, 25:1, 10:1, 5:1, 3:1, 2:1, 1:1, 0.5:1, 0.25:1, 0.1:1, 0.05:1 or 0.01:1 granulopoietic cells to non-granulocytic immune cells. Preferably, the population of granulopoietic cells and non-granulocytic immune cell are present at a ratio of 2:1, 1:1, or 0.5:1 granulopoietic cells to non-granulocytic immune cells. For example, the population of granulopoietic cells and non-granulocytic immune cell may be present at a ratio of 2:1 granulopoietic cells to non-granulocytic immune cells. The population of granulopoietic cells and non-granulocytic immune cell may be present at a ratio of 1:1 granulopoietic cells to non-granulocytic immune cells. The population of granulopoietic cells and non-granulocytic immune cell may be present at a ratio of 0.5:1 granulopoietic cells to non-granulocytic immune cells.

The compositions of the invention may be suitable for allogeneic administration. Accordingly, the compositions may be substantially or wholly free of any component which causes graft versus host disease, e.g. an αβ T cell. Thus, in one embodiment the composition does not comprise an αβ T cell. The term “does not comprise an αβ T cell” means that the composition comprises no, or substantially no, αβ T cells. The term “substantially no” as used in this context may mean that fewer than 10% of the cells in the composition may be αβ T cells; fewer than 5% of the cells in the composition may be αβ T cells; fewer than 4% of the cells in the composition may be αβ T cells; fewer than 3% of the cells in the composition may be αβ T cells; fewer than 2% of the cells in the composition may be αβ T cells; fewer than 1% of the cells in the composition may be αβ T cells; fewer than 0.1% of the cells in the composition may be αβ T cells; fewer than 0.01% of the cells in the composition may be αβ T cells; fewer than 0.001% of the cells in the composition may be αβ T cells; or fewer than 0.0001% of the cells in the composition may be αβ T cells. The term “substantially no” as used in this context may mean that the composition comprises up to about 1×10⁹ αβ T cells/kg of the subject to be administered; up to about 1×10⁸ αβ T cells/kg of the subject to be administered; up to about 1×10⁷ αβ T cells/kg of the subject to be administered; up to about 1×10⁶ αβ T cells/kg of the subject to be treated; preferably up to about 1×10⁵ αβ T cells/kg of the subject to be treated. The term “substantially no” as used in this context may mean that the composition comprises about 1×10¹-1×10⁹ αβ T cells/kg of the subject to be treated; about 1×10²-1×10⁸ αβ T cells/kg of the subject to be treated; about 1×10³-1×10⁷ αβ T cells/kg of the subject to be treated; about 1×10⁴-1×10⁶ αβ T cells/kg of the subject to be treated; preferably about 1×10⁴-1×10⁵ αβ T cells/kg of the subject to be treated. The term “substantially no” as used in this context may mean that the composition comprises up to about 7×10¹⁰ αβ T cells; up to about 7×10⁹ αβ T cells; up to about 7×10⁸ αβ T cells; up to about 7×10⁷ αβ T cells; preferably up to about 7×10⁶ αβ T cells. The term “substantially no” as used in this context may mean that the composition comprises about 7×10²-7×10¹⁰ αβ T cells/kg of the subject to be treated; about 7×10³-7×10⁹ αβ T cells/kg of the subject to be treated; about 7×10⁴-7×10⁸ αβ T cells/kg of the subject to be treated; about 7×10⁵-7×10⁷ αβ T cells/kg of the subject to be treated; preferably about 7×10⁵-7×10⁶ αβ T cells. Particularly preferably, the composition comprises no αβ T cells.

A “subject” or “patient” as used herein may be a mammal, such as a human or other mammal. Preferably “subject” means a human subject. Preferably, “patient” means a human patient.

In one aspect, there is provided a composition comprising one or more granulocytes differentiated from a population of granulopoietic cells capable of amplifying (preferably that amplifies) a therapeutic immune response of a non-granulocytic cell, and a non-granulocytic cell.

In various aspects, the composition may be a pharmaceutical composition, wherein the pharmaceutical composition further comprises a pharmaceutically acceptable carrier, excipient, adjuvant and/or salt.

In various aspects, the compositions (e.g. pharmaceutical compositions) are obtainable (e.g. obtained) by a method of preparing a composition as disclosed herein.

In one aspect, there is provided a method of preparing a composition of the invention, the method comprising culturing a non-granulocytic immune cell in the presence of a population of granulopoietic cells of the invention. Preferably, the population of granulopoietic cells is capable of amplifying (preferably amplifies) the therapeutic immune response of the non-granulocytic immune cell.

As set out above, granulopoietic cells of the invention are surprisingly capable of amplifying (preferably amplifies) the therapeutic immune response of different types of non-granulocytic immune cell. Accordingly, the method may comprise culturing an NK cell in the presence of a population of granulopoietic cells, thereby forming the composition. The method may comprise culturing a T cell (e.g. γδ T cell) in the presence of a population of granulopoietic cells, thereby forming the composition. Preferably, the method comprises culturing an NK cell and a T cell (e.g. γδ T cell) in the presence of a population of granulopoietic cells, thereby forming the composition. Particularly preferably, the method comprises culturing an NK cell and a γδ T cell (e.g. a Vδ1⁺ γδ T cell or a Vδ2⁺ γδ T cell) in the presence of a population of granulopoietic cells, thereby forming the composition. The non-granulocytic immune cell and population of granulopoietic cells may be cultured in the absence of an αβ T cell.

The inventors have surprisingly shown that particular cytokines may synergistically amplify the therapeutic immune response of a non-granulocytic immune cell. In particular, the inventors hypothesise that cytokines which signal through the common gamma chain, or interleukin-2 receptor subunit gamma (IL-2RG) may be particularly useful in amplifying the therapeutic immune response of non-granulocytic immune cells cultured in the presence of a population of granulopoietic cells. Such cytokines may include IL-15, IL-2, IL-4, IL-7, IL-9, IL-15 and IL-21. Accordingly, the method may comprise culturing the population of granulopoietic cells and non-granulocytic immune cell in the presence of a cytokine that signals through IL-2RG. The method may comprise culturing the population of granulopoietic cells and non-granulocytic immune cell in the presence of one or more cytokines selected from: IL-15, IL-2, IL-4, IL-7, IL-9, IL-15 and IL-21. Preferably, the method comprises culturing the population of granulopoietic cells and non-granulocytic immune cell in the presence of IL-15.

The population of granulopoietic cells and non-granulocytic cell may be cultured together at any suitable ratio. The population of granulopoietic cells and non-granulocytic immune cell may be cultured together at a ratio of 100:1 to 0.01:1 granulopoietic cells to non-granulocytic immune cells. The population of granulopoietic cells and non-granulocytic immune cell may be cultured together at a ratio of 100:1 to 0.01:1; 75:1 to 0.05:1; 50:1 to 0.1:1; 25:1 to 0.2:1; 10:1 to 0.25:1; 5:1 to 0.25:1; 3:1 to 0.25:1; or 2:1 to 0.5:1 granulopoietic cells to non-granulocytic immune cells. Preferably, the population of granulopoietic cells and non-granulocytic cell are cultured together at a ratio of 3:1 to 0.25:1 granulopoietic cells to non-granulocytic immune cells.

The population of granulopoietic cells and non-granulocytic immune cell may be cultured together at a ratio of less than or equal to 100:1, 75:1, 50:1, 25:1, 10:1, 5:1, 3:1, 2:1, 1:1, 0.5:1, 0.25:1, 0.1:1, 0.05:1 or 0.01:1 granulopoietic cells to non-granulocytic immune cells. The granulopoietic cells and non-granulocytic immune cells may be cultured together at a ratio of at least 0.01:1, 0.05:1 0.1:1, 0.25:1, 0.5:1, 1:1, 2:1, 3:1, 5:1, 10:1, 25:1, 50:1, 75:1, or 100:1 granulopoietic cells to non-granulocytic immune cells. The population of granulopoietic cells and non-granulocytic immune cell may be cultured together at a ratio of 100:1, 75:1, 50:1, 25:1, 10:1, 5:1, 3:1, 2:1, 1:1, 0.5:1, 0.25:1, 0.1:1, 0.05:1 or 0.01:1 granulopoietic cells to non-granulocytic immune cells. Preferably, the population of granulopoietic cells and non-granulocytic immune cell are cultured together at a ratio of 2:1, 1:1, or 0.5:1 granulopoietic cells to non-granulocytic immune cells. For example, the population of granulopoietic cells and non-granulocytic immune cell may be cultured together at a ratio of 2:1 granulopoietic cells to non-granulocytic immune cells. The population of granulopoietic cells and non-granulocytic immune cell may be cultured together at a ratio of 1:1 granulopoietic cells to non-granulocytic immune cells. The population of granulopoietic cells and non-granulocytic immune cell may be cultured together at a ratio of 0.5:1 granulopoietic cells to non-granulocytic immune cells.

A suitable source for the non-granulocytic immune cell may be PBMCs. Accordingly, the method may comprise culturing PBMCs in the presence of granulopoietic cells. The method may comprise:
(a) isolating PBMCs from a sample obtainable from a donor; and
(b) culturing the PBMCs in the presence of granulopoietic cells,
thereby forming the composition.

To improve the suitability of the composition for allogeneic administration, components that may cause graft versus host disease may be removed from the composition. The method may comprise removing components that cause graft versus host disease from the composition. For example, the method may comprise a step of depleting αβ T cells from the composition. Accordingly, the method may comprise culturing PBMCs in the presence of granulopoietic cells, and depleting αβ T cells from the PBMCs. Preferably, the method comprises culturing αβ T cell-depleted PBMCs in the presence of granulopoietic cells.

The method may comprise:
(a) isolating PBMCs from a sample obtainable from a donor;
(b) depleting αβ T cells from the isolated PBMCs; and
(c) culturing the αβ T cell-depleted PBMCs in the presence of granulopoietic cells,
thereby forming the composition.

αβ T cells may be depleted from PBMCs using any suitable means. For example, the step of depleting αβ T cells from PBMCs may comprise:
(a) incubating the PBMCs in the presence of a biotin-conjugated anti-TCR αβ antibody and anti-biotin microbeads such that the αβ T cells present in the PBMCs bind to the biotin-conjugated anti-TCR αβ antibody; and
(b) separating the antibody-bound αβ T cells from the PBMCs, e.g. using magnetic activated cell sorting (MACS).

The step of depleting αβ T cells from the isolated PBMCs may comprise:
(a) incubating the PBMCs in the presence of a biotin-conjugated anti-TCR αβ antibody (Clone BW242/412; 1:50 dilution) for 15 minutes at room temperature such that the αβ T cells present in the PBMCs bind to the biotin-conjugated anti-TCR αβ antibody;
(b) washing the antibody-bound cells in MACS buffer;
(c) centrifuging the antibody-bound cells at 300×g for 5 minutes;
(d) resuspending the centrifuged cells in MACS buffer (80 μl/1×10⁷ cells) containing anti-biotin microbeads (20 μl/1×10⁷ cells);
(e) incubating the resuspended cells at 4° C. for 15 minutes such that the antibody-bound cells bind to the anti-biotin microbeads;
(f) washing the microbead-bound cells in MACS buffer and centrifuging the microbead-bound cells at 300×g for 5 minutes; and
(g) resuspending up to 1.25×10⁸ microbead-bound cells in 500 μl of MACS buffer and applying the microbead-bound cells to an LD column placed in the magnetic field of the MACS MultiStand (Miltenyi Biotec),
wherein unlabelled cells (aBTCR) cells pass through the column and are collected.

As used herein, the term “αβ T cell-depleted PBMCs” and the like refers to a population of PBMCs which are substantially or wholly free of αβ T cells. Thus, in one embodiment the term “αβ T-cell depleted PBMCs” means a sample of PBMCs that does not comprise an αβ T cell. The term “a sample of PBMCs that does not comprise an αβ T cell” in this context means that the PBMCs comprises no, or substantially no, αβ T cells. The term “substantially no” as used in this context may refer to a sample of PBMCs wherein fewer than 10%, fewer than 5%, fewer than 4%, fewer than 3%, fewer than 2%, fewer than 1% of the cells, fewer than 0.1% of the cells, fewer than 0.01% of the cells, fewer than 0.001% of the cells, fewer than 0.0001% of the PBMCs are αβ T cells. The term “substantially no” as used in this context may mean that the PBMCs comprise up to about 1×10⁹ αβ T cells/kg of the subject to be administered; up to about 1×10⁸ αβ T cells/kg of the subject to be administered; up to about 1×10⁷ αβ T cells/kg of the subject to be administered; up to about 1×10⁶ αβ T cells/kg of the subject to be treated; preferably up to about 1×10⁵ αβ T cells/kg of the subject to be treated. The term “substantially no” as used in this context may mean that the PBMCs comprise about 1×10¹-1×10⁹ αβ T cells/kg of the subject to be treated; about 1×10²-1×10⁸ αβ T cells/kg of the subject to be treated; about 1×10³-1×10⁷ αβ T cells/kg of the subject to be treated; about 1×10⁴-1×10⁶ αβ T cells/kg of the subject to be treated; preferably about 1×10⁴-1×10⁵ αβ T cells/kg of the subject to be treated. The term “substantially no” as used in this context may mean that the PBMCs comprise up to about 7×10¹⁰ αβ T cells; up to about 7×10⁹ αβ T cells; up to about 7×10⁸ αβ T cells; up to about 7×10⁷ αβ T cells; preferably up to about 7×10⁶ αβ T cells. The term “substantially no” as used in this context may mean that the PBMCs comprise about 7×10²-7×10¹⁰ αβ T cells/kg of the subject to be treated; about 7×10³-7×10⁹ αβ T cells/kg of the subject to be treated; about 7×10⁴-7×10⁸ αβ T cells/kg of the subject to be treated; about 7×10⁵-7×10⁷ αβ T cells/kg of the subject to be treated; preferably about 7×10⁵-7×10⁶ αβ T cells. Particularly preferably, the PBMCs comprise no αβ T cells

The αβ T cells may be depleted at any suitable time, e.g. prior to administration to a subject. For example, the PBMCs may be cultured in the presence of granulopoietic cells before or after the step of depleting αβ T cells from the isolated PBMCs.

The granulopoietic cells and PBMCs or αβ T cell-depleted PBMCs (e.g. αβ T cell-depleted PBMCs), may be cultured together at any suitable ratio. The granulopoietic cells and PBMCs or αβ T cell-depleted PBMCs (e.g. αβ T cell-depleted PBMCs), may be cultured together at a ratio of 100:1 to 0.01:1 granulopoietic cells to PBMCs or αβ T cell-depleted PBMCs. The population of granulopoietic cells and PBMCs or αβ T cell-depleted PBMCs (e.g. αβ T cell-depleted PBMCs), may be cultured together at a ratio of 100:1 to 0.01:1; 75:1 to 0.05:1; 50:1 to 0.1:1; 25:1 to 0.2:1; 10:1 to 0.25:1; 5:1 to 0.25:1; 3:1 to 0.25:1; or 2:1 to 0.5:1 granulopoietic cells to PBMCs or αβ T cell-depleted PBMCs. Preferably, the granulopoietic cells and PBMCs or αβ T cell-depleted PBMCs (e.g. αβ T cell-depleted PBMCs) are cultured together at a ratio of 3:1 to 0.25:1 granulopoietic cells to PBMCs or αβ T cell-depleted PBMCs.

The granulopoietic cells and PBMCs or αβ T cell-depleted PBMCs (e.g. αβ T cell-depleted PBMCs), may be cultured together at a ratio of less than or equal to 100:1, 75:1, 50:1, 25:1, 10:1, 5:1, 3:1, 2:1, 1:1, 0.5:1, 0.25:1, 0.1:1, 0.05:1 or 0.01:1 granulopoietic cells to PBMCs or αβ T cell-depleted PBMCs (e.g. αβ T cell-depleted PBMCs). The granulopoietic cells and PBMCs or αβ T cell-depleted PBMCs may be cultured together at a ratio of at least 0.01:1, 0.05:1 0.1:1, 0.25:1, 0.5:1, 1:1, 2:1, 3:1, 5:1, 10:1, 25:1, 50:1, 75:1, or 100:1 granulopoietic cells to PBMCs or αβ T cell-depleted PBMCs (e.g. αβ T cell-depleted PBMCs). The granulopoietic cells and PBMCs or αβ T cell-depleted PBMCs (e.g. αβ T cell-depleted PBMCs), may be cultured together at a ratio of 100:1, 75:1, 50:1, 25:1, 10:1, 5:1, 3:1, 2:1, 1:1, 0.5:1, 0.25:1, 0.1:1, 0.05:1 or 0.01:1 granulopoietic cells to PBMCs or αβ T cell-depleted PBMCs (e.g. αβ T cell-depleted PBMCs). Preferably, the granulopoietic cells and PBMCs or αβ T cell-depleted PBMCs (e.g. αβ T cell-depleted PBMCs), are cultured together at a ratio of 2:1, 1:1, or 0.5:1 granulopoietic cells to PBMCs or αβ T cell-depleted PBMCs (e.g. αβ T cell-depleted PBMCs). For example, the granulopoietic cells and PBMCs or αβ T cell-depleted PBMCs (e.g. αβ T cell-depleted PBMCs), may be cultured together at a ratio of 2:1 granulopoietic cells to PBMCs or αβ T cell-depleted PBMCs. The granulopoietic cells and PBMCs or αβ T cell-depleted PBMCs (e.g. αβ T cell-depleted PBMCs), may be cultured together at a ratio of 1:1 granulopoietic cells to PBMCs or αβ T cell-depleted PBMCs. The granulopoietic cells and PBMCs or αβ T cell-depleted PBMCs (e.g. αβ T cell-depleted PBMCs), may be cultured together at a ratio of 0.5:1 granulopoietic cells to PBMCs or αβ T cell-depleted PBMCs.

PBMCs and αβ T cell-depleted PBMCs may comprise granulopoietic cells. Without being bound by theory, it is believed that there is a higher concentration of haematopoietic cells, which are capable of differentiating (preferably differentiate) into granulopoietic cells, in PBMCs obtainable (e.g. obtained) from mobilized blood, e.g. G-CSF mobilized blood. Preferably, the PBMCs or αβ T cell-depleted PBMCs are obtainable from mobilized blood, e.g. G-CSF mobilized blood. The method may comprise obtaining PBMCs or αβ T cell-depleted PBMCs from mobilized blood, e.g. G-CSF mobilized blood.

The method may comprise increasing the number of granulopoietic cells present in PBMCs or αβ T cell-depleted PBMCs. The method may comprise increasing the concentration of granulopoietic cells present in PBMCs or αβ T cell-depleted PBMCs. The number or concentration of granulopoietic cells present in PBMCs or αβ T cell-depleted PBMCs may be increased by any suitable means. Accordingly, the step of culturing the PBMCs or αβ T cell-depleted PBMCs in the presence of granulopoietic cells may comprise culturing the PBMCs or αβ T cell-depleted PBMCs under conditions suitable for expansion and/or differentiation of the haematopoietic cells present in the PBMCs or αβ T cell-depleted PBMCs.

Particularly preferably, the method of preparing a composition of the invention comprises culturing αβ T cell-depleted PBMCs (e.g. obtainable from a sample of mobilized blood, such as G-CSF mobilized blood) under conditions that promote differentiation of progenitor cells present in the αβ T cell-depleted PBMCs into granulopoietic cells, thereby forming the composition. Advantageously, this allows for the composition to be prepared using cells from a single source, thereby providing a significantly streamlined and efficient method of preparing a composition of the invention.

The method may comprise culturing αβ T cell-depleted PBMCs (e.g. obtainable from a sample of mobilized blood, such as G-CSF mobilized blood) under conditions to produce a progenitor cell from stem cells present in the αβ T cell-depleted PBMCs.

The method may optionally comprise depleting αβ T cells from PBMCs (e.g. obtainable from a sample of mobilized blood, such as G-CSF mobilized blood).

Accordingly, the method may comprise:
(a) depleting αβ T cells from PBMCs (e.g. obtainable from a sample of mobilized blood, such as G-CSF mobilized blood); and
(b) culturing the αβ T cell-depleted PBMCs under conditions that promote differentiation of progenitor cells present in the αβ T cell-depleted PBMCs into granulopoietic cells,
thereby forming the composition.

The method may comprise:
(a) depleting αβ T cells from PBMCs (e.g. obtainable from a sample of mobilized blood, e.g. G-CSF mobilized blood);
(b) culturing the αβ T cell-depleted PBMCs under conditions to produce progenitor cells from stem cells present in the αβ T cell-depleted PBMCs; and
(c) culturing the progenitor cell present in the αβ T cell-depleted PBMCs under conditions that promote differentiation of progenitor cells present in the αβ T cell-depleted PBMCs into granulopoietic cells,
thereby forming the composition.

The conditions that promote differentiation of progenitor cells present in PBMCs or αβ T cell-depleted PBMCs (e.g. αβ T cell-depleted PBMCs) into granulopoietic cells may be any suitable conditions. For example, the conditions that promote differentiation of progenitor cells present in PBMCs or αβ T cell-depleted PBMCs (e.g. αβ T cell-depleted PBMCs) into granulopoietic cells may be the conditions described herein that are suitable for obtaining a population of granulopoietic cells.

Similarly, the conditions to produce progenitor cells from stem cells present in PBMCs or αβ T cell-depleted PBMCs (e.g. αβ T cell-depleted PBMCs) may be any suitable conditions. For example, the conditions to produce progenitor cells from stem cells present in PBMCs or αβ T cell-depleted PBMCs (e.g. αβ T cell-depleted PBMCs) may be the conditions described herein that are used in a method of obtaining a population of granulopoietic cells comprising a step of culturing a stem cell in culture conditions to produce the progenitor cell.

The method may further comprise culturing the PBMCs or αβ T cell-depleted PBMCs (e.g. αβ T cell-depleted PBMCs) under conditions suitable for maintenance of NK cells. The method may further comprise culturing the PBMCs or αβ T cell-depleted PBMCs (e.g. αβ T cell-depleted PBMCs) under conditions suitable for maintenance of γδ T cells. Preferably, the method comprises culturing the PBMCs or αβ T cell-depleted PBMCs (e.g. αβ T cell-depleted PBMCs) under conditions suitable for maintenance of NK cells and γδ T cells.

Accordingly, the method may comprise:
(a) culturing αβ T cell-depleted PBMCs under conditions that promote differentiation of progenitor cells present in the αβ T cell-depleted PBMCs into granulopoietic cells; and
(b) culturing the αβ T cell-depleted PBMCs under conditions suitable for maintenance of NK cells and γδ T cells present in the αβ T cell-depleted PBMCs,
thereby forming the composition.

The method may comprise:
(a) culturing αβ T cell-depleted PBMCs under conditions to produce progenitor cells from stem cells present in the αβ T cell-depleted PBMCs and under conditions suitable for maintenance of NK cells and γδ T cells present in the αβ T cell-depleted PBMCs; and
(b) culturing the progenitor cells present in the αβ T cell-depleted PBMCs under conditions that promote differentiation of the progenitor cells present in the αβ T cell-depleted PBMCs into granulopoietic cells,
thereby forming the composition.

The conditions suitable for maintenance of NK cells and γδ T cells may comprise culturing the PBMCs or αβ T cell-depleted PBMCs (e.g. αβ T cell-depleted PBMCs) in the presence of one or more cytokines selected from: IL-15, IL-2, IL-7, IL-9, IL-4, and IL-21. The conditions suitable for maintenance of NK cells and γδ T cells may comprise culturing the PBMCs or αβ T cell-depleted PBMCs (e.g. αβ T cell-depleted PBMCs) in the presence of one or more cytokines selected from: IL-15, IL-2, IL-7, IL-9, IL-4, and IL-21 wherein the one or more cytokines is present at a concentration of 1-20 ng/ml, 5-15 ng/ml or 7.5-12.5 ng/ml. The conditions suitable for maintenance of NK cells and γδ T cells may comprise culturing the PBMCs or αβ T cell-depleted PBMCs (e.g. αβ T cell-depleted PBMCs) in the presence of one or more cytokines selected from: IL-15, IL-2, IL-7, IL-9, IL-4, and IL-21 wherein the one or more cytokines is present at a concentration of 1, 2, 3, 4, 5, 6, 7, 8, 9, 10, 11, 12, 13, 14, 15, 16, 17, 18, 19 or 20 ng/ml. Preferably, during the maintenance phase, the one or more cytokines is present at a concentration of 10 ng/mL. Accordingly, the method may comprise culturing the PBMCs or αβ T cell-depleted PBMCs (e.g. αβ T cell-depleted PBMCs) in the presence of one or more cytokines selected from: IL-15, IL-2, IL-7, IL-9, IL-4, and IL-21 wherein the one or more cytokines is present at a concentration of 10 ng/ml. The method may comprise culturing the PBMCs or αβ T cell-depleted PBMCs (e.g. αβ T cell-depleted PBMCs) in the presence of one or more cytokines selected from: IL-15, IL-2, IL-7, IL-9, IL-4, and IL-21 (e.g. at a concentration of 10 ng/ml) for an appropriate time. For example, the method may comprise culturing the PBMCs or αβ T cell-depleted PBMCs (e.g. αβ T cell-depleted PBMCs) in the presence of one or more cytokines selected from: IL-15, IL-2, IL-7, IL-9, IL-4, and IL-21 (e.g. at a concentration of 10 ng/mL) for at least 1 day, 2 days, 3 days, 4 days, 5 days, 6 days, 7 days, 8 days, 9 days or 10 days. The method may comprise culturing the PBMCs or αβ T cell-depleted PBMCs (e.g. αβ T cell-depleted PBMCs) in the presence of one or more cytokines selected from: IL-15, IL-2, IL-7, IL-9, IL-4, and IL-21 (e.g. at a concentration of 10 ng/ml) for up to 1 day, 2 days, 3 days, 4 days, 5 days, 6 days, 7 days, 8 days, 9 days or 10 days. The method may comprise culturing the PBMCs or αβ T cell-depleted PBMCs (e.g. αβ T cell-depleted PBMCs) in the presence of one or more cytokines selected from: IL-15, IL-2, IL-7, IL-9, IL-4, and IL-21 (e.g. at a concentration of 10 ng/ml) for 1 day, 2 days, 3 days, 4 days, 5 days, 6 days, 7 days, 8 days, 9 days or 10 days. The method may comprise culturing the PBMCs or αβ T cell-depleted PBMCs (e.g. αβ T cell-depleted PBMCs) in the presence of one or more cytokines selected from: IL-15, IL-2, IL-7, IL-9, IL-4, and IL-21 (e.g. at a concentration of 10 ng/mL) for 1-10 days, 2-10 days, 3-10 days, 4-10 days, 5-9 days, 6-9 days, or 7-9 days. Preferably, the method comprises culturing the PBMCs or αβ T cell-depleted PBMCs (e.g. αβ T cell-depleted PBMCs) in the presence of one or more cytokines selected from: IL-15, IL-2, IL-7, IL-9, IL-4, and IL-21 (e.g. at a concentration of 10 ng/ml) for 7-9 days.

The method may further comprise culturing the PBMCs or αβ T cell-depleted PBMCs (e.g. αβ T cell-depleted PBMCs) under conditions suitable for activation of NK cells. The method may further comprise culturing the PBMCs or αβ T cell-depleted PBMCs (e.g. αβ T cell-depleted PBMCs) under conditions suitable for activation of γδ T cells. Preferably, the method comprises culturing the PBMCs or αβ T cell-depleted PBMCs (e.g. αβ T cell-depleted PBMCs) under conditions suitable for activation of NK cells and γδ T cells.

Accordingly, the method may comprise:
(a) culturing αβ T cell-depleted PBMCs under conditions that promote differentiation of progenitor cells present in the αβ T cell-depleted PBMCs into granulopoietic cells; and
(b) culturing the αβ T cell-depleted PBMCs under conditions suitable for activation of NK cells and γδ T cells present in the αβ T cell-depleted PBMCs,
thereby forming the composition.

The conditions suitable for differentiation of haematopoietic cells present in the αβ T cell-depleted PBMCs may be suitable for activation of NK cells and γδ T cells present in the αβ T cell-depleted PBMCs.

The method may comprise:
(a) culturing αβ T cell-depleted PBMCs under conditions to produce progenitor cells from stem cells present in the αβ T cell-depleted PBMCs and culturing the αβ T cell-depleted PBMCs under conditions suitable for maintenance of NK cells and γδ T cells present in the αβ T cell-depleted PBMCs; and
(b) culturing the progenitor cells present in the αβ T cell-depleted PBMCs under conditions that promote differentiation of the progenitor cells present in the αβ T cell-depleted PBMCs into granulopoietic cells and culturing the αβ T cell-depleted PBMCs under conditions suitable for activation of NK cells and γδ T cells present in the αβ T cell-depleted PBMCs,
thereby forming the composition.

The conditions suitable for activation of NK cells and γδ T cells may comprise culturing the PBMCs or αβ T cell-depleted PBMCs (e.g. αβ T cell-depleted PBMCs) in the presence of one or more cytokines selected from: IL-15, IL-2, IL-7, IL-9, IL-4, and IL-21. The conditions suitable for activation of NK cells and γδ T cells may comprise culturing the PBMCs or αβ T cell-depleted PBMCs (e.g. αβ T cell-depleted PBMCs) in the presence of one or more cytokines selected from: IL-15, IL-2, IL-7, IL-9, IL-4, and IL-21 wherein the one or more cytokines is present at a concentration of 20-200 ng/ml, 50-150 ng/ml or 75-125 ng/ml. The conditions suitable for activation of NK cells and γδ T cells may comprise culturing the PBMCs or αβ T cell-depleted PBMCs (e.g. αβ T cell-depleted PBMCs) in the presence of one or more cytokines selected from: IL-15, IL-2, IL-7, IL-9, IL-4, and IL-21 wherein the one or more cytokines is present at a concentration of 20, 30, 40, 50, 60, 70, 80, 90, 100, 110, 120, 130, 140, 150, 160, 170, 180, 190, or 200 ng/ml. Preferably, during the activation phase, the one or more cytokines is present at a concentration of 100 ng/ml. Accordingly, the method may comprise culturing the PBMCs or αβ T cell-depleted PBMCs (e.g. αβ T cell-depleted PBMCs) in the presence of one or more cytokines selected from: IL-15, IL-2, IL-7, IL-9, IL-4, and IL-21 wherein the one or more cytokines is present at a concentration of 100 ng/ml. The method may comprise culturing the PBMCs or αβ T cell-depleted PBMCs (e.g. αβ T cell-depleted PBMCs) in the presence of one or more cytokines selected from: IL-15, IL-2, IL-7, IL-9, IL-4, and IL-21 (e.g. at a concentration of 100 ng/ml) for any appropriate time. For example, the method may comprise culturing the PBMCs or αβ T cell-depleted PBMCs (e.g. αβ T cell-depleted PBMCs) in the presence of one or more cytokines selected from: IL-15, IL-2, IL-7, IL-9, IL-4, and IL-21 (e.g. at a concentration of 100 ng/mL) for at least 1 day, 2 days, 3 days, 4 days, 5 days, or 6 days. The method may comprise culturing the PBMCs or αβ T cell-depleted PBMCs (e.g. αβ T cell-depleted PBMCs) in the presence of one or more cytokines selected from: IL-15, IL-2, IL-7, IL-9, IL-4, and IL-21 (e.g. at a concentration of 100 ng/ml) for up to 1 day, 2 days, 3 days, 4 days, 5 days, or 6 days. The method may comprise culturing the PBMCs or αβ T cell-depleted PBMCs (e.g. αβ T cell-depleted PBMCs) in the presence of one or more cytokines selected from: IL-15, IL-2, IL-7, IL-9, IL-4, and IL-21 (e.g. at a concentration of 100 ng/ml) for 1 day, 2 days, 3 days, 4 days, 5 days, or 6 days. The method may comprise culturing the PBMCs or αβ T cell-depleted PBMCs (e.g. αβ T cell-depleted PBMCs) in the presence of one or more cytokines selected from: IL-15, IL-2, IL-7, IL-9, IL-4, and IL-21 (e.g. at a concentration of 100 ng/ml) for 1-6 days, 2-6 days, 3-6 days, or 4-6 days. Preferably, the method comprises culturing the PBMCs or αβ T cell-depleted PBMCs (e.g. αβ T cell-depleted PBMCs) in the presence of one or more cytokines selected from: IL-15, IL-2, IL-7, IL-9, IL-4, and IL-21 (e.g. at a concentration of 100 ng/ml) for 4-6 days. The conditions suitable for activation of NK cells and γδ T cells may further comprise culturing the PBMCs or αβ T cell-depleted PBMCs (e.g. αβ T cell-depleted PBMCs) in the presence of a T cell receptor activator such as an OKT3 activator, e.g. a T cell receptor antibody such as an anti-CD3 antibody. Without being bound by theory, it is believed that culturing the PBMCs or αβ T cell-depleted PBMCs in the presence of a T cell receptor may synergistically enhance the expansion and activation of γδ T cells. Accordingly, the method may comprise culturing the PBMCs or αβ T cell-depleted PBMCs (e.g. αβ T cell-depleted PBMCs) in the presence of a T cell receptor activator such as an OKT3 activator, e.g. a T cell receptor antibody such as an anti-CD3 antibody.

Preferably, the method comprises culturing the PBMCs or αβ T cell-depleted PBMCs (e.g. αβ T cell-depleted PBMCs) in the presence of IL-15. The method may further comprise culturing the PBMCs or αβ T cell-depleted PBMCs (e.g. αβ T cell-depleted PBMCs) in the presence of a T cell receptor activator such as an OKT3 activator, e.g. a T cell receptor antibody such as an anti-CD3 antibody.

The method may comprise culturing a non-granulocytic immune cell differentiated from an iPSC (e.g. an iPSC-derived γδ T cell and/or an iPSC-derived NK cell) in the presence of a population of granulopoietic cells, thereby forming the composition. The method may comprise culturing a non-granulocytic immune cell in the presence of a population of granulopoietic cells differentiated from an iPSC, thereby forming the composition. The method may comprise culturing a non-granulocytic immune cell differentiated from an iPSC (e.g. an iPSC-derived yo T cell and/or an iPSC-derived NK cell) in the presence of a population of granulopoietic cells differentiated from an iPSC, thereby forming the composition. The method may comprise culturing an iPSC-derived αβ T cell, an iPSC-derived γδ T cell, an iPSC-derived NK cell or combinations thereof in the presence of an iPSC-derived granulopoietic cell, thereby forming the composition. For example, the method may comprise culturing an iPSC-derived NK cell in the presence of an iPSC-derived granulopoietic cell, thereby forming the composition. The method may comprise culturing an iPSC-derived γδ T cell in the presence of an iPSC-derived granulopoietic cell, thereby forming the composition. The method may comprise culturing an iPSC-derived γδ T cell and an iPSC-derived NK cell in the presence of an iPSC-derived granulopoietic cell, thereby forming the composition.

The method may comprise differentiating an iPSC into a population of granulopoietic cells, e.g. a population of granulopoietic cells as defined herein, and culturing a non-granulocytic immune cell in the presence of the iPSC-derived granulopoietic cell. The method may comprise differentiating an iPSC into a non-granulocytic immune cell, e.g. a γδ T cell and/or an NK cell, and culturing the iPSC-derived non-granulocytic immune cell in the presence of a population of granulopoietic cells. The method may comprise differentiating an iPSC into a population of granulopoietic cells and differentiating an iPSC into a non-granulocytic immune cell, e.g. a yo T cell and/or an NK cell, and culturing the iPSC-derived non-granulocytic immune cell in the presence of the iPSC-derived granulopoietic cell.

Accordingly, the method may comprise:
(a) differentiating an iPSC into an iPSC-derived granulopoietic cell;
(b) differentiating an iPSC into an iPSC-derived γδ T cell;
(c) differentiating an iPSC into an iPSC-derived NK cell,
and co-culturing the iPSC-derived granulopoietic cell, iPSC-derived γδ T cell, and iPSC-derived NK cell, thereby forming the composition.

The iPSC may be obtainable from any suitable donor. For example, the iPSC may be obtainable from a donor who produces granulocytes with the ability to kill cancer cells, as defined using an assay described herein.

The iPSC may be obtainable from any suitable source. For example, the iPSC may be obtainable from a somatic cell, such as an αβ T cell or γδ T cell. The iPSC may be obtainable from a stem cell. In embodiments when the method comprises differentiating an iPSC into a γδ T cell, the iPSC may be obtainable from a γδ T cell. In embodiments when the method comprises differentiating an iPSC into an αβ T cell, the iPSC may be obtainable from an αβ T cell.

Accordingly, the method may comprise:
(a) differentiating an iPSC into an iPSC-derived granulopoietic cell;
(b) differentiating an iPSC obtainable from a γδ T cell into an iPSC-derived γδ T cell; and
(c) differentiating an iPSC into an iPSC-derived NK cell,
and co-culturing the iPSC-derived granulopoietic cell, iPSC-derived αβ T cell, and iPSC-derived NK cell, thereby forming the composition.

The method may comprise:
(a) differentiating an iPSC obtainable from a γδ T cell into an iPSC-derived granulopoietic cell;
(b) differentiating an iPSC obtainable from a γδ T cell into an iPSC-derived γδ T cell; and
(c) differentiating an iPSC obtainable from a γδ T cell into an iPSC-derived NK cell,
and co-culturing the iPSC-derived granulopoietic cell, iPSC-derived αβ T cell, and iPSC-derived NK cell, thereby forming the composition.

Cell culture additives may enhance the amplification of therapeutic immune responses. Accordingly, the population of granulopoietic cells and non-granulocytic immune cell may be cultured in the presence of any suitable cell culture additive such as a growth factor, a cytokine, or a chemokine. For example, the population of granulopoietic cells and non-granulocytic immune cell may be cultured in the presence of a granulocyte-macrophage colony-stimulating factor (GM-CSF), a granulocyte colony-stimulating factor (G-CSF), a growth hormone; serotonin, vitamin C, vitamin D, glutamine (Gln), arachidonic acid, AGE-albumin, an interleukin, TNF-alpha, Flt-3 ligand, thrombopoietin, serum (e.g. foetal bovine serum [FBS]), retinoic acid, lipopolysaccharide (LPS), IFN-gamma, IFN-beta, or combinations thereof. The population of granulopoietic cells and non-granulocytic immune cell may be cultured in the presence of IFN-gamma and a GM-CSF. The population of granulopoietic cells and non-granulocytic immune cell may be cultured in the presence of TNF-alpha. The population of granulopoietic cells and non-granulocytic immune cell may be cultured in the presence of a granulocyte colony-stimulating factor (G-CSF), and a growth hormone, and serotonin, and vitamin C, and vitamin D, and glutamine (Gln), and arachidonic acid, and AGE-albumin, and an interleukin, and TNF-alpha, and Flt-3 ligand, and thrombopoietin, and foetal bovine serum (FBS). The population of granulopoietic cells and non-granulocytic immune cell may be cultured in the presence of a granulocyte-macrophage colony-stimulating factor (GM-CSF), and a granulocyte colony-stimulating factor (G-CSF), and a growth hormone, and serotonin, and vitamin C, and vitamin D, and glutamine (Gln), and arachidonic acid, and AGE-albumin, and an interleukin, and TNF-alpha, and Flt-3 ligand, and thrombopoietin, and foetal bovine serum (FBS), and retinoic acid, and lipopolysaccharide (LPS), and IFN-gamma, and IFN-beta. The population of granulopoietic cells and non-granulocytic immune cell may be cultured in the presence of an anti-CD3 agonist, such as an anti-OKT3 antibody. The population of granulopoietic cells and non-granulocytic immune cell may be cultured in the presence of an anti-OKT3 antibody.

A population of granulopoietic cells and non-granulocytic immune cell that may be used in the various aspects of the invention may be provided in the form of an enriched population of such granulopoietic cells and non-granulocytic immune cells. In one aspect, the invention provides a pharmaceutical composition comprising an enriched population of granulopoietic cells and non-granulocytic immune cells.

Merely by way of example, such an enriched population may be a population of cells in which the granulopoietic cells and non-granulocytic immune cells comprise at least 0.1%, at least 0.2%, at least 0.3%, at least 0.4%, at least 0.5%, at least 0.6%, at least 0.7%, at least 0.8%, at least 0.9%, or at least 1% of the total cell population. Such an enriched population may further be a population of cells in which the granulopoietic cells comprise at least 2%, at least 3%, at least 4%, at least 5%, at least 6%, at least 7%, at least 8%, at least 9%, or at least 10% of the total cell population. Indeed, an enriched population may be a population of cells in which the granulopoietic cells comprise at least at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or substantially 100% of the total cell population present.

The granulopoietic cells and non-granulocytic immune cells of such an enriched population may be as defined in any appropriate embodiment set out elsewhere in the specification. For example, the granulopoietic cells of an enriched population may be CD62L⁻.

In one aspect of the invention, there is provided a pharmaceutical composition comprising an enriched population of granulopoietic cells and non-granulocytic immune cells. The enriched population of granulopoietic cells and non-granulocytic immune cells incorporated in a pharmaceutical composition of the invention may be as considered above.

Suitably, the granulopoietic cells present in a pharmaceutical composition of the invention may be CD62L⁻. Suitably, the granulopoietic cells present in a pharmaceutical composition of the invention may be CD16⁻. Suitably, the granulopoietic cells present in a pharmaceutical composition of the invention may be CD10⁻. Suitably, the granulopoietic cells present in a pharmaceutical composition of the invention may be CD16⁻, CD10⁻ and CD62L⁻.

The pharmaceutical composition may be formulated in any manner conventional for its intended route of administration. For example, the pharmaceutical composition may be formulated for administration by injection or infusion.

Suitably, the compositions (e.g. pharmaceutical compositions) of the invention may comprise a granulocyte-macrophage colony-stimulating factor (GM-CSF), a granulocyte colony-stimulating factor (G-CSF), a growth hormone; serotonin, vitamin C, vitamin D, glutamine (Gln), arachidonic acid, AGE-albumin, an interleukin, TNF-alpha, Flt-3 ligand, thrombopoietin, serum (e.g. foetal bovine serum [FBS]), retinoic acid, lipopolysaccharide (LPS), IFN-gamma, IFN-beta, or combinations thereof. Suitably, the compositions (e.g. pharmaceutical compositions) comprise IFN-gamma and a GM-CSF. Preferably, the compositions (e.g. pharmaceutical compositions) comprise TNF-alpha. Particularly preferably, the compositions (e.g. pharmaceutical compositions) comprise a granulocyte-macrophage colony-stimulating factor (GM-CSF), and a granulocyte colony-stimulating factor (G-CSF), and a growth hormone, and serotonin, and vitamin C, and vitamin D, and glutamine (Gln), and arachidonic acid, and AGE-albumin, and an interleukin, and TNF-alpha, and Flt-3 ligand, and thrombopoietin, and foetal bovine serum (FBS). Preferably, the compositions (e.g. pharmaceutical compositions) comprise a granulocyte-macrophage colony-stimulating factor (GM-CSF), and a granulocyte colony-stimulating factor (G-CSF), and a growth hormone, and serotonin, and vitamin C, and vitamin D, and glutamine (Gln), and arachidonic acid, and AGE-albumin, and an interleukin, and TNF-alpha, and Flt-3 ligand, and thrombopoietin, and foetal bovine serum (FBS), and retinoic acid, and lipopolysaccharide (LPS), and IFN-gamma, and IFN-beta.

In the context of the present invention a host therapeutic immune response preferably should be taken as being an immune response that contributes to or achieves a desired therapeutic outcome. In a suitable embodiment, a host therapeutic immune response may be an immune response that leads (directly or indirectly) to the killing of cancer cells, thus allowing treatment of cancer. In a suitable embodiment, a host therapeutic immune response may be an immune response that leads (directly or indirectly) to the killing of infected cells or of cellular infectious agents, thus allowing treatment of an infection.

A host therapeutic immune response may involve the action of any cells of the immune system. A “non-granulocytic immune response” may involve the action of any cells of the immune system, other than granulocytes. Non-granulocytic immune responses may be beneficially amplified by any suitable population of granulopoietic cells in accordance with the invention, or any suitable pharmaceutical composition comprising such a population of granulopoietic cells. Suitable compositions may utilise populations of granulopoietic cells without including non-granulocytic immune cells. Equally, compositions comprising a population of granulopoietic cells and a non-granulocytic immune cell (or cells) may amplify a host therapeutic immune response, e.g. after administration to a subject. Merely by way of example, a host therapeutic immune response that may be amplified by the compositions (e.g. pharmaceutical compositions), medical uses, or methods of treatment of the invention may involve the action of one or more cell types selected from the group comprising (or consisting) of: T cells (including, but not limited to CD8⁺ T cells; CD4⁺ T cells; NK T cells; αβ T cells; γδ T cells; peripheral blood T cells; and tumour infiltrated T cells); NK cells; monocytes; macrophages; dendritic cells (DCs); and B cells.

Amplification of an immune response (e.g. a host therapeutic immune response) may be demonstrated by one or more of the following: increased activation of immune cells involved in the immune response; increased expression of degranulation markers by immune cells involved in the immune response; increased expression of costimulatory molecules by immune cells involved in the immune response; increased proliferation by immune cells involved in the immune response; increased survival by immune cells involved in the immune response; increased abundance of immune cells involved in the immune response; increased expression of cytokines by immune cells involved in the immune response; increased trafficking by immune cells involved in the immune response; increased recruitment into the TME of immune cells involved in the immune response; increased cytocidal activity by immune cells involved in the immune response; or increased tumour cell killing activity by immune cells involved in the immune response.

Alternatively, or additionally, amplification of a host therapeutic immune response may be assessed with reference to the outcome to be achieved by the therapeutic immune response.

For example, in the case of a host therapeutic immune response to be used in the treatment of cancer, amplification of the immune response may be demonstrated by an increase in the efficacy of the treatment of cancer. Such an increase in efficacy may be demonstrated by a reduction in symptoms; an increase in rate and/or duration of patient survival; a reduction of tumour burden; prevention or delay of relapse; a reduction in severity of relapse; a reduction in the number of incidences of relapse; a reduction in the number of incidences of metastasis; and/or a prevention or delay of metastasis.

In the case of a host therapeutic immune response to be used in the treatment of infection, amplification of the immune response may be demonstrated by an increase in the efficacy of the treatment of the infection. Such an increase may be demonstrated by reduction of symptoms; an increase in rate and/or duration of patient survival; reduction in infection burden; and/or a reduction of time to clearance of infection.

For the purposes of the present disclosure, references to “host” cells (such as host immune cells) or a “host” immune response may be taken as referring to the cells or immune response of a subject receiving treatment with, or putatively receiving treatment with, populations of granulopoietic cells or compositions in accordance with any of the various aspects of the invention. Except where the context requires otherwise, all references to immune cells or immune responses in connection with the various aspects and embodiments of the invention should be taken as applicable to host immune cells, or to host immune responses.

A population of granulopoietic cells or composition suitable for use in accordance with the various aspects of the present invention may be capable of increasing (preferably increase) activation of host immune cells. Accordingly, such a cell may be capable of amplifying (preferably amplify) a host therapeutic immune response by increasing activation of host immune cells. It will be appreciated that it is activated immune cells that are primarily responsible for providing the desired activity in a therapeutic immune response. Accordingly, the ability of the medical uses and methods of treatment to increase activation of host immune cells will be of benefit in almost all circumstances in which a therapeutically effective immune response is required. In particular, the amplification of a therapeutic immune response by increasing activation of host immune cells may, without limitation, be advantageous in the treatment of cancer or the treatment of infections.

Populations of granulopoietic cells suitable for use in accordance with the present invention may exhibit some or all of the properties set out above.

Suitably, a therapeutically effective amount of such a population of granulopoietic cells (or of a composition [e.g. a pharmaceutical composition] of the invention comprising a population of granulopoietic cells), for example for use in in accordance with the invention, is an amount sufficient to increase activation of immune cells, such as host immune cells. The extent of increase, relevant host immune cells, and suitable indicators of increased activation, may be as considered in the preceding paragraphs and/or as in those that follow.

A population of granulopoietic cells or composition suitable for use in accordance with the present invention may increase activation of host T cells. Accordingly, such a population of granulopoietic cells or composition may be capable of amplifying (preferably amplify) a host therapeutic immune response by increasing activation of host T cells.

It will be appreciated that increased activation of host T cells such as CD8⁺ and CD4⁺ T cells will significantly contribute to the desired activity in a therapeutic immune response. Cytotoxic T cells, such as CD8⁺ T cells, are known to have direct cytocidal activity, whilst helper T cells, such as CD4⁺ T cells, are known to help coordinate the immune response by further stimulating other immune cells. Accordingly, the use of a population of granulopoietic cells or composition to increase activation of host T cells will be of benefit in a wide range of circumstances in which a therapeutically effective immune response is required. In particular, the amplification of a host therapeutic immune response by increasing activation of host T cells may, without limitation, be advantageous in the treatment of cancer or the treatment of infections.

A host T cell, activation of which may be increased, may be selected from the group comprising (or consisting of): a CD8⁺ T cell; a CD4⁺ T cell; a NK T cell; an αβ T cell; a γδ T cell; a peripheral blood T cell; and a tumour infiltrated T cell.

Suitably, a therapeutically effective amount of such a population of granulopoietic cells (or of a composition [e.g. a pharmaceutical composition] of the invention), for example for use in accordance with the invention, is an amount sufficient to increase activation of host T cells. The extent of increase, and suitable indicators of increased activation, may be as considered in the preceding paragraphs and/or as in those that follow.

A population of granulopoietic cells or composition suitable for use in accordance with the present invention may increase activation of host CD8⁺ T cells. Accordingly, such a population of granulopoietic cells or composition may be capable of amplifying (preferably amplify) a host therapeutic immune response by increasing activation of host CD8⁺ T cells.

A population of granulopoietic cells or composition suitable for use in accordance with the present invention may increase activation of host CD4⁺ T cells, such as host CD4⁺ T cells. Accordingly, such a population of granulopoietic cells or composition may be capable of amplifying (preferably amplify) a host therapeutic immune response by increasing activation of host CD4⁺ T cells.

A population of granulopoietic cells or composition suitable for use in accordance with the present invention may increase activation of host NK T cells. Accordingly, such a population of granulopoietic cells or composition may be capable of amplifying (preferably amplify) a host therapeutic immune response by increasing activation of host NK T cells.

Increased activation of NK T cells may be associated with one or more of the following: an increase in expression by NK T cells of a degranulation marker (including, but not limited to, CD107a); an increase in expression by NK T cells of a costimulatory molecule (including, but not limited to, 4-1BB and/or OX40); and an increase in survival of NK T cells. Further relevant considerations in respect of these various properties are set out elsewhere in the present specification.

The host NK T cells, activation of which is increased, may be peripheral blood NK T cells or may be tumour infiltrated NK T cells.

Activation of such NK T cells may be increased by at least 5%. For example, activation of NK T cells may be increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, or more. Quantification of the increase in activation of NK T cells in accordance with such an embodiment may make use of comparison to an appropriate control.

A population of granulopoietic cells or composition suitable for use in accordance with the present invention may increase activation of host γδ T cells. Accordingly, such a population of granulopoietic cells or composition may be capable of amplifying (preferably amplify) a host therapeutic immune response by increasing activation of host γδ T cells.

A population of granulopoietic cells or composition suitable for use in accordance with the present invention may increase activation of host NK cells. Accordingly, such a population of granulopoietic cells or composition may be capable of amplifying (preferably amplify) a host therapeutic immune response by increasing activation of host NK cells.

The skilled person will appreciate that NK cells play an important role in providing the activity necessary to achieve a therapeutic immune response. NK cells show strong cytolytic activity against physiologically stressed cells such as tumour cells and virus-infected cells. Accordingly, the use of a population of granulopoietic cells to increase activation of NK cells will be of benefit in a wide range of circumstances in which a therapeutically effective immune response is required. In particular, the amplification of a therapeutic immune response by increasing activation of NK cells may, without limitation, be advantageous in the treatment of cancer or the treatment of infections.

The host NK cells, activation of which is increased, may be peripheral blood NK cells or may be tumour infiltrated NK cells.

Suitably, a therapeutically effective amount of such a population of granulopoietic cells (or of a composition [e.g. a pharmaceutical composition] of the invention), for example for use in accordance with the invention, is an amount sufficient to increase activation of host NK cells. The extent of increase, and suitable indicators of increased activation, may be as considered in the preceding paragraphs and/or as in those that follow.

A population of granulopoietic cells or composition suitable for use in accordance with the present invention may increase activation of host monocytes or macrophages. Accordingly, such a population of granulopoietic cells or composition may be capable of amplifying (preferably amplify) a host therapeutic immune response by increasing activation of host monocytes or macrophages.

A population of granulopoietic cells or composition suitable for use in accordance with the present invention may increase activation of host PBMCs. Accordingly, such a population of granulopoietic cells or composition may be capable of amplifying (preferably amplify) a host therapeutic immune response by increasing activation of host PBMCs.

It will be appreciated that PBMCs play an essential role in providing the cells that contribute to any effective therapeutic immune response. PBMCs may be taken as referring to any peripheral blood cell having a single round nucleus, such as T cells and NK cells. These cells have a variety of functions key to driving the immune response including cytocidal activity or activation of further immune cells. Accordingly, the use of a population of granulopoietic cells or composition to increase activation of host PBMCs will be of benefit in almost all circumstances in which a therapeutically effective immune response is required. In particular, the amplification of a therapeutic immune response by increasing activation of host PBMCs may, without limitation, be advantageous in the treatment of cancer or the treatment of infections.

The host PBMCs, activation of which is to be increased, include, but are not limited to, those selected from the group comprising (or consisting) of: peripheral blood T cells (such as: peripheral blood CD8⁺ T cells; peripheral blood CD4⁺ T cells; peripheral blood NK T cells; peripheral blood αβ T cells; or peripheral blood γδ T cells); and peripheral blood NK cells.

Increased activation of host PBMCs may be demonstrated by any appropriate marker of activation. Merely by way of example, increased activation of PBMCs may be demonstrated by increased expression of cytokines (such as: IFN-γ; and/or TNF). The ability to increase cytokine expression by host PBMCs exposed to populations of granulopoietic cells suitable for use in accordance with the present invention is shown in the Examples. Further relevant considerations in respect of these various properties are set out elsewhere in the present specification.

Activation of host PBMCs may be increased by at least 5%. For example, activation of PBMCs may be increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, or more. Quantification of the increase in activation of host PBMCs in accordance with such an embodiment may make use of comparison to an appropriate control.

Suitably, a therapeutically effective amount of such a population of granulopoietic cells (or of a composition [e.g. a pharmaceutical composition] of the invention), for example for use in accordance with the invention, is an amount sufficient to increase activation of host PBMCs. The extent of increase, and suitable indicators of increased activation, may be as considered in the preceding paragraphs and/or as in those that follow.

A population of granulopoietic cells or composition suitable for use in accordance with the present invention may increase activation of host TILs. Accordingly, such a population of granulopoietic cells or composition may be capable of amplifying (preferably amplify) a host therapeutic immune response by increasing activation of host TILs.

For the purposes of the present invention, host TILs may be taken as encompassing all lymphocytic cell populations that have invaded tumour tissue. With this in mind, it will be recognised that TILs play a key role in component exerting a therapeutic immune response against tumour cells. TILs can exert specific cytotoxic antitumour activity (for example CD8⁺ cells that have entered the tumour) and can promote an antitumour response through activation of other immune cells (such as by CD4⁺ cells within the tumour). Accordingly, the amplification of a therapeutic immune response by increasing activation of host TILs may play a highly advantageous role in the treatment of cancer.

In particular, the inventors have determined that a population of granulopoietic cells suitable for use in accordance with the present invention may increase activation of tumour infiltrated T cells and/or NK cells. Such granulopoietic cells may increase activation of tumour infiltrated CD8⁺ T cells and/or CD4⁺ T cells, as demonstrated in the Examples.

Increased activation of host TILs, such as increased activation of tumour infiltrated T cells or tumour infiltrated NK cells, may be demonstrated by any appropriate marker of activation. Merely by way of example, increased activation of TILs may be demonstrated by increased expression of degranulation markers (such as: CD107a; perforin; or granzymes). Alternatively, or additionally, increased activation of TILs may be demonstrated by increased expression of costimulatory molecules (such as: 4-1BB; OX40; CD27; CD28; ICOS; HVEM; LIGHT; CD40L; DR3; GITR; CD30; TIM1; CD2; or CD226). The ability to increase expression of degranulation markers or costimulatory molecules by TILs exposed to populations of granulopoietic cells suitable for use in accordance with the present invention is demonstrated in the Examples. Further relevant considerations in respect of these various properties are set out elsewhere in the present specification.

Activation of TILs may be increased by at least 5%. For example, activation of TILs may be increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, or more. Quantification of the increase in activation of TILs in accordance with such an embodiment may make use of comparison to an appropriate control.

Suitably, a therapeutically effective amount of a population of such granulopoietic cells (or of a composition [e.g. a pharmaceutical composition] of the invention), for example for use in accordance with the invention, is an amount sufficient to increase activation of host TILs. The extent of increase, and suitable indicators of increased activation, may be as considered in the preceding paragraphs and/or as in those that follow.

A population of granulopoietic cells suitable for use in accordance with the present invention may be able to increase expression by immune cells of degranulation markers. In particular, granulopoietic cells may be capable of increasing (preferably increase) expression of degranulation markers by the non-granulocytic immune cell present in a composition of the invention. A population of granulopoietic cells or composition may be capable of increasing (preferably increase) expression of degranulation markers by host immune cells. Accordingly, such a population of granulopoietic cells or composition may be capable of amplifying (preferably amplify) a host therapeutic immune response by increasing expression of degranulation markers by host immune cells.

Degranulation is a key process in cytocidal activity of immune cells such as CD8⁺ T cells or NK cells, that underpins their therapeutic immune activity. Accordingly, it will be appreciated that increased expression of degranulation markers, such as CD107, provides an indication that the therapeutic immune activity of such cells has been increased, and the therapeutic immune response amplified accordingly.

In a suitable embodiment, a degranulation marker, expression of which by host immune cells is increased, is selected from the group comprising (or consisting) of: CD107a; perforin; and granzymes. Suitably, expression of more than one of these degranulation markers may be increased. For example, expression of at least 2 such degranulation markers may be increased. In particular, expression by host immune cells of CD107a may be increased.

Increased expression of degranulation markers can be assessed, and if desired quantified, by any appropriate method.

In a suitable embodiment, expression of a degranulation marker is increased by at least 5%. For example, expression of a degranulation marker may be increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, or more. Quantification of increased expression of degranulation markers in accordance with such an embodiment may make use of comparison to an appropriate control.

Expression of degranulation markers may be increased in non-granulocytic immune cells present in a composition of the invention or host immune cells selected from the group comprising (or consisting) of: T cells and NK cells. In the case that expression of degranulation markers is increased in a T cell, such a T cell may be selected from the group comprising (or consisting) of: a CD8⁺ T cell; a NK T cell; an αβ T cell; and a γδ T cell.

Suitably, a therapeutically effective amount of such a population of granulopoietic cells (or of a composition [e.g. a pharmaceutical composition] of the invention), for example for use in accordance with the invention, is an amount sufficient to increase expression by immune cells, such as host immune cells, of one or more degranulation markers. The degranulation markers, extent of increase, and relevant host immune cells may be as considered in the preceding paragraphs.

A population of granulopoietic cells suitable for use in accordance with the present invention may be able to increase expression by immune cells of a costimulatory molecule. In particular, granulopoietic cells may be capable of increasing (preferably increase) expression of costimulatory molecules by the non-granulocytic immune cell present in a composition of the invention. A population of granulopoietic cells or composition may be capable of increasing (preferably increase) expression of costimulatory molecules by host immune cells. Accordingly, such a population of granulopoietic cells or composition may be capable of amplifying (preferably amplify) a host therapeutic immune response by increasing expression by host immune cells of a costimulatory molecule.

Costimulatory molecules act to amplify or counteract activating signals provided to T cells causing T cell differentiation. T-cell differentiation is a key process in the therapeutic immune response, giving rise to the production of cytotoxic T cells or helper T cells. Increased expression of costimulatory molecules can thus direct functional differentiation of T cells, hence causing the therapeutic immune response to be amplified. The use of a population of granulopoietic cells to increase expression of costimulatory molecules will be of benefit in a wide range of circumstances in which a therapeutically effective immune response is required. In particular, the amplification of a therapeutic immune response by increasing activation of costimulatory molecules may, without limitation, be advantageous in the treatment of cancer or of infections.

In a suitable embodiment, a costimulatory molecule, expression of which by non-granulocytic immune cells and/or host immune cells is increased, is selected from the group comprising (or consisting) of: 4-1BB; OX40; CD27; CD28; ICOS; HVEM; LIGHT; CD40L; DR3; GITR; CD30; TIM1; CD2; and CD226. Suitably, expression of more than one of these costimulatory molecules may be increased. For example, expression of at least 2, at least 3, at least 4, at least 5, at least 6, at least 7, at least 8, at least 9, at least 10, at least 11, at least 12, or at least 13 such costimulatory molecules may be increased. In particular, expression by non-granulocytic immune cells and/or host immune cells of both 4-1BB and OX40 may be increased.

Expression of a costimulatory molecule can be assessed, and if desired quantified, by any appropriate method.

In a suitable embodiment, expression of a costimulatory molecule is increased by at least 5%. For example, expression of a co-stimulatory molecule may be increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, or more. Quantification of the increase in expression of a costimulatory molecule in accordance with such an embodiment may make use of comparison to an appropriate control.

Expression of the costimulatory molecule may be increased in non-granulocytic immune cells and/or host immune cells selected from the group comprising (or consisting) of: T cells and NK cells. In the case that expression of the costimulatory molecule is increased in a T cell, such a T cell may be selected from the group comprising (or consisting) of: a CD8⁺ T cell; a CD4⁺ T cell; a NK T cell; an αβ T cell; a γδ T cell; a peripheral blood T cell; and a tumour infiltrated T cell.

Suitably, a therapeutically effective amount of such granulopoietic cells (or of a composition [e.g. a pharmaceutical composition] of the invention), for example for use in accordance with the invention, is an amount sufficient to increase expression by immune cells, such as non-granulocytic immune cells or host immune cells, of one or more costimulatory molecules. The costimulatory molecules, extent of increase, and relevant host immune cells may be as considered in the preceding paragraphs.

A population of granulopoietic cells suitable for use in accordance with the present invention may be able to increase expression by immune cells of cytokines. In particular, granulopoietic cells may be capable of increasing (preferably increase) expression of cytokines by the non-granulocytic immune cell present in a composition of the invention. A population of granulopoietic cells or composition may be capable of increasing (preferably increase) expression of cytokines by host immune cells. Accordingly, such a population of granulopoietic cells or composition may be capable of amplifying (preferably amplify) a host therapeutic immune response by increasing expression by host immune cells of a costimulatory molecule.

Cytokines are key chemical messengers in the immune response. Cytokines signal for cell activation (directing immune cells), differentiation of immune cells such as during T cell differentiation and proliferation of immune cells such as NK cells. The use of a population of granulopoietic cells to increase activation of cytokines will be of benefit in almost all circumstances in which a therapeutically effective immune response is required. In particular, the amplification of a therapeutic immune response by increasing activation of cytokines may, without limitation, be advantageous in the treatment of cancer or treatment of infection.

For the purposes of the present invention, cytokines should be taken as encompassing chemokines, interferons, interleukins, lymphokines, and TNFs.

In a suitable embodiment, a cytokine, expression of which by non-granulocytic immune cells and/or host immune cells is increased, is selected from the group comprising (or consisting) of: IFN-γ; and TNF. Suitably, expression of more than one of these costimulatory molecules may be increased. In particular, expression by non-granulocytic immune cells and/or host immune cells of IFN-γ may be increased.

Increased expression of cytokines can be assessed, and if desired quantified, by any appropriate method.

In a suitable embodiment, expression of a cytokine is increased by at least 5%. For example, expression of a cytokine may be increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, or more. Quantification of expression of a cytokine in accordance with such an embodiment may make use of comparison to an appropriate control.

Expression of a cytokine may be increased in host immune cells selected from the group comprising (or consisting) of: PBMCs; and TILs. The ability of populations of granulopoietic cells suitable for use in accordance with the invention to increase expression by PBMCs and TILs of cytokines (such as IFN-γ) is demonstrated in the Examples.

Suitably, a therapeutically effective amount of such a population of granulopoietic cells (or of a composition [e.g. a pharmaceutical composition] of the invention), for example for use in accordance with the invention, is an amount sufficient to increase expression by immune cells, such as non-granulocytic immune cells and/or host immune cells, of one or more cytokines. The cytokines, extent of increase, and relevant host immune cells may be as considered in the preceding paragraphs.

A population of granulopoietic cells suitable for use in accordance with the present invention may be able to increase immune cell trafficking. In particular, a population of granulopoietic cells of this sort may be capable of increasing (preferably increase) trafficking of host immune cells. Accordingly, such a population of granulopoietic cells may be capable of amplifying (preferably amplify) a host therapeutic immune response by increasing trafficking of host immune cells.

Trafficking of immune cells plays a vital role in their ability to access sites, such as sites of tumours or infections, at which they are needed to exert their therapeutic activity. It will therefore be appreciated that the ability of populations of granulopoietic cells or compositions suitable for use in accordance with the invention to increase immune cell trafficking confers clear advantages in terms of facilitating an effective therapeutic immune response.

Increased cell trafficking may be observed in respect of PBMCs, and particularly in respect of host PBMCs. As noted elsewhere, the inventors have demonstrated that populations of granulopoietic cells suitable for use in accordance with the invention may give rise to granulocytes that express CXCL10, which is known to act as a chemoattractant for CXCR3⁺ immune cells. Thus, the medical uses and methods of treatment of the invention, by giving rise to a population of cells that express CXCL10, may be of particular benefit in increasing trafficking of CXCR3⁺ T cells and CXCR3⁺ NK cells.

Increased immune cell trafficking can be assessed, and if desired quantified, by any appropriate method.

In a suitable embodiment, trafficking of immune cells is increased by at least 5%. For example, trafficking of immune cells may be increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, or more. Quantification of trafficking of immune cells in accordance with such an embodiment may make use of comparison to an appropriate control.

Suitably, a therapeutically effective amount of such populations of granulopoietic cells (or of a composition [e.g. a pharmaceutical composition] of the invention), for example for use in accordance with the invention, is an amount sufficient to increase trafficking of immune cells, such as host immune cells. The extent of increase in trafficking, and the relevant host immune cells, may be as considered in the preceding paragraphs.

In particular, the increased trafficking of immune cells may give rise to increased recruitment of immune cells into the TME.

As noted above, the inventors have noted that exposure to a population of granulopoietic cells suitable for use in accordance with the present invention increases immune cell trafficking. In particular, the inventors have noted that populations of granulopoietic cells or compositions suitable for use in accordance with the present invention may increase recruitment of immune cells into the TME. As demonstrated in the Examples, a population of granulopoietic cells or composition of this sort may be capable of increasing (preferably increase) recruitment into the TME of host immune cells. Accordingly, such a population of granulopoietic cells or composition may be capable of amplifying (preferably amplify) a host therapeutic immune response by increasing recruitment of host immune cells into the TME.

The low propensity for immune cells to enter the TME is well known. Many immune cells demonstrate little capacity to penetrate into tumours, and the TME has immunosuppressive properties. Accordingly, the ability to increase recruitment of immune cells, such as host immune cells, into the TME through the populations of granulopoietic cells or compositions suitable for use in accordance with the invention offers remarkable advantages in the treatment of tumours. By increasing the number of immune cells that are present in a tumour, anti-tumour activity of the cells exerting the therapeutic immune response can be dramatically increased.

Increased immune cell recruitment into the TME may be observed in respect of PBMCs, and particularly in respect of host PBMCs. The ability of populations of granulopoietic cells or compositions suitable for use in accordance with treatment of the invention to increase such recruitment into the TME is demonstrated in the Examples.

In the Examples the inventors also demonstrate that populations of granulopoietic cells and compositions suitable for use in accordance with the invention may differentiate to give rise to granulocytes that express CXCL10. CXCL10 is a chemoattractant for CXCR3⁺ immune cells, which may include CXCR3⁺ T cells and CXCR3⁺ NK cells. Thus, the populations of granulopoietic cells and compositions suitable for use in accordance with the invention may be of particular benefit in establishing a population of granulocyte progeny cells capable of increasing (preferably increase) recruitment of CXCR3⁺ T cells and CXCR3⁺ NK cells into the TME.

Increased immune cell recruitment into the TME can be assessed, and if desired quantified, by any appropriate method.

In a suitable embodiment, recruitment of immune cells into the TME is increased by at least 5%. For example, recruitment of immune cells into the TME may be increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, or more. Quantification of recruitment of immune cells into the TME in accordance with such an embodiment may make use of comparison to an appropriate control.

Suitably, a therapeutically effective amount of such a population of granulopoietic cells (or of a composition [e.g. a pharmaceutical composition] of the invention), for example for use in accordance with the invention, is an amount sufficient to increase recruitment of immune cells, such as host immune cells, into the TME. The extent of the increased recruitment of immune cells into the TME, and the relevant host immune cells, may be as considered in the preceding paragraphs.

A population of granulopoietic cells suitable for use in accordance with the present invention may be able to increase cytocidal activity of immune cells. In particular, granulopoietic cells may be capable of increasing (preferably increase) cytocidal activity of the non-granulocytic immune cell present in a composition of the invention. A population of granulopoietic cells or composition may be capable of increasing (preferably increase) cytocidal activity of host immune cells. Accordingly, such a population of granulopoietic cells or composition may be capable of amplifying (preferably amplify) a host therapeutic immune response by increasing cytocidal activity of host immune cells.

Cell killing of infected, cancerous, or other pathological cells is a key mechanism by which many immune cells exert their therapeutic activity. It will therefore be appreciated that the ability of the populations of granulopoietic cells and compositions suitable for use in accordance with the invention to increase cytocidal activity of immune cells will offer advantages in terms of increasing the effectiveness of therapeutic immune responses that may be used to treat a great number of conditions, including cancer and infections.

Increased cytocidal activity of immune cells may be observed in respect of PBMCs, and particularly in respect of host PBMCs.

Increased cytocidal activity of immune cells can be assessed, and if desired quantified, by any appropriate method.

In a suitable embodiment, cytocidal activity of immune cells is increased by at least 5%. For example, cytocidal activity of immune cells may be increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, or more. Quantification of cytocidal activity of immune cells in accordance with such an embodiment may make use of comparison to an appropriate control.

Suitably, a therapeutically effective amount of such a population of granulopoietic cells (or of a composition [e.g. a pharmaceutical composition] of the invention), for example for use in accordance with the invention, is an amount sufficient to increase cytocidal activity of immune cells, such as host immune cells. The extent of increased cytocidal activity of immune cells, and the relevant host immune cells, may be as considered in the preceding paragraphs.

In particular, the increased cytocidal of immune cells may give rise to increased tumour cell killing activity of immune cells, and especially of host immune cells.

A population of granulopoietic cells suitable for use in accordance with the present invention may be able to increase tumour cell killing activity of immune cells. The immune cells tumour killing activity of which is to be increased may be cells in vitro, or cells in vivo, for example immune cells present in a subject to whom a suitable population of granulopoietic cells is provided. In particular, granulopoietic cells may be capable of increasing (preferably increase) tumour cell killing activity of a non-granulocytic immune cell present in a suitable composition of the invention. A population of granulopoietic cells or composition may be capable of increasing (preferably increase) tumour cell killing activity of host immune cells. Accordingly, such a population of granulopoietic cells or composition may be capable of amplifying (preferably amplify) a host therapeutic immune response by increasing tumour cell killing activity of host immune cells.

The use of immune cells to target and kill cancer cells forms the basis for most anti-cancer immunotherapy. Accordingly, it will be readily appreciated that the ability of the population of granulopoietic cells and compositions suitable for use in accordance with the invention to increase the tumour cell killing activity of immune cells, such as host immune cells, provides clear and desirable advantages in anti-cancer treatments.

Increased tumour cell killing activity of immune cells may be observed in respect of PBMCs, and particularly in respect of host PBMCs. Such increases are demonstrated in the results provided in the Examples.

Increased tumour cell killing activity of immune cells can be assessed, and if desired quantified, by any appropriate method.

In a suitable embodiment, tumour cell killing activity of immune cells is increased by at least 5%. For example, tumour cell killing activity of immune cells may be increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, or more. Quantification of tumour cell killing activity of immune cells in accordance with such an embodiment may make use of comparison to an appropriate control.

Suitably, a therapeutically effective amount of such populations of granulopoietic cells (or of a composition [e.g. a pharmaceutical composition] of the invention), for example for use in accordance with the invention, is an amount sufficient to increase tumour cell killing activity of immune cells, such as host immune cells. The extent of increased tumour cell killing activity of immune cells, and the relevant host immune cells, may be as considered in the preceding paragraphs.

A population of granulopoietic cells suitable for use in accordance with the present invention may be able to increase proliferation of immune cells. In particular, granulopoietic cells may be capable of increasing (preferably increase) proliferation of the non-granulocytic immune cell present in a composition of the invention. A population of granulopoietic cells or composition may be capable of increasing (preferably increase) proliferation of host immune cells. Accordingly, such a population of granulopoietic cells or composition may be capable of amplifying (preferably amplify) a host therapeutic immune response by increasing proliferation of host immune cells.

Immune cell-based therapies rely upon the development of therapeutically effective quantities of suitable immune cells in order to be able to provide the required therapeutic immune response (for example in treatment of cancer or infections). It will therefore be appreciated that the ability of the population of granulopoietic cells and compositions suitable for use in accordance with the invention to increase proliferation of immune cells, such as host immune cells, is highly beneficial in achieving this. For example, by increasing proliferation of immune cells, granulopoietic cells and compositions suitable for use in accordance with the invention may be capable of amplifying (preferably amplify) immune responses that would not otherwise reach a therapeutic threshold, or to reduce the time taken for therapeutically effective quantity of immune cells to be produced.

In a suitable embodiment, proliferation of T cells, such as host T cells, may be increased. Suitable T cells may be selected from the group comprising (or consisting) of: an αβ T cell; a CD8⁺ T cell; a CD4⁺ T cell; a NK T cell; and a γδ T cell. In particular, the proliferation of αβ T cells may be increased, demonstrated by the data set out in the Examples. Merely by way of example, the αβ T cells may be CD4⁺ T cells, or may be CD8⁺ T cells.

Increased proliferation of immune cells can be assessed, and if desired quantified, by any appropriate method.

Suitably, proliferation of host immune cells may be increased by at least 5%. For example, proliferation of host immune cells may be increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, or more. Quantification of the increase in proliferation of host immune cells in accordance with such an embodiment may make use of comparison to an appropriate control.

Suitably, a therapeutically effective amount of such populations of granulopoietic cells (or of a composition [e.g. a pharmaceutical composition] of the invention), for example for use in accordance with the invention, is an amount sufficient to increase proliferation of immune cells, such as host immune cells. The extent of increased proliferation of immune cells, and the relevant host immune cells, may be as considered in the preceding paragraphs.

A population of granulopoietic cells suitable for use in accordance with the present invention may be able to increase survival of immune cells. In particular, granulopoietic cells may be capable of increasing (preferably increase) survival of the non-granulocytic immune cell present in a composition of the invention. A population of granulopoietic cells or composition may be capable of increasing (preferably increase) survival of host immune cells. Accordingly, such a population of granulopoietic cells or composition may be capable of amplifying (preferably amplify) a host therapeutic immune response by increasing survival of host immune cells.

It is well known that immune cells have a limited lifespan, being rapidly turned over within the body. This is heightened in contexts such as the TME, where immunosuppressive conditions further reduce the lifespan of immune cells entering the tumour. The inventors' finding that the granulopoietic cells and compositions suitable for use in accordance with treatment of the invention are able to increase survival of immune cells thus indicates that treatments utilising such granulopoietic cells may offer advantages in terms of prolonging the period during which immune cells are able to generate an effective therapeutic immune response. This may be of particular value in treatment of conditions, such as cancer, in which an immunosuppressive environment otherwise reduces longevity of immune cells.

In a suitable embodiment, survival of T cells (such as NK T cells) or NK cells may be increased. For example, survival of host T cells (such as NK T cells) or NK cells may be increased. Data illustrating the ability of granulopoietic cells and compositions useful in accordance with the invention to increase survival of NK T cells and NK cells are set out in the Examples.

Increased survival of immune cells can be assessed, and if desired quantified, by any appropriate method.

Suitably, survival of host immune cells may be increased by at least 5%. For example, survival of host immune cells may be increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, or more. Quantification of the increase in survival of host immune cells in accordance with such an embodiment may make use of comparison to an appropriate control.

Suitably, a therapeutically effective amount of such populations of granulopoietic cells (or of a composition [e.g. a pharmaceutical composition] of the invention), for example for use in accordance with the invention, is an amount sufficient to increase survival of immune cells, such as host immune cells. The extent of increased survival of immune cells, and the relevant host immune cells, may be as considered in the preceding paragraphs.

A population of granulopoietic cells suitable for use in accordance with the present invention may be able to increase the abundance of immune cells. In particular, a population of granulopoietic cells or composition of this sort may be capable of increasing (preferably increase) abundance of host immune cells. Accordingly, such a population of granulopoietic cells or composition may be capable of amplifying (preferably amplify) a host therapeutic immune response by increasing the abundance of host immune cells.

Without wishing to be bound by any hypothesis, the increase in abundance of immune cells observed on exposure of such cells to populations of granulopoietic cells and compositions suitable for use in accordance with the invention may arise as a result of a combination of the increased proliferation and increase survival of the immune cells discussed in more detail above. However it arises, it offers real benefits in terms of the medical uses and methods of the invention. By increasing the abundance of immune cells able to take part in a therapeutic immune response, the medical uses and methods of treatment of the invention have the capacity to amplify such a therapeutic immune response both in terms of its extent and its duration. This will clearly provide benefits in many therapeutic contexts.

Increased abundance of immune cells can be assessed, and if desired quantified, by any appropriate method.

Suitably, the abundance of host immune cells may be increased by at least 5%. For example, the abundance of host immune cells may be increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, or more. Quantification of the increase in the abundance of host immune cells in accordance with such an embodiment may make use of comparison to an appropriate control.

In a suitable embodiment, the abundance of T cells, such as host T cells, may be increased. T cells the abundance of which may be increased may be selected from the group comprising (or consisting) of: an αβ T cell; a CD8⁺ T cell; a CD4⁺ T cell; a NK T cell; and a γδ T cell. In particular, the abundance of host αβ T cells may be increased, as illustrated further in the Examples. The αβ T cells may be CD4⁺ T cells, or may be CD8⁺ T cells.

Suitably, a therapeutically effective amount of such populations of granulopoietic cells (or of a composition [e.g. a pharmaceutical composition] of the invention), for example for use in accordance with the invention, is an amount sufficient to increase abundance of immune cells, such as host immune cells. The extent of increased abundance of immune cells, and the relevant host immune cells, may be as considered in the preceding paragraphs.

Many of the properties of the cells and compositions suitable for use in the medical uses and methods of the invention indicate that these cells and compositions are also well suited to use in combination with other cell therapies, and in particular for use with further cell immunotherapies.

The ability of the cells and compositions of the invention to increase proliferation, abundance and survival of immune cells suggests that treatments employing the cells and compositions of the invention may be of particular advantage when used in combination with other cell therapies. These may be therapies that use the host's own cells, or therapies using allogeneic cells. By providing a treatment in accordance with the invention, cells involved in the further cell therapy may be induced to proliferate, survive longer, and accumulate with increased abundance. The effectiveness of such a therapy may thereby be improved.

As noted above, the inventors have identified the ability of populations of granulopoietic cells to provide “signal 2” (co-stimulation) and “signal 3” (cytokine simulation) to other immune cells, such as those constituting part of a further cell immunotherapy. The provision of these signals is important in generating effective immune responses to tumours, and in overcoming the immunosuppressive effects of the TME. This property of the populations of granulopoietic cells suggests that they may be used in combination with a further cell immunotherapy, and that by doing so the proliferation, survival and accumulation of cells involved with said further cell therapy may be improved.

The inventors' finding that populations of granulopoietic cells are able to generate granulocytes that secrete chemokines, such as CXCL10, also suggests utility in combination with a further cell immunotherapy. Chemokines play a vital part in the migration, positioning and release of immune cells during a therapeutic immune response. The ability of populations of granulopoietic cells to give rise to granulocyte progeny cells that secrete chemokines suggests that the use of the populations of granulopoietic cells in combination with a further cell immunotherapy may be expected to give rise to the production of granulocytes able to beneficially improve the activity of the cells of the further therapy.

The inventors have also identified that the granulocytes produced on differentiation of populations of granulopoietic cells suitable for use in the various aspects of the invention express ligands for costimulatory molecules, such as 4-1BBL and OX40L. The interaction of these ligands with their receptors play a vital part in regulating the activation of T cells and the generation of effector T cell responses. Accordingly, the expression of such receptors by progeny of the populations of granulopoietic cells suggests that use of the populations of granulopoietic cells in combination with further cell immunotherapies will enable the populations of granulopoietic cells to produce granulocytes that positively influence T cell responses in this manner.

Suitably, a therapeutically effective amount of such populations of granulopoietic cells (or of a composition [e.g. a pharmaceutical composition] of the invention), for example for use in in accordance with the invention, when in combination with a further cell therapy, is an amount sufficient to increase proliferation survival and/or abundance of immune cells associated with said further cell immunotherapy. The extent of increase, relevant immune cells, and suitable indicators of increased activation may be as considered elsewhere in the specification.

The skilled person will be aware of many examples of cell immunotherapies that may beneficially be used in combination with treatment using populations of granulopoietic cells or compositions in accordance with the invention. These include, but are not limited to: NK cell therapies; chimeric antigen receptor (CAR)-based therapies (including CAR-T cell therapies, such as CAR-γδ T cell therapies, and CAR-NK cell therapies); TIL therapies; and engineered T cell receptor (TCR) therapies.

The medical uses, methods of treatment and compositions (e.g. pharmaceutical compositions) may comprise populations of granulopoietic cells for use in the treatment of a subject by means of amplifying a non-granulocytic therapeutic immune response.

The term “treat” or “treating” as used herein encompasses prophylactic treatment (e.g. to prevent onset of a disease) as well as corrective treatment (treatment of a subject already suffering from a disease). Preferably “treat” or “treating” as used herein means corrective treatment.

The term “treat” or “treating” as used herein may refer to both the disorder and/or a symptom thereof.

A population of granulopoietic cells as part of a composition (e.g. a pharmaceutical composition) of the invention, may be administered to a subject in a therapeutically effective amount or a prophylactically effective amount.

Some considerations regarding specific therapeutically effective amounts, selected with respect to particular results to be achieved, have been set out above. However, in general terms, a “therapeutically effective amount” should be taken as being any amount of the compositions (e.g. pharmaceutical compositions) of the invention, which when administered alone or in combination with another agent to a subject for treating cancer or an infection (or a symptom thereof) is sufficient to effect such treatment of the disorder, or symptom thereof.

In the case that the therapeutically effective amount of a composition (e.g. a pharmaceutical composition) of the invention is administered alone, this may amplify a native immune response, thereby helping this to treat cancer or infection.

A “prophylactically effective amount” is any amount of the compositions (e.g. pharmaceutical compositions) of the invention that, when administered alone or in combination with another agent to a subject inhibits or delays the onset or reoccurrence of cancer or an infection (or a symptom thereof). In some embodiments, the prophylactically effective amount prevents the onset or reoccurrence of a cancer or an infection entirely. “Inhibiting” the onset means either lessening the likelihood of cancer onset or infection onset (or symptom thereof), or preventing the onset entirely.

An appropriate dosage range is one that produces the desired therapeutic effect (e.g. wherein the compositions (e.g. pharmaceutical compositions) of the invention are dosed in a therapeutically or prophylactically effective amount).

A typical treatment regimen may include administering from 10⁶, 10⁷, 10⁸ or 10⁹ cells (e.g. cells of a population of granulopoietic cells) to a subject, or up to 10¹², 10¹³ or 10¹⁴ cells to a subject. In a suitable embodiment a treatment regimen includes administering a dose of at least 1×10⁹ cells to a subject. Suitably, a treatment regimen may include administering a dose of at least 2×10⁹ cells or at least 5×10⁹ cells to a subject. In a suitable embodiment a treatment regimen may include administering a dose of at least 1×10¹⁰ cells or at least 5×10¹⁰ cells to a subject. At least 1×10¹¹ or at least 2×10¹¹ cells may be administered to a subject. In some embodiments between 1×10⁹ to 3×10¹¹ or 1×10¹⁰ to 3×10¹¹ cells are administered to a subject. Suitably, between 5×10¹⁰ to 2.5×10¹¹ cells are administered to a subject.

A subject for treatment may be dosed once, twice, three times, four times, five times, or six times per week. Alternatively, a subject may be dosed daily (e.g. once or twice daily). In other embodiments a subject may be dosed once weekly or bi-weekly. Preferably the dose is weekly. The skilled person will appreciate that the dose can be tailored based on the needs of the subject, and efficacy of the medicament. For example, where the medicament is highly efficacious, the dose may be lowered.

In a suitable embodiment a subject for treatment is dosed weekly (e.g. once weekly) with at least 2×10⁹ cells or at least 2×10¹⁰ cells. Suitably, a subject for treatment may be dosed weekly with at least 1×10¹¹ or at least 2×10¹¹ cells.

The treatment term can be varied based on the response of the subject to the treatment, and/or the type and/or severity of the cancer or the infection. For example, the subject for treatment may be dosed for at least 1 or 2 weeks. Suitably the subject for treatment may be dosed for at least 3 or 4 weeks. In a suitable embodiment the subject for treatment is dosed for at least 5 or 6 weeks, suitably at least 7 or 8 weeks.

In a suitable embodiment a subject for treatment is dosed for 4-8 weeks with at least 2×10⁹ cells, wherein said cells are administered once weekly. Suitably a subject for treatment is dosed for 8 weeks with at least 2×10⁹ cells (preferably at least 2×10¹⁰ or 2×10¹¹ cells), wherein said cells are administered once weekly.

Administration may be by any suitable technique or route, including but not limited to intravenous injection, intra-arterial injection, intraperitoneal injection, injection into a tumour resection cavity, intrathecal injection, or combinations thereof. Suitably the medicament may be administered intravenously.

A white blood cell growth factor may be administered with a medicament (e.g. composition) of the invention. The administration may be sequential or simultaneous (suitably simultaneous). Suitable white blood cell growth factors may include a granulocyte-macrophage colony-stimulating factor (GM-CSF), a granulocyte colony-stimulating factor (G-CSF), a growth hormone; serotonin, vitamin C, vitamin D, glutamine (Gln), arachidonic acid, AGE-albumin, an interleukin, TNF-alpha, Flt-3 ligand, thrombopoietin, foetal bovine serum (FBS), retinoic acid, lipopolysaccharide (LPS), IFN-gamma, IFN-beta, or combinations thereof. Suitably, the white blood cell growth factors comprise IFN-gamma and GM-CSF. Preferably, the white blood cell growth factors comprise TNF-alpha. Suitably the white blood cell growth factors may comprise a granulocyte-macrophage colony-stimulating factor (GM-CSF), and a granulocyte colony-stimulating factor (G-CSF), and a growth hormone, and serotonin, and vitamin C, and vitamin D, and glutamine (Gln), and arachidonic acid, and AGE-albumin, and an interleukin, and TNF-alpha, and Flt-3 ligand, and thrombopoietin, and foetal bovine serum (FBS). Suitably the white blood cell growth factors may comprise a granulocyte-macrophage colony-stimulating factor (GM-CSF), and a granulocyte colony-stimulating factor (G-CSF), and a growth hormone, and serotonin, and vitamin C, and vitamin D, and glutamine (Gln), and arachidonic acid, and AGE-albumin, and an interleukin, and TNF-alpha, and Flt-3 ligand, and thrombopoietin, and foetal bovine serum (FBS), and retinoic acid, and lipopolysaccharide (LPS), and IFN-gamma, and IFN-beta. Particular examples of the foregoing include but are not limited to LEUKINE® brand sargramostim, NEUPOGEN® brand filgrastim, and NEULAST A® brand 5 PEG-filgrastim.

In a suitable embodiment a composition may be administered (e.g. sequentially or simultaneously, preferably simultaneously) with a granulocyte-colony stimulating factor; and a growth hormone; and a serotonin; and an interleukin. In a suitable embodiment a population of granulopoietic cells or composition is administered (e.g. sequentially or simultaneously, preferably simultaneously) with a granulocyte-colony stimulating factor; and a growth hormone; and a serotonin; and an interleukin.

In some embodiments compositions (e.g. pharmaceutical compositions) of the invention may be used in combination with another therapeutic, e.g. in combination with an existing cancer or infection therapy, such as radiotherapy, chemotherapy, and/or immunotherapy.

By way of example, the compositions (e.g. pharmaceutical compositions) of the invention may be used in combination with a cell engaging therapy, such as a T cell engaging therapy. Examples of such therapies that may be used in combination with the compositions (e.g. pharmaceutical compositions) of the invention include those selected from the group comprising (or consisting) of: bispecific T cell engagers (BiTEs); checkpoint-inhibitory T cell engagers (CiTEs); simultaneous multiple interaction T cell engagers (SMITEs); trispecific killer engagers (TrikEs); and BiTE-expressing CAR-T cells (CART.BITE cells). In particular, the finding that granulopoietic cells, or compositions (e.g. pharmaceutical compositions) of the invention are able to increase expression by immune cells of costimulatory molecules such as 4-1BB and OX40, suggests that they may advantageously be used in combination with T cell engaging therapies such as mono/bispecific 4-1BB agonists, or TAA/4-1BB bispecific T cell engagers, or mono/bispecific OX40 agonists.

Prior to administration there may be a matching step between a medicament of the invention (e.g. compositions, such as pharmaceutical compositions, of the invention) and the subject to be treated. Matching may be based on data derived from the donor from which the population of granulopoietic cells is derived, and similar data obtained from the subject to be treated. Matching may be achieved on the basis of blood group type, human leukocyte antigen (HLA) type similarity, or combinations thereof.

In one aspect, the invention provides a method of treatment comprising amplifying a non-granulocytic therapeutic immune response, the method comprising providing a composition of the invention to a subject in need of such treatment.

The populations of granulopoietic cells provided may be cells in accordance with any of the embodiments described in this specification. The composition provided may be a composition in accordance with any of the embodiments described in this specification. Accordingly the granulopoietic cells may be provided by means of a composition (e.g. a pharmaceutical composition) of the invention.

Suitably such a subject may be a patient with cancer. A suitable patient may have any form of cancer, including those described further in this disclosure. For example, a patient may have pancreatic cancer.

Suitably such a patient may have an infection. A suitable patient may have any form of infection, including those described further in the present disclosure. Merely by way of example, a patient may have a viral infection.

In one aspect, the invention provides use of a composition of the invention in the manufacture of a medicament.

The composition used in such a manufacture may be a composition in accordance with any of the embodiments described herein. The composition may be for use in amplifying a non-granulocytic therapeutic immune response.

In one aspect, the invention provides a population of granulopoietic cells for use in the manufacture of a medicament for use in amplifying a non-granulocytic therapeutic immune response.

The populations of granulopoietic cells used in such a manufacture may be a population in accordance with any of the embodiments described herein. The medicament manufactured in accordance with this aspect of the invention may be a composition (e.g. pharmaceutical composition) of the invention.

In one aspect, the invention provides use of a composition of the invention in the manufacture of a medicament for treating cancer in a subject.

In one aspect, the invention provides use of a composition of the invention in the manufacture of a medicament for treating an infection in a subject.

The medical uses, methods of treatment or compositions (e.g. pharmaceutical compositions) of the invention may all be employed in the treatment of cancer. Cancer may be treated by killing or otherwise therapeutically reducing the activity of cancer cells. This may occur as a result of the activity of the non-granulocytic cells providing the therapeutic effective immune, and may also occur as a result of cancer killing activity on the part of granulocytes produced on differentiation of the granulopoietic cells employed in the medical uses, methods of treatment, or compositions (e.g. pharmaceutical compositions) of the invention.

In a suitable embodiment a cancer is a solid tumour cancer. The term “solid tumour cancer” refers to an abnormal, malignant mass of tissue that does not contain cysts or liquid inclusions. Examples of solid tumour cancers include carcinomas, sarcomas, and lymphomas.

A solid tumour cancer may be a carcinoma. A carcinoma may be selected from one or more of an adenocarcinoma, a basal cell carcinoma, a squamous cell carcinoma, an adenosquamous carcinoma, a renal cell carcinoma, a ductal carcinoma in situ (DCIS), an invasive ductal carcinoma, an anaplastic carcinoma, a large cell carcinoma, a small cell carcinoma or combinations thereof. A carcinoma may also be selected from epithelial neoplasms, squamous cell neoplasms, squamous cell carcinoma, basal cell neoplasms, basal cell carcinoma, transitional cell carcinomas, adenocarcinomas (such as Adenocarcinoma not otherwise specified (NOS), linitis plastica, vipoma, cholangiocarcinoma, hepatocellular carcinoma NOS, adenoid cystic carcinoma, renal cell carcinoma, Grawitz tumour), adnexal and skin appendage neoplasms, mucoepidermoid neoplasms, cystic mucinous and serous neoplasms, ductal lobular and medullary neoplasms, acinar cell neoplasms, or complex epithelial neoplasms.

Alternatively, a solid tumour cancer may be a sarcoma. A sarcoma may be selected from Askin's tumour, sarcoma botryoides, chondrosarcoma, Ewing's, malignant hemangioendothelioma, malignant schwannoma, osteosarcoma, or soft tissue sarcomas (including alveolar soft part sarcoma, angiosarcoma, cystosarcoma phyllodes, dermatofibrosarcoma protuberans (DFSP), desmoid tumour, desmoplastic small round cell tumour, epithelioid sarcoma, extraskeletal chondrosarcoma, extraskeletal osteosarcoma, fibrosarcoma, gastrointestinal stromal tumour (GIST), hemangiopericytoma, hemangiosarcoma, Kaposi's sarcoma, leiomyosarcoma, liposarcoma, lymphangiosarcoma, malignant fibrous histiocytoma, undifferentiated pleomorphic sarcoma, malignant peripheral nerve sheath tumour (MPNST), neurofibrosarcoma, rhabdomyosarcoma, and synovial sarcoma).

Alternatively, a solid tumour may be a lymphoma, such as a B-cell lymphoma, a T-cell lymphoma, a NK-cell lymphoma, or a Hodgkin's lymphoma.

In a suitable embodiment, a medical use, method of treatment, or composition (e.g. pharmaceutical composition) of the invention is for use in treating one or more of: pancreatic cancer, liver cancer, oesophageal cancer, stomach cancer, cervical cancer, ovarian cancer, lung cancer, bladder cancer, kidney cancer, brain cancer, prostate cancer, myeloma cancer, non-Hodgkin's lymphoma (NHL), larynx cancer, uterine cancer, or breast cancer.

In the case that the medical use, method of treatment, or composition (e.g. pharmaceutical composition) of the invention is for use in treating pancreatic cancer, the pancreatic cancer may be a pancreatic solid tumour cancer, such as a pancreatic adenocarcinoma (e.g. a pancreatic ductal adenocarcinoma).

The medical uses, methods of treatment, or compositions (e.g. pharmaceutical compositions) of the invention may all be employed in the treatment of infections. Such infections may be treated by killing or otherwise therapeutically reducing the activity of infectious agents (such as cellular infectious agents), or by killing or otherwise therapeutically reducing the activity of cells infected by infectious agents.

As used herein, a “cell infected by an infective agent” refers to a cell that is infected by an intracellular infective agent. Said intracellular infective agent may be a pathogen and the cell is therefore a “cell infected by a pathogen”. In a suitable embodiment a cell may be infected by an intracellular bacterium or a virus, preferably a virus.

In a suitable embodiment an infection to be treated is caused by a Gram-negative bacterium or a Gram-positive bacterium. Preferably, an infective agent is a Gram-positive bacterium, such as a bacterium from the genus Staphylococcus.

Suitably an infection to be treated is caused by a bacterium selected from one or more of Staphylococcus spp., multidrug resistant gram-negative bacteria (MRDGN bacteria), vancomycin-resistant Enterococcus (VRE), Mycobacterium spp., carbapenem-resistant Enterobacteriaceae (CRE) gut bacteria, Acinetobacter spp., Actinomyces spp., Propionibacterium spp., Anaplasma spp., Bacillus spp., Arcanobacterium spp., Bacteroides spp., Bartonella spp., Brucella spp., Yersinia spp., Burkholderia spp., Campylobacter spp., Streptococcus spp., Haemophilus spp., Clostridium spp., Corynebacterium spp., Echinococcus spp., Ehrlichia spp., Enterococcus spp., Rickettsia spp., Fusobacterium spp., Neisseria spp., Klebsiella spp., Helicobacter spp., Escherichia spp., Kingella spp., Legionella spp., Listeria spp., Borrelia spp., Mycoplasma spp., Chlamydia spp., Nocardia spp., Pasteurella spp., Bordetella spp., Prevotella spp., Chlamydophila spp., Coxiella spp., Salmonella spp., Group A Streptococcus spp., Shigella spp., Staphylococcus spp., Treponema spp., Vibrio spp., Francisella spp., Pseudomonas spp, and Ureaplasma spp.

In a suitable embodiment the bacterium is selected from one or more of methicillin resistant Staphylococcus aureus (MRSA), multi-drug resistant Mycobacterium tuberculosis (MDR-TB), Pseudomonas aeruginosa, Pseudomonas oryzihabitans, Pseudomonas plecoglossicida, Acinetobacter baumannii, Actinomyces israelii, Actinomyces gerencseriae, Propionibacterium propionicus, Bacillus anthracis, Arcanobacterium haemolyticum, Bacillus cereus, Yersinia pestis, Mycobacterium ulcerans, Campylobacter jejuni, Bartonella bacilliformis, Bartonella henselae, Haemophilus ducreyi, Clostridium difficile, Corynebacterium diphtheria, Burkholderia mallei, Neisseria gonorrhoeae, Klebsiella granulomatis, Streptococcus pyogenes, Streptococcus agalactiae, Haemophilus influenzae, Helicobacter pylori, Escherichia coli (e.g. O157:H7, O111 and O104:H4), Kingella kingae, Legionella pneumophila, Listeria monocytogenes, Burkholderia pseudomallei, Neisseria meningitidis, Mycoplasma pneumoniae, Mycoplasma genitalium, Chlamydia trachomatis, Bordetella pertussis, Streptococcus pneumoniae, Chlamydophila psittaci, Coxiella burnetii, Treponema pallidum, Clostridium tetani, Chlamydophila pneumoniae, Vibrio cholera, Mycobacterium tuberculosis, Salmonella enterica subsp. enterica, serovartyphi, Ureaplasma urealyticum, and Francisella tularensis. Preferably Mycobacterium tuberculosis.

Preferably, in a suitable embodiment the bacterium is selected from one or more of methicillin resistant Staphylococcus aureus (MRSA), multidrug resistant gram-negative bacteria (MRDGN bacteria), vancomycin-resistant Enterococcus (VRE), multi-drug resistant Mycobacterium tuberculosis (MDR-TB), and carbapenem-resistant Enterobacteriaceae (CRE) gut bacteria.

Suitably an infection to be treated is caused by a virus selected from one or more family selected from Adenoviridae, Picornaviridae, Herpesviridae, Coronaviridae, Hepadnaviridae, Flaviviridae, Retroviridae, Orthomyxoviridae, Paramyxoviridae, Papovaviridae, Polyomavirus, Rhabdoviridae, Togaviridae and Bunyaviridae.

In a suitable embodiment the virus may be selected from one or more of HIV-1 (Human immunodeficiency virus), HIV-2, Junin virus, BK virus, Machupo virus, Sabiá virus, Varicella zoster virus (VZV), Alphavirus, Colorado tick fever virus (CTFV), Rhinoviruses, Crimean-Congo hemorrhagic fever virus, Cytomegalovirus, Dengue virus, Ebolavirus (EBOV), Parvovirus B19, Human herpesvirus 6 (HHV-6), Human herpesvirus 7 (HHV-7), Enteroviruses (e.g. EV71), Coxsackie A virus, Sin Nombre virus, Heartland virus, Hanta virus, Hendra virus, Hepatitis A virus, Hepatitis B virus, Hepatitis C virus, Hepatitis D Virus, Hepatitis E virus, Herpes simplex virus 1 and 2 (HSV-1 and HSV-2), Human bocavirus (HBOV), Human metapneumovirus (hMPV), Human papillomaviruses, Human parainfluenza viruses (HPIV), Epstein-Barr virus (EBV), Lassa virus, Lymphocytic choriomeningitis virus (LCMV), Marburg virus, Measles virus, Middle East respiratory syndrome coronavirus, Molluscum contagiosum virus (MCV), Monkeypox virus, Mumps virus, Nipah virus, Norovirus, Poliovirus, JC virus, Respiratory syncytial virus (RSV), Rhinovirus, Rift Valley fever virus, Rotavirus, Rubella virus, SARS coronavirus, SARS-COV-2, Variola major, Variola minor, Venezuelan equine encephalitis virus, Guanarito virus, West Nile virus, Yellow fever virus, and Zika virus.

Suitably an infection to be treated is caused by a fungus selected from one or more of Aspergillus spp., Piedraia spp., Blastomyces spp., Candida spp., Fonsecaea spp., Coccidioides spp., Cryptococcus spp., Cryptosporidium spp., Geotrichum spp., Histoplasma spp., Microsporidia phylum, Paracoccidioides spp., Pneumocystis spp., Sporothrix spp., Trichophyton spp., Epidermophyton spp., Hortaea spp., Malassezia spp., Trichosporon spp., and Mucorales order.

In a suitable embodiment the pathogen is a fungus selected from one or more of Aspergillus fumigatus, Aspergillus flavus, Piedraia hortae, Blastomyces dermatitidis, Candida albicans, Fonsecaea pedrosoi, Coccidioides immitis, Coccidioides posadasii, Cryptococcus neoformans, Geotrichum candidum, Histoplasma capsulatum, Paracoccidioides brasiliensis, Pneumocystis jirovecii, Sporothrix schenckii, Trichophyton tonsurans, Epidermophyton floccosum, Hortaea werneckii, and Trichosporon beigelii.

A macroparasite may be one or more selected from Angiostrongylus spp., Entamoeba Anisakis spp., Ascaris spp., Babesia spp., Balantidium spp., Baylisascaris spp., Blastocystis spp., Capillaria spp., Trypanosoma spp., Clonorchis spp., Ancylostoma spp., Cyclospora spp., Taenia spp., Desmodesmus spp., Dientamoeba spp., Dracunculus spp,. Enterobius spp., Fasciola spp., Filarioidea superfamily, Giardia spp., Gnathostoma spp., Necator spp., Hymenolepis spp., Isospora spp., Leptospira spp., Wuchereria spp., Rhinosporidium spp., Brugia spp., Plasmodium spp., Onchocerca spp., Opisthorchis spp., Paragonimus spp., Naegleria spp., Schistosoma spp., Strongyloides spp., Toxocara spp., Toxoplasma spp., Trichinella spp., Trichomonas spp., and Trichuris spp.

In a suitable embodiment the macroparasite is selected from one or more of Entamoeba histolytica, Ascaris lumbricoides, Balantidium coli, Trypanosoma brucei, Trypanosoma cruzi, Clonorchis sinensis, Cyclospora cayetanensis, Taenia solium, Desmodesmus armatus, Dientamoeba fragilis, Dracunculus medinensis, Enterobius vermicularis, Fasciolopsis buski, Giardia lamblia, Necator americanus, Hymenolepis nana, Hymenolepis diminuta, Isospora belli, Wuchereria bancrofti, Rhinosporidium seeberi, Brugia malayi, Plasmodium vivax, Plasmodium falciparum, Plasmodium malariae, Plasmodium ovale, Plasmodium knowlesi Onchocerca volvulus, Opisthorchis viverrini, Opisthorchis felineus, Naegleria fowleri, Strongyloides stercoralis, Toxoplasma gondii, Trichinella spiralis, Trichuris trichiura, and Trichomonas vaginalis.

In a suitable embodiment, the infective agent is an antibiotic-resistant bacterium (e.g. MRSA), preferably a multi-antibiotic resistant bacterium. An antibiotic resistant bacterium may be resistant to beta-lactams, such as methicillin.

Antibiotic resistance may be assessed using any technique known in the art, such as the Kirby-Baure method, Stokes method, Etest, and/or agar and broth dilution methods for minimum inhibitory concentration (MIC) determination.

In a suitable embodiment a bacterium is resistant to one or more of a penicillin, a penicillinase-resistant penicillin, a cephalosporin, a beta-lactamase inhibitor, a tetracycline and combinations thereof, or pharmaceutically acceptable salts thereof.

In a suitable embodiment a bacterium is resistant to one or more of: vancomycin, nafcillin, oxacillin, teicoplanin, penicillin, methicillin, flucloxacillin, dicloxacillin, cefazolin, cephalothin, cephalexin, cefuroxime, clindamycin, cefazolin, amoxicillin/clavulanate, ampicillin/sulbactam, lincomycin, erythromycin, trimethoprim, sulfamethoxazole, daptomycin, linezolid, rifampin, ciprofloxacin, gentamycin, tetracycline, doxycycline, minocylcine, tigecycline and combinations thereof or pharmaceutically acceptable salts thereof. In a suitable embodiment a bacterium may be resistant to vancomycin and/or teicoplanin, or pharmaceutically acceptable salts thereof.

A multi-antibiotic resistant bacterium is resistant to at least 2, 3, 4, 5, 6, 7, 8, 9 or 10 antibiotics (e.g. chemical antibiotics).

In a suitable embodiment, a granulocyte (or granulocytes) produced on differentiation of a suitable population of granulopoietic cells kills an infective agent by phagocytosing a cell infected by the infective agent. For example, in a suitable embodiment, a granulocyte (or granulocytes) produced on differentiation of a suitable population of granulopoietic cells kills a virus by phagocytosing a cell infected by the virus. In a suitable embodiment, a granulocyte (or granulocytes) produced on differentiation of a suitable population of granulopoietic cells kills a bacterium by phagocytosing a cell infected by the bacterium. In a suitable embodiment, a granulocyte (or granulocytes) produced on differentiation of a suitable population of granulopoietic cells kills an infective agent by releasing one or more factors which kill the infective agent. For example, in a suitable embodiment, a granulocyte (or granulocytes) produced on differentiation of a suitable population of granulopoietic cells kills a virus by releasing one or more factors which kill the virus. In a suitable embodiment, a granulocyte (or granulocytes) produced on differentiation of a suitable population of granulopoietic cells kills a bacterium by releasing one or more factors which kill the bacterium. In some embodiments, a granulocyte (or granulocytes) produced on differentiation of a suitable population of granulopoietic cells kills an infective agent by a combination of the above.

Suitably, populations of granulopoietic cells for use in the various aspects of the invention may be capable of differentiating (preferably differentiate) to give rise to granulocytes that have cytocidal activity that may further contribute to a therapeutic immune response. In particular, such cells may produce granulocytes that are able to kill cancer cells, infected cells, or cellular infective agents.

The inventors have developed a number of ways in which granulocytes having such cytocidal activity may be identified.

For example, a population of granulopoietic cells, for use in accordance with the invention may be one that has the capacity to differentiate to produce granulocytes having the ability to kill at least 5% of cancer cells in a cancer killing assay, the cancer killing assay comprising:
a. admixing granulocytes with cancer cells to form an admixture;
b. incubating said admixture; and
c. measuring the % of cancer cells killed in said admixture.

Suitably, in an embodiment of this sort, the % of cancer cells killed in said admixture is the maximum % of cancer cells killed by 48 hours after forming the admixture. The granulocytes so produced may have the ability to kill at least 10%, 20%, 30%, 40%, 50%, 51.5%, 60%, 70% or 80% of cancer cells in the cancer killing assay.

In a suitable embodiment, the admixture of the assay comprises 1:1, 5:1, 10:1 or 20:1 granulocytes to cancer cells.

Suitably, the cancer cells used in such an assay are HeLa or PANC-1 cancer cells. Suitably, the cancer cells used in such an assay are A549 or A375 cancer cells.

The skilled person will be aware of many suitable cancer killing assays that may be used in assessing the capacity to kill cancer cells. Merely by way of example, in a suitable embodiment the cancer killing assay is carried out using an ACEA Biosciences xCELLigence RTCA DP Analyzer system® according to the manufacturer's instructions and as follows:
a. 6000 cancer cells are placed in the bottom of a 16 well plate;
b. cells are grown to confluence as determined by plateauing of Cell Index (CI) values (i.e. the ‘normalisation point’);
c. 60,000 granulocytes are added (i.e. giving a ratio of 10 granulocytes to 1 cancer cells) and incubated at 37° C.; and
d. the % of cancer cells killed is the maximum % of cancer cells killed by 48 hours after addition of the granulocytes as determined using the following formula: ((Cell Index_{no effector}−Cell Index_{effector})/Cell Index_{no effector})×100.

In one embodiment, the cancer killing assay is carried out using a luciferase cytotoxicity assay as follows:
a. cancer cells are placed in the bottom of (e.g. of a 96 well plate);
b. effector cells such as granulopoietic cells or granulocytes differentiated from the granulopoietic cells are added to the cancer cells (e.g. 17-24 hours later at a ratio of 10:1 or 20:1 effector cell to cancer cells) to form an admixture;
c. the admixture is incubated (e.g. for 48 hours at 37° C. in a 5% CO₂ atmosphere);
d. after the incubation a luciferase substrate (such as luciferin, preferably 5-fluoroluciferin) is added to the admixture (e.g. and incubated at room temperature until the luminescence signal is stabilised (e.g. 7-10 minutes)); and
e. the luminescence signal is measured and the % of cancer cells killed is determined.

The luciferase substrate may be added at any suitable concentration range, such 1-1000 μM, e.g. 10-500 μM or 100-400 μM.

In a suitable embodiment, the cancer killing assay is carried out using a luciferase cytotoxicity assay as follows:
a. 1.5×10⁴ cancer cells are placed in the bottom of a 96 well plate;
b. effector cells such as granulopoietic cells or granulocytes differentiated from the granulopoietic cells are added to the cancer cells 17-24 hours later at a ratio of 10:1 or 20:1 effector cell to cancer cells to form an admixture;
c. the admixture is incubated for 48 hours at 37° C. in a 5% CO₂ atmosphere;
d. after the incubation, 100 μl of ONEglo™ reagent is added to the admixture and incubated at room temperature until the luminescence signal is stabilised (e.g. 7-10 minutes); and
e. the luminescence signal is measured and the % of cancer cells killed is determined.

The following formula may be used to calculate the % of cancer cells killed:

100−((Sample Luminescence Background corrected)/(Target Only Luminescence Background corrected)*100).

In this instance “sample” may be the admixture referred to above comprising effector cells and cancer cells and “target only” may refer to a sample comprising cancer cells and not effector cells. The skilled person will appreciate that the “sample” and “target only” may have been exposed to the same steps, e.g. incubations, to allow comparability. The “background” correction may be achieved by usual normalisation techniques, for example by subtracting any luminescence signal observed with a “media only” sample. Preferably, “background” correction may be achieved by subtracting media only luminescence from “sample” or “target only” luminescence values.

A population of granulopoietic cells in accordance with any of the aspects or embodiments defined herein suitable for use in the various aspects of the invention may be one which differentiates, or has the capacity to differentiate, to produce granulocytes characterized by:
a. increased expression of one or more of GM2A, CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, and PSMB2 when compared to a reference standard, wherein the reference standard is from a neutrophil unsuitable for treating cancer; and/or
b. decreased expression of ANXA1 and/or PPP3CB when compared to a reference standard, wherein the reference standard is from a neutrophil unsuitable for treating cancer.

In a suitable embodiment a population of granulopoietic cells suitable for use in the various aspects of the invention may be characterized in that the granulocytes produced on differentiation of the population of granulopoietic cells have a positively charged cell surface.

Granulopoietic cells suitable for use in the various aspects of the invention may also be identified with respect to the expression profiles of the granulocytes that they are capable of producing (preferably produce).

In a suitable embodiment, a population of granulopoietic cells in accordance with any of the aspects or embodiments defined herein may differentiate, or is able to differentiate, to produce a granulocyte characterized by:
a. increased expression of one or more of GM2A, CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, and PSMB2 when compared to a reference standard, wherein the reference standard is from a granulocyte that does not have the ability to kill cancer cells, or an infective agent, or cells infected by an infective agent; and/or
b. decreased expression of ANXA1 and/or PPP3CB when compared to a reference standard, wherein the reference standard is from a granulocyte that does not have the ability to kill cancer cells, or an infective agent, or cells infected by an infective agent.

Representative sequences for the genes for use in such embodiments of the invention are described in the Sequence Listings and appropriate Ensembl Accession numbers set out in International Patent Application numbers: PCT/GB2020/053197 (published as WO 2021/116711) and PCT/GB2020/053199 (published as WO 2021/116713), the relevant disclosures of which, particularly relating to the sequence listings and identity of sequence suitable to be used in this embodiment of the invention, are incorporated herein by reference.

Determining whether or not a granulocyte has increased expression of one or more of GM2A, CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, and PSMB2; and/or decreased expression of ANXA1 and/or PPP3CB may be performed by measuring expression of said markers. Measuring expression may be carried out by any means known to the person skilled in the art. The term “measuring” as used in reference to expression of one or more genes of the invention encompasses measuring both negative (e.g. no expression) and positive expression (e.g. expression). In a suitable embodiment the expression is positive expression.

In some embodiments expression may be measured using high-throughput techniques. For example, measuring expression may be at the level of transcription (e.g. transcriptomic techniques) or translation (e.g. proteomic techniques). Alternatively, or additionally, the invention may employ the use of genomics, e.g. to detect the presence or absence of single nucleotide polymorphisms (SNPs), promoter sequences, gene copy number (e.g. duplications), and/or enhancer or other relevant genetic features, preferably those that determine the expression level of one or more genes of the invention. High-throughput techniques can be used to analyse whole genomes, proteomes and transcriptomes rapidly, providing data, including the expression levels, of all of the genes, polypeptides and transcripts in a cell. Proteomics is a technique for analysing the proteome of a cell (e.g. at a particular point in time). The proteome is different in different cell types. Typically, proteomics is carried out by mass-spectrometry, including tandem mass-spectrometry, and gel based techniques, including differential in-gel electrophoresis. Proteomics can be used to detect polypeptides expressed in a particular cell type and generate a proteomic profile to allow for the identification of specific cell types.

In a suitable embodiment, mRNA of a target gene can be detected and quantified by e.g. Northern blotting or by quantitative reverse transcription PCR (RT-PCR). Single cell gene expression analysis may also be performed using commercially available systems (e.g. Fluidigm Dynamic Array). Alternatively, or in addition, gene expression levels can be determined by analysing polypeptide levels e.g. by using Western blotting techniques such as ELISA-based assays.

Thus, in a suitable embodiment, gene expression levels are determined by measuring the mRNA/cDNA levels of the genes of the present invention, such as RNA sequencing (RNA-Seq).

In a preferred embodiment, gene expression levels are determined by measuring the polypeptide levels produced by the genes of the present invention, such as by way of mass spectrometry, e.g. liquid chromatography and mass spectrometry (LC-MS/MS).

In a suitable embodiment a granulocyte (or stem cell) for treating cancer may be detected using an enzyme-linked immunosorbent assay (ELISA) or a Luminex assay (commercially available from R&D Systems, USA).

Thus, in a suitable embodiment measuring expression comprises measuring and/or comparing an expression level of one or more polypeptides by a granulocyte, wherein the one or more polypeptides are selected from: CTSG, CAP37, ITGB1, CYBB, SYK, DOCK8, COMP, ATG7, SLC2A1, GZMK, CTSG, ATM, IKBKB, BCAP31, TAPBP, PPP3CB, ANXA1, PERM, PLEC, ACSL1, RAC1, GM2A, CAP37, and PSMB2.

In a suitable embodiment measuring expression comprises measuring and/or comparing an amount of one or more polypeptides produced by a granulocyte, wherein the one or more polypeptides are selected from: CTSG, CAP37, ITGB1, CYBB, SYK, DOCK8, COMP, ATG7, SLC2A1, GZMK, CTSG, ATM, IKBKB, BCAP31, TAPBP, PPP3CB, ANXA1, PERM, PLEC, ACSL1, RAC1, GM2A, CAP37, and PSMB2.

In a suitable embodiment measuring expression comprises measuring and/or comparing an expression level of one or more polypeptides by a stem cell, wherein the one or more polypeptides are selected from: CTSG, CAP37, ITGB1, CYBB, SYK, DOCK8, COMP, ATG7, SLC2A1, GZMK, CTSG, ATM, IKBKB, BCAP31, TAPBP, PPP3CB, ANXA1, PERM, PLEC, ACSL1, RAC1, GM2A, CAP37, and PSMB2.

In a suitable embodiment measuring expression comprises measuring and/or comparing an amount of one or more polypeptides produced by a stem cell, wherein the one or more polypeptides are selected from: CTSG, CAP37, ITGB1, CYBB, SYK, DOCK8, COMP, ATG7, SLC2A1, GZMK, CTSG, ATM, IKBKB, BCAP31, TAPBP, PPP3CB, ANXA1, PERM, PLEC, ACSL1, RAC1, GM2A, CAP37, and PSMB2.

In a suitable embodiment measuring expression employs a genome wide association study, which is compared to a reference standard (e.g. a reference standard from a reference population, such as a reference standard from: a suitable or unsuitable donor, or a suitable or unsuitable granulocyte, or a subject that is suitable or unsuitable for treatment with a population of granulopoietic cells in accordance with the invention, or a subject that is at risk or not at risk of cancer or combinations thereof).

Methods suitable for establishing a baseline or reference value for comparing expression levels are conventional techniques known to those skilled in the art.

The term “increased” as used herein in reference to expression of the one or more genes of the invention may refer to an expression level that is statistically-significantly increased when compared to a reference standard. Such a gene may be considered to be upregulated.

In a suitable embodiment increased expression means greater than 1-fold, 1.25-fold to about 10-fold or more expression relative to a reference standard. In some embodiments, increased expression means greater than at least about 1.1-fold, 1.2-fold, 1.25-fold, 1.5-fold, 1.75-fold, 2-fold, 4-fold, 5-fold, 10-fold, 15-fold, 20-fold, 25-fold, 30-fold, 35-fold, 40-fold, 50-fold, 75-fold, 100-fold, 150-fold, 200-fold, or at least about 300-fold expression when compared to a reference standard.

The term “decreased” as used herein in reference to expression of the one or more genes of the invention may refer to an expression level that is statistically-significantly decreased when compared to a reference standard. Such a gene may be considered to be downregulated.

In a suitable embodiment decreased expression means less than −1-fold, −1.25-fold to about −10-fold or more expression relative to a reference standard. In some embodiments, decreased expression means less than at least about −1.1-fold, −1.2-fold, −1.25-fold, −1.5-fold, −1.75-fold, —2-fold, −4-fold, −5-fold, −10-fold, −15-fold, −20-fold, 25-fold, −30-fold, −35-fold, −40-fold, −50-fold, −75-fold, −100-fold, −150-fold, −200-fold, or at least about −300-fold expression when compared to a reference standard.

The fold change difference can be in absolute terms (e.g. CPM: counts per million) or Log2CPM (a standard measure in the field) of the expression level in a sample. Preferably the fold change is Log2 fold change. In a suitable embodiment a Log2 change is an increase of at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6 or 2.7. In a suitable embodiment a Log2 change is a decrease of 0.1 or more, 0.2 or more, 0.3 or more, 0.4 or more, 0.5 or more, 0.6 or more, 0.7 or more, 0.8 or more, 0.9 or more, 1.0 or more, 1.1 or more, 1.2 or more or 1.3 or more. A decrease may be indicated by the presence of a “-” symbol prior to the value.

In a suitable embodiment said fold-change is measured and/or is determined by RNA sequencing (RNA-Seq), e.g. in toto.

The term “unchanged” or “the same” as used herein in reference to expression of the one or more genes of the invention may refer to an expression level that is not statistically-significantly different to a reference standard. Preferably, an expression level that is the same as a reference standard.

The expression level may be an average such as a mean expression level. In a suitable embodiment statistical significance is determined using two-way ANOVA, e.g. where n is at least 3 and data are presented as mean+/−standard error of mean.

In a suitable embodiment the methods of the invention comprise measuring expression of combinations of the genes described herein.

The term “one or more” when used in the context of a gene described herein may mean at least 2, 3, 4, 5, 6, 7, 8, 9, 10, 11, 12, 13, 14, 15, 16, 17, 18, 19, 20, 21 or 22 of the genes. Preferably, the term “one of more” means all of the genes. Likewise, the term “one or more” when used in the context of a list of polypeptides described herein may mean at least 2, 3, 4, 5, 6, 7, 8, 9, 10, 11, 12, 13, 14, 15, 16, 17, 18, 19, 20, 21 or 22 of the polypeptides. Preferably, the term “one of more” means all of the polypeptides.

The expression of one or more of ITGB1, CYBB, SYK, DOCK8, COMP, ATG7, SLC2A1, GZMK, CTSG, ATM, IKBKB, BCAP31, TAPBP, PPP3CB, ANXA1, PERM, PLEC, ACSL1, RAC1, GM2A, CAP37, and PSMB2 may correlate with a granulocyte's ability to kill cancer cells. Said genes may therefore be referred to herein as genes associated with the ability to kill cancer cells. Thus, the term “one or more genes associated with the ability to kill cancer cells” (and the like) may in be synonymous with (and thus replaced with) the term “one or more of ITGB1, CYBB, SYK, DOCK8, COMP, ATG7, SLC2A1, GZMK, CTSG, ATM, IKBKB, BCAP31, TAPBP, PPP3CB, ANXA1, PERM, PLEC, ACSL1, RAC1, GM2A, CAP37, and PSMB2”. Thus, the term “one or more polypeptides associated with the ability to kill cancer cells” (and the like) may in be synonymous with (and thus replaced with) the term “one or more of ITGB1, CYBB, SYK, DOCK8, COMP, ATG7, SLC2A1, GZMK, CTSG, ATM, IKBKB, BCAP31, TAPBP, PPP3CB, ANXA1, PERM, PLEC, ACSL1, RAC1, GM2A, CAP37, and PSMB2”.

In a suitable embodiment expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 may be increased in a granulocyte that has the ability to kill cancer cells when compared to a granulocyte that does not have the ability to kill cancer cells. Alternatively, or additionally, in a suitable embodiment expression of ANXA1 and/or PPP3CB is decreased in a granulocyte that has the ability to kill cancer cells when compared to a granulocyte that does not have the ability to kill cancer cells.

In a suitable embodiment expression of S100A9 and/or S100A8 may be increased in a granulocyte derived from a population of granulopoietic cells of the invention when compared to a reference standard, when the reference standard is from a granulocyte that does not have the ability to kill cancer cells.

The expression of one or more of ITGB1, CYBB, SYK, DOCK8, COMP, ATG7, SLC2A1, GZMK, CTSG, ATM, IKBKB, BCAP31, TAPBP, PPP3CB, ANXA1, PERM, PLEC, ACSL1, RAC1, GM2A, CAP37, and PSMB2 correlates with a granulocyte's ability to kill an infective agent or cells infected by an infective agent. Said genes are therefore referred to herein as genes associated with ability to kill an infective agent or cells infected by an infective agent. Thus, the term “one or more genes associated with ability to kill an infective agent or cells infected by an infective agent” (and the like) may in be synonymous with (and thus replaced with) the term “one or more of ITGB1, CYBB, SYK, DOCK8, COMP, ATG7, SLC2A1, GZMK, CTSG, ATM, IKBKB, BCAP31, TAPBP, PPP3CB, ANXA1, PERM, PLEC, ACSL1, RAC1, GM2A, CAP37, and PSMB2”.

In a suitable embodiment expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is increased in a granulocyte that has the ability to kill an infective agent or cells infected by an infective agent when compared to a granulocyte that does not have the ability to kill an infective agent or cells infected by an infective agent. Alternatively or additionally, in a suitable embodiment expression of ANXA1 and/or PPP3CB is decreased in a granulocyte that has the ability to kill an infective agent or cells infected by an infective agent when compared to a granulocyte that does not have the ability to kill an infective agent or cells infected by an infective agent.

In a suitable embodiment a method of the invention may further comprise measuring expression of one or more genes selected from: S100A9 and S100A8. In a suitable embodiment expression of S100A9 and/or S100A8 may be increased in a granulocyte of the invention when compared to a reference standard, when the reference standard is from a granulocyte that does not have the ability to kill an infective agent or cells infected by an infective agent.

The expression level of one or more genes of the invention may be compared to a reference standard. The comparison may be carried out by any suitable technique known to the person skilled in the art, e.g. a bioinformatics technique. The expression level of the genes described herein is suitably known in said reference standard.

The reference standard may be a proteomic profile (indicating an amount of polypeptide expressed by a granulocyte), a transcriptomic profile (indicating an amount of gene expression by a granulocyte, e.g. measured by way of RNA produced by said granulocyte) or a genomic profile. A genomic profile may be used to detect the presence or absence of SNPs, promoter sequences, gene copy number (e.g. duplications), and/or enhancer or other relevant genetic features, preferably those that determine the expression level of one or more genes of the invention. The skilled person will appreciate that both the proteomic and transcriptomic profiles are measures of gene expression and will employ the appropriate reference standard depending on the technique used to measure gene expression in accordance with the invention. For example, where proteomics is used in practising the present invention the skilled person will employ a reference standard that is a proteomic profile, where transcriptomics is used in practising the present invention the skilled person will employ a reference standard that is a transcriptomic profile, and where genomics is used in practicing the present invention the skilled person will employ a reference standard that is a genomic profile. A reference standard may refer to a database (e.g. a genomic database), e.g. which may include data from one or more sources, such as one or more subjects and/or cells.

A reference standard is preferably a reference standard for a granulocyte that does not have the ability to kill cancer cells (e.g. a transcriptomic or proteomic profile of a granulocyte that is unsuitable for treating cancer). Such a reference standard may be from a subject that does not have cancer (a healthy subject) or from a subject that has cancer. Preferably, such a reference standard is from a subject that does not have cancer.

In a suitable embodiment expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is increased when compared to a reference standard when the reference standard is from a granulocyte that does not have the ability to kill cancer cells. In a suitable embodiment expression of ANXA1 and/or PPP3CB is decreased when compared to a reference standard, when the reference standard is from a granulocyte that does not have the ability to kill cancer cells. In a suitable embodiment expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is increased when compared to a reference standard when the reference standard is from a granulocyte that does not have the ability to kill cancer cells and expression of ANXA1 and/or PPP3CB is decreased when compared to a reference standard, when the reference standard is from a granulocyte that does not have the ability to kill cancer cells.

A reference standard may be a reference standard for a granulocyte that is suitable for treating cancer (e.g. a transcriptomic or proteomic profile of a granulocyte that is suitable for treating cancer). In a suitable embodiment expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is increased or the same when compared to a reference standard, when the reference standard is from a granulocyte that has the ability to kill cancer cells. In a suitable embodiment expression of ANXA1 and/or PPP3CB is decreased or the same when compared to a reference standard, when the reference standard is from a granulocyte that has the ability to kill cancer cells.

In some embodiments the present invention may comprise the use of a reference standard for a granulocyte that does not have the ability to kill cancer cells and a reference standard for a granulocyte that has the ability to kill cancer cells.

A reference standard is preferably a reference standard for a granulocyte that that does not have the ability to kill an infective agent or cells infected by an infective agent does not have the ability to kill an infective agent or cells infected by an infective agent (e.g. a transcriptomic or proteomic profile of a granulocyte that that does not have the ability to kill an infective agent or cells infected by an infective agent does not have the ability to kill an infective agent or cells infected by an infective agent).

In a suitable embodiment expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is increased when compared to a reference standard when the reference standard is from a granulocyte that does not have the ability to kill an infective agent or cells infected by an infective agent. In a suitable embodiment expression of ANXA1 and/or PPP3CB is decreased when compared to a reference standard, when the reference standard is from a granulocyte that does not have the ability to kill an infective agent or cells infected by an infective agent. In a suitable embodiment expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is increased when compared to a reference standard when the reference standard is from a granulocyte that does not have the ability to kill an infective agent or cells infected by an infective agent and expression of ANXA1 and/or PPP3CB is decreased when compared to a reference standard, when the reference standard is from a granulocyte that does not have the ability to kill an infective agent or cells infected by an infective agent.

A reference standard may be a reference standard for a granulocyte that has the ability to kill an infective agent or cells infected by an infective agent (e.g. a transcriptomic or proteomic profile of a granulocyte that is suitable for treating infection). In a suitable embodiment expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is increased or the same when compared to a reference standard, when the reference standard is from a granulocyte that has the ability to kill an infective agent or cells infected by an infective agent. In a suitable embodiment expression of ANXA1 and/or PPP3CB is decreased or the same when compared to a reference standard, when the reference standard is from a granulocyte that has the ability to kill an infective agent or cells infected by an infective agent.

In some embodiments the present invention may comprise the use of a reference standard for a granulocyte that does not have the ability to kill an infective agent or cells infected by an infective agent and a reference standard for a granulocyte that has the ability to kill an infective agent or cells infected by an infective agent.

In a suitable embodiment, a population of granulopoietic cells suitable for use in the various aspects of the invention is able to give rise to granulocytes that have a positively charged cell surface.

The inventors believe that granulocyte cell surface charge may correlate with suitability for treating cancer and/or with suitability for treating an infection, with granulocytes (e.g. neutrophils) that are more positively charged (or less negatively charged) being suitable for treating cancer and/or more efficacious in treating cancer and/or being suitable for treating an infection and/or more efficacious in treating an infection. The level of cell surface charge may be determined when compared to a reference standard, preferably wherein the reference standard is from a granulocyte that does not have the ability to kill cancer cells and/or does not have the ability to kill an infective agent or cells infected by an infective agent.

In a suitable embodiment a population of granulopoietic cells may be considered as suitable for use in accordance with the various aspects of the invention if it is capable of differentiating (and preferably if it differentiates) into granulocytes having a positively charged (or less negatively charged) cell surface. A cell surface charge can be determined using any suitable technique known in the art. In a suitable embodiment the cell surface charge is determined using electrophoresis. An electrophoretic mobility assay may be one described in “Cell Electrophoresis” edited by Johann Bauer (ISBN 0-8493-8918-6 published by CRC Press, Inc.) the teaching of which is incorporated herein in its entirety. In another embodiment cell surface charge can be determined using negatively and/or positively charged means. In a suitable embodiment, a granulocyte has a positive cell surface charge when it can be bound by a negatively charged means, and not a positively charged means. In a suitable embodiment, a granulocyte has a negative cell surface charge when it can be bound by a positively charged means, and not a negatively charged means. Such negatively and/or positively charged means may also be used to measure the concentration of a granulocyte cell in a sample. A positively charged means may be a positively charged particle, nanoprobe or nanoparticle, or a cation exchange media. Suitable nanoparticles may be prepared by conjugating superparamagnetic Iron(II,III) oxide (Fe₃O₄) nanoparticles (NPs) with (3-Aminopropyl)triethoxysilane (APTES) to form a thin layer of Silicon dioxide (SiO₂) shell on the NPs' surface upon reaction with Tetraethyl orthosilicate (TEOS) and ammonium hydroxide (NH₄OH). Fluorescein isothiocyanates (FITCs) may be embedded in the SiO₂ shell, thus exposing the Si-linked hydroxyl groups (SiO₂—OH) and creating the negative surface charge. Branched poly(ethylene imine) (PEI) molecules may be used to not only to cover the SiO₂—OH groups in a non-covalently manner but also to expose the additional amine groups that carry the positive charges. Thus, in a suitable embodiment a negatively charged nanoparticle is prepared by conjugating Fe₃O₄ nanoparticles with APTES to form a thin layer of SiO₂ shell on the nanoparticle surface upon reaction with Tetraethyl orthosilicate (TEOS) and ammonium hydroxide (NH₄OH), and embedding a FITC in the SiO₂ shell, thus exposing the SiO₂—OH groups (creating the negative surface charge). In another embodiment, a positively charged nanoparticle is prepared by contacting a negatively charged nanoparticle (as described herein) with a PEI molecule (e.g. to expose additional amine groups that carry a positive charge). In a suitable embodiment, the negatively charged means (e.g. nanoparticle) may have a negative surface charge of at least −5 mV, −10 mV, −20 mV, −30 mV, or −40 mV. Preferably, the negatively charged means (e.g. nanoparticle) has may have a negative surface charge of at least −35 mV. In a suitable embodiment, the positively charged means (e.g. nanoparticle) may have a positive surface charge of at least ⁺5 mV, ⁺10 mV, ⁺20 mV, ⁺30 mV, or ⁺40 mV. Preferably, the positively charged means (e.g. nanoparticle) has may have a positive surface charge of at least ⁺35 mV. The surface charge of said positively or negatively charged means (e.g. nanoparticle) may refer to the surface zeta potential of the positively or negatively charged means (e.g. nanoparticle). The surface zeta potential may be measured with a Dynamic light scattering particle size analyser (e.g. the Zetasizer Nano-ZS90, Malvern, UK).

In a suitable embodiment, a population of granulopoietic cells described herein may be able to differentiate to produce granulocytes with the ability to kill cancer cells.

The “ability to kill cancer cells” may be determined by admixing a cell (e.g. a granulocyte, such as a neutrophil) with a cancer cell, and measuring (e.g. after incubation) viability of said cancer cell. If the cancer cell is no longer viable (i.e. has been killed), the cell exhibits an ability to kill cancer cells. In a suitable embodiment the ability to kill cancer cells is determined using a Cancer Killing Activity (CKA) assay described herein.

In a suitable embodiment a CKA assay comprises:
a. contacting cancer cells with granulocytes to form a test sample (preferably at a ratio of 10:1 granulocytes to cancer cells);
b. incubating said test sample; and
c. measuring the % of cancer cells killed in said test sample.

In a suitable embodiment a CKA assay comprises:
a. admixing granulocytes with cancer cells to provide an admixture (preferably at a ratio of 10:1 granulocytes to cancer cells);
b. incubating said admixture; and
c. measuring the % of cancer cells killed in said admixture

The term “admixing” as used herein means mixing one or more components together in any order, whether sequentially or simultaneously. In a suitable embodiment “admixing” means contacting a first component with a second component (e.g. a granulocyte and cancer cell).

The cancer cell for use in an assay may be one or more selected from a pancreatic cancer cell line, a liver cancer cell line, an oesophageal cancer cell line, a stomach cancer cell line, a cervical cancer cell line, an ovarian cancer cell line, a lung cancer cell line, a bladder cancer cell line, a kidney cancer cell line, a brain cancer cell line, a prostate cancer cell line, a myeloma cancer cell line, a non-Hodgkin's lymphoma (NHL) cell line, a larynx cancer cell line, a uterine cancer cell line, or a breast cancer cell line. Suitable cell lines are available commercially from the American Type Culture Collection United Kingdom (U.K.), Guernsey, Ireland, Jersey and Liechtenstein, LGC Standards, Queens Road, Teddington, Middlesex, TW11 OLY, UK. For example, a pancreatic cell line may be one or more of Capan-2, ATCC HTB-80; Panc 10.05, ATCC CRL-2547; CFPAC-1, ATCC CRL-1918; HPAF-II, ATCC CRL-1997; SW 1990, ATCC CRL-2172; BxPC-3, ATCC CRL-1687; AsPC-1, ATCC CRL-1682; ATCC® TCP-1026TM; SW1990, ATCC CRL-2172; SU.86.86, ATCC CRL-1837; BXPC-3, ATCC CRL-1687; Panc 10.05, ATCC CRL-2547; MIA-PaCa-2, ATCC CRL-1420; PANC-1, ATCC CRL-1469; or ATCC® TCP-2060™. Preferably the cancer cell line is pancreatic cancer cell line, such as PANC-1. In a suitable embodiment the cancer cell line is a cervical cancer cell line, such as a HeLa cell.

The incubation step may be carried out for between 1 hour and 100 hours. Suitably, the incubation step may be carried out for between 5 hours and 75 hours, for example between hours and 20 hours. The incubation step may be carried out for between 6 hours to 6 days. Suitably, the incubation step may be carried out for between 6 hours and 2 days, for example for between 12 hours to 36 hours, such as between 16 to 24 hours. In a suitable embodiment the incubation step is carried out for 24 hours. In another embodiment the incubation step is carried out for 48 hours. The incubation step may be carried out at any temperature suitable for cell growth and viability, for example at a temperature between 35° C. to 42° C., suitably at 37 or 39° C. Preferably the incubation step is carried out at 37 or 39° C. for 24 hours. Preferably the incubation step is carried out for 16-24 hours at 30-40° C. (e.g. 37° C.).

The % of cancer cells killed can be measured by reference to the total number of starting cancer cells. The number of cancer cells killed can be measured using any suitable means, for example by viability staining (e.g. trypan blue staining), and microscopy, or using other automated means, for example by cell electronic sensing equipment, such as the RT-CES™ system available from ACEA Biosciences, Inc. (11585 Sorrento Valley Rd., Suite 103, San Diego, CA 92121, USA). In some embodiments the % of cancer cells killed may be determined within 24 hours (e.g. of incubating a cancer cell line and a granulocyte). The % of cancer cells killed is preferably the maximum number of cancer cells killed when carrying out a method of the invention. The % of cancer cells killed in said admixture may be the maximum % of cancer cells killed by 48 hours after forming the admixture.

A ratio of at least 1:1, 5:1 or 10:1 of granulocytes to cancer cells may be used. Preferably a 5:1 ratio of granulocytes to cancer cells is used. More preferably a 10:1 ratio of granulocytes to cancer cells is used.

The number of cancer cells killed can also be measured using the ACEA Biosciences xCELLigence RTCA DP Analyzer system®. The xCELLigence System is a real-time cell analyser, allowing for label-free and dynamic monitoring of cellular phenotypic changes continuously by measuring electrical impedance. Such measurements may be carried out as detailed in Example 11. Said System is commercially available from ACEA Biosciences 6779 Mesa Ridge Road #100, San Diego, CA 92121 USA.

In a suitable embodiment a CKA assay is carried out using an ACEA Biosciences xCELLigence RTCA DP Analyzer system® according to the manufacturer's instructions and as follows:
e. 6000 cancer cells are placed in the bottom of a 16 well plate;
f. cells are grown to confluence as determined by plateauing of Cell Index (CI) values (i.e. the ‘normalisation point’);
g. 60,000 granulocytes are added (i.e. giving a ratio of 10 granulocytes to 1 cancer cells) and incubated at 37° C.; and
h. the % of cancer cells killed is the maximum % of cancer cells killed by 48 hours after addition of the granulocytes as determined using the following formula: ((Cell Index_{no effector}−Cell Index_{effector})/Cell Index_{no effector})×100.

The maximum % of cancer cells killed may be referred to herein as “% CKA”.

Preferably the cancer cells are PANC-1 cells, which are commercially available from the American Type Culture Collection United Kingdom (U.K.), Guernsey, Ireland, Jersey and Liechtenstein, LGC Standards, Queens Road, Teddington, Middlesex, TW11 OLY, UK and have catalogue number ATCC CRL-1469.

In a particularly preferred embodiment, the granulocyte having the ability to kill cancer cells, kills less than 15% of non-cancer cells in the “non-cancer killing activity (NCKA) assay” described herein. Preferably a granulocyte kills less than 10% (e.g. less than 5% or less than 1%) of non-cancer cells in the “non-cancer killing activity (NCKA) assay” described herein.

The “non-cancer killing activity (NCKA) assay” or “NCKA assay” may be carried out using an ACEA Biosciences xCELLigence RTCA DP Analyzer system® according to the manufacturer's instructions and as follows:
a. 6000 non-cancer cells are placed in the bottom of a 16 well plate;
b. cells are grown to confluence as determined by plateauing of Cell Index (CI) values (i.e. the ‘normalisation point’);
c. 60,000 granulocytes are added (i.e. giving a ratio of 10 granulocytes to 1 non-cancer cells) and incubated at 37° C.; and
d. the % of non-cancer cells killed is the maximum % of non-cancer cells killed by 48 hours after addition of the granulocytes as determined using the following formula:

((Cell Index_{no effector}−Cell Index_{effector})/Cell Index_{no effector})×100.

Preferably the non-cancer cells are MCF-12F non-cancer cells, which are commercially available from the American Type Culture Collection, 10801 University Boulevard. Manassas, VA 20110 USA and have catalogue number ATCC® CRL-10783™. In another embodiment the non-cancer cells are liver cells (e.g. primary non-transplantable liver tissue cells).

In a suitable embodiment a granulocyte may be considered “a granulocyte with the ability to kill cancer cells” if it kills at least 5% of cancer cells in a method described herein. A granulocyte may be considered “a granulocyte with the ability to kill cancer cells” if it kills at least 10%, 20%, 30%, 40%, 50%, or 51.5% of the cancer cells present. In a suitable embodiment a granulocyte may be considered “a granulocyte with the ability to kill cancer cells” if it kills at least 60% of the cancer cells present. In a suitable embodiment a granulocyte may be considered “a granulocyte with the ability to kill cancer cells” if it kills at least 70% of the cancer cells present. For example, a granulocyte may be considered “a granulocyte with the ability to kill cancer cells” if it kills at least 80% or 90% of the cancer cells present. In a particularly preferred embodiment, a granulocyte may be considered “a granulocyte with the ability to kill cancer cells” if it kills at least 51.5% of the cancer cells present. Reference in this specification to a population of granulopoietic cells that “with the ability to kill cancer cells” may be taken as referring to a population of granulopoietic cells that is able to differentiate into granulocytes that have the ability to kill cancer cells in line with the definitions set out above.

In contrast, a granulocyte that “does not have the ability to kill cancer cells” or is “unable to kill cancer cells” may be a granulocyte that is not capable of killing (preferably does not kill) at least 5% of cancer cells in a method described herein. A granulocyte that “does not have the ability to kill cancer cells” or is “unable to kill cancer cells” may be a granulocyte that is not capable of killing (preferably does not kill) at least 10%, 20%, 30%, 40%, 50%, or 51.5% of the cancer cells present. In a suitable embodiment a granulocyte that “does not have the ability to kill cancer cells” or is “unable to kill cancer cells” may be a granulocyte that is not capable of killing (preferably does not kill) at least 60% of the cancer cells present. In a suitable embodiment a granulocyte that “does not have the ability to kill cancer cells” or is “unable to kill cancer cells” may be a granulocyte that is not capable of killing (preferably does not kill) at least 70% of the cancer cells present. For example, a granulocyte that “does not have the ability to kill cancer cells” or is “unable to kill cancer cells” may be a granulocyte that is not capable of killing (preferably does not kill) at least 80% or 90% of the cancer cells present. In a suitable embodiment, a granulocyte that “does not have the ability to kill cancer cells” or is “unable to kill cancer cells” may be a granulocyte that is not capable of killing (preferably does not kill) at least 51.5% of the cancer cells present. Likewise, reference to a population of granulopoietic cells that “does not have the ability to kill cancer cells” or is “unable to kill cancer cells” may be taken as referring to a population of granulopoietic cells that does not differentiate into granulocytes that have the ability to kill cancer cells and/or that differentiates into granulocytes that “do not have the ability to kill cancer cells” or are “unable to kill cancer cells”.

In a suitable embodiment, a population of granulopoietic cells suitable described herein may be able to differentiate to produce granulocytes with the ability to kill an infective agent, or a cell infected by an infective agent.

The “ability to kill an infective agent or a cell infected by an infective agent” may be determined by admixing a cell (e.g. a granulocyte, such as a neutrophil) with an infective agent or a cell infected by an infective agent, and measuring (e.g. after incubation) viability of said infective agent or cell infected by the infective agent. If the infective agent or cell infected by the infective agent is no longer viable (i.e. has been killed), the cell exhibits an ability to kill an infective agent or a cell infected by an infective agent. In a suitable embodiment the ability to kill an infective agent or a cell infected by an infective agent is determined using an Infection Killing Activity (IKA) assay described herein.

In a suitable embodiment an IKA assay comprises:
a. contacting an infective agent or cell infected by an infective agent with granulocytes to form a test sample;
b. incubating said test sample; and
c. measuring the % of infective agent or cells infected by the infective agent killed in said test sample.

In a suitable embodiment an IKA assay comprises:
a. admixing granulocytes with an infective agent or a cell infected by an infective agent to provide an admixture;
b. incubating said admixture; and
c. measuring the % of infective agent or cells infected by the infective agent killed in said admixture.

The incubation step or contacting between a granulocyte and an infective agent/infective agent-infected cell may be carried out for between 1 hour and 100 hours. Preferably, the incubation step or contacting between a granulocyte and an infective agent/infective agent-infected cell may be carried out for between 5 hours and 75 hours, for example between 10 hours and 20 hours. The incubation step or contacting between a granulocyte and an infective agent/infective agent-infected cell may be carried out for between 6 hours to 6 days. Suitably, the incubation step or contacting between a granulocyte and an infective agent/infective agent-infected cell may be carried out for between 6 hours and 2 days, for example for between 12 hours to 36 hours, such as between 16 to 24 hours. In a suitable embodiment the incubation step is carried out for 24 hours. In another embodiment the incubation step or contacting between a granulocyte and an infective agent/infective agent-infected cell is carried out for 48 hours. The incubation step or contacting between a granulocyte and an infective agent/infective agent-infected cell may be carried out at any temperature suitable for cell growth and viability, for example at a temperature between 35° C. to 42° C., suitably at 37 or 39° C. Preferably the incubation step or contacting between a granulocyte and an infective agent/infective agent-infected cell step is carried out at 37 or 39° C. for 24 hours. Preferably the incubation step or contacting between a granulocyte and an infective agent/infective agent-infected cell is carried out for 16-24 hours at 30-40° C. (e.g. 37° C.).

The above-mentioned conditions may be particularly suitable when incubating/contacting a granulocyte with a cell infected by an infective agent.

The incubation step or contacting between a granulocyte and an infective agent/infective agent-infected cell may be carried out for between 30 minutes and 24 hours (e.g. prior to assessing % killing). Preferably, the incubation step or contacting between a granulocyte and an infective agent/infective agent-infected cell may be carried out for between 1-3 hours, for example for 2 hours. In other words, the assessment of % killing may be determined following contacting/incubating for 2 hours. The incubation step or contacting between a granulocyte and an infective agent/infective agent-infected cell may be carried out at any temperature suitable for cell growth and viability, for example at a temperature between 35° C. to 42° C., suitably at 37° C.

The above-mentioned conditions may be particularly suitable when incubating/contacting a granulocyte with an infective agent, such as a bacterium.

In a suitable embodiment a contacting or incubation step is carried out in solution. In other words, the infective agent or cells infected with an infective agent may be growing in solution (i.e. not adhered to/growing on a surface, such as a surface of a plate).

Preferably, where the infective agent is a bacterium a contacting or incubation step is carried out in solution. In contrast, where the method employs cells infected with an infective agent it is preferred that said cells are growing on or adhered to a surface, such as a surface of a plate.

In a suitable embodiment said contacting or incubation step is carried out under agitation, e.g. at 100-250 rpm, such as 120 rpm.

In a suitable embodiment, where the method employs cells infected with an infective agent, the methods of the invention may comprise the use of at least a 1:1, 5:1 or 10:1 ratio of granulocytes to cells. Preferably the methods comprise the use of a 5:1 ratio of granulocytes to cells. More preferably the methods comprise the use of a 10:1 ratio of granulocytes to cells.

The % of cells killed can be measured by reference to the total number of starting cells. The number of cells killed can be measured using any suitable means, for example by viability staining (e.g. trypan blue staining), and microscopy, or using other automated means, for example by cell electronic sensing equipment, such as the RT-CEST system available from ACEA Biosciences, Inc. (11585 Sorrento Valley Rd., Suite 103, San Diego, CA 92121, USA).

In some embodiments the % of cells killed may be determined within 24 hours (e.g. of incubating a cell and a granulocyte). The % of cells killed is preferably the maximum number of cells killed when carrying out a method of the invention.

The number of cells killed can be also be measured using the ACEA Biosciences xCELLigence RTCA DP Analyzer system®. The xCELLigence System is a real-time cell analyser, allowing for label-free and dynamic monitoring of cellular phenotypic changes continuously by measuring electrical impedance. Such measurements may be carried out as detailed in the Examples. Said System is commercially available from ACEA Biosciences 6779 Mesa Ridge Road #100, San Diego, CA 92121 USA.

In a suitable embodiment, where an infective agent is a bacterium, a ratio of at least 1:10, 1:5, 1:3 or 1:2 granulocytes to colony forming units may be used. Preferably a 1:2 ratio of granulocytes to colony forming units is used. More preferably a 1:1 ratio of granulocytes to colony forming units is used.

In a suitable embodiment the ability to kill an infective agent or a cell infected by an infective agent is determined using an MRSA assay described herein.

In a suitable embodiment, the MRSA assay comprises:
a. admixing granulocytes with MRSA cells to form an admixture;
b. incubating said admixture; and
c. measuring the % of MRSA cells killed in said admixture.

The “MRSA assay” may be carried out as follows:
a. admixing 100 μl of a 1×10⁷ CFU/ml solution of MRSA strain USA300 in RPMI 1640 with 100 μl of a solution containing 1×10⁷ granulocytes/ml;
b. incubating the admixture at 37° C. under shaking at 120 rpm;
c. taking a sample at 2 hours (diluting in sterile RPMI as needed) and plating on Tryptic Soy Agar;
d. incubating the plated sample at 37° C. for 24 hours;
e. counting the bacterial colonies; and
f. quantifying the total CFU content; and
g. calculating the % of MRSA cells killed based on the CFU content in steps a. and f using the formula ((CFU content_{no effector}−CFU content_{effector})/CFU content_{no effector})×100.

In a particularly preferred embodiment the term “having the ability to kill an infective agent or cell infected by an infective agent” as used herein further means that a granulocyte kills less than 15% of healthy (non-infected) cells in the “healthy (non-infected) cell assay” described herein. Preferably a granulocyte kills less than 10% (e.g. less than 5% or less than 1%) of healthy (non-infected) cells in the “healthy (non-infected) cell assay” described herein.

The “healthy (non-infected) cell assay” may be carried out using an ACEA Biosciences xCELLigence RTCA DP Analyzer system® according to the manufacturer's instructions and as follows:
a. 6000 healthy (non-infected) cells are placed in the bottom of a 16 well plate;
b. cells are grown to confluence as determined by plateauing of Cell Index (CI) values (i.e. the ‘normalisation point’);
c. 60,000 granulocytes are added (i.e. giving a ratio of 10 granulocytes to 1 non-pathogen-infected cells) and incubated at 37° C.; and
d. the % of healthy (non-infected) cells killed is the maximum % of non-pathogen-infected cells killed by 48 hours after the addition of the granulocytes as determined using the following formula: ((Cell Index_{no effector}−Cell Index_{effector})/Cell Index_{no effector})×100.

Preferably the healthy (non-infected) cells are MCF-12F, which are commercially available from the American Type Culture Collection, 10801 University Boulevard. Manassas, VA 20110 USA and have catalogue number ATCC® CRL-10783™. In another embodiment the healthy (non-infected) cells are liver cells (e.g. primary non-transplantable liver tissue cells).

In a suitable embodiment a granulocyte may be considered “a granulocyte with the ability to kill an infective agent or cells infected by an infective agent” if it kills at least 5% of the infective agent or the cells infected by an infective agent in a method described herein. A granulocyte may be considered “a granulocyte with the ability to kill an infective agent or cells infected by an infective agent” if it kills at least 10%, 20%, 30%, 40%, or 50% of the infective agent or cells infected by an infective agent present. In a suitable embodiment a granulocyte may be considered “a granulocyte with the ability to kill an infective agent or cells infected by an infective agent” if it kills at least 60% of the infective agent or the cells infected by an infective agent present. In a suitable embodiment a granulocyte may be considered “a granulocyte with the ability to kill an infective agent or cells infected by an infective agent” if it kills at least 70% of the infective agent or the cells infected by an infective agent present. Preferably a granulocyte may be considered “a granulocyte with the ability to kill an infective agent or cells infected by an infective agent” if it kills at least 80% or 90% of the infective agent or the cells infected by an infective agent present. In a particularly preferred embodiment, a granulocyte may be considered “a granulocyte with the ability to kill an infective agent or cells infected by an infective agent” if it kills greater than 41.23% of the infective agent or the cells infected by an infective agent present.

In contrast, a granulocyte that “does not have the ability to kill an infective agent or cells infected by an infective agent” or is “unable to kill an infective agent or cells infected by an infective agent” may be a granulocyte that is not capable of killing (preferably does not kill) at least 5% of an infective agent or cells infected by an infective agent in a method described herein. A granulocyte that “does not have the ability to kill an infective agent or cells infected by an infective agent” or is “unable to kill an infective agent or cells infected by an infective agent” may be a granulocyte that is not capable of killing (preferably does not kill) at least 10%, 20%, 30%, 40%, or 50% of the infective agent or the cells infected by an infective agent present. In a suitable embodiment a granulocyte that “does not have the ability to kill an infective agent or cells infected by an infective agent” or is “unable to kill an infective agent or cells infected by an infective agent” is a granulocyte that is not capable of killing (preferably does not kill) at least 60% of the infective agent or the cells infected by an infective agent present. In a suitable embodiment a granulocyte that “does not have the ability to kill an infective agent or cells infected by an infective agent” or is “unable to kill an infective agent or cells infected by an infective agent” is a granulocyte that is not capable of killing (preferably does not kill) at least 70% of the infective agent or cells infected by an infective agent present. Preferably a granulocyte that “does not have the ability to kill an infective agent or cells infected by an infective agent” or is “unable to kill an infective agent or cells infected by an infective agent” is a granulocyte that is not capable of killing (preferably does not kill) at least 80% or 90% of the infective agent or the cells infected by an infective agent present. In a particularly preferred embodiment, a granulocyte that “does not have the ability to kill an infective agent or cells infected by an infective agent” or is “unable to kill an infective agent or cells infected by an infective agent” is a granulocyte that is not capable of killing (preferably does not kill) greater than 41.23% of the infective agent or the cells infected by an infective agent present. Likewise, reference to a population of granulopoietic cells that “does not have the ability to kill an infective agent or cells infected by an infective agent” or is “unable to kill an infective agent or cells infected by an infective agent” is a population of granulopoietic cells that does not differentiate into a granulocyte that has the ability to kill an infective agent or cells infected by an infective agent and/or that differentiates into a granulocyte that “does not have the ability to kill an infective agent or cells infected by an infective agent” or is “unable to kill an infective agent or cells infected by an infective agent”.

An infective agent may refer to a bacterium, a fungus, a virus, a macroparasite (e.g. a helminth), or a combination thereof. Preferably, an infective agent is a bacterium or a virus. For example, in a suitable embodiment, an infective agent is a bacterium. In an alternative embodiment, an infective agent is a virus. Suitably an infective agent is a pathogen.

Granulopoietic cells that may be employed in the aspects of the invention described herein include those capable of giving rise (preferably give rise) to granulocytes able to express desirable chemokines. Merely by way of example, the inventors have shown that granulopoietic cells suitable for use in the various aspects of the present invention are able to differentiate and give rise to granulocytes that secrete CXCL10.

Granulopoietic cells that may be employed in the aspects of the invention described herein include those capable of giving rise (preferably give rise) to granulocytes able to express advantageous ligands for costimulatory receptors. Merely by way of example, the inventors have shown that granulopoietic cells suitable for use in the various aspects of the present invention are able to differentiate and give rise to granulocytes that express costimulatory receptor ligands, such as 4-1BBL and OX40L.

Granulopoietic cells suitable for use in the various aspects of the invention may be obtained from any suitable source. The granulopoietic cells may be allogeneic with reference to their intended recipient. They may be obtained from or derived from any suitable donor.

A population of granulopoietic cells suitable for use in the various embodiments of the invention may be produced by in vitro differentiation of a stem cell. For example, as considered above, a suitable population of granulopoietic cells may be produced by in vitro differentiation of a stem cell, or population of stem cells, into progenitor cells, and then further into a population of granulopoietic cells. The term “stem cell” as used herein encompasses any cell that is capable of differentiating (preferably that differentiates) into a population of progenitors able to give rise to granulopoietic cells (and preferably a population of progenitors that give rise to granulopoietic cells capable of generating (preferably that generate) neutrophils). For example, the term “stem cell” may encompass totipotent, pluripotent, multipotent, or unipotent cells. In a suitable embodiment the term “stem cell” encompasses a haematopoietic stem cell, as well as a precursor cell (e.g. differentiated from a haematopoietic stem cell), wherein said precursor cell is capable of differentiating (preferably differentiate) into a granulocyte (preferably a neutrophil).

A stem cell may be part of a stem cell culture.

The “stem cell” may be a natural stem cell or an artificial stem cell. In a suitable embodiment a natural stem cell may be a cell of the haematopoiesis pathway or a cell equivalent thereto. In a suitable embodiment a population of granulopoietic cells is derived from an artificial stem cell which is an induced pluripotent stem cell (iPSC) or a cell equivalent thereto.

In a suitable embodiment, an iPSC is obtainable from a somatic cell, such as a somatic cell of a donor. Generation of iPSCs is a well-known technique in the art, see Yu et al (2007), Science, 318:1917-1920 the teaching of which is incorporated herein by reference.

In another embodiment, an iPSC is obtainable from a stem cell (e.g. obtainable from a donor), such as from a stem cell of the hematopoietic pathway. Preferably an iPSC is obtainable from a hematopoietic stem cell or a precursor cell described herein.

In a suitable embodiment, a stem cell is a nuclear transfer embryonic stem cell (NT-ESC) or equivalent thereto. In a suitable embodiment, an NT-ESC is obtainable by injecting the nucleus of a cell from the donor into an egg cell from which the original nucleus has been removed. Generation of NT-ESCs is a well-known technique in the art, see Tachibana M, Amato P, Sparman M, et al (2013), Cell, 154 (2): 465-466 the teaching of which is incorporated herein by reference.

A stem cell may be immortalised. The person skilled in the art is familiar with immortalisation techniques, which include inter alia introduction of a viral gene that deregulates the cell cycle (e.g. the adenovirus type 5 E1 gene), and artificial expression of telomerase. Immortalisation advantageously allows for the preparation of a cell line which can be stably cultured in vitro. Thus, in one aspect the invention provides an immortalised cell line obtainable (e.g. obtained) from a selected stem cell, as well as a stable stem cell culture. Suitably an immortalised cell line or stable stem cell culture is obtainable (e.g. obtained) by a method of the present invention.

The term “stable” as used in reference to a stem cell culture or cell line means that the cell culture or cell line has been modified such that it is more amenable to in vitro cell culture than an unmodified cell (i.e. a cell obtained from a donor and subjected directly to in vitro cell culture). Said “stable” cell culture or cell line is therefore capable of undergoing (preferably undergoes) more rounds of replication (preferably for prolonged periods of time) when compared to an unmodified cell.

In one aspect the invention provides a method of promoting therapeutic activity of non-granulocytic immune cells, the method comprising incubating a non-granulocytic immune cell with a population of granulopoietic cells as herein defined.

A method in accordance with this aspect of the invention may be practiced in vitro or in vivo. Suitably the method is practiced in vivo. A method in accordance with this aspect of the invention may be used to promote therapeutic activity of non-granulocytic immune cells prior to their administration to a patient as a therapeutic agent.

A method in accordance with this aspect of the invention may be practiced in respect of any non-granulocytic immune cells. The method may be practiced in respect of host non-granulocytic immune cells. Suitably the method is practiced in respect of NK cells.

The increase in therapeutic activity may be demonstrated by an increase in activation, in accordance with any of the parameters discussed further herein.

In one aspect, the invention provides a method of increasing survival of immune cells in culture, the method comprising, culturing the immune cells in the presence of a feeder layer of granulopoietic cells.

In one aspect, the invention provides a method of increasing proliferation of immune cells in culture, the method comprising, culturing the immune cells in the presence of a feeder layer of granulopoietic cells.

The immune cells cultured in a method of various aspects of the invention may be selected from the group comprising (or consisting of): a T cell; and an NK cell. In an embodiment where the cultured immune cell comprises a T cell, the cell may be selected from the group comprising (or consisting) of: a CD8⁺ T cell; a CD4⁺ T cell; a NK T cell; an αβ T cell; a γδ T cell; a peripheral blood T cell; and a tumour infiltrated T cell.

The methods may be well suited to use in the culture of NK or NK T cells. The methods may be well suited to use in the culture of αβ T cells.

In one aspect the invention provides a method of selecting a suitable treatment regimen for a patient, the method comprising:
identifying whether the patient has an impaired non-granulocytic immune response; and
if the patient is identified as having an impaired non-granulocytic immune response, then treatment with a population of granulopoietic cells is selected as an appropriate treatment; and
if the patient is identified as lacking an impaired non-granulocytic immune response, then treatment with a therapy other than a population of granulopoietic cells is selected.

The skilled person will be aware of many suitable methods by which the impairment (or otherwise) of a non-granulocytic immune response of a patient may be assessed.

Such methods may be of particular relevance in the case of a patient suspected of having an impaired non-granulocytic immune response. A patient having, or suspected of having, an impaired non-granulocytic immune response may be a patient with a disease, or receiving treatment, resulting in immune suppression.

In one aspect of the invention provides a method of selecting a suitable treatment regimen for a patient, the method comprising:
incubating a non-granulocytic immune cell from the patient with a population of granulopoietic cells; wherein
if the activation of the non-granulocytic immune cell from the patient is increased in response to the incubation, then treatment with a population of granulopoietic cells is selected as an appropriate treatment; and
if the activation of the non-granulocytic immune cell from the patient is increased in response to the incubation, then treatment with a therapy other than a population of granulopoietic cells is selected.

Activation of a patient's non-granulocytic cells may be assessed with reference to any suitable indication of activation, and by any suitable means, including (but not limited to) those indications and means discussed further in this specification.

In the case that treatment with a population of granulopoietic cells is selected as an appropriate treatment, this treatment may be put into practice using granulopoietic cells as considered in any of the aspects or embodiment of the invention. Such cells may be provided by means of a pharmaceutical composition of the invention.

Some aspects of the invention relate to screening methods for identifying granulopoietic cells suitable for therapeutic use.

Respectively, one aspect provides a method of identifying whether or not a population of granulopoietic cells is suitable for use in the treatment of cancer by beneficially modulating the tumour microenvironment, the method comprising:
assessing whether the population of granulopoietic cells, or a cell derived from the population of granulopoietic cells, is able to express proinflammatory cytokines; and/or.
assessing whether the population of granulopoietic cells, or a cell derived from the population of granulopoietic cells, is able to stimulate expression of proinflammatory cytokines by non-granulocytic immune cells;
and identifying whether or not a population of granulopoietic cells is suitable for use in the treatment of cancer by beneficially modulating the tumour microenvironment on the basis of this assessment.

In one aspect the invention provides a method of identifying whether or not a population of granulopoietic cells is suitable for use in the treatment of cancer by increasing recruitment of immune cells into a tumour and/or immune cell activation, the method comprising:
assessing whether the population of granulopoietic cella, or a cell derived from the population of granulopoietic cells, is able to express a chemokine associated with promoting cell trafficking; and/or
assessing whether the population of granulopoietic cells, or a cell derived from the population of granulopoietic cells, is able to stimulate expression of degranulation markers by non-granulocytic immune cells;
and identifying whether or not a population of granulopoietic cells is suitable for use in the treatment of cancer by increasing recruitment of immune cells into a tumour and/or immune cell activation on the basis of this assessment.

In one aspect the invention provides a method of identifying whether or not a population of granulopoietic cells is suitable for use in the treatment of cancer by directly promoting killing of cancer cells, the method comprising:
incubating the population of granulopoietic cells, or a cell derived from the population of granulopoietic cells, with cells of a cancer cell line; and
assessing whether the population of granulopoietic cells, or a cell derived from the population of granulopoietic cells, is able to increase death of the cells of the cancer cell line to a greater extent than death of non-cancer cells;
and identifying whether or not a population of granulopoietic cells is suitable for use in the treatment of cancer by directly promoting killing of cancer cells on the basis of this assessment.

In one aspect the invention provides a method of identifying whether or not a population of granulopoietic cells is suitable for use in the treatment of infection by directly promoting killing of cellular infectious agents or infected cells, the method comprising:
incubating the population of granulopoietic cells, or a cell derived from the population of granulopoietic cells, with a sample of a cellular infectious agent or of infected cells; and
assessing whether the population of granulopoietic cells, or a cell derived from the population of granulopoietic cells, is able to increase death of the cellular infectious agent or of infected cells;
and identifying whether or not a population of granulopoietic cells is suitable for use in the treatment of infection by directly promoting killing of cellular infectious agents or infected cells on the basis of this assessment.

In one aspect the invention provides a method of identifying whether or not a population of granulopoietic cells is suitable for use in treatment by amplifying a therapeutic immune response, the method comprising:
incubating the population of granulopoietic cells, or a cell derived from the population of granulopoietic cells, with immune cells; and
assessing whether the population of granulopoietic cells, or the cell derived from the population of granulopoietic cells, is able to increase activation of the immune cells;
and identifying whether or not a population of granulopoietic cells is suitable for use in the treatment by amplifying a therapeutic immune response on the basis of this assessment.

In a suitable embodiment, a method in accordance with a method of identifying whether or not a population of granulopoietic cells is suitable for use in the treatment of cancer by beneficially modulating the tumour microenvironment may comprise assessing expression of proinflammatory cytokines selected from the group comprising (or consisting) of: IFN-γ and TNF.

Suitably a method in accordance with a method of identifying whether or not a population of granulopoietic cells is suitable for use in the treatment of cancer by increasing recruitment of immune cells into a tumour and/or immune cell activation may comprise assessing expression of the chemokine CXL10.

In a suitable embodiment, a method in accordance with a method of identifying whether or not a population of granulopoietic cells is suitable for use in the treatment of cancer by increasing recruitment of immune cells into a tumour and/or immune cell activation may comprise assessing expression of degranulation markers selected from the group comprising (or consisting) of: CD107a; perforin; and granzymes.

Suitably a method in accordance with a method of identifying whether or not a population of granulopoietic cells is suitable for use in the treatment of cancer by directly promoting killing of cancer cells may involve positively identifying the population of granulopoietic cells as suitable for use in the treatment of cancer by directly promoting killing of cancer cells in the case that the rate of death of cancer cells incubated with the population of granulopoietic cells, or a cell derived from the population of granulopoietic cells, is at least three-fold higher than the rate of death of non-cancer cells.

A method in accordance with the a method of identifying whether or not a population of granulopoietic cells is suitable for use in the treatment of infection by directly promoting killing of cellular infectious agents or infected cells may involve positively identifying the population of granulopoietic cells as suitable for use in the treatment of infection when the rate of death of cellular infectious agents or infected cells incubated with the population of granulopoietic cells, or a cell derived from the population of granulopoietic cells, is at least three-fold higher than the rate of death of non-infected cells.

A method in accordance with the a method of identifying whether or not a population of granulopoietic cells is suitable for use in treatment by amplifying a therapeutic immune response may involve identifying a population of granulopoietic cells as suitable for use in treatment when activation of the immune cells is increased in accordance with any of the considerations set out in respect of this disclosure. The population of granulopoietic cells may be incubated with any form of immune cells. For example, the population of granulopoietic cells may be incubated with non-granulocytic cells. The immune cells may be derived from an individual requiring therapy.

In the event that a method of screening in accordance with any of these aspects of the invention identifies a population of granulopoietic cells as suitable for use in treatment, the method may comprise a further step of identifying the donor from whom the population of granulopoietic cells was taken or derived as a donor capable of providing (and preferably that provides) therapeutically effective populations of granulopoietic cells. Alternatively, or additionally, the method may comprise a further step of obtaining a stem cell from the donor from whom the population of granulopoietic cells was taken or derived. The stem cell may be a naturally occurring cell, such as a haematopoietic stem cell, or may be an artificial stem cell, such as an iPSC. Such a stem cell may be stored. Such a stem cell may be used to produce further therapeutically effective populations of granulopoietic stem cells, such as for incorporation in pharmaceutical compositions of the invention.

Any of the methods disclosed herein may be an in vivo method. Preferably, the methods disclosed herein are in vitro methods.

Embodiments related to the various compositions of the invention are intended to be applied equally to the kits, methods, and/or uses, and vice versa.

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

Amino acids are referred to herein using the name of the amino acid, the three letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “a population of granulopoietic cells” includes a plurality of such populations and reference to “the population of granulopoietic cell” includes reference to one or more granulopoietic cell populations and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

### BRIEF DESCRIPTION OF THE DRAWINGS

Embodiments of the invention will now be described, by way of example only, with reference to the following Figures and Examples. Many of the Figures submitted herein are better understood in colour. The colour versions of the drawings are part of the application as filed and the right to present colour images of the drawings in later proceedings is hereby reserved.

FIG. 1 illustrates the effect of granulopoietic cells on activation of blood-derived CD8⁺ T cells assessed with reference to expression of degranulation markers or costimulatory molecules;

FIG. 2 illustrates the effect of granulopoietic cells on activation of blood-derived CD4⁺ T cells assessed with reference to expression of costimulatory molecules;

FIG. 3 illustrates the effect of granulopoietic cells on activation of αβ T cells assessed with reference to proliferation of the T cells;

FIG. 4 illustrates the effect of granulopoietic cells on activation of blood-derived NK and NKT cells assessed with reference to survival of the cells;

FIG. 5 illustrates the effect of granulopoietic cells on activation of blood-derived natural killer (NK) and natural killer T (NKT) cells assessed with reference to expression of degranulation markers or costimulatory molecules;

FIG. 6 illustrates the effect of granulopoietic cells on activation of CD8⁺ T cells, CD4⁺ T cells and NK cells, assessed with reference to expression of degranulation markers or costimulatory molecules;

FIG. 7 illustrates the effect of granulopoietic cells on activation of peripheral blood mononuclear cells (PBMCs), with reference to expression of cytokines;

FIG. 8 illustrates the effect of granulopoietic cells on activation of tumour infiltrating lymphocytes (TILs), with reference to expression of cytokines;

FIG. 9 illustrates the effect of granulopoietic cells on activation of immune cells, with reference to immune cell trafficking;

FIG. 10 illustrates the effect of granulopoietic cells on activation of immune cells, with reference to cytocidal activity;

FIG. 11 illustrates expression of chemokines by granulocytes formed on differentiation of granulopoietic cells; and

FIG. 12 illustrates expression of ligands for costimulatory molecules by granulocytes formed on differentiation of granulopoietic cells.

FIG. 13 illustrates that priming granulopoietic cells after cryopreservation and subsequent thawing increases their cytolytic activity. 1=unprimed controls; 2=GM-CSF and IL-15; 3=GM-CSF and TNFα; Stauro=staurosporine positive control.

FIG. 14 illustrates that priming granulopoietic cells during the differentiation stage increases their cytolytic activity. 1=GM-CSF and IL-15; 2=GM-CSF and TNFα; 3=GM-CSF; Stauro=staurosporine positive control; D3-4=priming during days 3-4 of differentiation; D4-5=priming during days 4-5 of differentiation; D5-6=priming during days 5-6 of differentiation.

FIG. 15 illustrates the cellular composition of PBMCs before and after αβ T cell depletion.

FIG. 16 illustrates the effect of granulopoietic cells on activation of Vδ1⁺ γδ T cells assessed with reference to expression of 4-1BB and CD25.

FIG. 17 illustrates the effect of granulopoietic cells on activation of Vδ2⁺ γδ T cells assessed with reference to expression of 4-1BB.

FIG. 18 illustrates the effect of granulopoietic cells on blood-derived myeloid cells assessed with reference to expression of CD11b.

FIG. 19 illustrates the effect of αβ T cell-depleted PBMCs on granulopoietic cells with reference to expression of CD54.

FIG. 20 illustrates the effect of granulopoietic cells on αβ T cell-depleted PBMC cell co-stimulatory molecules.

FIG. 21 illustrates an exemplary protocol for generating a composition of the invention.

FIG. 22 illustrates the relative proportions of subpopulations of granulopoietic cells produced by methods of the invention without priming, or with various priming steps.

FIG. 23 further characterises the first subpopulation of granulopoietic cells.

FIG. 24 further characterises the second subpopulation of granulopoietic cells.

FIG. 25 further characterises the third subpopulation of granulopoietic cells.

FIG. 26 further characterises the fourth subpopulation of granulopoietic cells.

### EXAMPLES

### Materials and Methods

### Preparation of Populations of Granulopoietic Cells (IMANp)

The preparation of populations of granulopoietic cells (designated “IMANps” by the inventors, as referred to in the Figures) from haematopoietic stem cells (HSCs) consists of three main stages following collection of donor leukapheresis:
CD34+ Isolation and cryopreservation from donor leukapheresis
Expansion of isolated CD34+ cells (E0 to E8) for 9 days to generate intermediate progenitor cells. On day 9 (E8DO), expansion media is replaced with differentiation media.
Differentiation of intermediate/primitive progenitor cells (D0-D5) for 5 days into a heterogenous mix of cells which are primarily granulopoietic progenitors termed IMANp.
Optional cryopreservation of IMANp.

The materials used to prepare IMANp are as follows:

TABLE 1
Reagents used to prepare IMANp

| | | |
|---|---|---|
| Material | Supplier | Cat. No |

Materials for manufacturing process

| | | |
|---|---|---|
| IMDM with High Glucose, | Life Technologies | 12440053 (or equivalent) |
| Glutamine, HEPES, Phenol | | |
| Red and Sodium Pyruvate | | |
| (Referred to as IMDM in rest | | |
| of the document) | | |
| Penicillin/streptomycin | Life Technologies | 15140122 (or equivalent) |
| G-CSF | PeproTech EC | 300-23 |
| SCF | PeproTech EC | 300-07 |
| FLT-3 | PeproTech EC | 300-19 |
| TPO | PeproTech EC | 300-18 |
| IL-3 | PeproTech EC | 200-03 |
| IL-6 | PeproTech EC | 200-06 |
| ITS | Life Technologies | 41400045 |
| HSA | Stratech Scientific Limited | 30-2026-FIT-100ml |
| Tissue Culture Grade Water | Merck Life Science UK | W3500 (or equivalent) |
| | Limited | |
| G-Rex6 Well Plate | Wilson Wolf | 80240M |
| G-Rex6M Well Plate | Wilson Wolf | 80660M |
| G-Rex10M Gas Permeable | Wilson Wolf | 80110, 80110-CS & 80110S |
| Cell Culture Device | | |
| G-Rex100M Gas Permeable | Wilson Wolf | 81100, 81100-CS & |
| Cell Culture Device | | RU81100 |
| CryoStor CS10 | Merck Life Science UK | C2874-100ML |
| | Limited | |

Materials for analytical testing

| | | |
|---|---|---|
| 96 well u-bottom plate | VWR | 734-2080 (or equivalent) |
| BD Cellfix 10X | BD | 349202 (or equivalent) |
| 50ml Tubes | Appleton Woods | AB034 (or equivalent) |
| Efluor780 Live/dead stain | Life Technologies | 65-0865-14 (or equivalent) |
| Solution 18, AO-DAPI | Chemometec | 910-3018 |
| Staining Reagent | | |

TABLE 2
GMP reagents used to prepare IMANp

| | | Minimum | | Activity of the lot | |
|---|---|---|---|---|---|
| | RUO | Activity | GMP | available for | |
| Cytokine | Supplier | (IU/mg) | Supplier | experiments (IU/mg) | |
| FLT3-L | PeproTech | ≥1E+06 | PeproTech₁ | Not available on CoA | |
| SCF | PeproTech | ≥5E+05 | PeproTech₁ | Not available on CoA | |
| TPO | PeproTech | ≥1E+06 | BioTechne | >1E+07 | 2.54E+08 |
| IL-3 | PeproTech | ≥1E+07 | PeproTech₁ | Not available on CoA | |
| IL-6 | PeproTech | ≥1E+07 | PeproTech₁ | Not available on CoA | |
| G-CSF | PeproTech | ≥1E+07 | BioLegend | Not available on CoA | |
| GM-CSF | PeproTech | ≥1E+07 | BioTechne | >1E+07 | 2.00E+07 |
| | BioTechne | NA | | | |
| TNFa | ThermoFisher | ≥2E+07 | BioTechne | >4.3E+07 | 4.90E+07 |
| | Scientific | | | | |

₁—Alternative supplier - BioTechne

### Media Preparation

The cytokines are reconstituted in cell culture grade water with 5% HSA and aliquots are stored at −80° C. prior to addition to media.

### Expansion Media

CD34⁺ HSCs are expanded in expansion media containing Iscove's Modified Dulbecco's Medium (IMDM) with cytokines including SCF, FLT-3, TPO, IL3 and IL6 as well as ITS and HAS, at the following concentrations:

TABLE 3
Expansion Media constituents

| Expansion Media | Final | Unit |
|---|---|---|
| IMDM with Glutamax | | |
| SCF | 0.2 | μg/mL |
| FLT-3 | 0.2 | μg/mL |
| TPO | 0.02 | μg/mL |
| IL3 | 0.015 | μg/mL |
| IL6 | 0.015 | μg/mL |
| ITS | 1 | x |
| HSA | 1 | % |

### Differentiation Media

Cells are differentiated in differentiation media containing IMDM, SCF, TPO, GCSF, ITS and HSA, at the following concentrations:

TABLE 4
Differentiation Media constituents

| Differentiation Media | Final | Unit |
|---|---|---|
| IMDM with Glutamax | | |
| SCF | 0.13 | μg/mL |
| TPO | 0.13 | μg/mL |
| G-CSF | 0.13 | μg/mL |
| GM-CSF | 0.01 | μg/mL |
| IL-3 | 0.13 | μg/mL |
| TNF | 0.001 | μg/mL |
| ITS | 1 | x |
| HSA | 1 | % |

### Expansion of CD34⁺ HSCs

Donor CD34 HSCs were thawed on Day 0 (E0) at 37° C. in the water bath and transferred into thaw medium consisting of IMDM and 1% HSA. Cell count and viability measurements for all donor samples were performed immediately post thaw. Cells were subsequently seeded at 5e5/mL and 5e5/cm² in expansion media in G-Rex 6M or G-Rex 10M with 10 cm² surface area in a volume 10 mL per well.

On day 1 (E1), samples were taken for cell count, viability and flow cytometry analysis for phenotypic characterisation using progenitor and neutrophil panels. Wells were topped up with 40 mL expansion media to increase volume to 4 mL/cm². On E2 and E3, cells were left undisturbed in G-Rex for continued expansion.

On E4, cells were transferred to G-Rex with greater surface area for example 1 G-Rex100M seeded from 1 G-Rex 6M or G-Rex 10M. The G-Rex was carefully removed from incubator, and expansion media was removed to 15 mL per well. Cells were resuspended in residual volume by swirling, following which a sample was taken for cell count, viability and flow cytometry analysis for phenotypic characterisation using progenitor, neutrophil and mature neutrophil panel phenotypes. Cells were subsequently transferred to a G-Rex 100M, and 85 mL fresh expansion media was added to the G-Rex. Cells were left undisturbed on E5, and on E6, an optional sample may be taken for cell count, viability and flow cytometry analysis for phenotypic characterisation using progenitor, neutrophil, mature neutrophil, and off-target myeloid and lymphoid panels. In addition, each well was fed with 100 mL expansion media and left for 48 hours. Cells were left undisturbed on E7. On E8, the G-Rex was carefully removed from the incubator, and expansion media was removed, for volume to be 100 mL per well. Cells were resuspended by swirling and samples taken for cell count, viability and flow cytometry analysis for phenotypic characterisation using progenitor, neutrophil, mature neutrophil, off-target myeloid and lymphoid panels. Media exchange was subsequently performed to begin differentiation process.

### Differentiation of Intermediate Progenitor Cells

On day 9 of the manufacturing process, (E8D0), following removal of expansion media to leave 100 mL per well, 400 mL fresh differentiation media was added to each well, and the G-Rex was returned to the incubator and left undisturbed for D1 and D2. On D3, an optional sample may be taken for cell count, viability and flow staining of progenitor, neutrophil, mature neutrophil, and off-target myeloid and lymphoid panels. In addition, on D3, differentiation media volume per well was doubled to 1L total volume per well. Cells were left undisturbed to differentiate through D4 and on D5 cell harvest was performed.

Fold expansion of cells (from stem cells at E0 to progenitor cells at E8DO to granulopoietic cells at E8D5) achieved using this exemplary method of the invention was as set out in Table 5.

TABLE 5

| | Manufacturing | Mean Fold |
|---|---|---|
| | Process | Expansion |
| Day | Timepoint | (n = 11) |
| 0 | E0 | 1.00 |
| 1 | E1 | 0.79 |
| 4 | E4 | 6.60 |
| 8 | E8D0 | 73.66 |
| 14 | E8D5 | 258.55 |

### Harvest of IMANp; Heterogenous Mix of Progenitors Primarily Granulopoietic Progenitor Cells

On day 14 of manufacturing process (E8D5), G-Rex was carefully removed from the incubator, and media was aspirated to 100 mL. Cells were resuspended by swirling and transferred into sterile centrifuge tubes. Samples may be taken for cell count, viability and staining of progenitor, neutrophil, mature neutrophil, off-target myeloid and lymphoid panels. Cells were washed by centrifugation at 350 g for 10 minutes, and spent medium was aspirated off. Cells were then resuspended in cryoformulation medium (CS10) containing 10% DMSO concentration at required density with the cell concentration below 100E6 cells/mL. The resulting samples were aliquoted into cryogenic containers (bags and vials) and immediately frozen. The samples were then stored in vapour phase liquid nitrogen freezers (≤−130° C.). Post 24 hours storage, a cryovial was removed for post-thaw analysis to evaluate cell viability, cell recovery as well as flow cytometry analysis for phenotypic characterisation using progenitor, neutrophil, mature neutrophil, off-target myeloid and lymphoid panels.

### IMANp Characterisation

Populations of granulopoietic cells were characterised with respect to the following panels of markers.

TABLE 6
Panel used for characterisation of Progenitor cell/Stem cell (HSC) populations

| | Fluorochrome | Marker | Clone | Isotype | Supplier | Cat # |
|---|---|---|---|---|---|---|
| V1 | BV421 | CD49f | GoH3 | Rat IgG2a | BD | 747725 |
| V4 | BV605 | CD10 | HI10a | Mouse | BioLegend | 312222 |
| | | | | IgG1 | | |
| V5 | BV650 | CD90 | 5 E10 | Mouse | BioLegend | 328144 |
| | | | | IgG1 | | |
| B1 | FITC | Lineage | | | BD | 340546 |
| B2 | PE | CD34 | 581 | Mouse | BD | 555822 |
| | | | | IgG1 | | |
| B5 | PerCP-Vio700 | CD123 | REA918 | REA | Miltenyi | 130-115- |
| | | | | | | 268 |
| B6 | PE-Vio770 | CD38 | REA572 | REA | Miltenyi | 130-113- |
| | | | | | | 432 |
| R1 | APC | CD133 | AC133 | Mouse | Miltenyi | 130-113- |
| | | | | IgG1 | | 668 |
| R2 | AF700 | CD45RA | REA562 | REA | Miltenyi | 130-113- |
| | | | | | | 369 |
| R3 | AF780 | L/D | | | | |

Lineage cocktail: CD3 (SK7); CD16 (3G8); CD19 (SJ25C1); CD20 (L27); CD14 (MoP9); CD56 (NCAM16.2)

TABLE 7
Panel of Neutrophil markers used in characterisation of the cell populations

| | Fluorochrome | Antigen | Clone | Isotype | Supplier | Cat # |
|---|---|---|---|---|---|---|
| V1 | BV421 | CD34 | 561 | Mouse | BioLegend | 343610 |
| | | | | IgG2a | | |
| V4 | BV605 | CD11b | M1/70 | Rat IgG2b | BioLegend | 101257 |
| V5 | BV650 | CD71 | BV650 | Mouse | BioLegend | 334116 |
| | | | | IgG2a | | |
| B1 | VioBright B515 | CD66b | REA306 | REA | Miltenyi | 130-126- |
| | | | | | | 905 |
| B2 | PE | CD49d | 9F10 | Mouse | BioLegend | 304304 |
| | | | | IgG1 | | |
| B5 | PerCP-Cy5.5 | CD117 | 104D2 | Mouse | BioLegend | 313214 |
| | | | | IgG1 | | |
| B6 | PE-Cy7 | CD10 | HI10a | Mouse | BioLegend | 312214 |
| | | | | IgG1 | | |
| R1 | APC | CD15 | HI98 | Mouse | BioLegend | 301908 |
| | | | | IgM | | |
| R2 | AF700 | CD38 | HIT2 | Mouse | BioLegend | 303524 |
| | | | | IgG1 | | |
| R3 | AF780 | Viability | | | | |

TABLE 8
Panel Mature Neutrophil markers used in characterisation of the cell populations

| | Fluorochrome | Antigen | Clone | Isotype | Supplier | Cat # |
|---|---|---|---|---|---|---|
| V1 | BV421 | CD177 | MEM-166 | Mouse | BD | 564240 |
| | | | | IgG1 | | |
| V4 | BV605 | CD11b | M1/70 | Rat | BioLegend | 101257 |
| | | | | IgG2b | | |
| V5 | BV650 | CD62L | DREG-56 | Mouse | BioLegend | 304832 |
| | | | | IgG1 | | |
| B1 | VioBright B515 | CD66b | REA306 | REA | Miltenyi | 130-126- |
| | | | | | | 905 |
| B2 | PE | CD54 | HA58 | Mouse | BioLegend | 353106 |
| | | | | IgG1 | | |
| B5 | PerCP-Cy5.5 | CD63 | H5C6 | Mouse | BioLegend | 353020 |
| | | | | IgG1 | | |
| B6 | PE-Cy7 | CD18 | TS1/18 | Mouse | BioLegend | 302118 |
| | | | | IgG1 | | |
| R1 | APC | CD15 | HI98 | Mouse | Biolegend | 301908 |
| | | | | IgM | | |
| R2 | AF700 | CD11c | Bu15 | Mouse | BioLegend | 337220 |
| | | | | lgG1 | | |
| R3 | AF780 | Viability | | | | |

TABLE 9
Panel of Off-target Myeloid markers used in
the characterisation of the cell populations

| | Fluorochrome | Antigen | Clone | Isotype | Supplier | Cat # |
|---|---|---|---|---|---|---|
| V1 | BV421 | CD14 | M5E2 | Mouse | BioLegend | 301830 |
| | | | | IgG2a | | |
| V4 | BV605 | CD11b | ICRF44 | Mouse | BioLegend | 301332 |
| | | | | IgG1 | | |
| V5 | BV650 | HLA-DR | L243 | Mouse | BioLegend | 307650 |
| | | | | IgG2a | | |
| B1 | FITC | CD66b | REA306 | REA | Miltenyi | 130-126- |
| | | | | | | 905 |
| B2 | PE | CD16 | 3G8 | Mouse | BioLegend | 302008 |
| | | | | IgG1 | | |
| B5 | PerCP-Cy5.5 | CD206 | 15-2 | Mouse | BioLegend | 321122 |
| | | | | IgG1 | | |
| B6 | PE-Cy7 | CD68 | Y1/82A | Mouse | BioLegend | 333816 |
| | | | | IgG2b | | |
| R1 | APC | CD15 | HI98 | Mouse | Biolegend | 301908 |
| | | | | IgM | | |
| R2 | AF700 | CD11c | Bu15 | Mouse | BioLegend | 337220 |
| | | | | IgG1 | | |
| R3 | AF780 | Viability | | | | |

TABLE 10
Panel of Off-target Lymphoid markers used in
the characterisation of the cell populations

| | Fluorochrome | Antigen | Clone | Isotype | Supplier | Cat # |
|---|---|---|---|---|---|---|
| V1 | BV421 | CD4 | RPA-T4 | Mouse | BioLegend | 300532 |
| | | | | IgG1 | | |
| V4 | BV605 | CD8 | RPA-T8 | Mouse | BioLegend | 301040 |
| | | | | IgG1 | | |
| B2 | PE | CD56 | HCD56 | Mouse | BioLegend | 318306 |
| | | | | IgG1 | | |
| B6 | PE-Cy7 | CD3 | UCHT1 | Mouse | BioLegend | 300420 |
| | | | | IgG1 | | |
| R1 | APC | CD19 | HIB19 | Mouse | BioLegend | 302212 |
| | | | | IgG1 | | |
| R3 | AF780 | L/D | | | | |

TABLE 11
panel of markers used for characterisation of the
populations of granulopoietic cells produced.
Neut phenotype v2

| | Fluorochrome | Antigen | Clone | Isotype | Supplier | Cat # |
|---|---|---|---|---|---|---|
| V1 | BV421 | CD177 | MEM-166 | Mouse IgG1 | BD | 564240 |
| V4 | BV605 | CD11b | M1/70 | Rat IgG2b | BioLegend | 101257 |
| V5 | BV650 | CD71 | CY1G4 | Mouse IgG2a | BioLegend | 334116 |
| B1 | VioBright B515 | CD66b | REA306 | REA | Miltenyi | 130-126- |
| | | | | | | 905 |
| B2 | PE | HLA-DR | L243 | Mouse IgG2a | BioLegend | 307606 |
| B5 | PerCpCy5.5 | CD115 | 9-4D2-1E4 | Rat IgG1 | BioLegend | 347310 |
| B6 | PE-Cy7 | CD49d | 9F10 | Mouse IgG1 | BioLegend | 304314 |
| R1 | APC | CD15 | HI98 | Mouse IgM | BioLegend | 301908 |
| R2 | AF700 | | | | | |
| R3 | A780 | Live/dead | | | | |

FC/Chemokine receptor

| | Fluorochrome | Marker | Clone | Isotype | Suppplier | Cat # |
|---|---|---|---|---|---|---|
| V1 | BV421 | CD34 | 561 | Mouse IgG2a | BioLegend | 343610 |
| V4 | BV605 | CD11b | M1/70 | Rat IgG2b | BioLegend | 101257 |
| V5 | BV650 | HLA-DR | L243 | Mouse IgG2a | BioLegend | 307650 |
| B1 | VioBright B515 | CXCR4 | REA649 | REA | Miltenyi | 130-120- |
| | | | | | | 025 |
| B2 | PE | CD64 | 10.1 | Mouse IgG1 | BioLegend | 305008 |
| B5 | PerCP-Cy5.5 | CD32 | FUN-2 | Mouse IgG2b | Biolegend | 303216 |
| B6 | PE-Cy7 | CXCR2 | 5 | Mouse IgG1 | BioLegend | 320716 |
| | | (CD182) | E8/CXCR2 | | | |
| R1 | APC | CD15 | HI98 | Mouse IgM | BioLegend | 301908 |
| R2 | AF700 | CD38 | HIT2 | Mouse IgG1 | BioLegend | 303524 |
| R3 | AF780 | L/D | | | | |

Neutrophil Activation v2

| | Fluorochrome | Antigen | Clone | Isotype | Supplier | Cat # |
|---|---|---|---|---|---|---|
| V1 | BV421 | CD40 | 5C3 | Mouse IgG1 | BioLegend | 334332 |
| V4 | BV605 | CD11b | M1/70 | Rat IgG2b | BioLegend | 101257 |
| V5 | BV650 | CD62L | DREG-56 | Mouse IgG1 | BioLegend | 304832 |
| B1 | VioBright B515 | CD66b | REA306 | REA | Miltenyi | 130-126- |
| | | | | | | 905 |
| B2 | PE | HLA-DR | L243 | Mouse IgG2a | BioLegend | 307606 |
| B5 | PerCP-Cy5.5 | CD54 | HA58 | Mouse IgG1 | BioLegend | 353106 |
| B6 | PE-Cy7 | CD18 | TS1/18 | Mouse IgG1 | BioLegend | 302118 |
| R1 | APC | CD15 | HI98 | Mouse IgM | Biolegend | 301908 |
| R2 | AF700 | | | | | |
| R3 | AF780 | Viability | | | | |

Residuals v2

| | Fluorochrome | Antigen | Clone | Isotype | Supplier | Cat # |
|---|---|---|---|---|---|---|
| V1 | BV421 | CD14 | M5E2 | Mouse IgG2a | BioLegend | 301830 |
| V4 | BV605 | CD11b | M1/70 | Rat IgG2b | BioLegend | 101257 |
| V5 | BV650 | HLA-DR | L243 | Mouse IgG2a | BioLegend | 307650 |
| B1 | VioBright B515 | | | | | |
| B2 | PE | CD16 | 3G8 | Mouse IgG1 | BioLegend | 302008 |
| B5 | PerCP-Cy5.5 | CD206 | 15-2 | Mouse IgG1 | BioLegend | 321122 |
| B6 | PE-Cy7 | CD68 | Y1/82A | Mouse IgG2b | BioLegend | 333816 |
| R1 | APC | CD15 | HI98 | Mouse IgM | Biolegend | 301908 |
| R2 | AF700 | CD11c | Bu15 | Mouse IgG1 | BioLegend | 337220 |
| R3 | AF780 | Viability | | | | |

### Results of Characterisation of the Granulopoietic Cell Populations

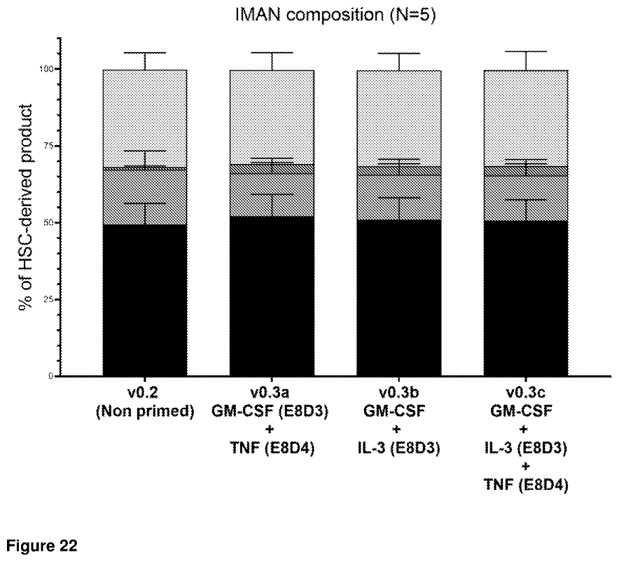

The results of characterisation of populations of granulopoietic cells in accordance with the invention, manufactured by methods of the invention, are set out in FIGS. 22 to 26.

As set out above, in connection with the sixth aspect of the invention, four subpopulations of granulopoietic cells were identified within the population of granulopoietic cells as a whole.

FIG. 22 illustrates the relative proportions of these subpopulations within granulopoietic cell populations generated by exemplary methods of the invention. v0.2 is a method of the invention without a priming step, whereas v0.3a-c incorporate optional priming steps as follows: v0.3a (priming with GM-CSF on the penultimate day of culture, and TNF on the final day of culture); v0.3b (priming with GM-CSF and IL-3 both on the penultimate day of culture); and v0.3c (priming with GM-CSF and IL-3 both on the penultimate day of culture, and TNF on the final day of culture).

In each of these conditions, the first population represented the largest proportion of the whole, the third population the second largest proportion of the whole, the second population the third largest proportion of the whole granulopoietic cell population. The fourth subpopulation represented the smallest subpopulation generated by each of the protocols, and was hardly present in the population of granulopoietic cells produced using the v0.2 (no priming) protocol.

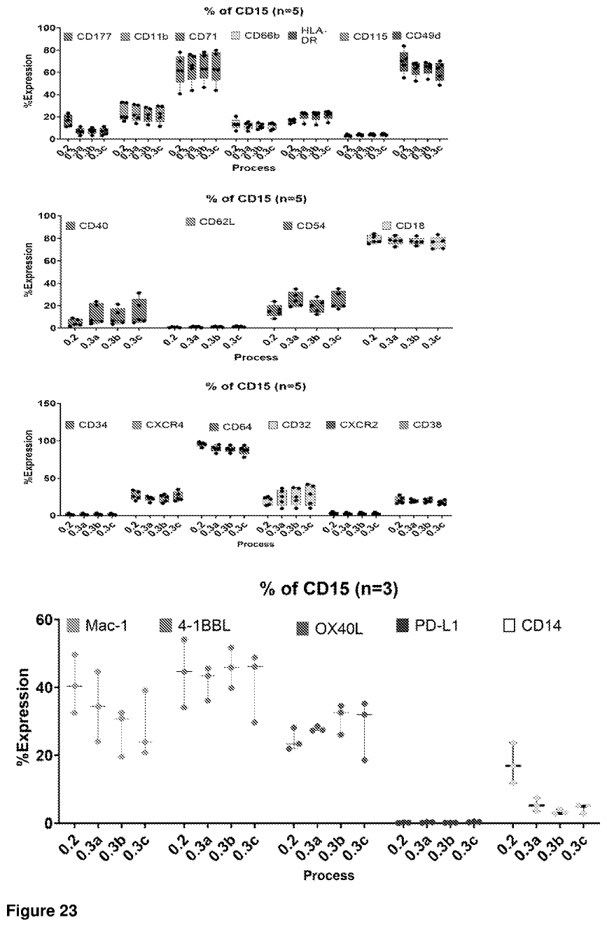

FIG. 23 further characterises the first subpopulation of granulopoietic cells (a population in accordance with the seventh aspect of the invention) with reference to their expression of various markers.

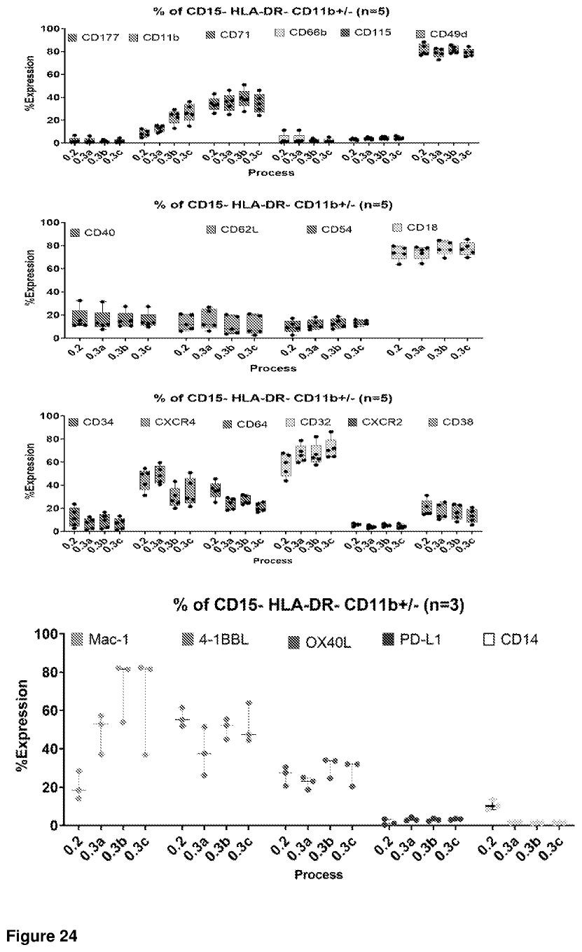

FIG. 24 further characterises the second subpopulation of granulopoietic cells (a population in accordance with the eighth aspect of the invention) with reference to their expression of various markers.

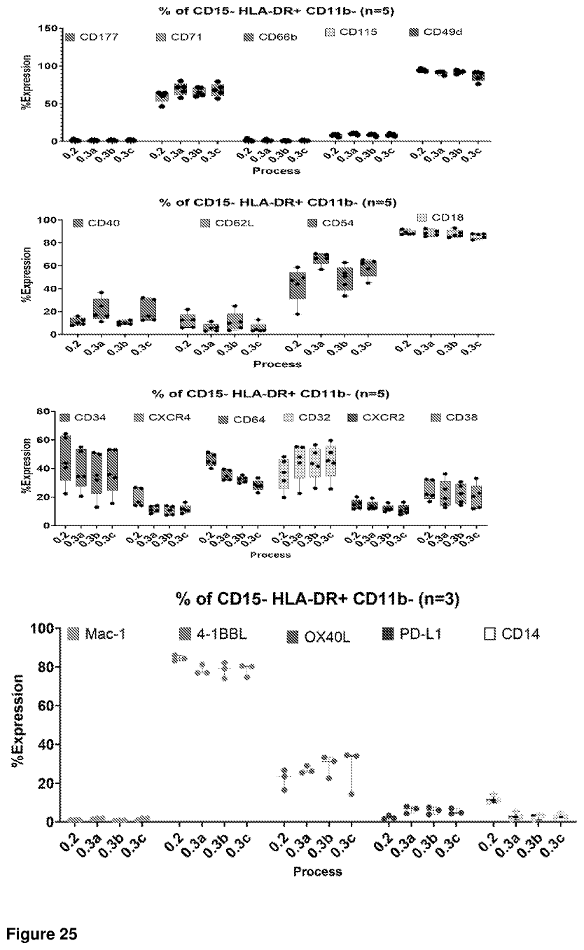

FIG. 25 further characterises the third subpopulation of granulopoietic cells (a population in accordance with the ninth aspect of the invention) with reference to their expression of various markers.

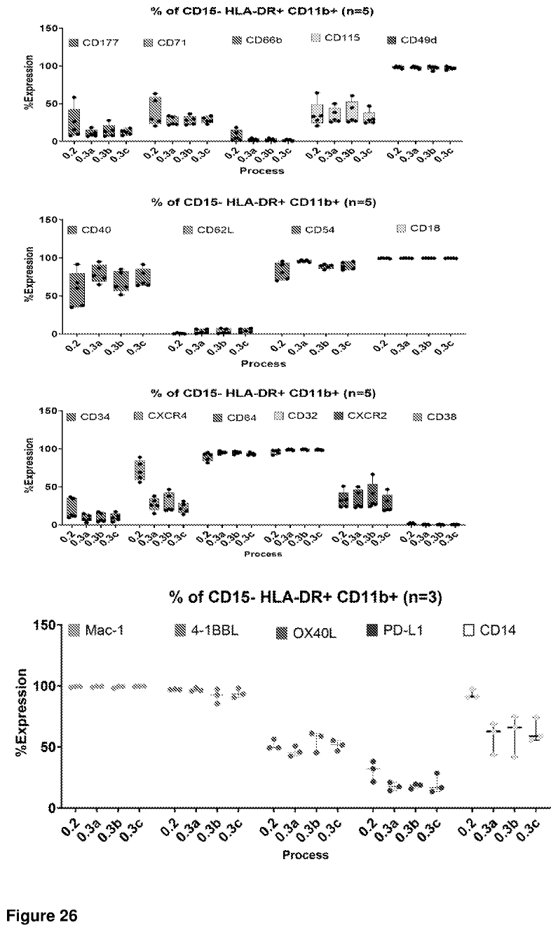

FIG. 26 further characterises the fourth subpopulation of granulopoietic cells (a population in accordance with the tenth aspect of the invention) with reference to their expression of various markers.

### Optional Priming Step in Respect of Populations of Granulopoietic Cells (IMANp)

IMANp may be primed post-thaw with additional cytokines to enhance their cytotoxicity.

TABLE 12
Additional materials used in optional priming steps

| Material | Supplier | Cat. No |
|---|---|---|
| GM-CSF | Peprotech EC | 300-03-500uG |
| TNFα | Peprotech EC | 300-01A-50uG |
| IFNα | Miltenyi | 130-093-873 |
| IFNβ | Peprotech EC | 300-02BC-20uG |
| IL-15 | Peprotech EC | 200-15-500uG |
| IL-18 | Biolegend | 592104 |
| IL-3 | PeproTech EC | 200-03 |

After thawing of E8D5 cells, the IMANp are cultured in the presence of GM-CSF (10-130 ng/ml) alone or in combination with TNFα (0.01-1 ng/ml), IFNα (10 ng/ml), IFNβ (10 ng/ml), IL-3 (130 ng/ml), IL-15 (10 ng/ml), or IL-18 (10 ng/ml) for 48 hours.

### Optional Priming During Differentiation of IMANp

IMANp may be primed during the differentiation phase to enhance their cytotoxicity.

Donor HSCs were thawed and expanded as described previously.

After expansion (E8D0), the HSCs are differentiated for up to 6 days of differentiation (E8DO-E8D6). Between D3-D4, D4-D5 or D5-D6, GM-CSF (10-130 ng/mL) alone or in combination with TNFα (0.01-1 ng/ml), IFNα (10 ng/ml), IFNβ (10 ng/ml), IL-3 (130 ng/ml), IL-15 (10 ng/ml), or IL-18 (10 ng/ml), is used to prime the cells in either 1% or 2% HSA.

### Example 1

Co-Culture with IMANp Granulopoietic Cells Increases Activation of Blood-Derived CD8⁺ T Cells

Method: PBMCs from a healthy donor were cultured with granulopoietic cells (n=4) or blood-derived neutrophils from a PDAC donor (n=1) at different ratios indicated. Co-cultures were performed in the presence or absence of anti-CD3 stimulation (OKT3; 1 μg/ml). After 72 h activation of CD8⁺ T cells was investigated by flow cytometry. PBMCs were labelled with cell trace far red (CTFR) dye prior to co-culture and CD8⁺ cells were gated as live, singlets, CTFR⁺ CD3⁺ CD8⁺. Activation of CD8 cells was investigated by measuring expression of degranulation markers such as CD107a and costimulatory molecules such as 4-1BB and OX40 on the cell surface.

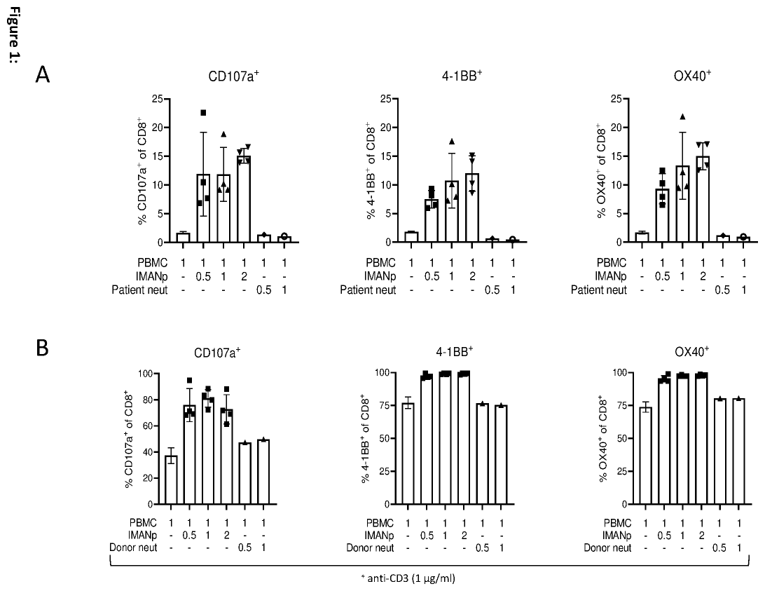

Results: Results show % expression of CD107a, 4-1BB and OX40 on (FIG. 1A) unstimulated and (FIG. 1B) anti-CD3 stimulated CD8⁺ T cells.

(FIG. 1A) Co-culture with granulopoietic cells, and not patient donor neutrophils, increased activation of blood-derived CD8⁺ T cells as demonstrated by the increased expression of CD107a, 4-1BB and OX40 on CD8⁺ T cells in the absence of TCR stimulus. (FIG. 1B) Stimulation with anti-CD3 increased expression of CD107a, 4-1BB and OX40 on CD8 T cells, and expression of these activation markers was further enhanced in the presence of granulopoietic cells, but not patient donor neutrophils. These results indicate that the presence of granulopoietic cells enhances activation as indicated by degranulation (CD107a) and expression of further activation markers (costimulatory molecules OX40 and 4-1BB) of activated CD8 T cells. OX40 and 4-1BB are co-stimulatory markers expressed on activated T cells. Ligation of these co-stimulatory receptors on activated CD8 T cells should increase effector function of these cells (e.g. increase cytotoxicity and IFN-γ production).

Furthermore, the data suggest that granulopoietic cells are providing signal 2 (co-stimulation) and/or signal 3 (cytokine stimulation) of T cell activation.

The data also suggests that granulopoietic cells could be used in combination therapy with T cell engagers e.g. mono/bispecific 4-1BB agonist, or TAA/4-1BB bispecific T cell engager.

### Example 2

Co-Culture with Granulopoietic Cells Increases Activation of Blood-Derived CD4⁺ T Cells

Method: PBMCs from a healthy donor were cultured with granulopoietic cells (n=4) or blood-derived neutrophils from PDAC donor (n=1) at different ratios indicated. Co-cultures were performed in the presence or absence of anti-CD3 stimulation (OKT3; 1 μg/ml). After 72 h activation of CD4⁺ T cells was investigated by flow cytometry. PBMCs were labelled with cell trace far red (CTFR) dye prior to co-culture and CD4⁺ cells were gated as live, singlets, CTFR⁺ CD3⁺ CD4⁺. Activation of CD4 cells was investigated by measuring expression of costimulatory molecules (particularly 4-1BB and OX40) on the cell surface.

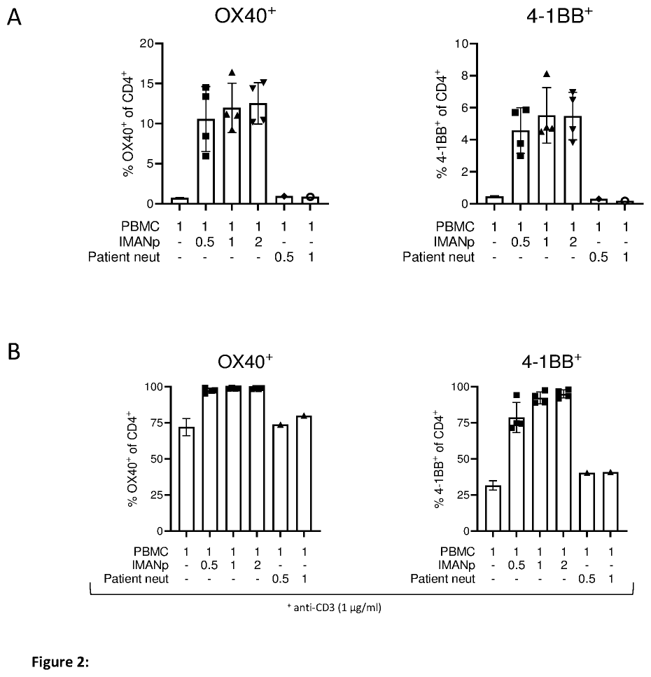

Results: Results show % expression of 4-1BB and OX40 on (FIG. 2A) unstimulated and (FIG. 2B) anti-CD3 stimulated CD4⁺ T cells. (FIG. 2A) Co-culture with granulopoietic cells, and not patient donor neutrophils, increased activation of blood-derived CD4⁺ T cells as demonstrated by increased expression of 4-1BB and OX40 on CD4⁺ T cells in the absence of TCR stimulus. (FIG. 2B) Stimulation with anti-CD3 increased expression of 4-1BB and OX40 on CD4 T cells, and expression of these activation markers was further enhanced in the presence of granulopoietic cells, but not patient donor neutrophils. These results indicate that the presence of granulopoietic cells enhances activation (OX40 and 4-1BB) of CD4 T cells. OX40 and 4-1BB are co-stimulatory markers expressed on activated T cells. Ligation of these co-stimulatory receptors on CD4 T cells should increase effector function of these cells (e.g. increase cytokine production).

The data suggests that granulopoietic cells are providing signal 2 (co-stimulation) and/or signal 3 (cytokine stimulation) of T cell activation.

### Example 3

Co-Culture with Granulopoietic Cells Enhances Proliferation and Accumulation of αβ T Cells

Method: PBMCs from a healthy donor were cultured with granulopoietic cells (n=4) or blood-derived neutrophils from PDAC donor (n=1) at 1:1 ratio. Co-cultures were performed in the presence of anti-CD3 stimulation (OKT3; 1 μg/ml). After 72 h proliferation of CD4⁺ and CD8⁺ T cells was investigated by flow cytometry. PBMCs were labelled with cell trace far red (CTFR) dye (Invitrogen; C34572) prior to co-culture and T cells were gated as live, singlets, CTFR⁺ CD3⁺ CD8⁺ or CD3⁺ CD4⁺. Proliferating cells were identified as having reduced median fluorescence intensity (MFI) of CTFR, which occurs as cells divide and the dye gets diluted.

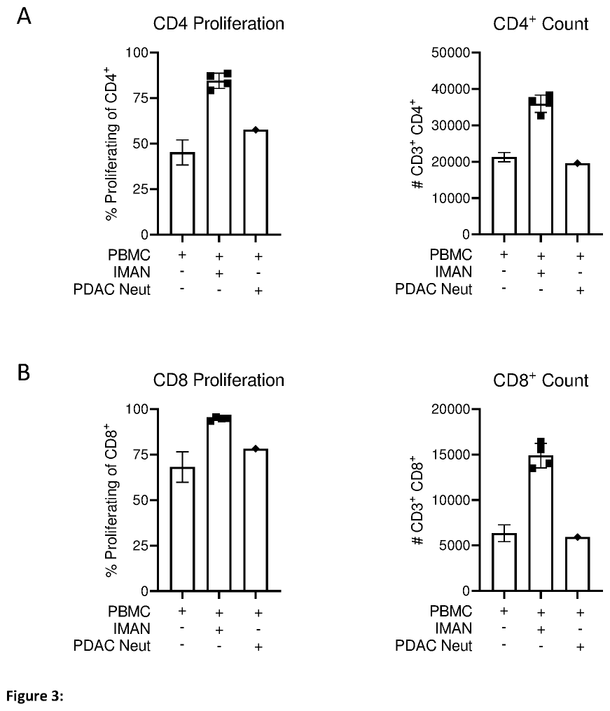

Results: Results show (FIG. 3A) % proliferating CD4⁺ and CD8⁺ T cells and (FIG. 3B) absolute counts of both cell types following 72 h culture. Co-culture with granulopoietic cells, and not patient donor neutrophils, enhanced proliferation of αβ T cells (as demonstrated by increased proliferation of anti-CD3 stimulated CD4 and CD8 T cells), and accumulation of such cells (as demonstrated by increased absolute counts of CD4 and CD8 T cells present following 72 h culture). Granulopoietic cells are capable of amplifying (preferably amplifies) TCR-driven proliferation of αβ T cells and increasing accumulation of immune cells.

The data suggests that granulopoietic cells are providing signal 2 (co-stimulation) and/or signal 3 (cytokine stimulation) of T cell activation.

### Example 4

Co-Culture with Granulopoietic Cells Promotes Survival of Blood-Derived NK Cells and NKT Cells

Method: PBMCs from a healthy donor were cultured with granulopoietic cells (n=4) or blood-derived neutrophils from PDAC donor (n=1) at different ratios indicated. After 72 h absolute counts of NK and NKT cells were quantified by flow cytometry. PBMCs were labelled with cell trace far red (CTFR) dye prior to co-culture and gated as live, singlets, CTFR⁺. NK cells were gated as CD3 CD56⁺, and NKT cells were gated as CD3⁺ CD56⁺.

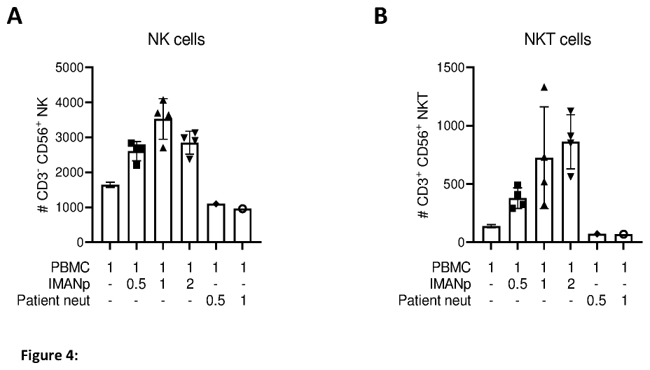

Results: Results show absolute counts of (FIG. 4A) NK cells and (FIG. 4B) NKT cells in both PBMC donors. In both donors, co-culture with granulopoietic cells, and not with blood neutrophils from PDAC patient, promoted the survival of NK and NKT cells as demonstrated by the absolute counts.

Results demonstrate that granulopoietic cells have favourable effect on immune cell survival, as exemplified by their effect on NK and NKT cell survival. This further suggests that medical uses or methods of treatment employing granulopoietic cells may be used in conjunction with NK cell therapy, for example as a feeder cell for NK cell therapy production, or in combination with NK cell therapy to support NK cell therapy function in vivo.

### Example 5

Co-Culture with Granulopoietic Cells Promotes Activation of Blood-Derived NK Cells and NKT Cells

Method: PBMCs from a healthy donor were cultured with granulopoietic cells (n=4) or blood-derived neutrophils from PDAC donor (n=1) at different ratios indicated. After 72 h activation of NK and NKT cells was investigated by flow cytometry. PBMCs were labelled with cell trace far red (CTFR) dye prior to co-culture and gated as live, singlets, CTFR⁺. NK cells were gated as CD3 CD56⁺, and NKT cells were gated as CD3⁺ CD56⁺. Activation of NK and NKT cells was investigated by measuring expression of degranulation markers such as CD107a and costimulatory molecules such as 4-1BB and OX40 on the cell surface.

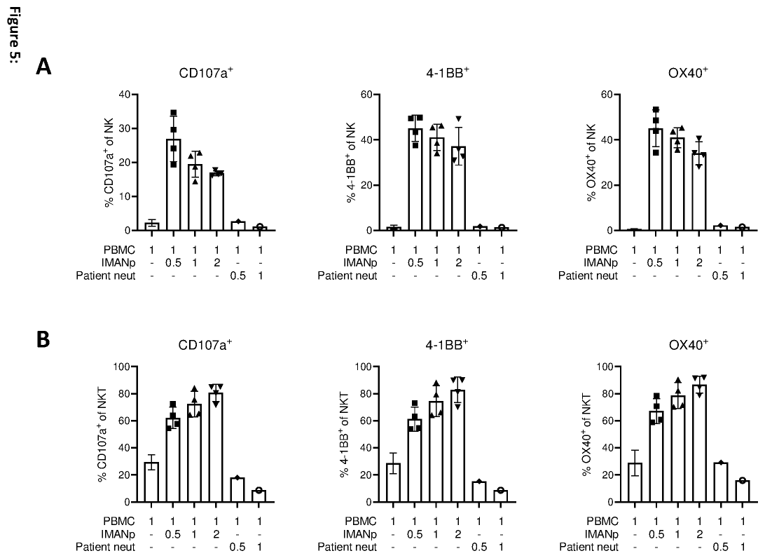

Results: Results show % expression of CD107a, 4-1BB and OX40 on (FIG. 5A) NK cells and (FIG. 5B) NKT cells. Co-culture with granulopoietic cells, and not patient donor neutrophils, increased activation of blood-derived NK cells and NKT cells as demonstrated by increased expression of CD107a, 4-1BB and OX40 on (FIG. 5A) NK and (FIG. 5B) NKT cells. These results indicate that the presence of granulopoietic cells enhances degranulation (CD107a) and activation (OX40 and 4-1BB) of NK and NKT cells.

Results demonstrate that granulopoietic cells have favourable effect on NK and NKT cell activation. This suggests that treatments using granulopoietic cells may be used in conjunction with NK cell therapy, for example as a feeder cell for NK cell therapy production, or in combination with NK cell therapy to support NK cell therapy function in vivo.

### Example 6

Co-Culture with Granulopoietic Cells Enhances Activation of Tumour-Infiltrated Leucocytes (CD8, CD4 and NK Cells)

Method: A tumour digest from a PDAC patient (n=1) was cultured±granulopoietic cells (n=2). After 72 h, activation of tumour-infiltrated αβ T cells and NK cells was investigated by flow cytometry. Digested tumour cells were labelled with cell trace far red (CTFR) dye prior to co-culture and were gated as live, singlets, CTFR⁺. Effector populations were then gated as CD3⁺ CD8⁺, CD3⁺ CD4⁺ or CD3⁻ CD56⁺. Activation of TILs was investigated by measuring expression of degranulation markers such as CD107a and costimulatory molecules such as 4-1BB and/or OX40 on the cell surface as indicated.

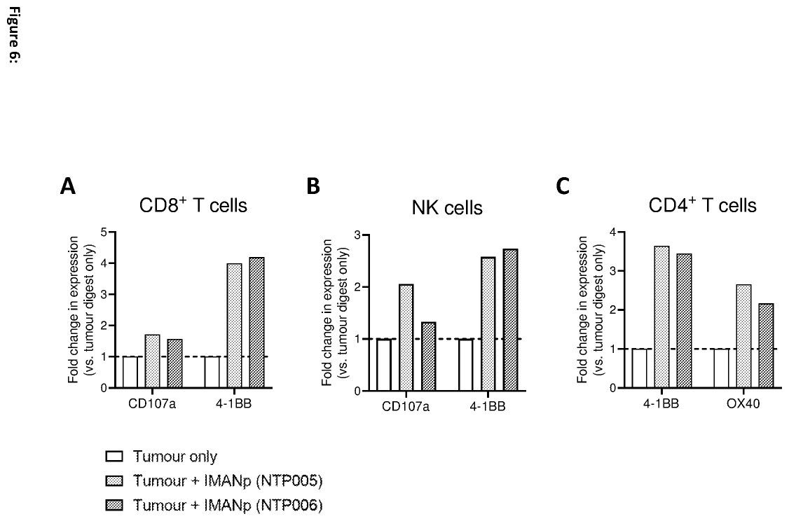

Results: Results show fold change in expression of indicated activation markers on tumour-infiltrated (FIG. 6A) CD8 T cells, (FIG. 6B) NK cells, and (FIG. 6C) CD4 T cells. Data are shown as fold increase in expression versus tumour digest only conditions. (FIGS. 6A and 6B) Data show that co-culture with granulopoietic cells there was increased activation of tumour-infiltrated leucocytes (exemplified by CD8 and NK cells) as demonstrated by increased expression of CD107a and 4-1BB on CD8 T cells and NK cells, indicating granulopoietic cells' ability to promote degranulation of cytotoxic effector cells in the TME as well as enhance their activation through increased 4-1BB expression. (FIG. 6C) Data show that co-culture with granulopoietic cells increased activation of tumour-infiltrated CD4 as demonstrated by increased expression of 4-1BB and OX40 co-stimulatory receptors on tumour-infiltrated CD4 T cells. Ligation of these co-stimulatory receptors results in increased effector function of T cells, e.g. increased cytokine production.

### Example 7

Co-Culture with Granulopoietic Cells Increases Cytokine Production by PBMCs

Method: PBMCs from healthy donors (n=2) were cultured with granulopoietic cells (n=4) or blood-derived neutrophils from PDAC donor (n=1) at different ratios indicated. Co-cultures were performed in the presence of anti-CD3 stimulation (OKT3; 1 μg/ml). After 72 h supernatants were collected and the concentration of secreted cytokines such as IFN-γ was measured by quantitative sandwich ELISA (ab174443) according to manufacturer's instructions.

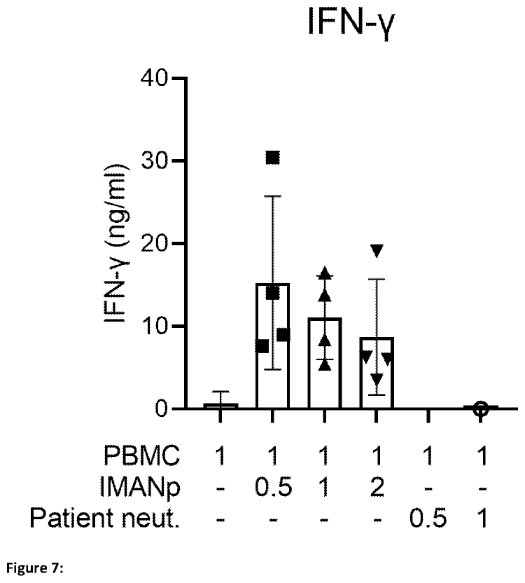

Results: Results show (FIG. 7) the concentration of IFN-γ detected in supernatants. Co-culture with granulopoietic cells, and not patient donor neutrophils, increased production of IFN-γ by PBMCs. This data shows granulopoietic cells boosting T cell effector functions for potent anti-tumour immunity as demonstrated by the increased production of IFN-γ by PBMCs.

The data suggests that granulopoietic cells are providing signal 2 (co-stimulation) and/or signal 3 (cytokine stimulation) of T cell activation. Furthermore, they indicate that the granulopoietic cells will not drive uncontrolled T cell activation, which is important in terms of safety of the medical uses or methods of treatment.

Activation noted in the absence of anti-CD3 may reflect activation of a small population of memory T cells that do not require TCR stimulation for their activation.

### Example 8

Co-Culture with Granulopoietic Cells Increases Cytokine Production by Tumour infiltrating lymphocytes (TILs)

Method: Tumour digest from PDAC patient (n=1) was cultured±granulopoietic cells (n=2). Co-cultures were performed in the presence of anti-CD3 stimulation (OKT3; 1 μg/ml). After 72 h supernatants were collected and the concentration of secreted cytokines such as IFN-γ was measured by quantitative sandwich ELISA (ab174443) according to manufacturer's instructions.

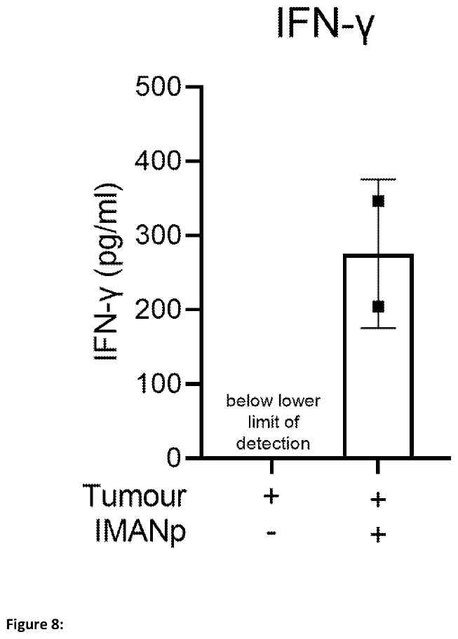

Results: (FIG. 8) Results show the concentration of IFN-γ detected in cell culture supernatants. Co-culture with granulopoietic cells increased production of IFN-γ by TILs. This data shows granulopoietic cells boosting T cell effector functions for potent anti-tumour immunity as demonstrated by the increased production of IFN-γ by TILs.

The data suggests that granulopoietic cells are providing signal 2 (co-stimulation) and/or signal 3 (cytokine stimulation) of T cell activation.

### Example 9

Granulopoietic Cells Promote Immune Cell Recruitment into the Tumour Microenvironment

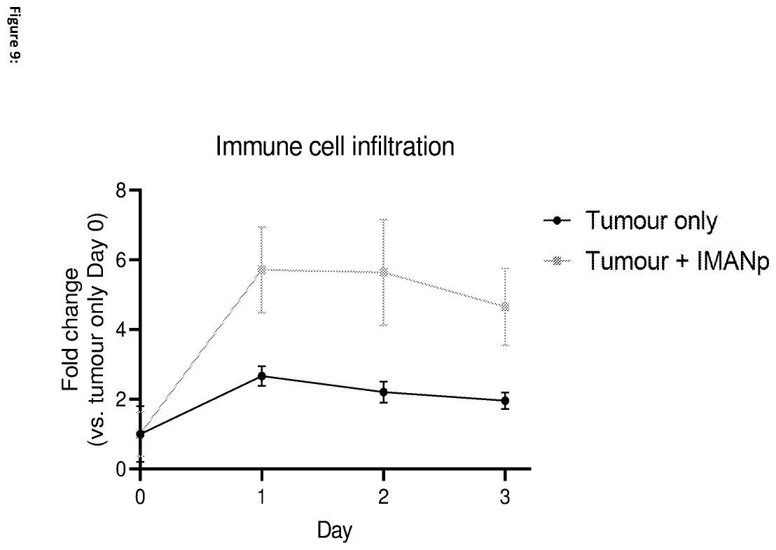

Method: Fresh patient tumour biopsy (RCC)±granulopoietic cells were encapsulated into tumour-on-a-chip model, and co-cultured with matched donor PBMCs. (FIG. 9) PBMC recruitment into the microtumour was measured daily for 3 days via live cell imaging.

Results: Results show (FIG. 9) fold increase in immune cell infiltration into the microtumour at timepoints indicated versus day 0 tumour only at timepoints indicated. These data suggest that granulopoietic cells are immunomodulatory through their ability to recruit immune cells into the tumour microenvironment.

### Example 10

### Granulopoietic Cells Promote Enhanced Tumour Killing

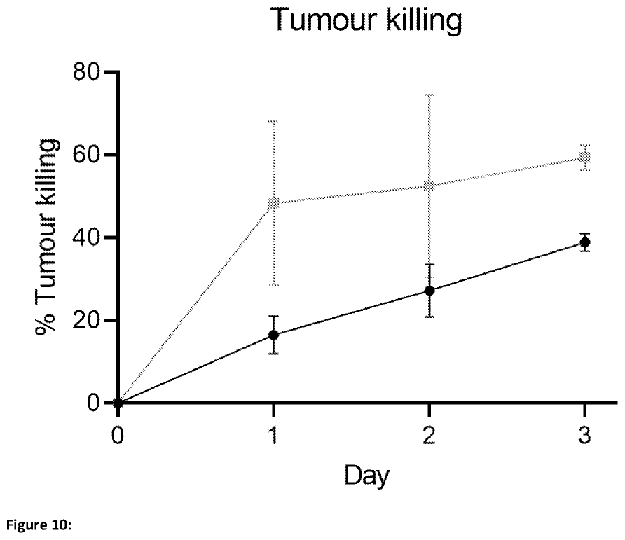

Method: Fresh patient tumour biopsy (RCC)±granulopoietic cells were encapsulated into tumour-on-a-chip model, and co-cultured with matched donor PBMCs. (FIG. 10) Tumour cell cytotoxicity was measured daily for 3 days via live cell imaging.

Results: Results show (FIG. 10) % tumour killing at timepoints indicated. These data suggest that granulopoietic cells increased tumour cell killing.

It will be recognised that the killing of tumour cells is a key aim of anti-cancer treatments. Accordingly, the increased tumour killing activity noted on treatment with granulopoietic cells clearly indicates that the medical uses, methods of treatment, and pharmaceutical compositions of the invention will be able to exert therapeutic anti-cancer activity. As demonstrated in the preceding Examples, this is achieved by amplifying the immune response, and in particular the effects of non-granulocytic cells in the immune response.

### Example 11

Granulocytes Produced on Differentiation of Granulopoietic Cells Promote Immune Cell Recruitment into the Tumour Microenvironment Via Secretion of Chemokines.

Method: Granulopoietic cells were differentiated, and the resultant granulocytes (“IMANs”) (n=4) were stimulated±IFN-α, IFN-β or TNF (all 10 ng/ml) for 24 h. Data show concentration of chemokines such as CXCL10 in the cell culture supernatants quantified by LEGENDplex according to manufacturer's instructions.

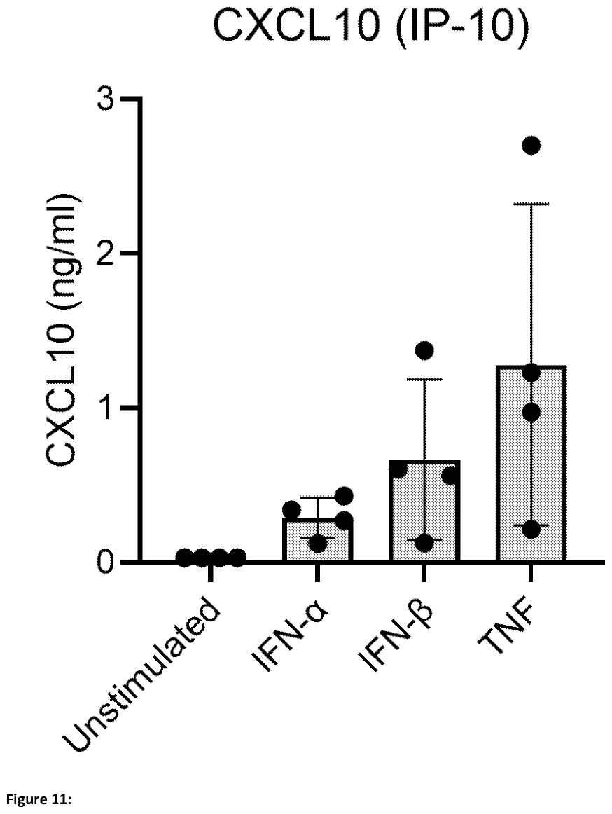

Results: Results show (FIG. 11) that granulocytes produced on differentiation of granulopoietic cells release CXCL10 when activated by various cytokines. These data suggest that treatment using granulopoietic cells may play a further role in promoting immune cell recruitment into the tumour microenvironment through their production of granulocytes able to release chemokines such as CXCL10. CXCL10 is known to be a powerful chemoattractant for CXCR3⁺ T cells and NK cells.

The data indicate that the granulocytes produced on differentiation of granulopoietic cells may be activated via many different pathways. Data suggests that it may be possible to combine granulopoietic cell therapy, and particularly the granulocytes produced as a result of such therapy, with mono/bispecific antibodies that activate innate immune cells. For example, combination with anti-CD40 mAb or anti-CD40/TAA bispecific for combined granulopoietic cell activation and tumour targeting.

### Example 12

Granulocytes Derived from Granulopoietic Cells Express Ligands for T and NK Cell Co-Stimulatory Receptors

Method: Granulocytes produced on differentiation of granulopoietic cells suitable for use in the medical uses or methods of treatment of the invention (n=3) were analysed for expression of T and NK cell co-stimulatory receptors, 4-1BBL and OX40L, by flow cytometry.

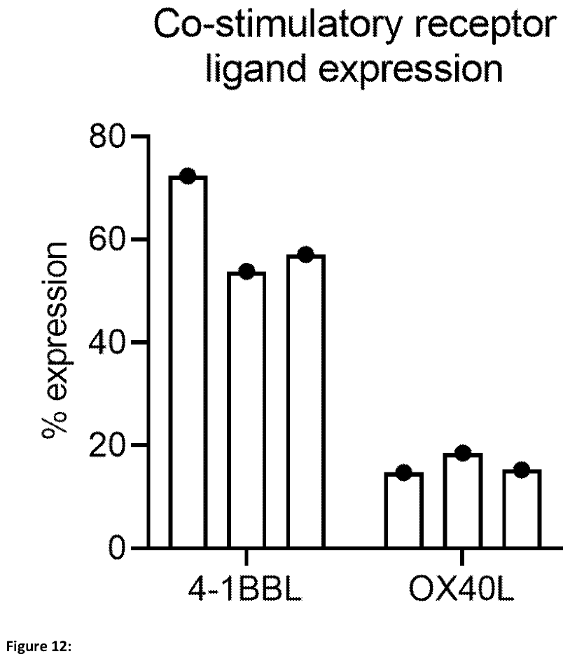

Results: Results show (FIG. 12) % expression of 4-1BBL and OX40L on the granulocytes derived from granulopoietic cells from 3 individual donors. These data suggest that granulopoietic cells enhance T and NK cell effector functions through co-stimulation as demonstrated by the expression of both 4-1BBL and OX40L.

### Example 13

### Priming of Granulopoietic Cells Enhances Cytotoxicity

Method: Granulopoietic cells (IMANp) were prepared and primed post-thaw as described above.

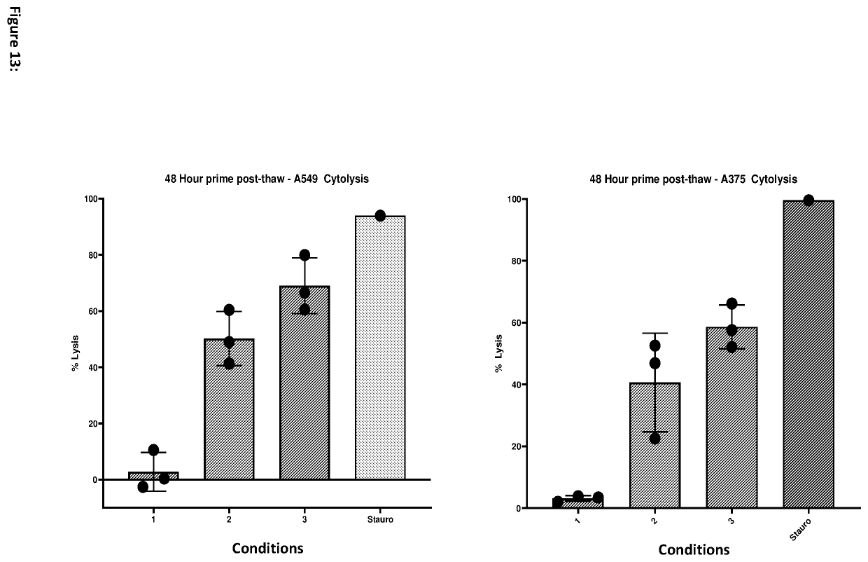

Results: Results show (FIG. 13) that culture of IMANp in the presence of 10 ng/ml GM-CSF and 10 ng/ml IL-15 (condition ‘2’) for 48 hours post-thaw increases their cytotoxicity against A549 cells by approximately 50-fold and increases their cytotoxicity against A375 cells by approximately 40-fold compared to unprimed controls (condition ‘1’). Meanwhile, culture of IMANp in the presence of 100 ng/ml GM-CSF and 10 ng/mL TNFα (condition ‘3’) for 48 hours post-thaw increases their cytotoxicity against A549 cells by approximately 70-fold and increases their cytotoxicity against A375 cells by approximately 60-fold compared to unprimed controls.

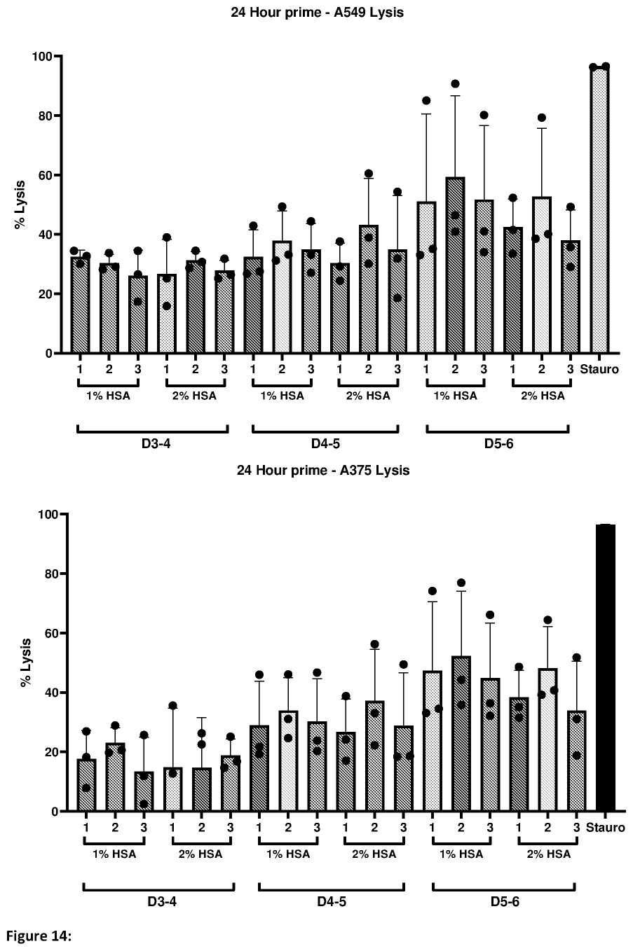

Results show (FIG. 14) that priming with 10 ng/mL GM-CSF+10 ng/ml IL-25 (condition ‘1’); 10 ng/ml GM-CSF+1 ng/mL TNFα (condition ‘2’) or 10 ng/mL GM-CSF (condition ‘3’) at D3-4, D4-5 or D5-6 of the differentiation phase provides IMANp with enhanced cytotoxicity.

Cytotoxicity assays were performed for 48 hours with a 20:1 IMANp: target cell ratio.

The immunomodulatory properties shown in Examples 1-12 are unaffected by priming during or after differentiation.

### Example 14

Depletion of αβ T Cells from PBMCs/Leukopaks

PBMCs depleted of αβ T cells used in the examples below were prepared using the following methods.

αβ T cells were depleted from PBMCs/Leukopaks utilizing biotin-conjugated anti-TCRαβ antibody and anti-biotin microbeads according to the manufacturer's instructions (Miltenyi Biotec). Briefly, cells were resuspended at 1×10⁷ cells/ml in PBS containing 0.5% BSA and 2 mM EDTA (MACS buffer). Cells were incubated with biotin-conjugated anti-TCRαβ antibody (Clone BW242/412; 1:50 dilution) for 15 minutes at room temperature. Cells were washed in MACS buffer and centrifuged at 300×g for 5 minutes. Cells were resuspended in MACS buffer (80 μl/1×10⁷ cells) containing anti-biotin microbeads (20 μl/1×10⁷ cells). Cells were incubated at 4° C. for 15 minutes. Cells were washed in MACS buffer and centrifuged at 300×g for 5 minutes. Up to 1.25×10⁸ cells were resuspended in 500 μl of MACS buffer for αβ T cell depletion using LD columns. LD columns were placed in the magnetic field of the MACS MultiStand (Miltenyi Biotec). Each column was prepared by rinsing with 2 ml of MACS buffer. The cell suspension was then applied to the column. Unlabelled cells (αβTCR) passed through the column and were collected in a 50 ml tube. The column was then washed with 2×1 ml of MACS buffer, with effluent also collected in 50 ml tube. Total effluent contained unlabelled αβTCR cells.

### Flow Cytometry

PBMCs and αβ-depleted PBMCs were labelled with cell trace far red (CTFR) dye prior to co-culture for identification. Granulopoietic cells were unlabelled prior to co-culture. Following culture, cells were washed in PBS and incubated with live/dead stain (Fixable Viability Dye eFluor 780; 1:500 dilution) and FcγR block (Human TruStain FcX; 1:50 dilution) for 20 minutes. Cells were then washed in flow cytometry buffer (PBS+2% FCS) and surface stained with fluorochrome-conjugated anti-human antibodies. All antibodies were used at 1:50 dilution, with staining performed in 50 μl/sample. Following surface staining, cells were fixed using 100 μl 1× BD CellFix, before being acquired on a MACSQuant 16 (Miltenyi). Data were analysed using FlowLogic software. Analysis of the stained populations was performed by gating on single, live cells.

### Materials

### Antibodies

| Target | Conjugation | Clone | Supplier | Cat# |
|---|---|---|---|---|
| TCRαβ | Biotin | BW242/412 | Miltenyi | 130-113-529 |
| CD3 | BV421 | OKT3 | BioLegend | 317344 |
| CD3 | PerCP-Cy5.5 | OKT3 | BioLegend | 317336 |
| TCR Vδ1 | PerCP-Vio700 | REA173 | Miltenyi | 130-120-441 |
| TCR Vδ2 | VioBright | REA771 | Miltenyi | 130-126-908 |
| | B515 | | | |
| TCR γδ | PE | REA591 | Miltenyi | 130-113-512 |
| CD56 | PE | HCD56 | BioLegend | 318306 |
| 4-1BB | BV650 | 4B4-1 | BioLegend | 309828 |
| 4-1BB | BV421 | 4B4-1 | BioLegend | 309820 |
| CD25 | PE-Vio770 | REA570 | Miltenyi | 130-114-541 |
| OX40 | FITC | Ber-ACT35 | BioLegend | 350006 |
| CD11b | BV650 | M1/70 | BioLegend | 101259 |
| CD54 | PerCP-Cy5.5 | HA58 | BioLegend | 353120 |
| 4-1BBL | PE-Cy7 | 5F4 | BioLegend | 311512 |

### Other Reagents

| Reagent | Supplier | Cat# |
|---|---|---|
| Anti Biotin Microbeads | Miltenyi | 130-090-485 |
| LD columns | Miltenyi | |
| Cell trace far red kit | Thermo Fisher Scientific | C34564 |
| Fixable Viability Dye | Thermo Fisher Scientific | 65-0865-14 |
| eFluor780 | | |
| Human TruStain FcX | BioLegend | 422302 |
| BD CellFIX | BD | 340181 |

### PBMC Composition Pre- and Post-αβ T Cell Depletion

PBMCs were depleted of αβ T cells. PBMCs were stained for analysis by flow cytometry with antibodies specific for CD3, γδ TCR and CD56 before and after αβ T cell depletion. Different populations were gated as follows:
αβ T cells: CD3⁺ γδ TCR⁻
γδ T cells: CD3⁺ γδ TCR⁺
NK cells: CD3⁻ γδ TCR CD56⁺.
Other cells: CD3⁻ γδ TCR⁻ CD56⁻ (population consisting of monocytes, B cells, DCs, macrophages, progenitor cells etc.)

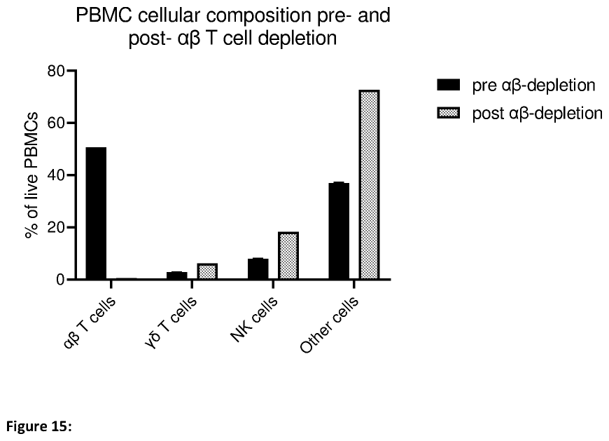

FIG. 15 shows successful depletion of αβ T cells, and enrichment of γδ and NK cells in total PBMC population post αβ T cell depletion.

### Example 15

Co-Culture with Granulopoietic Cells Immunomodulates Blood-Derived Vδ1⁺ γδ T Cells

PBMCs from a healthy donor were depleted of αβ T cells and cultured±granulopoietic cells (n=4) at a ratio of 1:2 αβ⁻ PBMC: granulopoietic cells. Co-cultures were performed+IL-15 (10 ng/ml). Activation of Vδ1⁺ γδ T cells was investigated after 24 hours by flow cytometry. αβ⁻ PBMCs were labelled with cell trace far red (CTFR) dye prior to co-culture and gated as live, singlets, CTFR⁺. Vδ1⁺ γδ T cells were gated as CD3⁺ γδTCR⁺ Vδ1⁺. Activation of Vδ1⁺ γδ T cells was investigated by measuring expression of 4-1BB and CD25 on the cell surface.

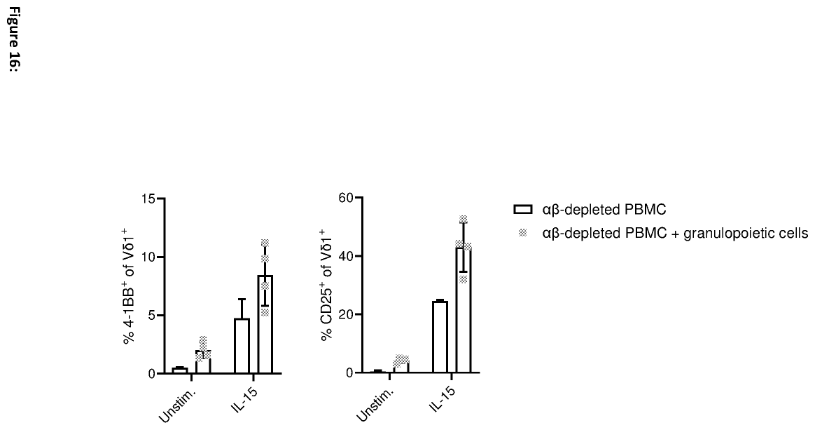

FIG. 16 shows % expression of 4-1BB and CD25 on the surface of Vδ1⁺ γδ T cells following 24 h co-culture with granulopoietic cells±IL-15. Results demonstrate that co-culture with granulopoietic cells drives expression of both 4-1BB and CD25 on Vδ1⁺ γδ T cells. Expression of these activation markers is also driven by IL-15, with further enhanced expression occurring in the presence of both granulopoietic cells and IL-15. Upregulation of these surface molecules is associated with activation and enhanced effector function of γδ T cells.

These data demonstrate the advantageous and unexpected properties of a multi-cellular composition, whereby culture with granulopoietic cells (optionally in the presence of IL-15) promotes activation of innate T cells, such as Vδ1⁺ γδ T cells.

### Example 16

Co-Culture with Granulopoietic Cells Immunomodulates Blood-Derived Vδ2⁺ γδ T Cells.

PBMCs from a healthy donor were depleted of αβ T cells and cultured±granulopoietic cells (n=4) at a ratio of 1:2 αβ PBMC: granulopoietic cells. Co-cultures were performed±IL-15 (10 ng/ml). Activation of Vδ2⁺ γδ T cells was investigated after 48 hours by flow cytometry. αβ⁻ PBMCs were labelled with cell trace far red (CTFR) dye prior to co-culture and gated as live, singlets, CTFR⁺. Vδ2⁺ γδ T cells were gated as CD3⁺ γδTCR⁺ Vδ2⁺. Activation of Vδ2⁺ γδ T cells was investigated by measuring expression of 4-1BB on the cell surface.

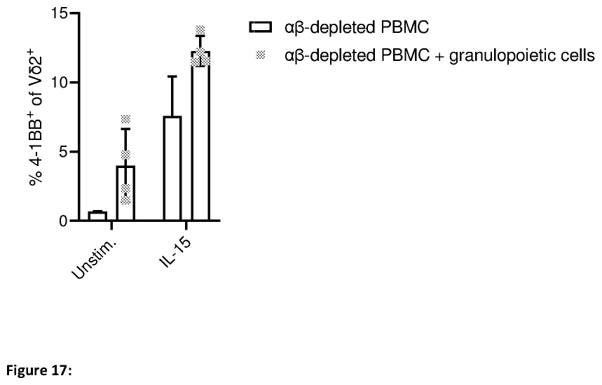

FIG. 17 shows % expression of 4-1BB on the surface of Vδ2⁺ γδ T cells following 48 h co-culture with granulopoietic cells±IL-15. Results demonstrate that co-culture with granulopoietic cells drives expression 4-1BB on Vδ2⁺ γδ T cells. Expression of 4-1BB is also driven by IL-15, with further enhanced expression occurring in the presence of both granulopoietic cells and IL-15. Upregulation of these surface molecules is associated with activation and enhanced effector function of γδ T cells.

These data demonstrate the advantageous and unexpected properties of a multi-cellular composition, whereby culture with granulopoietic cells (optionally in the presence of IL-15) promotes activation of innate T cells, such as Vδ2⁺ γδ T cells.

### Example 17

Granulopoietic Cells Promote the Proliferation and/or Survival of γδ T Cells.

PBMCs from a healthy donor are depleted of αβ T cells and cultured±granulopoietic cells. Co-cultures are performed±IL-15 (10 ng/ml). After 72 h absolute counts of Vδ1⁺ and Vδ2⁺ γδ T cells are quantified by flow cytometry. αβ-depleted PBMCs are labelled with cell trace far red (CTFR) dye prior to co-culture and gated as live, singlets, CTFR⁺. γδ T cells are gated as CD3⁺γδTCR⁺ Vδ1⁺ or CD3⁺ γδTCR⁺ Vδ2⁺. Enhanced proliferation and/or survival of γδ T cells following co-culture with granulopoietic cells is measured by reduction in median fluorescence intensity (MFI) of CTFR in Vδ1⁺ and Vδ2⁺ γδ T cells, which occurs as cells divide and the dye gets diluted and/or increased absolute counts of Vδ1⁺ and Vδ2⁺ γδ T cells following 72 hours co-culture.

### Example 18

### Granulopoietic Cells Support Survival of Blood-Derived Myeloid Cells

PBMCs from a healthy donor were depleted of αβ T cells and cultured±granulopoietic cells (n=4) at a ratio of 1:2 αβ⁻ PBMC: granulopoietic cells. Co-cultures were performed±IL-15 (10 ng/ml). After 24 hours absolute counts of CD11b⁺ myeloid cells were quantified by flow cytometry. PBMCs were labelled with cell trace far red (CTFR) dye prior to co-culture and gated as live, singlets, CTFR⁺. Myeloid cells were gated as CTFR⁺ CD11b⁺.

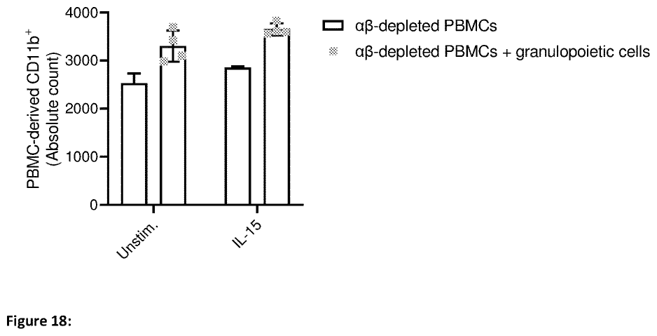

FIG. 18 shows absolute counts of CD11b⁺ myeloid cells are enhanced in conditions containing granulopoietic cells, suggesting a role for granulopoietic cells in the survival of blood-derived innate cells in vitro.

### Example 19

Co-Culture with αβ-Depleted PBMCs Enhances Activation of Granulopoietic Cells

Granulopoietic cells (n=4) were cultured±PBMCs from a healthy donor that were depleted of αβ T cells at a ratio of 1:2 αβ⁻ PBMC: granulopoietic cells. Co-cultures were performed±IL-15 (10 ng/ml). Activation of granulopoietic cells was investigated after 24 hours by flow cytometry. αβ⁻ PBMCs were labelled with cell trace far red (CTFR) dye prior to co-culture. Granulopoietic cells were gated as live, singlets, CTFR. Activation status of granulopoietic cells was investigated by measuring expression of CD54 on the cell surface.

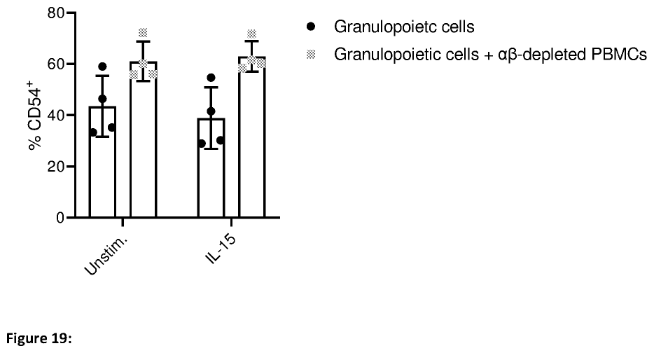

FIG. 19 shows % expression of CD54 on the surface of granulopoietic cells following 24 hours co-culture±αβ-depleted PBMCs±IL-15. Results demonstrate that co-culture with αβ -depleted PBMCs drives expression of CD54 on granulopoietic cells, indicating heightened activation status of granulopoietic cells.

### Example 20

### Granulopoietic Cells Express Ligands for γδ T and NK Cell Co-Stimulatory Receptors

Granulopoietic cells (n=4) were cultured±PBMCs from a healthy donor that were depleted of αβ T cells at a ratio of 1:2 αβ⁻ PBMC: granulopoietic cells. Co-cultures were performed±IL-15 (10 ng/ml). Expression of 4-1BBL, the ligand for T and NK cell co-stimulatory receptor 4-1BB was analysed on the surface of granulopoietic cells following 24 hours co-culture by flow cytometry. αβ PBMCs were labelled with cell trace far red (CTFR) dye prior to co-culture. Granulopoietic cells were gated as live, singlets, CTFR.

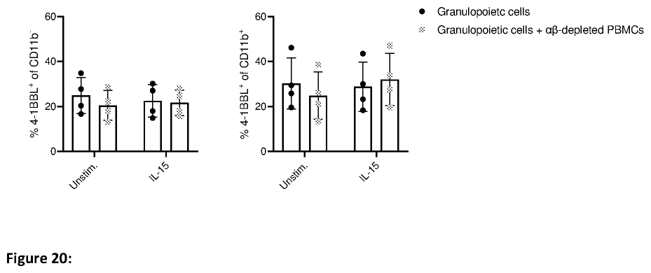

FIG. 20 shows % expression of 4-1BBL on CD11b⁻ and CD11b⁺ granulopoietic cells. These data suggest that granulopoietic cells enhance γδ T cell and NK cell effector functions through co-stimulation as demonstrated by the expression of 4-1BBL.

### Example 21

### Granulopoietic Cells Enhance Maturation of Blood-Derived Dendritic Cells

Immature monocyte-derived dendritic cells (DCs) are co-cultured with granulopoietic cells for 24 hours. Co-cultures are performed±IL-15 (10 ng/ml). DCs are labelled with cell trace far red (CTFR) prior to co-culture and gated as live, singlets, CTFR⁺, CD11c⁺ HLA-DR⁺. After 24 hours surface expression of molecules associated with DC maturation are analysed by flow cytometry. Upregulation of maturation-associated molecules including CD83 and/or CD86 and/or CD80 are analysed. Maturation of DCs is associated with enhanced ability to trigger T cell proliferation, cytotoxicity and Th1 polarization.

### Example 22

### Granulopoietic Cells Drive a Pro-Inflammatory ‘M1’ Phenotype in Blood-Derived Macrophages

Monocyte-derived macrophages are co-cultured with granulopoietic cells for 24 hours. Co-cultures are performed±IL-15 (10 ng/ml). Macrophages are labelled with cell trace far red (CTFR) prior to co-culture and gated as live, singlets, CTFR⁺. After 24 hours surface expression of molecules associated with M1 phenotype are analysed by flow cytometry, and the production of pro-inflammatory cytokine TNF is measured in cell culture supernatants by ELISA. Upregulation of co-stimulatory molecules CD86 and/or CD40, and/or enhanced secretion of TNF are associated with M1 proinflammatory macrophages.

### Example 23

### Generation of Composition Comprising Granulopoietic Cells and Innate Non-Granulocytic Immune Cells of a Single Donor

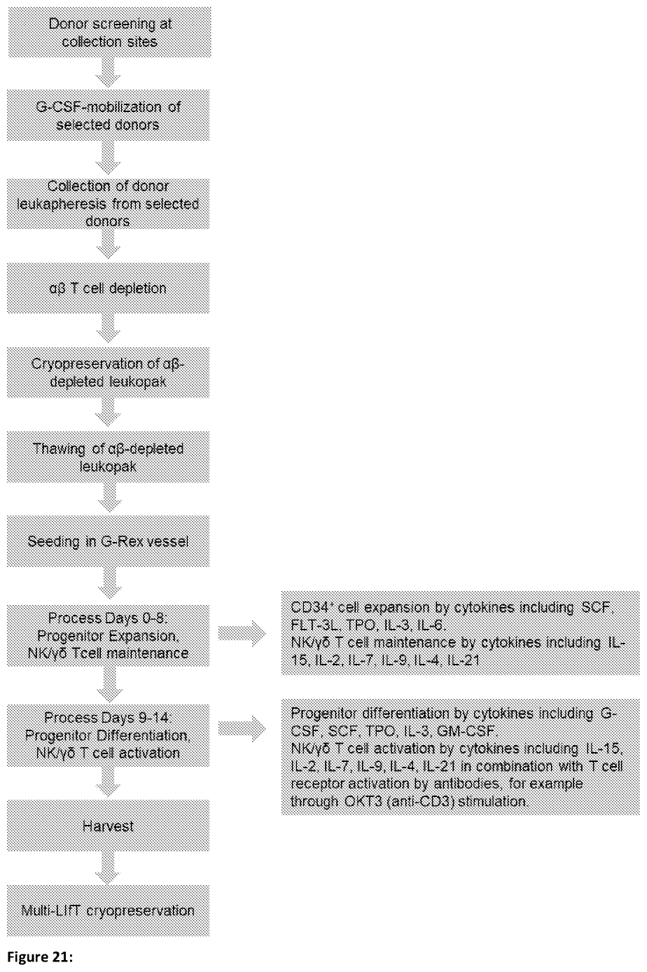

Compositions of the invention may be prepared using the method shown in FIG. 21. Donors may be identified via a screening process (e.g. donors whose granulocytes demonstrate high cytotoxic potential via a killing assay, such as a cancer killing assay) and are invited to donate their PBMCs which are enriched with haematopoietic stem cells (HSCs). Prior to donation, donor CD34⁺ HSCs cells are mobilized from the bone marrow into blood via a course of 5 consecutive daily Neupogen (G-CSF) injections to a total dose of 10 μg/kg/day of donor body weight. PBMCs are procured on the 5th day of mobilization using a Continuous MonoNuclear Cell (CMNC) program on a Spectra Optias apheresis machine or similar. Collected material is stored or transported at 2-8° C., and diluted to a white blood cell concentration of <200×10⁹ cells/mL using donor autologous plasma added either as part of the collection process or after.

Depletion of αβ T cells from the collected donor leukapheresis product is performed using CliniMACS TCRα/β kit (Miltenyi Biotec) on the CliniMACS Prodigy (Miltenyi Biotec) according to manufacturers instructions. Following depletion of αβ T cells, the remaining leukapheresis product is cryopreserved for future use.

On Day 0, the donor αβ-depleted leukapheresis product is thawed and seeded in G-Rex bioreactors in medium (for example IMDM with GlutaMAX+1% HAS+1% ITS) containing cytokines promoting expansion of CD34⁺ HSCs such as SCF (200 ng/ml), FLT-3L (200 ng/ml), TPO (20 ng/ml), IL-3 (15 ng/ml) and IL-6 (15 ng/ml). One or more cytokines selected from the group comprising (or consisting of) IL-15, IL-2, IL-7, IL-9, IL-4 and IL-21 are included at this time to maintain NK and γδ T cells throughout the HSC stem cell expansion phase. On Day 8, differentiation of expanded HSCs towards the granulocytic lineage is initiated via media exchange to differentiation media consisting of IMDM (supplemented as above) containing 130 ng/ml SCF, G-CSF and TPO and optionally 10 ng/ml GM-CSF, 1 ng/ml TNF and 130 ng/ml IL-3. Further cytokine supplementation with IL-15, IL-2, IL-7, IL-9, IL-4 and/or IL-21 is included at this time to promote expansion and activation of NK and γδ T cells. Anti-CD3 antibody (OKT3) is also included for activation/proliferation of γδ T cells. On day 14 the resulting cell product is harvested and resuspended in CS10 prior to cryopreservation.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A method of preparing cells for therapeutic use, the method comprising:
culturing a population of progenitor cells in cell culture conditions that promote differentiation of the progenitor cells comprising the presence of:
G-CSF,
GM-CSF,
IL-3 and
TNF;
to produce a population of granulopoietic cells.

2. A method according to claim 1, wherein the cell culture conditions that promote differentiation of the progenitor cells further comprise at least one supplement from the group consisting of: SCF; TPO; ITS; and HSA.

3. A method according to claim 2, wherein the cell culture condition that promote differentiation of the progenitor cells comprise:
GM-CSF at a concentration of approximately 0.01 μg/mL; and
G-CSF at a concentration of approximately 0.13 μg/mL; and
SCF at a concentration of approximately 0.13 μg/mL; and
TPO at a concentration of approximately 0.13 μg/mL; and
IL-3 at a concentration of approximately 0.13 μg/mL; and
TNF at a concentration of approximately 0.001 μg/mL; and
ITS; and
HSA at approximately 1%.

4. A method according to any of claims 1 to 3, wherein the progenitor cells are cultured for 4 to 6 days in conditions to produce a population of granulopoietic cells.

5. A method according to claim 4, wherein the progenitor cells are cultured for 4 or 5 days in conditions to produce a population of granulopoietic cells.

6. A method according to any preceding claim, wherein GM-CSF and IL-3 are provided to the cells for the final 48 hours of the period for which they are in culture.

7. A method according to any preceding claim, wherein TNF is provided to the cells for the final 24 hours of the period for which they are in culture.

8. A method according to any preceding claim, further comprising a step of culturing a population of stem cells in cell culture conditions to produce the population of progenitor cells:
wherein the cell culture conditions for producing the progenitor cells comprise the presence of
SCF,
Flt-3 Ligand,
IL-3,
IL-6, and
TPO.

9. A method according to claim 8, wherein the cell culture conditions for producing the progenitor cells comprise:
SCF at a concentration of approximately 0.2 μg/mL; and
Flt-3 Ligand at a concentration of approximately 0.2 μg/mL; and
IL-3 at a concentration of approximately 0.015 μg/mL; and
IL-6 at a concentration of approximately 0.015 μg/mL; and
TPO at a concentration of approximately 0.02 μg/mL; and
ITS; and
HSA at approximately 1%.

10. A method according to claim 5 or claim 6, wherein the stem cells are cultured for 8-9 days in conditions to produce a population of progenitor cells.

11. A method according to claim 10, wherein the stem cells are cultured for 8 days in conditions to produce a population of progenitor cells.

12. A method according to any of claims 8 to 11, wherein the stem cells are HSCs.

13. A method according to any preceding claim, further comprising a step of purifying the population of granulopoietic cells produced.

14. A method according to any preceding claim, further comprising a step of formulating the population of granulopoietic cells produced for medical use.

15. A method according to any preceding claim, further comprising priming the granulopoietic cells for therapeutic use, by a method comprising culturing the population of granulopoietic cells in the presence of GM-CSF, and optionally one or more cytokines selected from the group consisting of: TNF, IFN-α, IFN-β, IL-15, and IL-18.

16. A method according to claim 15, further comprising a step of purifying the population of primed granulopoietic cells produced, and/or formulating this population of cells for medical use.

17. A cell culture medium for use in a method in accordance with any of claims 1 to 16, the medium comprising: G-CSF at a concentration of approximately 0.13 μg/mL; and SCF at a concentration of approximately 0.13 μg/mL; and TPO at a concentration of approximately 0.13 μg/mL; and ITS; and HSA at approximately 1%.

18. A cell culture medium according to claim 17, further comprising: GM-CSF at a concentration of approximately 0.01 μg/mL; and IL-3 at a concentration of approximately 0.13 μg/mL.

19. A cell culture medium according to claim 17 or claim 18, further comprising: TNF at a concentration of approximately 0.001 μg/mL.

20. A cell culture medium, for use in a method in accordance with any of claims 8 to 19, the medium comprising: SCF at a concentration of approximately 0.2 μg/mL; and Flt-3 Ligand at a concentration of approximately 0.2 μg/mL; and IL-3 at a concentration of approximately 0.015 μg/mL; and IL-6 at a concentration of approximately 0.015 μg/mL; and TPO at a concentration of approximately 0.02 μg/mL; and ITS; and HSA at approximately 1%.

21. A population of granulopoietic cells prepared for therapeutic use by a method of any of claims 1 to 14.

22. A population of granulopoietic cells prepared for therapeutic use by a method of any of claims 1 to 20.

23. A population of granulopoietic cells according to claim 21 or claim 22, comprising:
a first subpopulation of cells that are CD15+ CD64+ CD18+ CD49d+ CD71+;
a second subpopulation of cells that are CD15− CD11b+/− CD18+ CD49d+ CD32+ HLA-DR−; and
a third subpopulation of cells that are CD15− CD11b− HLA-DR+ CD18+ CD49d+ and CD71+.

24. An isolated population of granulopoietic cells comprising:
a first subpopulation of cells that are CD15+ CD64+ CD18+ CD49d+ CD71+;
a second subpopulation of cells that are CD15− CD11b+/− CD18+ CD49d+ CD32+ HLA-DR−; and
a third subpopulation of cells that are CD15− CD11b− HLA-DR+ CD18+ CD49d+ and CD71+.

25. A population of granulopoietic cells according to claim 23 or claim 24, further comprising:
a fourth subpopulation of cells that are CD15− CD11b+ HLA−DR+.

26. A population of granulopoietic cells that are CD15+ CD64+ CD18+ CD49d+ CD71+.

27. A population of granulopoietic cells according to any of claims 23 to 26, that are CD64+ and/or CD16− and/or CD62L−.

28. A population of granulopoietic cells according to any of claims 23 to 27, wherein the first and/or second subpopulation of cells are also positive for one, more than one, or all of the markers selected from the group consisting of: CD177, CD11b, CD71, CD66b, HLA-DR, CD115, CD49d, CD40, CD62L, CD54, CD18, CD34, CXCR4, CD64, CD32, CXCR2, CD38, Mac1, 4-1BBL, OX40L, PD-L1, and CD14.

29. A population of granulopoietic cells according to any of claims 23 to 28, wherein the third subpopulation of cells are also positive for one, more than one, or all of the markers selected from the group consisting of: CD177, CD71, CD66b, CD115, CD49d, CD40, CD62L, CD54, CD18, CD34, CXCR4, CD64, CD32, CXCR2, CD38, Mac1, 4-1BBL, OX40L, PD-L1, and CD14.

30. A granulopoietic cell that is a CD64+ granulopoietic cell, or a population of such cells.

31. A granulopoietic cell, or a population of such cells, according to claim 30, which is a CD64+ and CD16− granulopoietic cell.

32. A granulopoietic cell, or a population of such cells, according to claim 30 or claim 31, which is a CD64+ and CD62L− granulopoietic cell.

33. A granulopoietic cell, or a population of such cells, according to any of claims 30 to 32, which is a CD64+, CD16− and CD62L− granulopoietic cell.

34. A granulopoietic cell that is a CD16− granulopoietic cell, or a population of such cells.

35. A granulopoietic cell according to claim 34, or a population of such cells, which is a CD16− and CD62L− granulopoietic cell.

36. A CD62L− granulopoietic cell, or a population of such cells.

37. An isolated population of granulopoietic cells comprising:
more than 90% Lin-cells (for example, approximately 97% Lin-cells);
less than 30% CD34+ cells (for example, approximately 14% CD34+ cells);
more than 30% CD38+ cells (for example, approximately 65% CD38+ cells);
less than 1% cells with an HSC phenotype (for example approximately 0.04% cells with an HSC phenotype);
less than 1% cells with an LT-HSC phenotype (for example approximately 0.02% cells with an LT-HSC phenotype;
less than 20% cells with an LMPP phenotype (for example approximately 5% cells with an LMPP phenotype); and
less than 10% cells with an MPP phenotype (for example approximately 2.5% cells with an MPP phenotype).

38. A pharmaceutical composition comprising a population of granulopoietic cells according to any of claims 23 to 37.

39. A pharmaceutical composition of claim 38, or a population of granulopoietic cells according to any of claims 23 to 37, for use as a medicament.

40. A pharmaceutical composition or population of granulopoietic cells for use according to claim 39, in the treatment of cancer.

41. A pharmaceutical composition or population of granulopoietic cells for use according to claim 40, for use in treating one or more of: pancreatic cancer, liver cancer, oesophageal cancer, stomach cancer, cervical cancer, ovarian cancer, lung cancer, bladder cancer, kidney cancer, brain cancer, prostate cancer, myeloma cancer, non-Hodgkin's lymphoma (NHL), larynx cancer, uterine cancer, or breast cancer.

42. A pharmaceutical composition or population of granulopoietic cells for use according to claim 41, for use in treating pancreatic cancer.

43. A pharmaceutical composition or population of granulopoietic cells for use according to claim 39, for use in the treatment of an infection.

44. A pharmaceutical composition or population of granulopoietic cells for use according to claim 39, in promoting a non-granulocytic immune response.

45. Use of a population of granulopoietic cells according to any of claims 23 to 37 in the manufacture of a medicament.

46. A method of treating a disease or disorder in a subject, the method comprising administering a pharmaceutical composition in accordance with the sixth aspect of the invention, or a population of granulopoietic cells in accordance with the second, or fourth to tenth, aspects of the invention, to the subject.

47. A method of treating cancer in a subject comprising administering a pharmaceutical composition of the invention to the subject.

48. Use of a pharmaceutical composition of the invention in the manufacture of a medicament for treating cancer in a subject.

49. A method of treating an infection in a subject comprising administering a pharmaceutical composition of the invention to the subject

50. Use of a pharmaceutical composition of the invention in the manufacture of a medicament for treating an infection in a subject.

51. A pharmaceutical composition of the invention, for use to amplify a non-granulocytic therapeutic immune response

52. A method of treatment comprising amplifying a non-granulocytic therapeutic immune response, the method comprising providing a pharmaceutical composition of the invention to a subject in need of such treatment

53. A pharmaceutical composition of the invention for use in the manufacture of a medicament for use in amplifying a non-granulocytic therapeutic immune response.

54. A composition comprising a population of granulopoietic cells as herein defined and a non-granulocytic immune cell.

55. A composition comprising a population of granulopoietic cells as herein defined and a non-granulocytic immune cell, wherein the composition does not comprise an αβ T cell

56. A composition comprising a granulopoietic cell as herein defined and a non-granulocytic immune cell, wherein the granulopoietic cell is capable of modulating (preferably modulates) the therapeutic immune response of the non-granulocytic immune cell.

57. A composition comprising a population of granulopoietic cells as herein defined and a non-granulocytic immune cell, wherein the population of granulopoietic cells is capable of amplifying (preferably amplifies) the therapeutic immune response of the non-granulocytic immune cell.

58. A kit comprising:
(a) the composition according to the invention; or
(b) a population of granulopoietic cells as herein defined and a non-granulocytic immune cell (e.g. a terminally differentiated non-granulocytic immune cell); and
(c) optionally instructions for the use of the same (e.g. in treating cancer).

59. A method for manufacturing a composition (e.g. a composition of the invention), the method comprising: culturing PBMCs in the presence of granulopoietic cells as herein defined, thereby forming the composition; and optionally depleting αβ T cells before, during, or after the culturing.

60. A method for manufacturing a composition (e.g. a composition of the invention), the method comprising: culturing αβ T cell-depleted PBMCs under conditions that promote differentiation of progenitor cells present in the αβ T cell-depleted PBMCs into granulopoietic cells by a method as herein defined, thereby forming the composition.

61. A method of treating a disease or disorder in a subject comprising administering a composition of the invention to the subject.

62. A composition of the invention, for use to modulate a non-granulocytic therapeutic immune response.

63. A composition of the invention, for use to amplify a non-granulocytic therapeutic immune response.

64. A method of treatment comprising modulating a non-granulocytic therapeutic immune response, the method comprising providing a composition of the invention to a subject in need of such treatment.

65. A method of treatment comprising amplifying a non-granulocytic therapeutic immune response, the method comprising providing a composition of the invention to a subject in need of such treatment.

66. A composition of the invention for use in the manufacture of a medicament for use in modulating a non-granulocytic therapeutic immune response.

67. A composition of the invention for use in the manufacture of a medicament for use in amplifying a non-granulocytic therapeutic immune response.

68. A method of preparing a composition of the invention, the method comprising culturing a non-granulocytic immune cell in the presence of a population of granulopoietic cells of the invention.

69. A composition comprising a population of granulopoietic cells as herein defined and a non-granulocytic immune cell (e.g. a terminally differentiated non-granulocytic immune cell), wherein the population of granulopoietic cells is capable of amplifying (preferably amplifies) the therapeutic immune response of the non-granulocytic immune cell.

70. A composition comprising one or more granulocytes differentiated from a population of granulopoietic cells as herein defined capable of amplifying (preferably that amplifies) a therapeutic immune response of a non-granulocytic cell, and a non-granulocytic cell.

71. A method of treatment comprising modulating a non-granulocytic therapeutic immune response, the method comprising providing a composition of the invention to a subject in need of such treatment.

72. A method of treatment comprising amplifying a non-granulocytic therapeutic immune response, the method comprising providing a composition of the invention to a subject in need of such treatment.

73. A method of promoting therapeutic activity of non-granulocytic immune cells, the method comprising incubating a non-granulocytic immune cell with a population of granulopoietic cells as herein defined.

74. A method of increasing survival of immune cells in culture, the method comprising, culturing the immune cells in the presence of a feeder layer of granulopoietic cells as herein defined.

75. A method of increasing proliferation of immune cells in culture, the method comprising, culturing the immune cells in the presence of a feeder layer of granulopoietic cells as herein defined.

76. A method of selecting a suitable treatment regimen for a patient, the method comprising:
identifying whether the patient has an impaired non-granulocytic immune response; and
if the patient is identified as having an impaired non-granulocytic immune response, then treatment with a population of granulopoietic cells as herein defined is selected as an appropriate treatment; and
if the patient is identified as lacking an impaired non-granulocytic immune response, then treatment with a therapy other than a population of granulopoietic cells as herein defined is selected.

77. A method of selecting a suitable treatment regimen for a patient, the method comprising:
incubating a non-granulocytic immune cell from the patient with a population of granulopoietic cells as herein defined; wherein
if the activation of the non-granulocytic immune cell from the patient is increased in response to the incubation, then treatment with a population of granulopoietic cells as herein defined is selected as an appropriate treatment; and
if the activation of the non-granulocytic immune cell from the patient is increased in response to the incubation, then treatment with a therapy other than a population of granulopoietic cells as herein defined is selected.

78. A method of identifying whether or not a population of granulopoietic cells as herein defined is suitable for use in the treatment of cancer by beneficially modulating the tumour microenvironment, the method comprising:
assessing whether the population of granulopoietic cells as herein defined, or a cell derived from the population of granulopoietic cells, is able to express proinflammatory cytokines; and/or
assessing whether the population of granulopoietic cells as herein defined, or a cell derived from the population of granulopoietic cells, is able to stimulate expression of proinflammatory cytokines by non-granulocytic immune cells;
and identifying whether or not a population of granulopoietic cells as herein defined is suitable for use in the treatment of cancer by beneficially modulating the tumour microenvironment on the basis of this assessment.

79. A method of identifying whether or not a population of granulopoietic cells as herein defined is suitable for use in the treatment of cancer by increasing recruitment of immune cells into a tumour and/or immune cell activation, the method comprising:
assessing whether the population of granulopoietic cells as herein defined, or a cell derived from the population of granulopoietic cells, is able to express a chemokine associated with promoting cell trafficking; and/or
assessing whether the population of granulopoietic cells as herein defined, or a cell derived from the population of granulopoietic cells, is able to stimulate expression of degranulation markers by non-granulocytic immune cells;
and identifying whether or not a population of granulopoietic cells as herein defined is suitable for use in the treatment of cancer by increasing recruitment of immune cells into a tumour and/or immune cell activation on the basis of this assessment.

80. A method of identifying whether or not a population of granulopoietic cells as herein defined is suitable for use in the treatment of cancer by directly promoting killing of cancer cells, the method comprising:
incubating the population of granulopoietic cells as herein defined, or a cell derived from the population of granulopoietic cells, with cells of a cancer cell line; and
assessing whether the population of granulopoietic cells as herein defined, or a cell derived from the population of granulopoietic cells, is able to increase death of the cells of the cancer cell line to a greater extent than death of non-cancer cells;
and identifying whether or not a population of granulopoietic cells as herein defined is suitable for use in the treatment of cancer by directly promoting killing of cancer cells on the basis of this assessment.

81. A method of identifying whether or not a population of granulopoietic cells as herein defined is suitable for use in the treatment of infection by directly promoting killing of cellular infectious agents or infected cells, the method comprising:
incubating the population of granulopoietic cells as herein defined, or a cell derived from the population of granulopoietic cells, with a sample of a cellular infectious agent or of infected cells; and
assessing whether the population of granulopoietic cells as herein defined, or a cell derived from the population of granulopoietic cells, is able to increase death of the cellular infectious agent or of infected cells;
and identifying whether or not a population of granulopoietic cells as herein defined is suitable for use in the treatment of infection by directly promoting killing of cellular infectious agents or infected cells on the basis of this assessment.

82. A method of identifying whether or not a population of granulopoietic cells as herein defined is suitable for use in treatment by amplifying a therapeutic immune response, the method comprising:
incubating the population of granulopoietic cells as herein defined, or a cell derived from the population of granulopoietic cells, with immune cells; and
assessing whether the population of granulopoietic cells as herein defined, or the cell derived from the population of granulopoietic cells, is able to increase activation of the immune cells;
and identifying whether or not a population of granulopoietic cells as herein defined is suitable for use in the treatment by amplifying a therapeutic immune response on the basis of this assessment.
